(19) United States
(12) Patent Application Publication
(10) Pub. No.: US 2020/0203630 A1
(43) Pub. Date: Jun. 25, 2020

(54) HETEROCYCLIC COMPOUND AND ORGANIC LIGHT-EMITTING DEVICE INCLUDING THE SAME

(71) Applicants: SAMSUNG DISPLAY CO., LTD., Yongin-si (KR); Research & Business Foundation SUNGKYUNKWAN UNIVERSITY, Suwon-si (KR)

(72) Inventors: Hyejeong PARK, Yongin-si (KR); Seungsoo YOON, Yongin-si (KR); Hyein JEONG, Yongin-si (KR)

(21) Appl. No.: 16/715,311

(22) Filed: Dec. 16, 2019

(30) Foreign Application Priority Data
Dec. 19, 2018 (KR) 10-2018-0165472

(51) Int. Cl. H01L 51/00

(52) U.S. Cl. CPC H01L 51/0072; H01L 51/0094; H01L 51/0067

(57) ABSTRACT

Provided are a heterocyclic compound and an organic light-emitting device including the same. The heterocyclic compound may be represented by Formula 1:

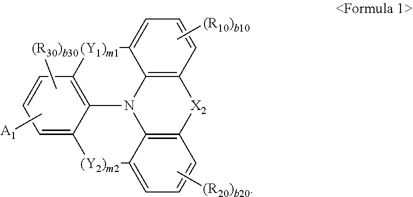

### CROSS-REFERENCE TO RELATED APPLICATION

Korean Patent Application No. 10-2018-0165472, filed on Dec. 19, 2018, in the Korean Intellectual Property Office, and entitled: “Heterocyclic Compound and Organic Light-Emitting Device Including the Same,” is incorporated by reference herein in its entirety.

### BACKGROUND

### 1. Field

Embodiments relate to a heterocyclic compound and an organic light-emitting device including the same.

### 2. Description of the Related Art

Organic light-emitting devices are self-emission devices that produce full-color images, and also have wide viewing angles, high contrast ratios, short response times, and excellent characteristics in terms of brightness, driving voltage, and response speed, compared to devices in the art.

An example of such organic light-emitting devices may include a first electrode disposed on a substrate, and a hole transport region, an emission layer, an electron transport region, and a second electrode, which are sequentially disposed on the first electrode. Holes provided from the first electrode may move toward the emission layer through the hole transport region, and electrons provided from the second electrode may move toward the emission layer through the electron transport region. Carriers, such as holes and electrons, recombine in the emission layer to produce excitons. These excitons transit from an excited state to a ground state, thereby generating light.

### SUMMARY

Embodiments are directed to an organic light-emitting device, including a first electrode, a second electrode facing the first electrode, and an organic layer between the first electrode and the second electrode and including an emission layer, the organic layer including a heterocyclic compound represented by Formula 1:

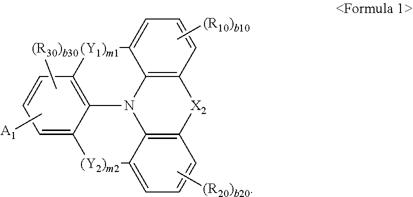

wherein, in Formula 1,

A₁ may be a group represented by Formula 2,

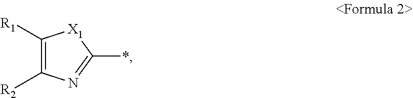

in Formulae 1 and 2,

X₁ may be O, S, or N(R₃),

X₂ may be O, S, N(R₄), C(R₄)(R₅), or Si(R₄)(R₅),

Y₁ and Y₂ may each independently be C(R₆)(R₇) or Si(R₆)(R₇),

m1 may be 0 or 1,

m2 may be 0 or 1,

R₃ may be hydrogen, deuterium, —F, —Cl, —Br, —I, a hydroxyl group, a cyano group, a nitro group, an amidino group, a hydrazino group, a hydrazono group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₃-C₁₀ cycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted monovalent non-aromatic condensed polycyclic group, —Si(Q₁)(Q₂)(Q₃), —B(Q₁)(Q₂), —C(═O)(Q₁), —N(Q₁)(Q₂), —P(═O)(Q₁)(Q₂), or —S(═O)₂(Q₁),

R₁, R₂, R₄, R₅, R₆, R₇, R₁₀, R₂₀, and R₃₀ may each independently be hydrogen, deuterium, —F, —Cl, —Br, —I, a hydroxyl group, a cyano group, a nitro group, an amidino group, a hydrazino group, a hydrazono group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, a substituted or unsubstituted monovalent non-aromatic condensed heteropolycyclic group, —Si(Q₁)(Q₂)(Q₃), —B(Q₁)(Q₂), —C(═O)(Q₁), —N(Q₁)(Q₂), —P(═O)(Q₁)(Q₂), or —S(═O)₂(Q₁),

two or more neighboring substituents of R₁ to R₇, R₁₀, R₂₀, and R₃₀ may be linked to form a substituted or unsubstituted C₅-C₆₀ carbocyclic group or a substituted or unsubstituted C₁-C₆₀ heterocyclic group,

when m1 and m2 are each 1, R₁ and R₂ may be linked to form a substituted or unsubstituted C₇-C₆₀ carbocyclic group or a substituted or unsubstituted C₁-C₆₀ heterocyclic group,

b10, b20, and b30 may each independently be an integer from 1 to 4,

at least one substituent of the substituted C₅-C₆₀ carbocyclic group, the substituted C₇-C₆₀ carbocyclic group, the substituted C₁-C₆₀ heterocyclic group, the substituted C₁-C₆₀ alkyl group, the substituted C₂-C₆₀ alkenyl group, the substituted C₂-C₆₀ alkynyl group, the substituted C₁-C₆₀ alkoxy group, the substituted C₃-C₁₀ cycloalkyl group, the substituted C₁-C₁₀ heterocycloalkyl group, the substituted C₃-C₁₀ cycloalkenyl group, the substituted C₁-C₁₀ heterocycloalkenyl group, the substituted C₆-C₆₀ aryl group, the substituted C₆-C₆₀ aryloxy group, the substituted C₆-C₆₀ arylthio group, the substituted C₁-C₆₀ heteroaryl group, the substituted C₁-C₆₀ heteroaryloxy group, the substituted C₁-C₆₀ heteroarylthio group, the substituted monovalent non-aromatic condensed polycyclic group, and the substituted monovalent non-aromatic condensed heteropolycyclic group may be:

deuterium, —F, —Cl, —Br, —I, a hydroxyl group, a cyano group, a nitro group, an amidino group, a hydrazino group, a hydrazono group, a C₁-C₆₀ alkyl group, a C₂-C₆₀ alkenyl group, a C₂-C₆₀ alkynyl group, or a C₁-C₆₀ alkoxy group;

a C₁-C₆₀ alkyl group, a C₂-C₆₀ alkenyl group, a C₂-C₆₀ alkynyl group, or a C₁-C₆₀ alkoxy group, each substituted with deuterium, —F, —Cl, —Br, —I, a hydroxyl group, a cyano group, a nitro group, an amidino group, a hydrazino group, a hydrazono group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), or —P(═O)(Q₁₁)(Q₁₂);

a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, or a monovalent non-aromatic condensed heteropolycyclic group;

a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, or a monovalent non-aromatic condensed heteropolycyclic group, each substituted with deuterium, —F, —Cl, —Br, —I, a hydroxyl group, a cyano group, a nitro group, an amidino group, a hydrazino group, a hydrazono group, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), or —P(═O)(Q₂₁)(Q₂₂); or

—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and

Q₁ to Q₃, Q₁₁ to Q₁₃, Q₂₁ to Q₂₃, and Q₃₁ to Q₃₃ may each independently be hydrogen, deuterium, —F, —Cl, —Br, —I, a hydroxyl group, a cyano group, a nitro group, an amidino group, a hydrazino group, a hydrazono group, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₁-C₆₀ heteroaryl group, a monovalent non-aromatic condensed polycyclic group, a monovalent non-aromatic condensed heteropolycyclic group, a biphenyl group, or a terphenyl group.

Embodiments are also directed to a heterocyclic compound represented by Formula 1.

### BRIEF DESCRIPTION OF THE DRAWINGS

Features will become apparent to those of skill in the art by describing in detail example embodiments with reference to the attached drawings in which:

FIG. 1 illustrates a schematic view of an organic light-emitting device according to an example embodiment;

FIG. 2 illustrates a schematic view of an organic light-emitting device according to another example embodiment;

FIG. 3 illustrates a schematic view of an organic light-emitting device according to another example embodiment; and

FIG. 4 illustrates a schematic view of an organic light-emitting device according to another example embodiment.

### DETAILED DESCRIPTION

Example embodiments will now be described more fully hereinafter with reference to the accompanying drawings; however, they may be embodied in different forms and should not be construed as limited to the embodiments set forth herein. Rather, these embodiments are provided so that this disclosure will be thorough and complete, and will fully convey example implementations to those skilled in the art. In the drawing figures, the dimensions of layers and regions may be exaggerated for clarity of illustration. Like reference numerals refer to like elements throughout.

As used herein, the singular forms “a,” “an” and “the” are intended to include the plural forms as well, unless the context clearly indicates otherwise.

It will be further understood that the terms “comprises” and/or “comprising” used herein specify the presence of stated features or components, but do not preclude the presence or addition of one or more other features or components.

It will be understood that when a layer, region, or component is referred to as being “on” or “onto” another layer, region, or component, it may be directly or indirectly formed on the other layer, region, or component. That is, for example, intervening layers, regions, or components may be present.

An example embodiment is directed to a heterocyclic compound represented by Formula 1:

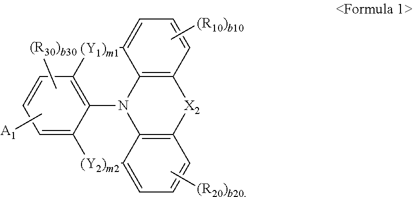

In Formula 1, A₁ may be a group represented by Formula 2:

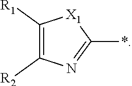

In Formula 2, X₁ may be O, S, or N(R₃).

In an example embodiment, A₁ in Formula 1 may be represented by one of Formulae 2-1 to 2-3:

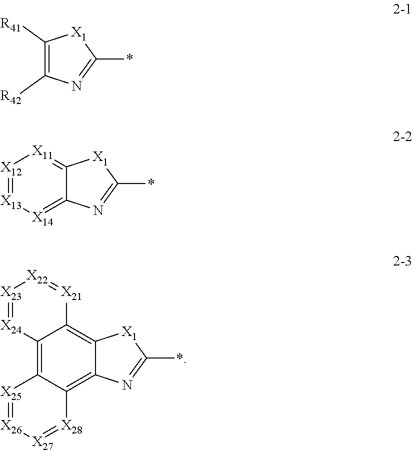

In Formulae 2-1 to 2-3,

X₁ is the same as described above,

X₁₁ may be N or C(R₅₁), X₁₂ may be N or C(R₅₂), X₁₃ may be N or C(R₅₃), and X₁₄ may be N or C(R₅₄),

X₂₁ may be N or C(R₆₁), X₂₂ may be N or C(R₆₂), X₂₃ may be N or C(R₆₃), X₂₄ may be N or C(R₆₄), X₂₅ may be N or C(R₆₅), X₂₆ may be N or C(R₆₆), X₂₇ may be N or C(R₆₇), and X₂₈ may be N or C(R₆₈),

when m1 and m2 in Formula 1 are each 1, at least one of X₁₁ to X₁₄ may be N, and

* indicates a binding site to a neighboring atom,

R₄₁, R₄₂, R₅₁ to R₅₄, and R₆₁ to R₆₈ may each independently be:

hydrogen, deuterium, —F, —Cl, —Br, —I, a methyl group, an ethyl group, an n-propyl group, an isopropyl group, an n-butyl group, a sec-butyl group, an isobutyl group, a tert-butyl group, an ethenyl group, a propenyl group, a butenyl group, a methoxy group, an ethoxy group, an n-propoxy group, an isopropoxy group, an n-butoxy group, a sec-butoxy group, an isobutoxy group, or a tert-butoxy group;

a methyl group, an ethyl group, an n-propyl group, an isopropyl group, an n-butyl group, a sec-butyl group, an isobutyl group, a tert-butyl group, a methoxy group, an ethoxy group, an n-propoxy group, an isopropoxy group, an n-butoxy group, a sec-butoxy group, an isobutoxy group, or a tert-butoxy group, each substituted with deuterium, —F, —Cl, —Br, —I, a cyano group, a phenyl group, or a biphenyl group; or

a cyclopentyl group, a cyclohexyl group, a cycloheptyl group, a cyclopentenyl group, a cyclohexenyl group, a phenyl group, a biphenyl group, a terphenyl group, a pentalenyl group, an indenyl group, a naphthyl group, an azulenyl group, an indacenyl group, an acenaphthyl group, a fluorenyl group, a spiro-bifluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a perylenyl group, a pentacenyl group, a pyrrolyl group, a thiophenyl group, a furanyl group, a silolyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an indolyl group, an isoindolyl group, an indazolyl group, a purinyl group, a quinolinyl group, an isoquinolinyl group, a benzoquinolinyl group, a phthalazinyl group, a naphthyridinyl group, a quinoxalinyl group, a benzoquinoxalinyl group, a quinazolinyl group, a benzoquinazolinyl group, a cinnolinyl group, a phenanthridinyl group, an acridinyl group, a phenanthrolinyl group, a phenazinyl group, a benzimidazolyl group, a benzofuranyl group, a benzothiophenyl group, a benzosilolyl group, a benzothiazolyl group, a benzoisothiazolyl group, a benzoxazolyl group, a benzoisoxazolyl group, a triazolyl group, a tetrazolyl group, a thiadiazolyl group, an oxadiazolyl group, a triazinyl group, a carbazolyl group, a dibenzofuranyl group, a dibenzothiophenyl group, a dibenzosilolyl group, a benzocarbazolyl group, a naphthobenzofuranyl group, a naphthobenzothiophenyl group, a naphthobenzosilolyl group, a dibenzocarbazolyl group, a dinaphthofuranyl group, a dinaphthothiophenyl group, a dinaphtho silolyl group, an imidazopyridinyl group, an imidazopyrimidinyl group, an oxazolopyridinyl group, a thiazolopyridinyl group, a benzonaphthyridinyl group, an azafluorenyl group, an azaspiro-bifluorenyl group, an azacarbazolyl group, an azadibenzofuranyl group, an azadibenzothiophenyl group, an azadibenzosilolyl group, an indenopyrrolyl group, an indolopyrrolyl group, an indenocarbazolyl group, or an indolocarbazolyl group.

In Formula 1, X₂ may be O, S, N(R₄), C(R₄)(R₅), or Si(R₄)(R₅).

In Formula 1, Y₁ and Y₂ may each independently be C(R₆)(R₇) or Si(R₆)(R₇), m1 may be 0 or 1, and m2 may be 0 or 1.

In an example embodiment, in Formula 1, m1 and m2 may each be 0, or m1 and m2 may each be 1.

When m1 in Formula 1 is 0, Y₁ does not exist. For example, when m1 is 0, Formula 1 may be represented by Formula 1A:

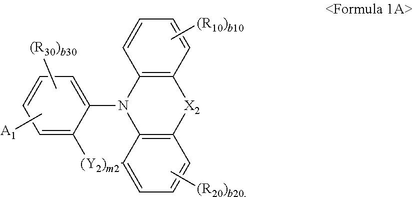

When m2 in Formula 1 is 0, Y₂ does not exist. For example, when m2 is 0, Formula 1 may be represented by Formula 1B:

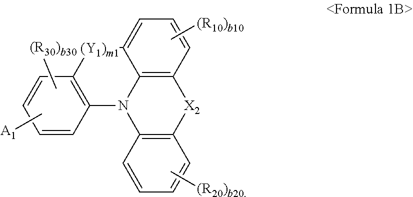

In an example embodiment, when m1 and m2 are each 0, Formula 1 may be represented by Formula 1C:

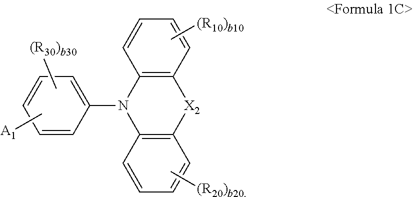

In an example embodiment, when m1 and m2 are each 1, Formula 1 may be represented by Formula 1D:

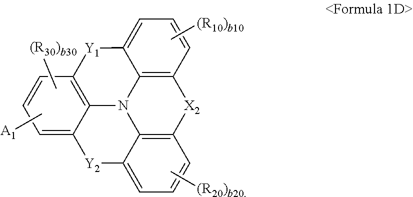

In Formulae 1A to 1D,

A₁, X₂, Y₁, Y₂, m1, m2, R₁₀, R₂₀, R₃₀, b10, b20, and b30 are the same as described above.

In Formula 2, R₃ may be hydrogen, deuterium, —F, —Cl, —Br, —I, a hydroxyl group, a cyano group, a nitro group, an amidino group, a hydrazino group, a hydrazono group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₃-C₁₀ cycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted monovalent non-aromatic condensed polycyclic group, —Si(Q₁)(Q₂)(Q₃), —B(Q₁)(Q₂), —C(═O)(Q₁), —N(Q₁)(Q₂), —P(═O)(Q₁)(Q₂), or —S(═O)₂(Q₁).

In an example embodiment, R₃ may be:

hydrogen, deuterium, —F, —Cl, —Br, —I, a methyl group, an ethyl group, an n-propyl group, an isopropyl group, an n-butyl group, a sec-butyl group, an isobutyl group, a tert-butyl group, an ethenyl group, a propenyl group, a butenyl group, a methoxy group, an ethoxy group, an n-propoxy group, an isopropoxy group, an n-butoxy group, a sec-butoxy group, an isobutoxy group, or a tert-butoxy group;

a methyl group, an ethyl group, an n-propyl group, an isopropyl group, an n-butyl group, a sec-butyl group, an isobutyl group, a tert-butyl group, a methoxy group, an ethoxy group, an n-propoxy group, an isopropoxy group, an n-butoxy group, a sec-butoxy group, an isobutoxy group, or a tert-butoxy group, each substituted with deuterium, —F, —Cl, —Br, —I, a cyano group, a phenyl group, or a biphenyl group;

a cyclopentyl group, a cyclohexyl group, a cycloheptyl group, a cyclopentenyl group, a cyclohexenyl group, a phenyl group, a biphenyl group, a terphenyl group, a pentalenyl group, an indenyl group, a naphthyl group, an azulenyl group, an indacenyl group, an acenaphthyl group, a fluorenyl group, a spiro-bifluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a perylenyl group, or a pentacenyl group; or

a cyclopentyl group, a cyclohexyl group, a cycloheptyl group, a cyclopentenyl group, a cyclohexenyl group, a phenyl group, a biphenyl group, a terphenyl group, a pentalenyl group, an indenyl group, a naphthyl group, an azulenyl group, an indacenyl group, an acenaphthyl group, a fluorenyl group, a spiro-bifluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a perylenyl group, or a pentacenyl group, each substituted with deuterium, —F, —Cl, —Br, —I, a cyano group, a methyl group, an ethyl group, an n-propyl group, an isopropyl group, an n-butyl group, a sec-butyl group, an isobutyl group, a tert-butyl group, a methoxy group, an ethoxy group, an n-propoxy group, an isopropoxy group, an n-butoxy group, a sec-butoxy group, an isobutoxy group, a tert-butoxy group, a cyclopentyl group, a cyclohexyl group, a cycloheptyl group, a cyclopentenyl group, a cyclohexenyl group, a phenyl group, a biphenyl group, a terphenyl group, a pentalenyl group, an indenyl group, a naphthyl group, an azulenyl group, an indacenyl group, an acenaphthyl group, a fluorenyl group, a spiro-bifluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a perylenyl group, a pentacenyl group, a pyrrolyl group, a thiophenyl group, a furanyl group, a silolyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an indolyl group, an isoindolyl group, an indazolyl group, a purinyl group, a quinolinyl group, an isoquinolinyl group, a benzoquinolinyl group, a phthalazinyl group, a naphthyridinyl group, a quinoxalinyl group, a benzoquinoxalinyl group, a quinazolinyl group, a benzoquinazolinyl group, a cinnolinyl group, a phenanthridinyl group, an acridinyl group, a phenanthrolinyl group, a phenazinyl group, a benzimidazolyl group, a benzofuranyl group, a benzothiophenyl group, a benzosilolyl group, a benzothiazolyl group, a benzoisothiazolyl group, a benzoxazolyl group, a benzoisoxazolyl group, a triazolyl group, a tetrazolyl group, a thiadiazolyl group, an oxadiazolyl group, a triazinyl group, a carbazolyl group, a dibenzofuranyl group, a dibenzothiophenyl group, a dibenzosilolyl group, a benzocarbazolyl group, a naphthobenzofuranyl group, a naphthobenzothiophenyl group, a naphthobenzosilolyl group, a dibenzocarbazolyl group, a dinaphthofuranyl group, a dinaphthothiophenyl group, a dinaphtho silolyl group, an imidazopyridinyl group, an imidazopyrimidinyl group, an oxazolopyridinyl group, a thiazolopyridinyl group, a benzonaphthyridinyl group, an azafluorenyl group, an azaspiro-bifluorenyl group, an azacarbazolyl group, an azadibenzofuranyl group, an azadibenzothiophenyl group, an azadibenzosilolyl group, an indenopyrrolyl group, an indolopyrrolyl group, an indenocarbazolyl group, an indolocarbazolyl group, —Si(Q₃₁)(Q₃₂)(Q₃₃), —N(Q₃₁)(Q₃₂), —B(Q₃₁)(Q₃₂), —C(═O)(Q₃₁), —S(═O)(Q₃₁), —S(═O)₂(Q₃₁), —P(═O)(Q₃₁)(Q₃₂), or —P(═S)(Q₃₁)(Q₃₂).

In an example embodiment, R₃ may be:

hydrogen, deuterium, —F, —Cl, —Br, —I, a cyano group, a C₁-C₂₀ alkyl group, or a C₁-C₂₀ alkoxy group;

a C₁-C₂₀ alkyl group or a C₁-C₂₀ alkoxy group, each substituted with deuterium, —F, —Cl, —Br, —I, a cyano group, a phenyl group, or a biphenyl group; or

a group represented by one of Formulae 7-1 to 7-12:

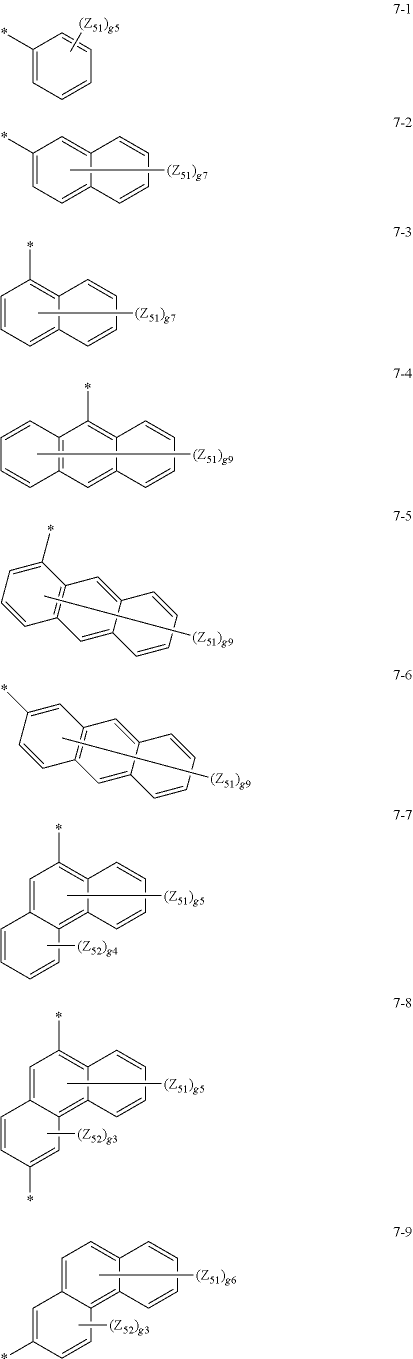

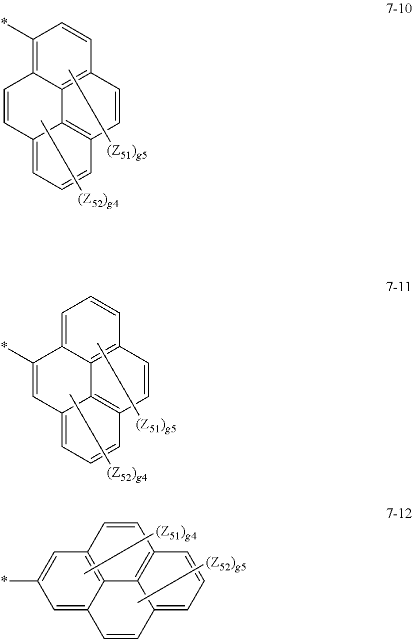

In Formulae 7-1 to 7-12,

Z₅₁ and Z₅₂ may each independently be hydrogen, deuterium, —F, —Cl, —Br, —I, a hydroxyl group, a cyano group, a nitro group, an amidino group, a hydrazino group, a hydrazono group, a C₁-C₂₀ alkyl group, a C₁-C₂₀ alkenyl group, a C₁-C₂₀ alkynyl group, a C₁-C₂₀ alkoxy group, a phenyl group, a biphenyl group, a terphenyl group, a naphthyl group, a fluorenyl group, a spiro-bifluorenyl group, a phenanthrenyl group, an anthracenyl group, a triphenylenyl group, a pyridinyl group, a pyrimidinyl group, a carbazolyl group, or a triazinyl group,

g3 may be an integer from 1 to 3,

g4 may be an integer from 1 to 4,

g5 may be an integer from 1 to 5,

g6 may be an integer from 1 to 6,

g7 may be an integer from 1 to 7,

g9 may be an integer from 1 to 9, and

* indicates a binding site to a neighboring atom.

In Formulae 1 and 2, R₁, R₂, R₄, R₅, R₆, R₇, R₁₀, R₂₀, and R₃₀ may each independently be hydrogen, deuterium, —F, —Cl, —Br, —I, a hydroxyl group, a cyano group, a nitro group, an amidino group, a hydrazino group, a hydrazono group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, a substituted or unsubstituted monovalent non-aromatic condensed heteropolycyclic group, —Si(Q₁)(Q₂)(Q₃), —B(Q₁)(Q₂), —C(═O)(Q₁), —N(Q₁)(Q₂), —P(═O)(Q₁)(Q₂), or —S(═O)₂(Q₁), or

two or more neighboring substituents among R₁ to R₇, R₁₀, R₂₀, and R₃₀ may be linked to form a substituted or unsubstituted C₅-C₆₀ carbocyclic group or a substituted or unsubstituted C₁-C₆₀ heterocyclic group.

In Formula 1, b10, b20, and b30 may each independently be an integer from 1 to 4.

In an example embodiment, R₁, R₂, R₄, R₅, R₆, R₇, R₁₀, R₂₀, and R₃₀ may each independently be:

hydrogen, deuterium, —F, —Cl, —Br, —I, a methyl group, an ethyl group, an n-propyl group, an isopropyl group, an n-butyl group, a sec-butyl group, an isobutyl group, a tert-butyl group, an ethenyl group, a propenyl group, a butenyl group, a methoxy group, an ethoxy group, an n-propoxy group, an isopropoxy group, an n-butoxy group, a sec-butoxy group, an isobutoxy group, or a tert-butoxy group; or

a group represented by one of Formulae 5-1 to 5-26 and Formulae 6-1 to 6-55, or

two or more neighboring substituents among R₁, R₂, R₄, R₅, R₆, R₇, R₁₀, R₂₀, and R₃₀ may be linked to form a substituted or unsubstituted C₅-C₆₀ carbocyclic group or a substituted or unsubstituted C₁-C₆₀ heterocyclic group:

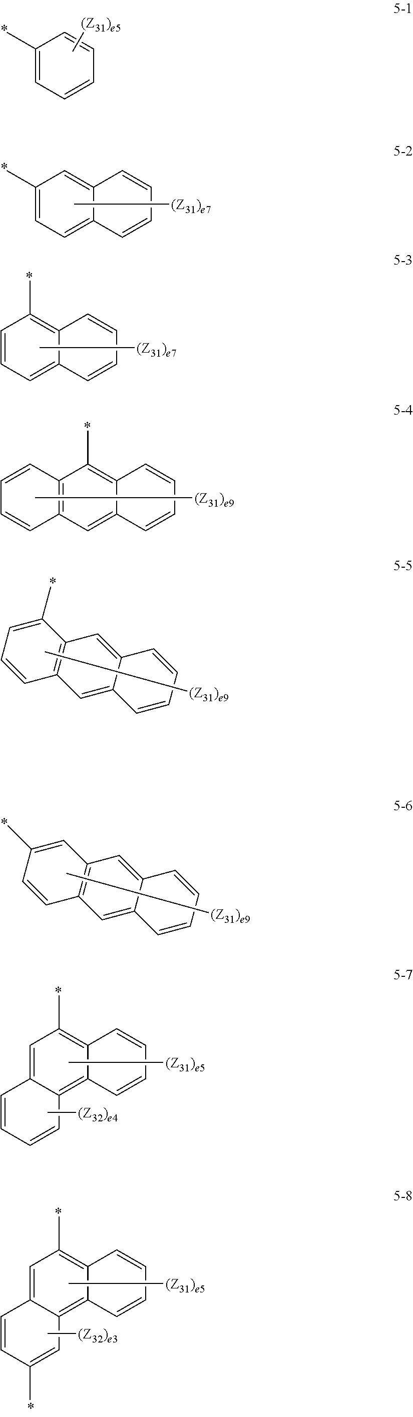

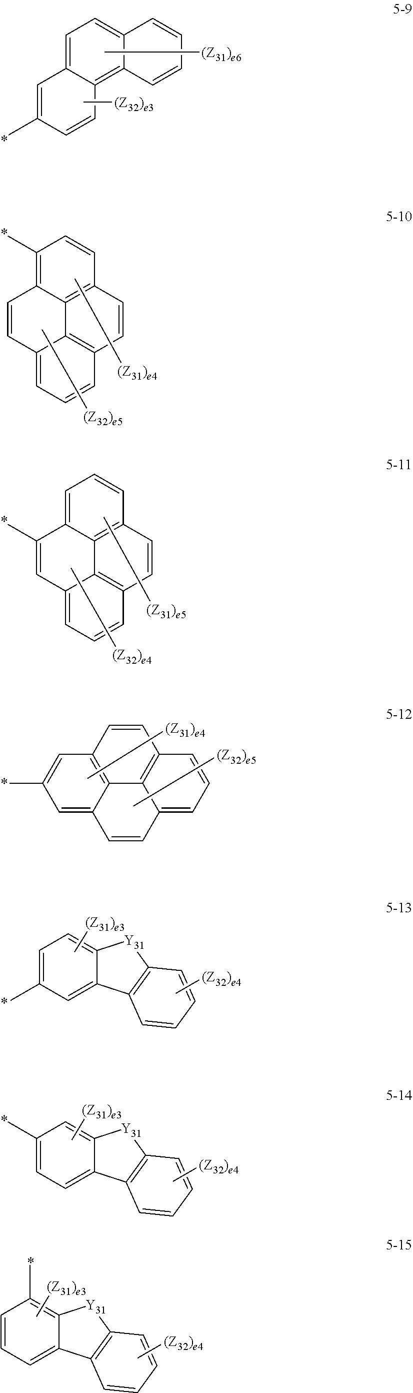

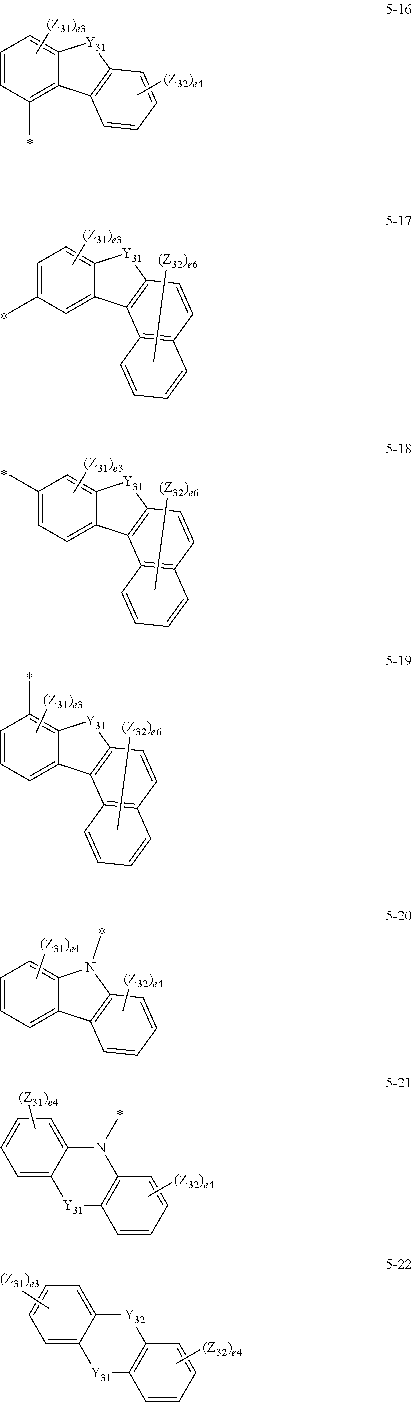

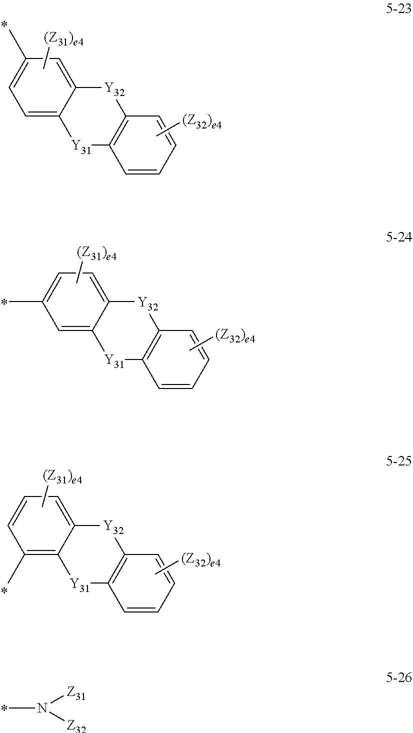

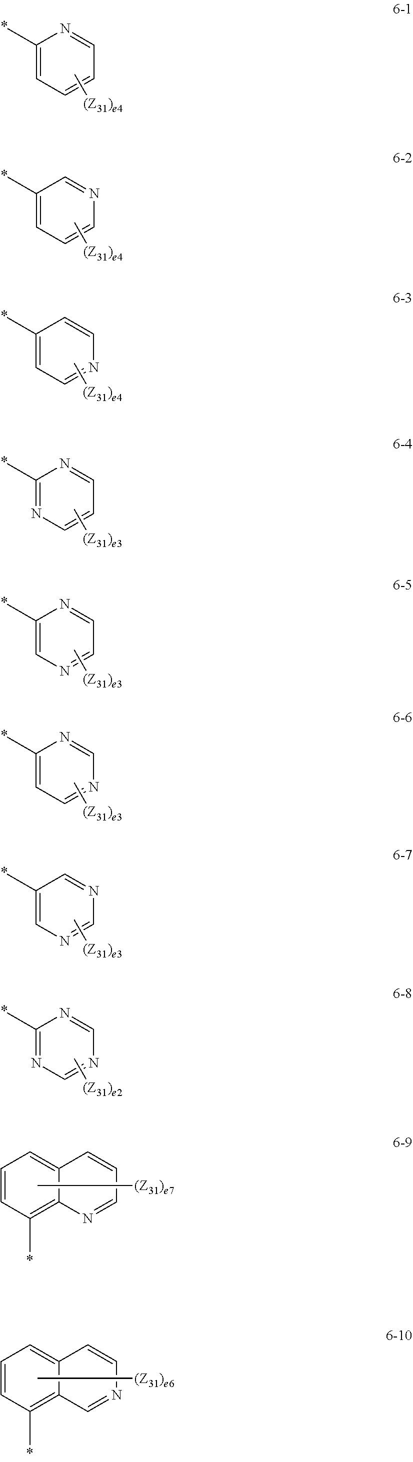

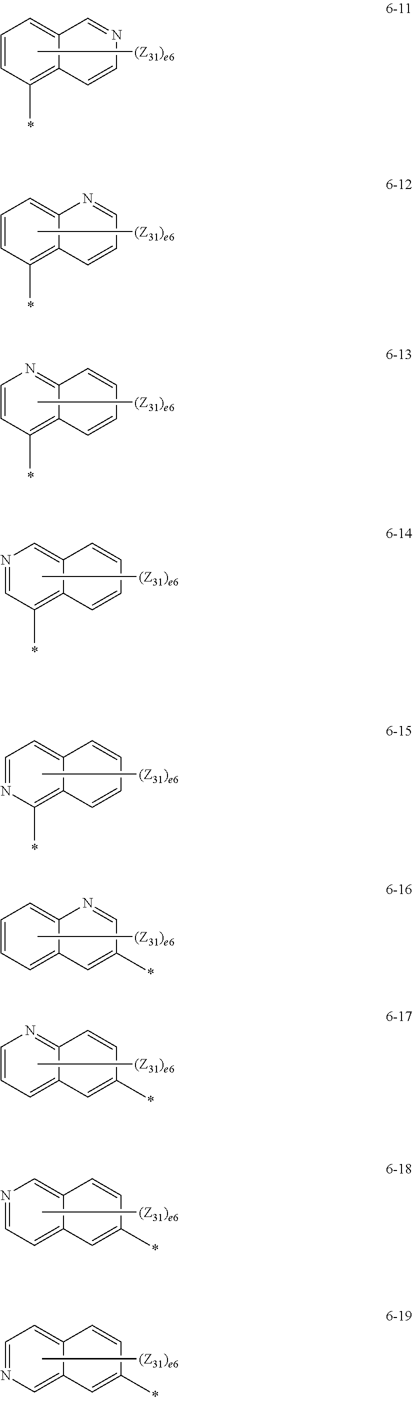

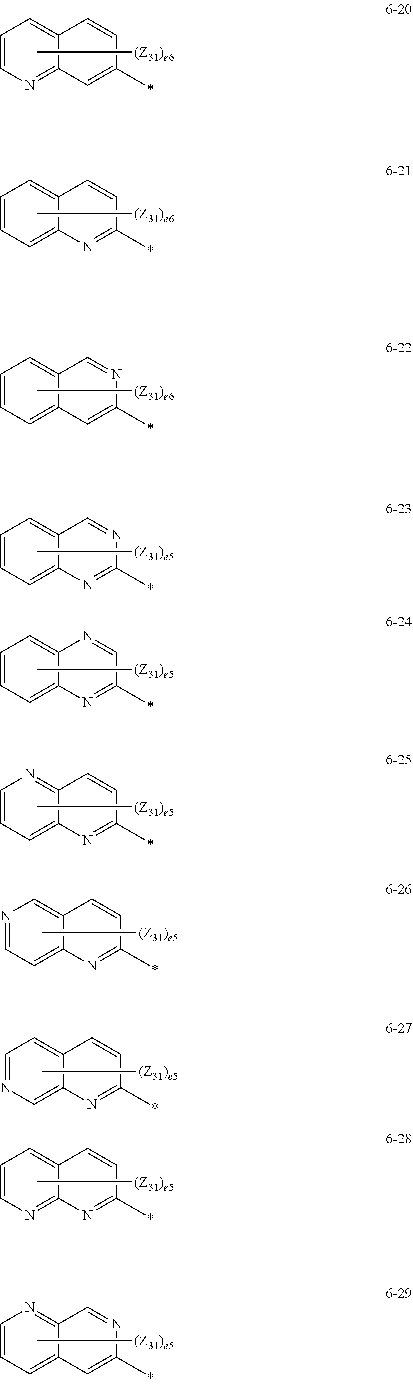

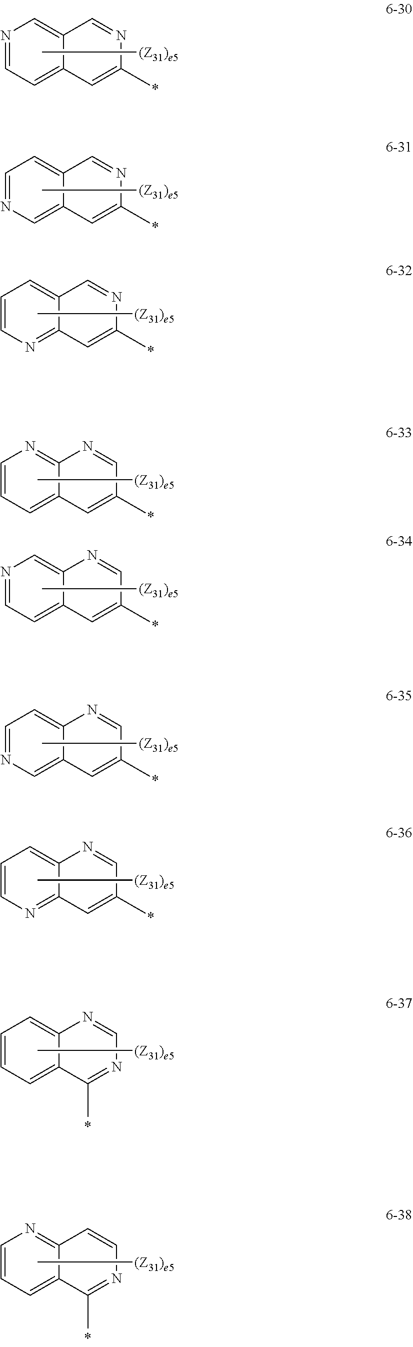

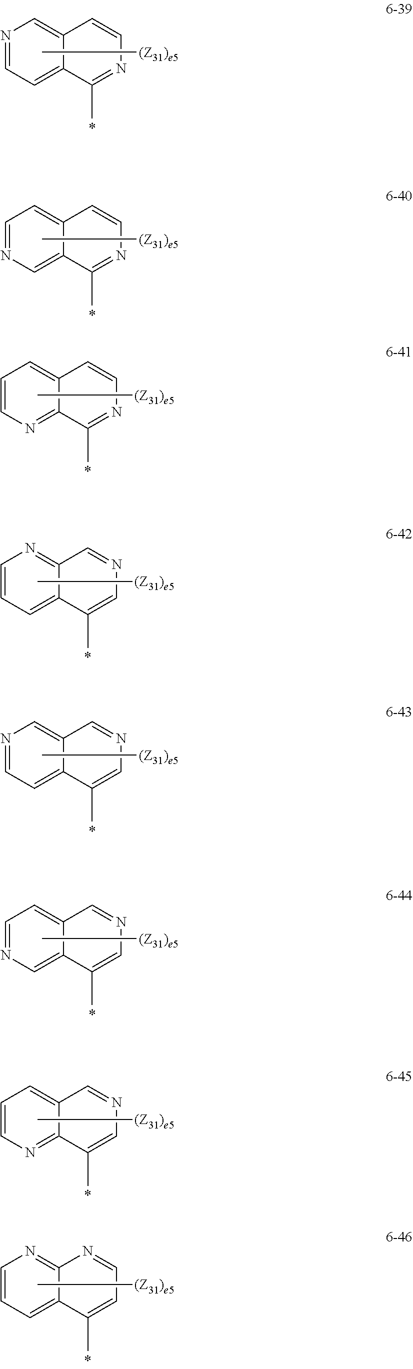

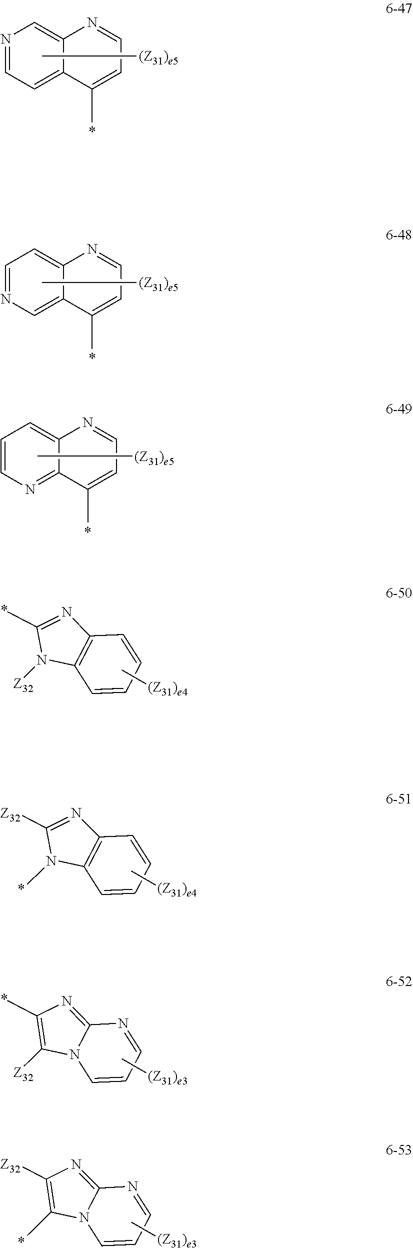

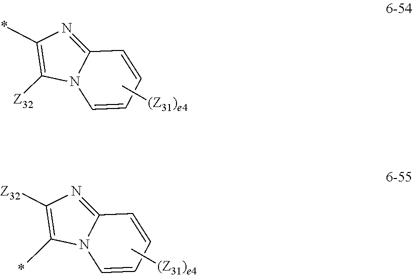

In Formulae 5-1 to 5-26 and Formulae 6-1 to 6-55,

Y₃₁ may be 0, S, C(Z₃₄)(Z₃₅), N(Z₃₄), or Si(Z₃₄)(Z₃₅),

Z₃₁ to Z₃₅ may each independently be hydrogen, deuterium, —F, —Cl, —Br, —I, a hydroxyl group, a cyano group, a nitro group, an amidino group, a hydrazino group, a hydrazono group, a C₁-C₂₀ alkyl group, a C₁-C₂₀ alkenyl group, a C₁-C₂₀ alkynyl group, a C₁-C₂₀ alkoxy group, a phenyl group, a biphenyl group, a terphenyl group, a naphthyl group, a fluorenyl group, a spiro-bifluorenyl group, a phenanthrenyl group, an anthracenyl group, a triphenylenyl group, a pyridinyl group, a pyrimidinyl group, a carbazolyl group, or a triazinyl group,

e2 may be 1 or 2,

e3 may be an integer from 1 to 3,

e4 may be an integer from 1 to 4,

e5 may be an integer from 1 to 5,

e6 may be an integer from 1 to 6,

e7 may be an integer from 1 to 7,

e9 may be an integer from 1 to 9, and

* indicates a binding site to a neighboring atom.

In an example embodiment, R₁ and R₂ may independently be a group represented by one of Formulae 5-1 to 5-26 and Formulae 6-1 to 6-55, or

R₁ and R₂ may be linked to form a ring group, the ring group being:

a benzene group, a naphthalene group, a phenanthrene group, an anthracene group, a triphenylene group, a pyrene group, a chrysene group, a pyridine group, a pyrazine group, a pyrimidine group, a triazine group, a quinoline group, an isoquinoline group, a benzoquinoline group, a benzoisoquinoline group, a 2,6-naphthyridine group, a 1,8-naphthyridine group, a 1,5-naphthyridine group, a 1,6-naphthyridine group, a 1,7-naphthyridine group, a 2,7-naphthyridine group, a quinoxaline group, a quinazoline group, a phenanthridine group, or a phenanthroline group; or

a benzene group, a naphthalene group, a phenanthrene group, an anthracene group, a triphenylene group, a pyrene group, a chrysene group, a pyridine group, a pyrazine group, a pyrimidine group, a triazine group, a quinoline group, an isoquinoline group, a benzoquinoline group, a benzoisoquinoline group, a 2,6-naphthyridine group, a 1,8-naphthyridine group, a 1,5-naphthyridine group, a 1,6-naphthyridine group, a 1,7-naphthyridine group, a 2,7-naphthyridine group, a quinoxaline group, a quinazoline group, a phenanthridine group, or a phenanthroline group, each substituted with deuterium, —F, —Cl, —Br, —I, a cyano group, a methyl group, an ethyl group, an n-propyl group, an isopropyl group, an n-butyl group, a sec-butyl group, an isobutyl group, a tert-butyl group, a methoxy group, an ethoxy group, an n-propoxy group, an isopropoxy group, an n-butoxy group, a sec-butoxy group, an isobutoxy group, or a tert-butoxy group.

In an example embodiment, R₄ and R₅ may each independently be:

hydrogen, deuterium, —F, —Cl, —Br, —I, a methyl group, an ethyl group, an n-propyl group, an isopropyl group, an n-butyl group, a sec-butyl group, an isobutyl group, a tert-butyl group, an ethenyl group, a propenyl group, a butenyl group, a methoxy group, an ethoxy group, an n-propoxy group, an isopropoxy group, an n-butoxy group, a sec-butoxy group, an isobutoxy group, or a tert-butoxy group;

a methyl group, an ethyl group, an n-propyl group, an isopropyl group, an n-butyl group, a sec-butyl group, an isobutyl group, a tert-butyl group, an ethenyl group, a propenyl group, a butenyl group, a methoxy group, an ethoxy group, an n-propoxy group, an isopropoxy group, an n-butoxy group, a sec-butoxy group, an isobutoxy group, or a tert-butoxy group, each substituted with deuterium, —F, —Cl, —Br, —I, a cyano group, a phenyl group, or a biphenyl group;

a cyclopentyl group, a cyclohexyl group, a cycloheptyl group, a cyclopentenyl group, a cyclohexenyl group, a phenyl group, a biphenyl group, a terphenyl group, a pentalenyl group, an indenyl group, a naphthyl group, an azulenyl group, an indacenyl group, an acenaphthyl group, a fluorenyl group, a spiro-bifluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a perylenyl group, a pentacenyl group, a carbazolyl group, a dibenzofuranyl group, a dibenzothiophenyl group, or a dibenzosilolyl group; or

a cyclopentyl group, a cyclohexyl group, a cycloheptyl group, a cyclopentenyl group, a cyclohexenyl group, a phenyl group, a biphenyl group, a terphenyl group, a pentalenyl group, an indenyl group, a naphthyl group, an azulenyl group, an indacenyl group, an acenaphthyl group, a fluorenyl group, a spiro-bifluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a perylenyl group, a pentacenyl group, a carbazolyl group, a dibenzofuranyl group, a dibenzothiophenyl group, or a dibenzosilolyl group, each substituted with deuterium, —F, —Cl, —Br, —I, a cyano group, a methyl group, an ethyl group, an n-propyl group, an isopropyl group, an n-butyl group, a sec-butyl group, an isobutyl group, a tert-butyl group, a methoxy group, an ethoxy group, an n-propoxy group, an isopropoxy group, an n-butoxy group, a sec-butoxy group, an isobutoxy group, or a tert-butoxy group, and

R₄ and R₅ may be linked to form a group represented by Formula 9:

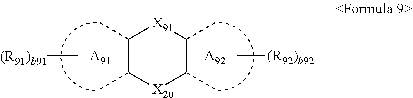

In Formula 9,

X₂₀ may be C or Si in X₂ of Formula 1,

X₉₁ may be a single bond, O, S, Se, N(R₉₃), B(R₉₃) C(R₉₃)(R₉₄), or Si(R₉₃)(R₉₄),

A₉₁ and A₉₂ may each independently be a benzene group, a naphthalene group, a phenanthrene group, an anthracene group, a pyridine group, a pyrazine group, a pyrimidine group, a quinoline group, an isoquinoline group, a benzoquinoline group, a benzoisoquinoline group, a dibenzofuran group, a dibenzothiophene group, a fluorene group, or a carbazole group,

R₉₁ to R₉₄ may each independently be hydrogen, deuterium, —F, —Cl, —Br, —I, a hydroxyl group, a cyano group, a nitro group, an amidino group, a hydrazino group, a hydrazono group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted C₁-C₆₀ heteroaryloxy group, a substituted or unsubstituted C₁-C₆₀ heteroarylthio group, a substituted or unsubstituted monovalent non-aromatic condensed polycyclic group, a substituted or unsubstituted monovalent non-aromatic condensed heteropolycyclic group, —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),

Q₁ to Q₃ may each independently be hydrogen, deuterium, —F, —Cl, —Br, —I, a hydroxyl group, a cyano group, a nitro group, an amidino group, a hydrazino group, a hydrazono group, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₁-C₆₀ heteroaryl group, a monovalent non-aromatic condensed polycyclic group, a monovalent non-aromatic condensed heteropolycyclic group, a biphenyl group, or a terphenyl group, and

b91 and b92 may each independently be an integer from 1 to 10.

In an example embodiment, A₉₁ and A₉₂ in Formula 9 may each independently be a benzene group, a naphthalene group, a phenanthrene group, an anthracene group, a dibenzofuran group, a dibenzothiophene group, a fluorene group, or a carbazole group.

In an example embodiment, R₉₁ to R₉₄ in Formula 9 may each independently be hydrogen, deuterium, —F, —Cl, —Br, —I, a methyl group, an ethyl group, an n-propyl group, an isopropyl group, an n-butyl group, a sec-butyl group, an isobutyl group, a tert-butyl group, an ethenyl group, a propenyl group, a butenyl group, a methoxy group, an ethoxy group, an n-propoxy group, an isopropoxy group, an n-butoxy group, a sec-butoxy group, an isobutoxy group, a tert-butoxy group, a phenyl group, a biphenyl group, a terphenyl group, a pentalenyl group, an indenyl group, a naphthyl group, an azulenyl group, an indacenyl group, an acenaphthyl group, a fluorenyl group, a spiro-bifluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a perylenyl group, a pentacenyl group, a carbazolyl group, a dibenzofuranyl group, a dibenzothiophenyl group, or a dibenzosilolyl group.

In an example embodiment, R₆ and R₇ may each independently be hydrogen, deuterium, —F, —Cl, —Br, —I, a cyano group, a methyl group, an ethyl group, an n-propyl group, an isopropyl group, an n-butyl group, a sec-butyl group, an isobutyl group, a tert-butyl group, an ethenyl group, a propenyl group, a butenyl group, a methoxy group, an ethoxy group, an n-propoxy group, an isopropoxy group, an n-butoxy group, a sec-butoxy group, an isobutoxy group, or a tert-butoxy group; or

a methyl group, an ethyl group, an n-propyl group, an isopropyl group, an n-butyl group, a sec-butyl group, an isobutyl group, a tert-butyl group, an ethenyl group, a propenyl group, a butenyl group, a methoxy group, an ethoxy group, an n-propoxy group, an isopropoxy group, an n-butoxy group, a sec-butoxy group, an isobutoxy group, or a tert-butoxy group, each substituted with deuterium, —F, —Cl, —Br, —I, a cyano group, a phenyl group, or a biphenyl group.

In an example embodiment, R₁₀, R₂₀, and R₃₀ may each independently be:

hydrogen, deuterium, —F, —Cl, —Br, —I, a methyl group, an ethyl group, an n-propyl group, an isopropyl group, an n-butyl group, a sec-butyl group, an isobutyl group, or a tert-butyl group;

a methyl group, an ethyl group, an n-propyl group, an isopropyl group, an n-butyl group, a sec-butyl group, an isobutyl group, or a tert-butyl group, each substituted with deuterium, —F, —Cl, —Br, —I, a cyano group, a phenyl group, or a biphenyl group;

a phenyl group, a biphenyl group, a terphenyl group, a naphthyl group, a fluorenyl group, a spiro-bifluorenyl group, a benzofluorenyl group, a dibenzofluorenyl group, a carbazolyl group, a dibenzofuranyl group, a dibenzothiophenyl group, or a dibenzosilolyl group;

a phenyl group, a biphenyl group, a terphenyl group, a naphthyl group, a fluorenyl group, a spiro-bifluorenyl group, a benzofluorenyl group, a dibenzofluorenyl group, a carbazolyl group, a dibenzofuranyl group, a dibenzothiophenyl group, or a dibenzosilolyl group, each substituted with deuterium, —F, —Cl, —Br, —I, a cyano group, a methyl group, an ethyl group, an n-propyl group, an isopropyl group, an n-butyl group, a sec-butyl group, an isobutyl group, a tert-butyl group, a phenyl group, a biphenyl group, a terphenyl group, a naphthyl group, a fluorenyl group, a spiro-bifluorenyl group, a benzofluorenyl group, a dibenzofluorenyl group, a carbazolyl group, a dibenzofuranyl group, a dibenzothiophenyl group, or a dibenzosilolyl group; or

—N(Q₁₁)(Q₁₂), and

Q₁₁ and Q₁₂ are the same as described above.

In an example embodiment, the heterocyclic compound represented by Formula 1 may be represented by Formula 10A or 10B:

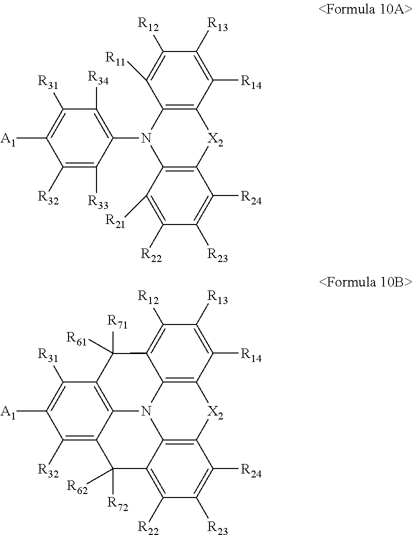

In Formulae 10A and 10B,

A₁ and X₂ are the same as described above,

R₁₁ to R₁₄ may each independently be defined the same as R₁₀,

R₂₁ to R₂₄ may each independently be defined the same as R₂₀,

R₃₁ to R₃₄ may each independently be defined the same as R₃₀,

R₆₁ and R₆₂ may each independently be defined the same as R₆, and

R₇₁ and R₇₂ may each independently be defined the same as R₇.

In an example embodiment, the heterocyclic compound may be one of Compounds 1 to 94:

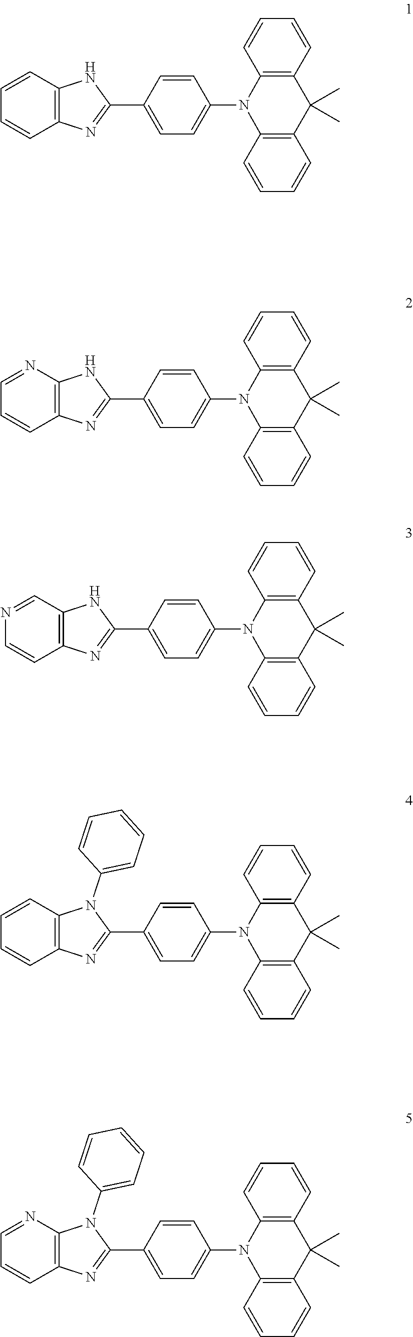 [SMILES: CC1(C)c2ccccc2N(c2ccc(-c3nc4ccccc4[nH]3)cc2)c2ccccc21 ; CC1(C)c2ccccc2N(c2ccc(-c3nc4cccnc4[nH]3)cc2)c2ccccc21 ; CC1(C)c2ccccc2N(c2ccc(-c3nc4ccncc4[nH]3)cc2)c2ccccc21 ; CC1(C)c2ccccc2N(c2ccc(-c3nc4ccccc4n3-c3ccccc3)cc2)c2ccccc21 ; CC1(C)c2ccccc2N(c2ccc(-c3nc4cccnc4n3-c3ccccc3)cc2)c2ccccc21]

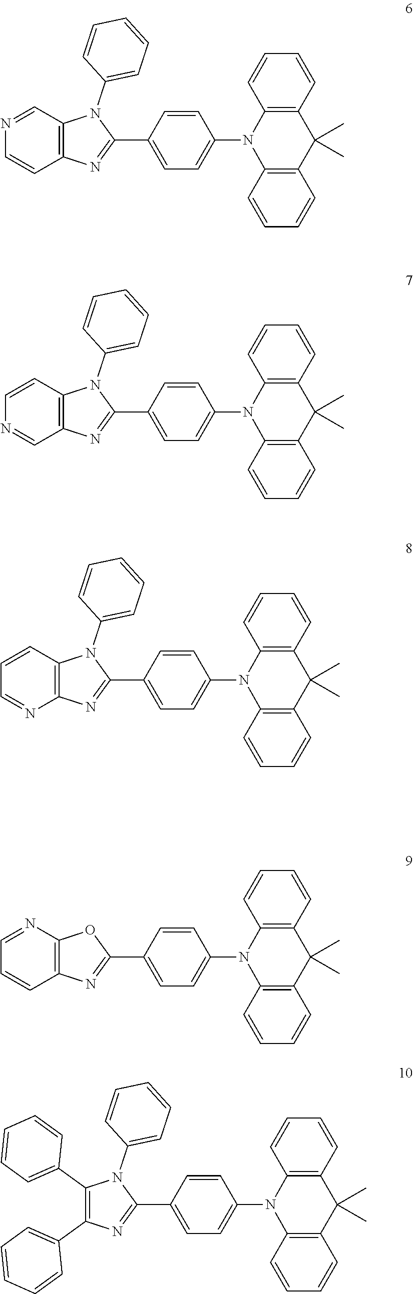 [SMILES: CC1(C)c2ccccc2N(c2ccc(-c3nc4ccncc4n3-c3ccccc3)cc2)c2ccccc21 ; CC1(C)c2ccccc2N(c2ccc(-c3nc4cnccc4n3-c3ccccc3)cc2)c2ccccc21 ; CC1(C)c2ccccc2N(c2ccc(-c3nc4ncccc4n3-c3ccccc3)cc2)c2ccccc21 ; CC1(C)c2ccccc2N(c2ccc(-c3nc4cccnc4o3)cc2)c2ccccc21 ; CC1(C)c2ccccc2N(c2ccc(-c3nc(-c4ccccc4)c(-c4ccccc4)n3-c3ccccc3)cc2)c2ccccc21]

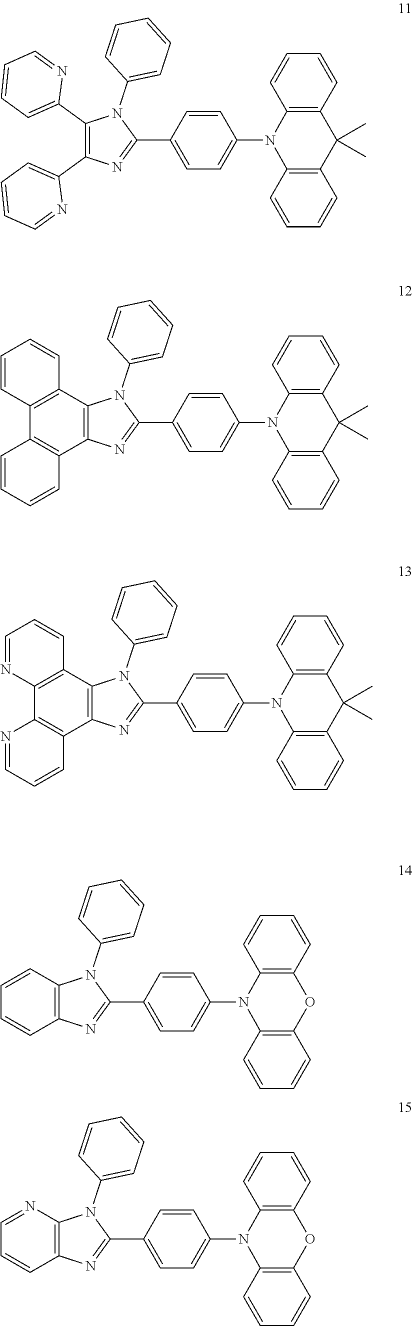 [SMILES: CC1(C)c2ccccc2N(c2ccc(-c3nc(-c4ccccn4)c(-c4ccccn4)n3-c3ccccc3)cc2)c2ccccc21 ; CC1(C)c2ccccc2N(c2ccc(-c3nc4c5ccccc5c5ccccc5c4n3-c3ccccc3)cc2)c2ccccc21 ; CC1(C)c2ccccc2N(c2ccc(-c3nc4c5cccnc5c5ncccc5c4n3-c3ccccc3)cc2)c2ccccc21 ; c1ccc(-n2c(-c3ccc(N4c5ccccc5Oc5ccccc54)cc3)nc3ccccc32)cc1 ; c1ccc(-n2c(-c3ccc(N4c5ccccc5Oc5ccccc54)cc3)nc3cccnc32)cc1]

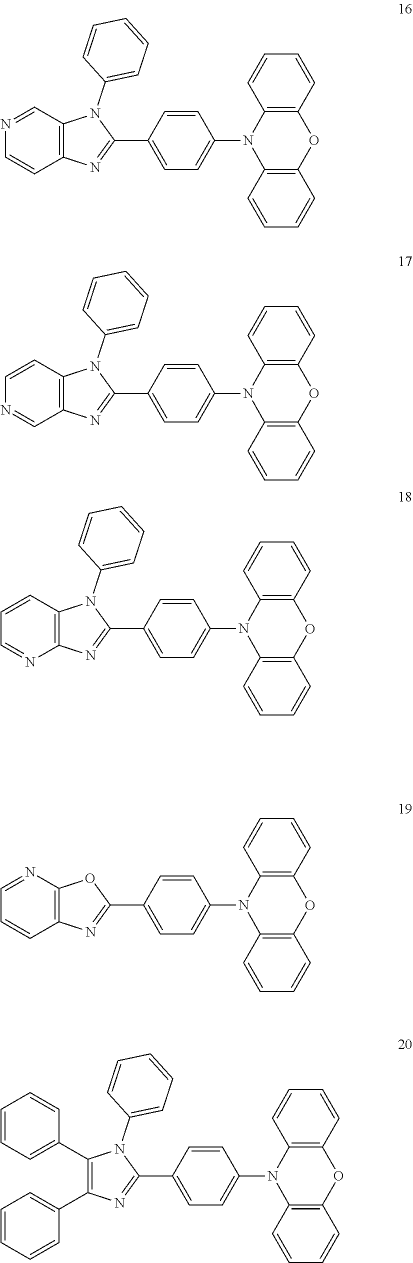 [SMILES: c1ccc(-n2c(-c3ccc(N4c5ccccc5Oc5ccccc54)cc3)nc3ccncc32)cc1 ; c1ccc(-n2c(-c3ccc(N4c5ccccc5Oc5ccccc54)cc3)nc3cnccc32)cc1 ; c1ccc(-n2c(-c3ccc(N4c5ccccc5Oc5ccccc54)cc3)nc3ncccc32)cc1 ; c1ccc2c(c1)Oc1ccccc1N2c1ccc(-c2nc3cccnc3o2)cc1 ; c1ccc(-c2nc(-c3ccc(N4c5ccccc5Oc5ccccc54)cc3)n(-c3ccccc3)c2-c2ccccc2)cc1]

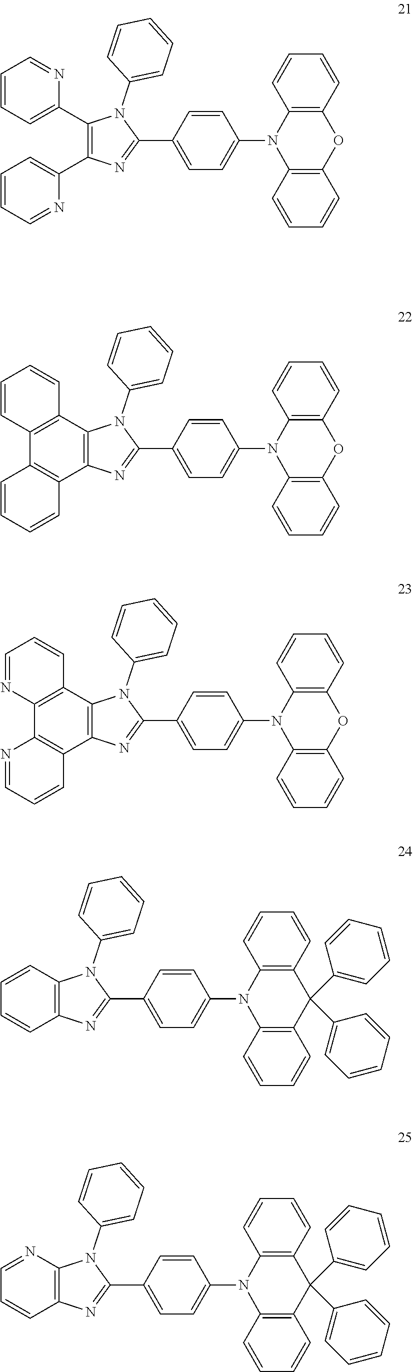 [SMILES: c1ccc(-n2c(-c3ccc(N4c5ccccc5Oc5ccccc54)cc3)nc(-c3ccccn3)c2-c2ccccn2)cc1 ; c1ccc(-n2c(-c3ccc(N4c5ccccc5Oc5ccccc54)cc3)nc3c4ccccc4c4ccccc4c32)cc1 ; c1ccc(-n2c(-c3ccc(N4c5ccccc5Oc5ccccc54)cc3)nc3c4cccnc4c4ncccc4c32)cc1 ; c1ccc(-n2c(-c3ccc(N4c5ccccc5C(c5ccccc5)(c5ccccc5)c5ccccc54)cc3)nc3ccccc32)cc1 ; c1ccc(-n2c(-c3ccc(N4c5ccccc5C(c5ccccc5)(c5ccccc5)c5ccccc54)cc3)nc3cccnc32)cc1]

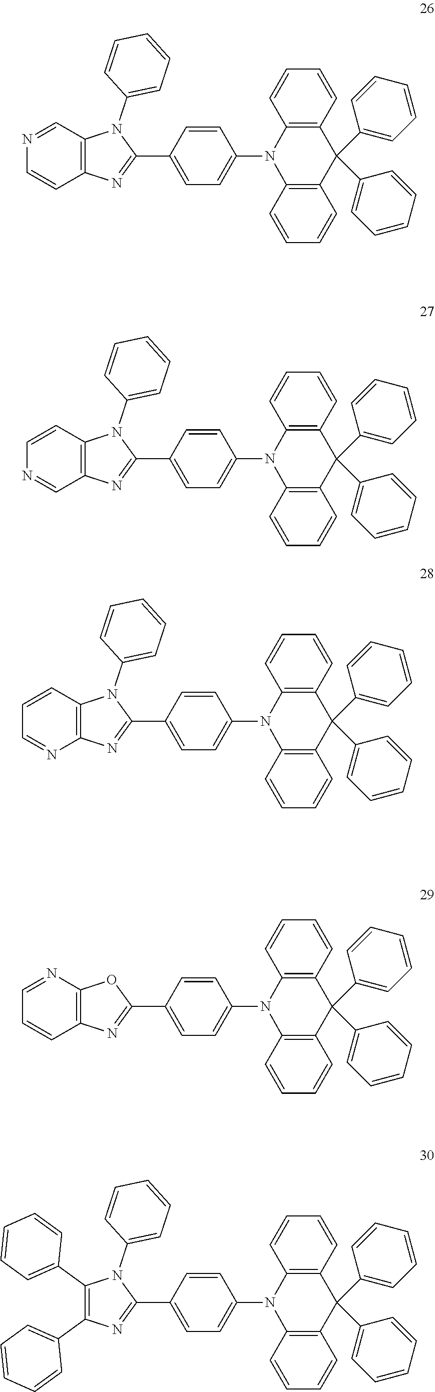 [SMILES: c1ccc(-n2c(-c3ccc(N4c5ccccc5C(c5ccccc5)(c5ccccc5)c5ccccc54)cc3)nc3ccncc32)cc1 ; c1ccc(-n2c(-c3ccc(N4c5ccccc5C(c5ccccc5)(c5ccccc5)c5ccccc54)cc3)nc3cnccc32)cc1 ; c1ccc(-n2c(-c3ccc(N4c5ccccc5C(c5ccccc5)(c5ccccc5)c5ccccc54)cc3)nc3ncccc32)cc1 ; c1ccc(C2(c3ccccc3)c3ccccc3N(c3ccc(-c4nc5cccnc5o4)cc3)c3ccccc32)cc1 ; c1ccc(-c2nc(-c3ccc(N4c5ccccc5C(c5ccccc5)(c5ccccc5)c5ccccc54)cc3)n(-c3ccccc3)c2-c2ccccc2)cc1]

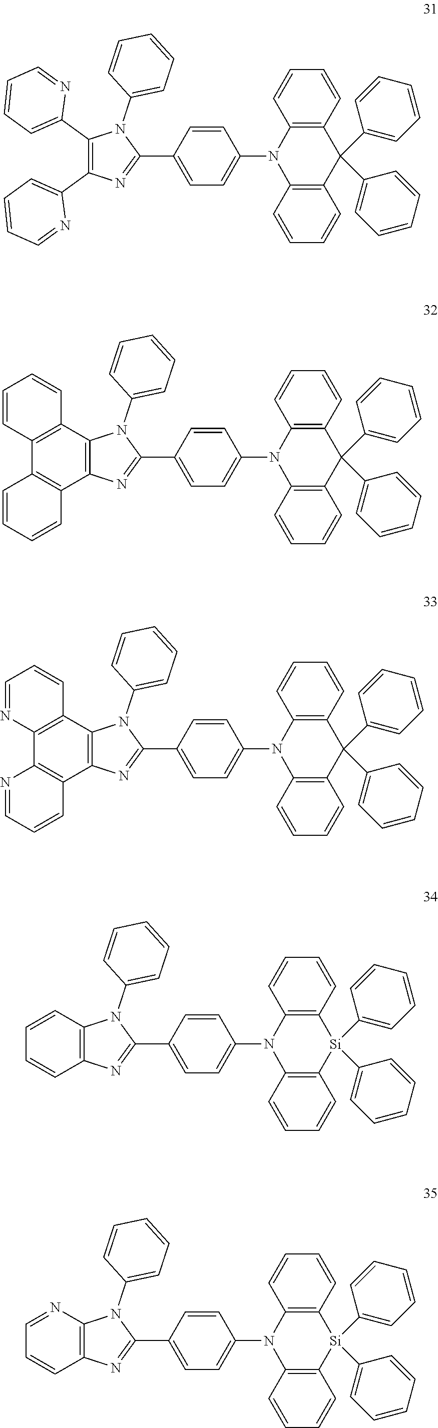 [SMILES: c1ccc(-n2c(-c3ccc(N4c5ccccc5C(c5ccccc5)(c5ccccc5)c5ccccc54)cc3)nc(-c3ccccn3)c2-c2ccccn2)cc1 ; c1ccc(-n2c(-c3ccc(N4c5ccccc5C(c5ccccc5)(c5ccccc5)c5ccccc54)cc3)nc3c4ccccc4c4ccccc4c32)cc1 ; c1ccc(-n2c(-c3ccc(N4c5ccccc5C(c5ccccc5)(c5ccccc5)c5ccccc54)cc3)nc3c4cccnc4c4ncccc4c32)cc1 ; c1ccc(-n2c(-c3ccc(N4c5ccccc5[Si](c5ccccc5)(c5ccccc5)c5ccccc54)cc3)nc3ccccc32)cc1 ; c1ccc(-n2c(-c3ccc(N4c5ccccc5[Si](c5ccccc5)(c5ccccc5)c5ccccc54)cc3)nc3cccnc32)cc1]

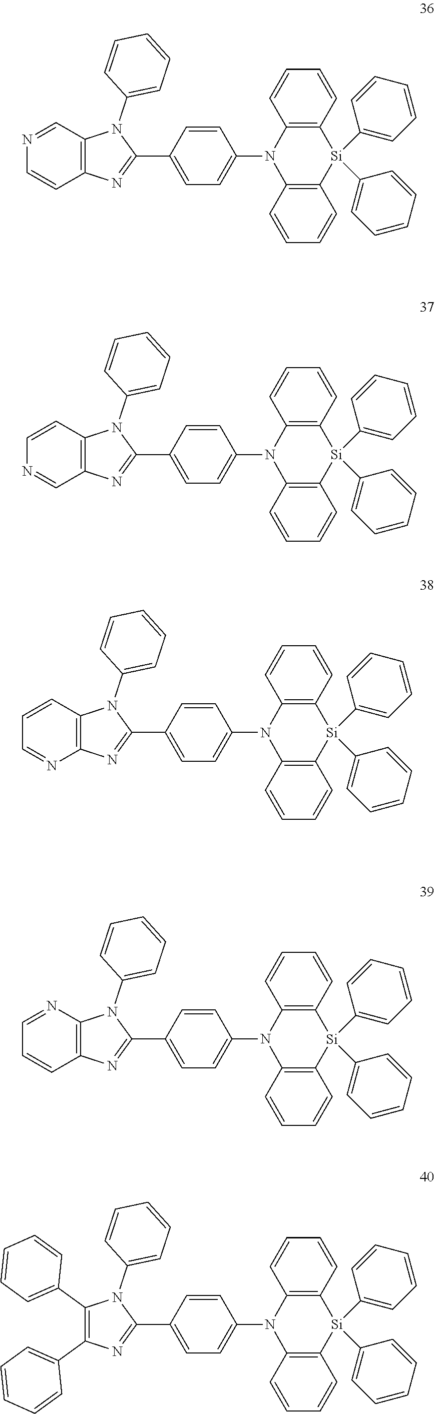 [SMILES: c1ccc(-n2c(-c3ccc(N4c5ccccc5[Si](c5ccccc5)(c5ccccc5)c5ccccc54)cc3)nc3ccncc32)cc1 ; c1ccc(-n2c(-c3ccc(N4c5ccccc5[Si](c5ccccc5)(c5ccccc5)c5ccccc54)cc3)nc3cnccc32)cc1 ; c1ccc(-n2c(-c3ccc(N4c5ccccc5[Si](c5ccccc5)(c5ccccc5)c5ccccc54)cc3)nc3ncccc32)cc1 ; c1ccc(-n2c(-c3ccc(N4c5ccccc5[Si](c5ccccc5)(c5ccccc5)c5ccccc54)cc3)nc3cccnc32)cc1 ; c1ccc(-c2nc(-c3ccc(N4c5ccccc5[Si](c5ccccc5)(c5ccccc5)c5ccccc54)cc3)n(-c3ccccc3)c2-c2ccccc2)cc1]

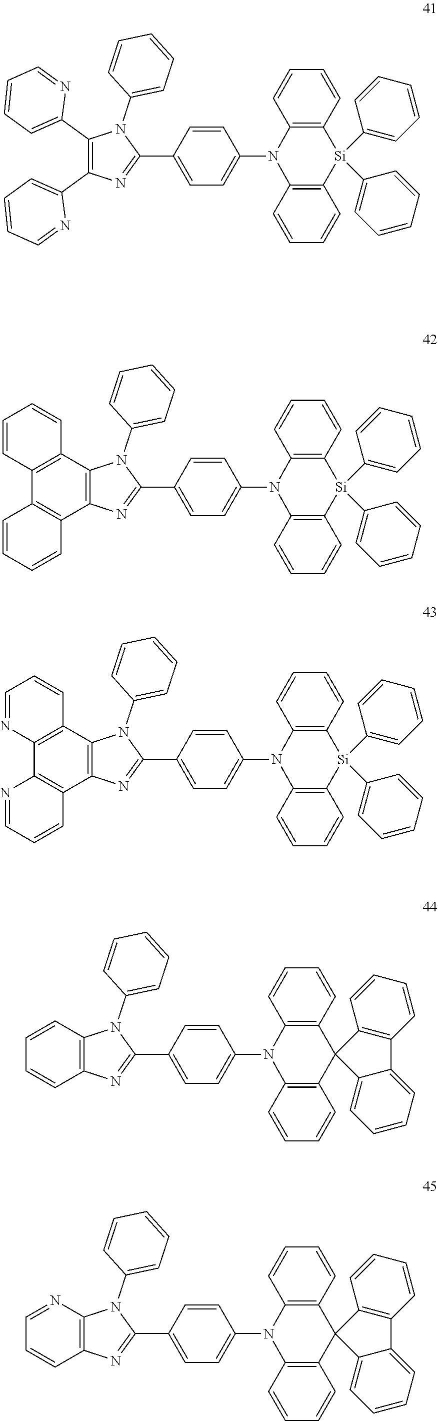 [SMILES: c1ccc(-n2c(-c3ccc(N4c5ccccc5[Si](c5ccccc5)(c5ccccc5)c5ccccc54)cc3)nc(-c3ccccn3)c2-c2ccccn2)cc1 ; c1ccc(-n2c(-c3ccc(N4c5ccccc5[Si](c5ccccc5)(c5ccccc5)c5ccccc54)cc3)nc3c4ccccc4c4ccccc4c32)cc1 ; c1ccc(-n2c(-c3ccc(N4c5ccccc5[Si](c5ccccc5)(c5ccccc5)c5ccccc54)cc3)nc3c4cccnc4c4ncccc4c32)cc1 ; c1ccc(-n2c(-c3ccc(N4c5ccccc5C5(c6ccccc6-c6ccccc65)c5ccccc54)cc3)nc3ccccc32)cc1 ; c1ccc(-n2c(-c3ccc(N4c5ccccc5C5(c6ccccc6-c6ccccc65)c5ccccc54)cc3)nc3cccnc32)cc1]

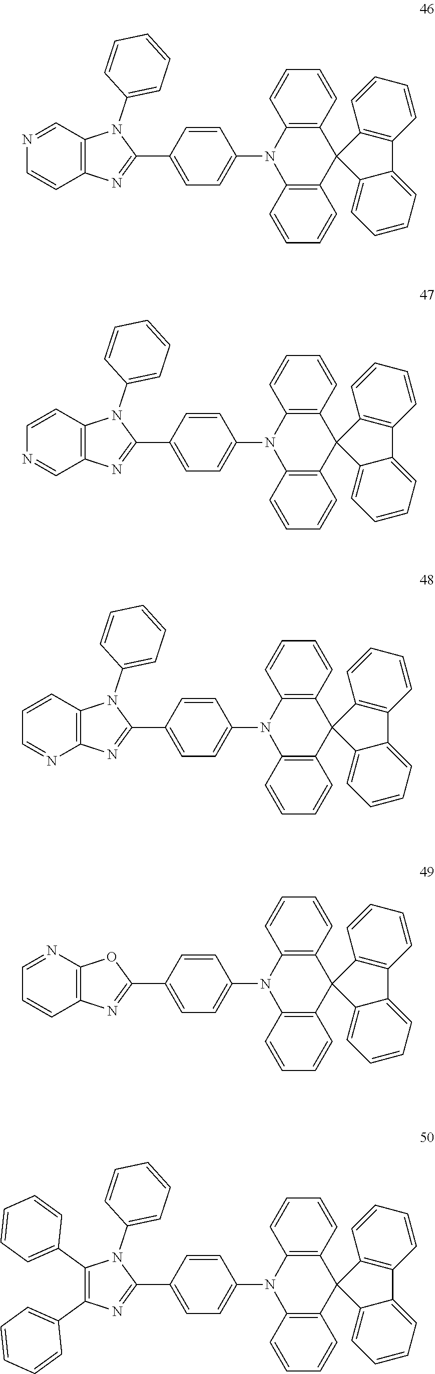 [SMILES: c1ccc(-n2c(-c3ccc(N4c5ccccc5C5(c6ccccc6-c6ccccc65)c5ccccc54)cc3)nc3ccncc32)cc1 ; c1ccc(-n2c(-c3ccc(N4c5ccccc5C5(c6ccccc6-c6ccccc65)c5ccccc54)cc3)nc3cnccc32)cc1 ; c1ccc(-n2c(-c3ccc(N4c5ccccc5C5(c6ccccc6-c6ccccc65)c5ccccc54)cc3)nc3ncccc32)cc1 ; c1ccc2c(c1)-c1ccccc1C21c2ccccc2N(c2ccc(-c3nc4cccnc4o3)cc2)c2ccccc21 ; c1ccc(-c2nc(-c3ccc(N4c5ccccc5C5(c6ccccc6-c6ccccc65)c5ccccc54)cc3)n(-c3ccccc3)c2-c2ccccc2)cc1]

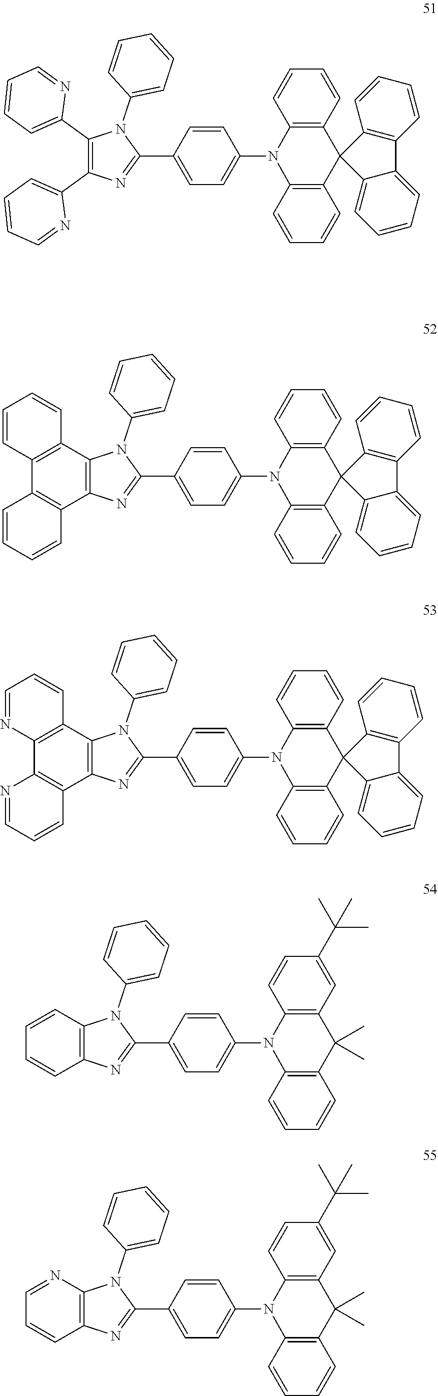

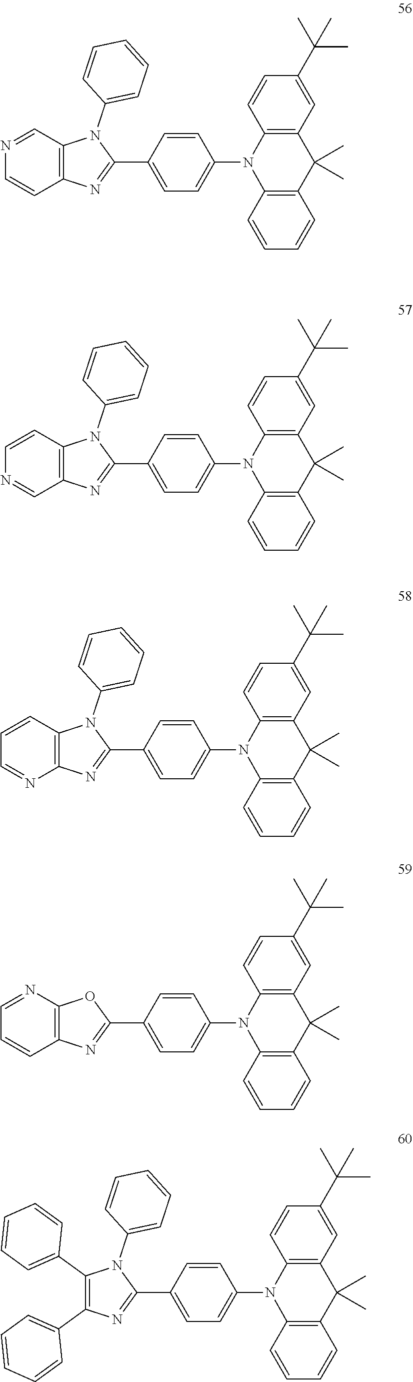

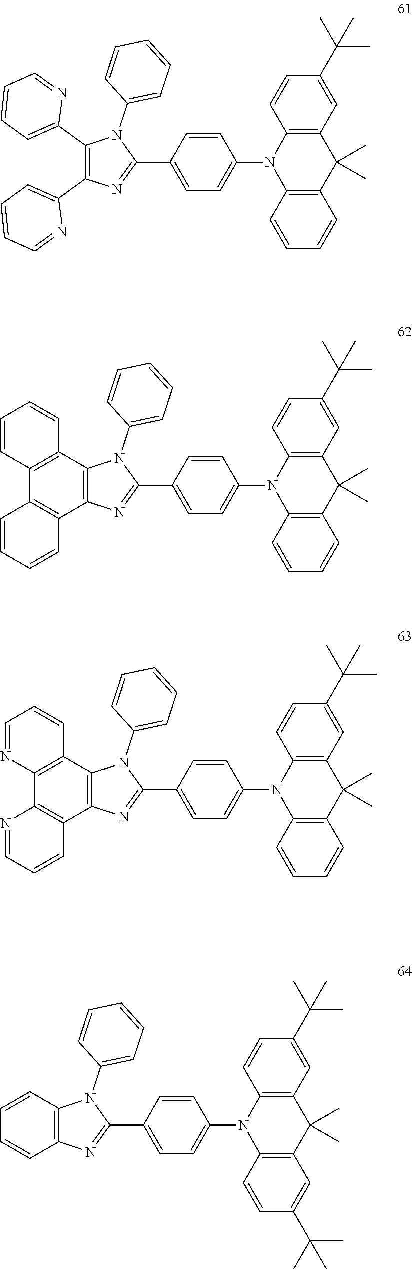 [SMILES: CC(C)(C)c1ccc2c(c1)C(C)(C)c1ccccc1N2c1ccc(-c2nc(-c3ccccn3)c(-c3ccccn3)n2-c2ccccc2)cc1 ; CC(C)(C)c1ccc2c(c1)C(C)(C)c1ccccc1N2c1ccc(-c2nc3c4ccccc4c4ccccc4c3n2-c2ccccc2)cc1 ; CC(C)(C)c1ccc2c(c1)C(C)(C)c1ccccc1N2c1ccc(-c2nc3c4cccnc4c4ncccc4c3n2-c2ccccc2)cc1 ; CC(C)(C)c1ccc2c(c1)C(C)(C)c1cc(C(C)(C)C)ccc1N2c1ccc(-c2nc3ccccc3n2-c2ccccc2)cc1]

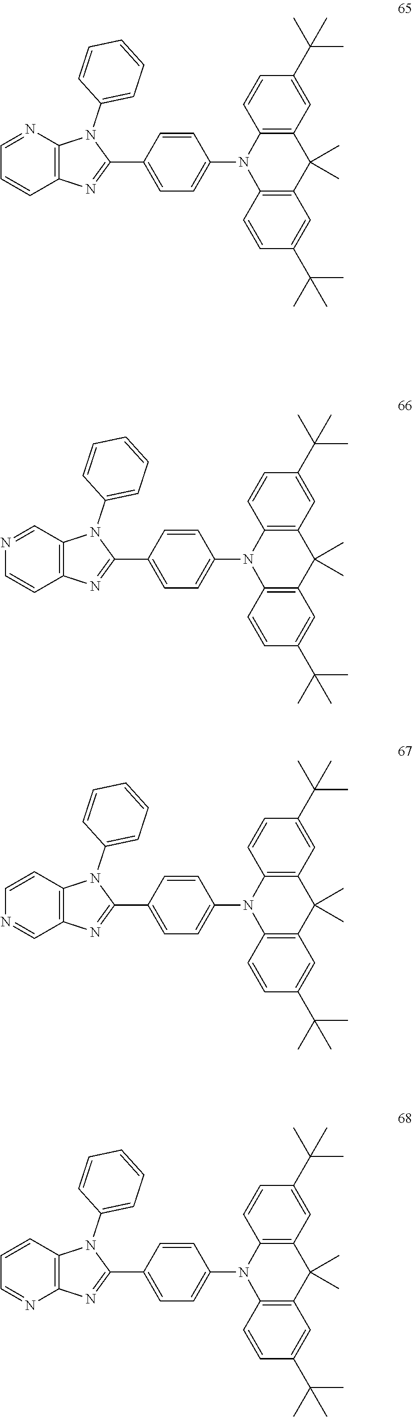 [SMILES: CC(C)(C)c1ccc2c(c1)C(C)(C)c1cc(C(C)(C)C)ccc1N2c1ccc(-c2nc3cccnc3n2-c2ccccc2)cc1 ; CC(C)(C)c1ccc2c(c1)C(C)(C)c1cc(C(C)(C)C)ccc1N2c1ccc(-c2nc3ccncc3n2-c2ccccc2)cc1 ; CC(C)(C)c1ccc2c(c1)C(C)(C)c1cc(C(C)(C)C)ccc1N2c1ccc(-c2nc3cnccc3n2-c2ccccc2)cc1 ; CC(C)(C)c1ccc2c(c1)C(C)(C)c1cc(C(C)(C)C)ccc1N2c1ccc(-c2nc3ncccc3n2-c2ccccc2)cc1]

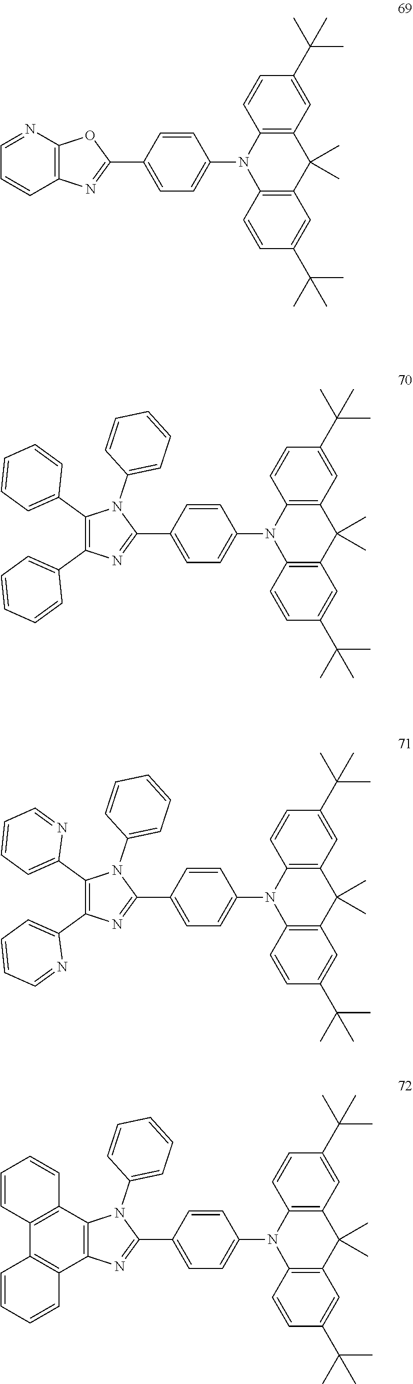 [SMILES: CC(C)(C)c1ccc2c(c1)C(C)(C)c1cc(C(C)(C)C)ccc1N2c1ccc(-c2nc3cccnc3o2)cc1 ; CC(C)(C)c1ccc2c(c1)C(C)(C)c1cc(C(C)(C)C)ccc1N2c1ccc(-c2nc(-c3ccccc3)c(-c3ccccc3)n2-c2ccccc2)cc1 ; CC(C)(C)c1ccc2c(c1)C(C)(C)c1cc(C(C)(C)C)ccc1N2c1ccc(-c2nc(-c3ccccn3)c(-c3ccccn3)n2-c2ccccc2)cc1 ; CC(C)(C)c1ccc2c(c1)C(C)(C)c1cc(C(C)(C)C)ccc1N2c1ccc(-c2nc3c4ccccc4c4ccccc4c3n2-c2ccccc2)cc1]

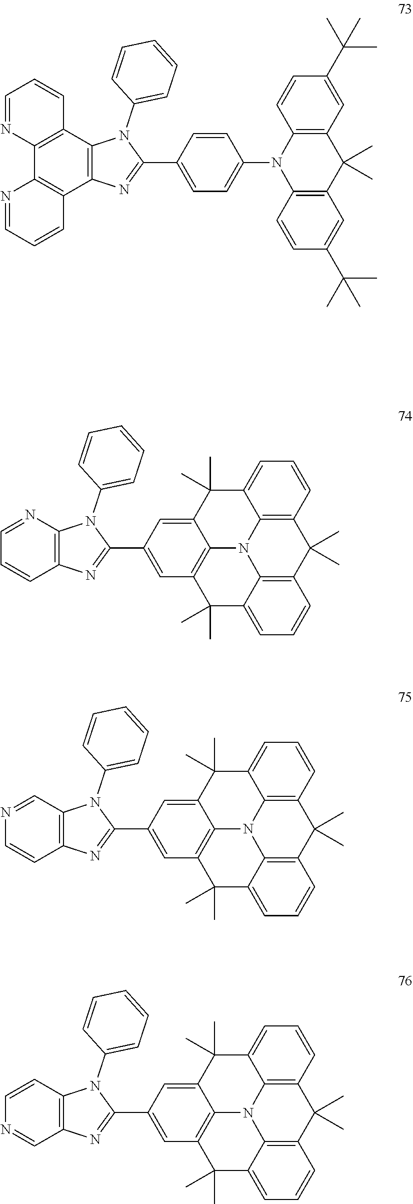 [SMILES: CC(C)(C)c1ccc2c(c1)C(C)(C)c1cc(C(C)(C)C)ccc1N2c1ccc(-c2nc3c4cccnc4c4ncccc4c3n2-c2ccccc2)cc1 ; CC1(C)c2cccc3c2N2c4c1cccc4C(C)(C)c1cc(-c4nc5cccnc5n4-c4ccccc4)cc(c12)C3(C)C ; CC1(C)c2cccc3c2N2c4c1cccc4C(C)(C)c1cc(-c4nc5ccncc5n4-c4ccccc4)cc(c12)C3(C)C ; CC1(C)c2cccc3c2N2c4c1cccc4C(C)(C)c1cc(-c4nc5cnccc5n4-c4ccccc4)cc(c12)C3(C)C]

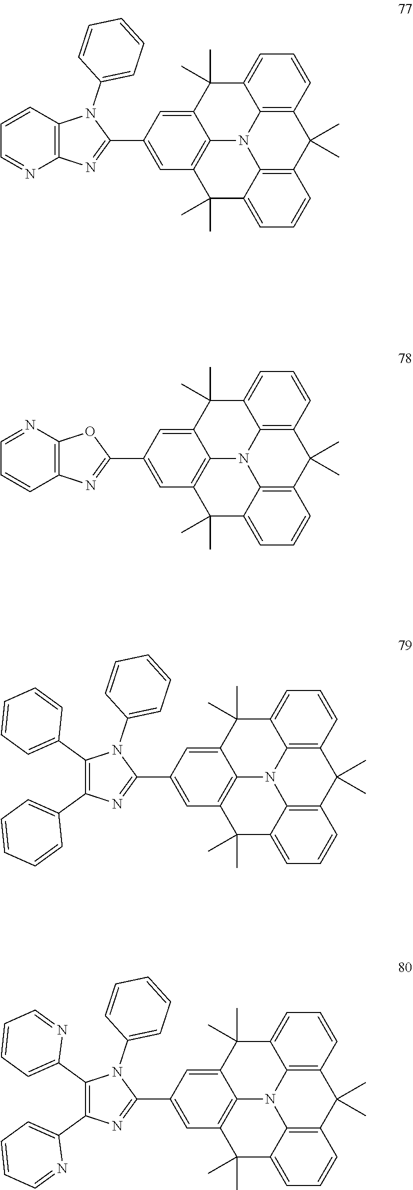 [SMILES: CC1(C)c2cccc3c2N2c4c1cccc4C(C)(C)c1cc(-c4nc5ncccc5n4-c4ccccc4)cc(c12)C3(C)C ; CC1(C)c2cccc3c2N2c4c1cccc4C(C)(C)c1cc(-c4nc5cccnc5o4)cc(c12)C3(C)C ; CC1(C)c2cccc3c2N2c4c1cccc4C(C)(C)c1cc(-c4nc(-c5ccccc5)c(-c5ccccc5)n4-c4ccccc4)cc(c12)C3(C)C ; CC1(C)c2cccc3c2N2c4c1cccc4C(C)(C)c1cc(-c4nc(-c5ccccn5)c(-c5ccccn5)n4-c4ccccc4)cc(c12)C3(C)C]

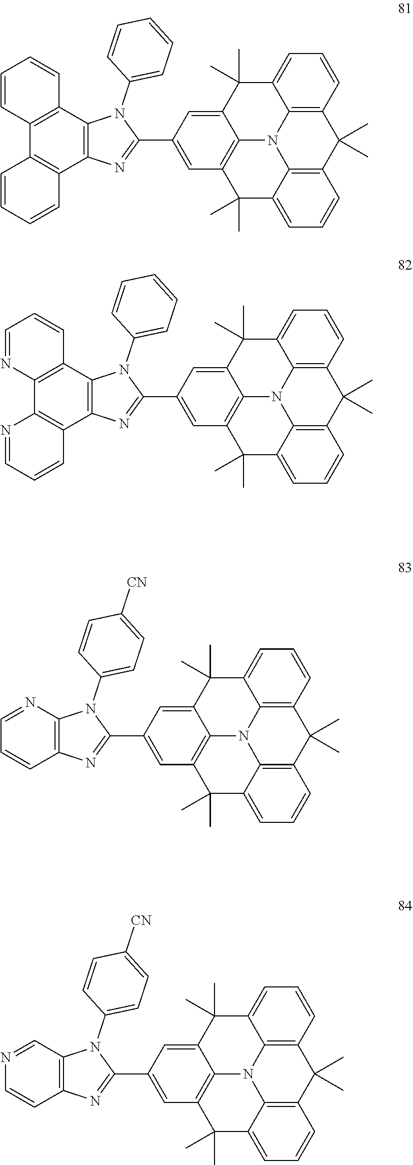 [SMILES: CC1(C)c2cccc3c2N2c4c1cccc4C(C)(C)c1cc(-c4nc5c6ccccc6c6ccccc6c5n4-c4ccccc4)cc(c12)C3(C)C ; CC1(C)c2cccc3c2N2c4c1cccc4C(C)(C)c1cc(-c4nc5c6cccnc6c6ncccc6c5n4-c4ccccc4)cc(c12)C3(C)C ; CC1(C)c2cccc3c2N2c4c1cccc4C(C)(C)c1cc(-c4nc5cccnc5n4-c4ccc(C#N)cc4)cc(c12)C3(C)C ; CC1(C)c2cccc3c2N2c4c1cccc4C(C)(C)c1cc(-c4nc5ccncc5n4-c4ccc(C#N)cc4)cc(c12)C3(C)C]

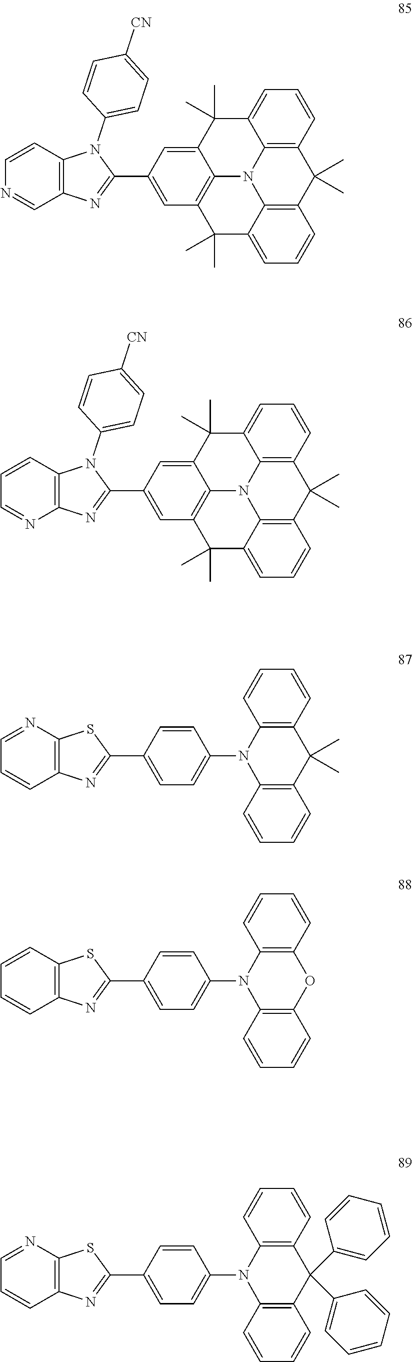 [SMILES: CC1(C)c2cccc3c2N2c4c1cccc4C(C)(C)c1cc(-c4nc5cnccc5n4-c4ccc(C#N)cc4)cc(c12)C3(C)C ; CC1(C)c2cccc3c2N2c4c1cccc4C(C)(C)c1cc(-c4nc5ncccc5n4-c4ccc(C#N)cc4)cc(c12)C3(C)C ; CC1(C)c2ccccc2N(c2ccc(-c3nc4cccnc4s3)cc2)c2ccccc21 ; c1ccc2c(c1)Oc1ccccc1N2c1ccc(-c2nc3ccccc3s2)cc1 ; c1ccc(C2(c3ccccc3)c3ccccc3N(c3ccc(-c4nc5cccnc5s4)cc3)c3ccccc32)cc1]

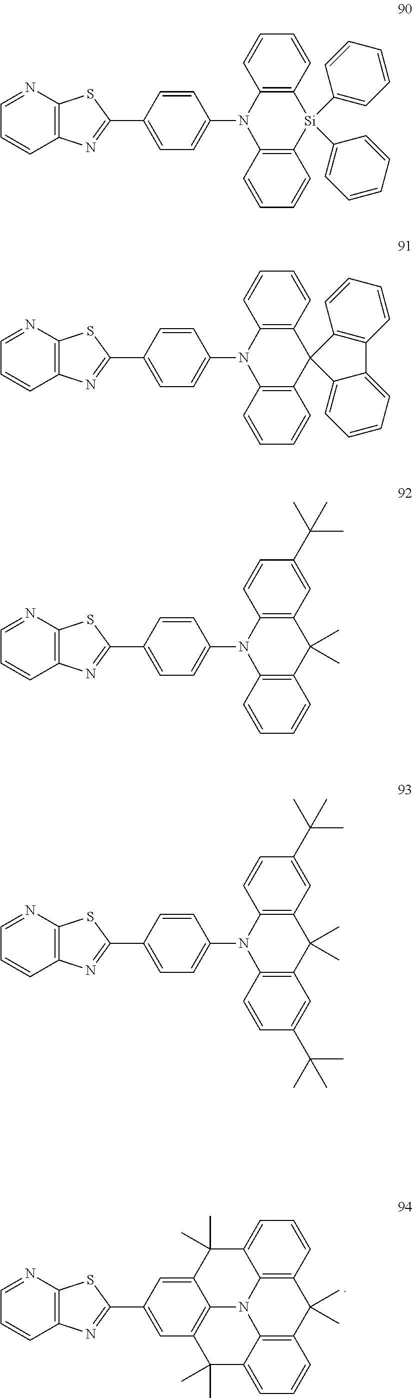 [SMILES: c1ccc([Si]2(c3ccccc3)c3ccccc3N(c3ccc(-c4nc5cccnc5s4)cc3)c3ccccc32)cc1 ; c1ccc2c(c1)-c1ccccc1C21c2ccccc2N(c2ccc(-c3nc4cccnc4s3)cc2)c2ccccc21 ; CC(C)(C)c1ccc2c(c1)C(C)(C)c1ccccc1N2c1ccc(-c2nc3cccnc3s2)cc1 ; CC(C)(C)c1ccc2c(c1)C(C)(C)c1cc(C(C)(C)C)ccc1N2c1ccc(-c2nc3cccnc3s2)cc1 ; CC1(C)c2cccc3c2N2c4c1cccc4C(C)(C)c1cc(-c4nc5cccnc5s4)cc(c12)C3(C)C]

According to an example embodiment, a heterocyclic compound includes an electron acceptor moiety represented by A₁ in Formula 1 and an electron donor moiety represented by a core structure in Formula 1. Without being bound by theory, it is believed that, in such a structure, inter-molecular charge movement is facilitated, thereby improving luminescent efficiency. In addition, since a dipole is formed from the electron donor moiety to the electron acceptor moiety, the dipole moment in the molecule increases, thereby further increasing luminescence efficiency.

In addition, in the heterocyclic compound, the electron donor moiety and the electron acceptor moiety are separated from each other. Without being bound by theory, it is believed that, in such a structure, orbital overlap in a molecule may be effectively blocked. Therefore, since singlet and triplet states of a molecule are not overlapped, the heterocyclic compound may have a very low ΔEₛₜ. In this manner, since reverse intersystem crossing from a triplet excited state to a singlet excited state through thermal activity is possible even at room temperature, delayed fluorescence may be emitted. Since exciton having a triplet state is used for light emission, luminescent efficiency may be improved.

In addition, without being bound by theory, it is believed that, since the heterocyclic compound has a relatively high hole or electron transport capability, it is possible to improve an exciton formation ratio in an emission layer in an organic light-emitting device including the heterocyclic compound represented by Formula 1. Therefore, the organic light-emitting device may have a low driving voltage, high efficiency, a long lifespan, and high maximum quantum efficiency.

A synthesis method for the heterocyclic compound represented by Formula 1 will be apparent to those of skill in the art by referring to the examples below.

At least one of the heterocyclic compound of Formula 1 may be used between a pair of electrodes of an organic light-emitting device. For example, the heterocyclic compound may be included in at least one layer selected from a hole transport region, an electron transport region, and an emission layer. In an example embodiment, the heterocyclic compound of Formula 1 may be used as a material for a capping layer located outside a pair of electrodes of an organic light-emitting device.

According to an example embodiment, an organic light-emitting device includes a first electrode, a second electrode facing the first electrode, and an organic layer between the first electrode and the second electrode. The organic layer may include an emission layer. The organic layer may include at least one heterocyclic compound represented by Formula 1.

The expression “(an organic layer) includes at least one heterocyclic compound” used herein may include a case in which “(an organic layer) includes identical heterocyclic compounds represented by Formula 1” and a case in which “(an organic layer) includes two or more different heterocyclic compounds represented by Formula 1.”

For example, the organic layer may include, as the heterocyclic compound, only Compound 1. In this regard, Compound 1 may exist in an emission layer of the organic light-emitting device. In an example embodiment, the organic layer may include, as the heterocyclic compound, Compound 1 and Compound 2. In this regard, Compound 1 and Compound 2 may exist in an identical layer (for example, Compound 1 and Compound 2 may both exist in an emission layer), or different layers (for example, Compound 1 may exist in an emission layer and Compound 2 may exist in an electron transport layer).

In an example embodiment, the first electrode may be an anode, and the second electrode may be a cathode, and the organic layer may further include a hole transport region between the first electrode and the emission layer and an electron transport region between the emission layer and the second electrode, and the hole transport region may include a hole injection layer, a hole transport layer, an emission auxiliary layer, an electron blocking layer, or a combination thereof.

The electron transport region may include a hole blocking layer, an electron transport layer, an electron injection layer, or a combination thereof.

In an example embodiment, the emission layer of the organic light-emitting device may include the heterocyclic compound.

In an example embodiment, the emission layer of the organic light-emitting device includes the heterocyclic compound, the heterocyclic compound included in the emission layer is a thermally activated delayed fluorescence emitter (TADF emitter), and the emission layer may emit delayed fluorescence.

In an example embodiment, the emission layer may consist of the heterocyclic compound, or the emission layer may further include a host. An amount of the heterocyclic compound may be in a range of about 0.1 parts by weight to about 50 parts by weight based on 100 parts by weight of the emission layer.

For example, the emission layer may consist of the heterocyclic compound, the heterocyclic compound may be a TADF emitter, and the TADF emitter may satisfy Equation 1:

|E_{D, S1}−E_{D, T1}|≤0.3 eV.< Equation 1>

In Equation 1,

E_{D, S1} is singlet energy level (eV) of the TADF emitter, and

E_{D, T1} is triplet energy level (eV) of the TADF emitter.

Without being bound by theory, it is believed that, since a difference between the singlet energy level (E_{D, S1}) and the triplet energy level (E_{D, T1}) of the TADF emitter, that is, the heterocyclic compound represented by Formula 1, is about 0.3 eV or less, singlet state exciton and triplet state exciton, which are generated by electric field, may easily transition to an intermediate state. Therefore, an organic light-emitting device including the heterocyclic compound may have excellent luminescent efficiency.

In an example embodiment, the emission layer may include the heterocyclic compound and a host, the heterocyclic compound may be a TADF emitter, and the TADF emitter and the host may satisfy Equation 2 or 3.

|E_{H, HOMO}−E_{D, HOMO}|≤0.5 eV <Equation 2>

|E_{H, LUMO}−E_{D, LUMO}|≤0.5 eV. <Equation 3>

In Equations 2 and 3,

E_{H, HOMO} is a highest occupied molecular orbital (HOMO) energy level (eV) of the host,

E_{D, HOMO} is a HOMO energy level (eV) of the dopant,

E_{H, LUMO} is a lowest unoccupied molecular orbital (LUMO) energy level (eV) of the host, and

E_{D, LUMO} is a LUMO energy level (eV) of the dopant.

Without being bound by theory, it is believed that, since a difference in HOMO energy level or LUMO energy level between the TADF emitter, that is, the heterocyclic compound represented by Formula 1, and the host is about 0.5 eV or less, charge transfer from the host to the TADF emitter is facilitated. Therefore, an organic light-emitting device including the heterocyclic compound and the host may have excellent luminescent efficiency.

In the emission layer, the host may include at least one selected from an anthracene-based compound, a pyrene-based compound, a spiro-bifluorene-based compound, a carbazole-based compound, a benzimidazole-based compound, and a phosphine oxide-based compound.

In an example embodiment, the emission layer in the organic light-emitting device may include the heterocyclic compound, and the emission layer may emit blue light having a maximum emission wavelength in a range of about 400 nm to about 500 nm.

In an example embodiment, the hole transport region of the organic light-emitting device may include a p-dopant, and the p-dopant may have a lowest unoccupied molecular orbital (LUMO) energy level of about −3.5 eV or less.

In an example embodiment, the electron transport region of the organic light-emitting device may include at least one selected from a phosphine oxide-based compound and a benzimidazole-based compound, and

may further include an alkali metal, an alkaline earth metal, a rare earth metal, an alkali metal compound, an alkaline earth-metal compound, a rare earth metal compound, an alkali metal complex, an alkaline earth-metal complex, a rare earth metal complex, or a combinations thereof.

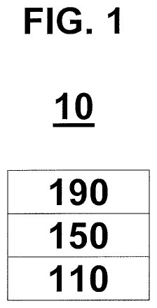

The term “organic layer” used herein refers to a single layer and/or a plurality of layers disposed between the first electrode and the second electrode of the organic light-emitting device. A material included in the “organic layer” is not limited to an organic material.

[Description of FIG. 1]

FIG. 1 is a schematic view of an organic light-emitting device 10 according to an example embodiment. The organic light-emitting device 10 includes a first electrode 110, an organic layer 150, and a second electrode 190.

Hereinafter, the structure of the organic light-emitting device 10 according to an example embodiment and a method of manufacturing the organic light-emitting device 10 will be described in connection with FIG. 1.

[First Electrode 110]

In FIG. 1, a substrate may be additionally disposed under the first electrode 110 or above the second electrode 190. The substrate may be a glass substrate or a plastic substrate, each having excellent mechanical strength, thermal stability, transparency, surface smoothness, ease of handling, and water resistance.

The first electrode 110 may be formed by depositing or sputtering a material for forming the first electrode 110 on the substrate. When the first electrode 110 is an anode, the material for a first electrode may be selected from materials with a high work function to facilitate hole injection.

The first electrode 110 may be a reflective electrode, a semi-transmissive electrode, or a transmissive electrode. When the first electrode 110 is a transmissive electrode, a material for forming a first electrode may be selected from indium tin oxide (ITO), indium zinc oxide (IZO), tin oxide (SnO₂), zinc oxide (ZnO), and a combinations thereof. In an example embodiment, when the first electrode 110 is a semi-transmissive electrode or a reflective electrode, a material for forming a first electrode may be selected from magnesium (Mg), silver (Ag), aluminum (Al), aluminum-lithium (Al—Li), calcium (Ca), magnesium-indium (Mg—In), magnesium-silver (Mg—Ag), and a combinations thereof.

The first electrode 110 may have a single-layered structure, or a multi-layered structure including two or more layers. For example, the first electrode 110 may have a three-layered structure of ITO/Ag/ITO.

[Organic layer 150]

The organic layer 150 is disposed on the first electrode 110. The organic layer 150 may include an emission layer.

The organic layer 150 may further include a hole transport region between the first electrode 110 and the emission layer, and an electron transport region between the emission layer and the second electrode 190.

[Hole Transport Region in Organic Layer 150]

The hole transport region may have i) a single-layered structure including a single layer including a single material, ii) a single-layered structure including a single layer including a plurality of different materials, or iii) a multi-layered structure having a plurality of layers including a plurality of different materials.

The hole transport region may include at least one layer selected from a hole injection layer, a hole transport layer, an emission auxiliary layer, and an electron blocking layer.

For example, the hole transport region may have a single-layered structure including a single layer including a plurality of different materials, or a multi-layered structure having a hole injection layer/hole transport layer structure, a hole injection layer/hole transport layer/emission auxiliary layer structure, a hole injection layer/emission auxiliary layer structure, a hole transport layer/emission auxiliary layer structure, or a hole injection layer/hole transport layer/electron blocking layer structure, wherein for each structure, constituting layers are sequentially stacked from the first electrode 110 in this stated order.

The hole transport region may include at least one selected from m-MTDATA, TDATA, 2-TNATA, NPB(NPD), β-NPB, TPD, Spiro-TPD, Spiro-NPB, methylated-NPB, TAPC, HMTPD, 4,4′,4″-tris(N-carbazolyl)triphenylamine (TCTA), polyaniline/dodecylbenzenesulfonic acid (PANI/DBSA), poly(3,4-ethylenedioxythiophene)/poly(4-styrenesulfonate) (PEDOT/PSS), polyaniline/camphor sulfonic acid (PANI/CSA), polyaniline/poly(4-styrenesulfonate) (PANI/PSS), a compound represented by Formula 201, and a compound represented by Formula 202:

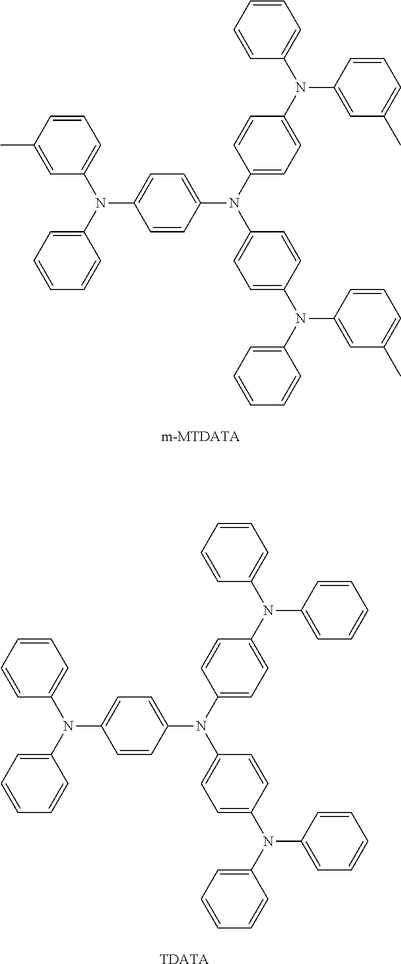

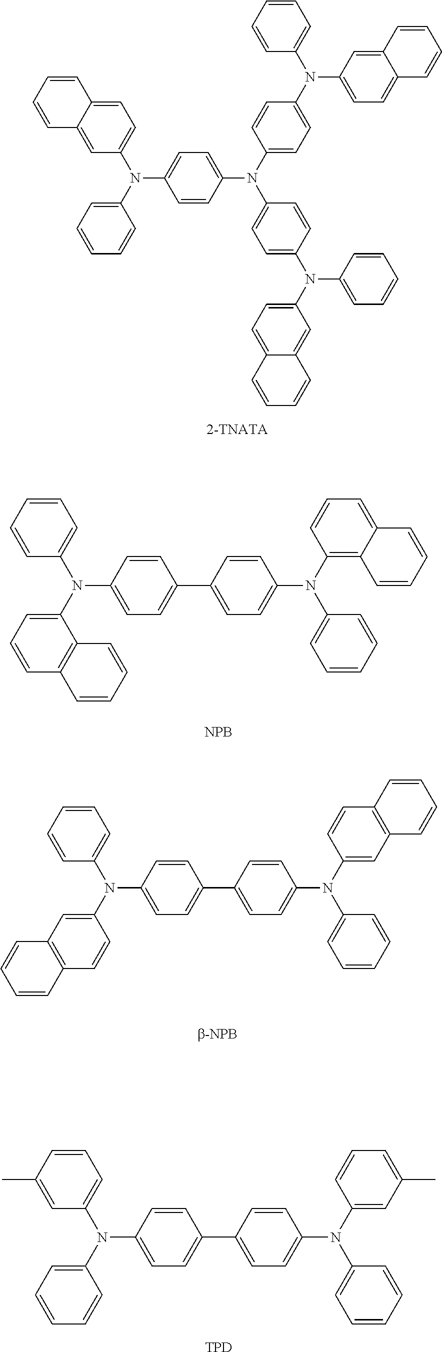

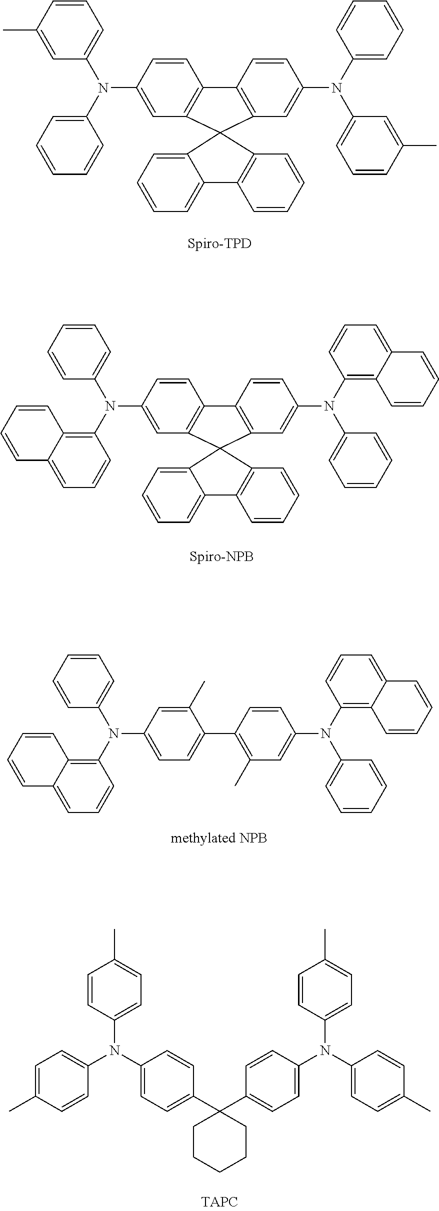

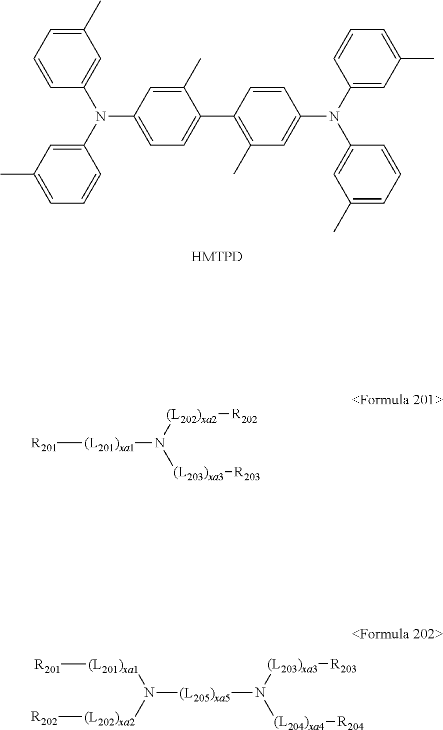

In Formulae 201 and 202,

L₂₀₁ to L₂₀₄ may each independently be selected from a substituted or unsubstituted C₃-C₁₀ cycloalkylene group, a substituted or unsubstituted C₁-C₁₀ heterocycloalkylene group, a substituted or unsubstituted C₃-C₁₀ cycloalkenylene group, a substituted or unsubstituted C₁-C₁₀ heterocycloalkenylene group, a substituted or unsubstituted C₆-C₆₀ arylene group, a substituted or unsubstituted C₁-C₆₀ heteroarylene group, a substituted or unsubstituted divalent non-aromatic condensed polycyclic group, and a substituted or unsubstituted divalent non-aromatic condensed heteropolycyclic group,

L₂₀₅ may be selected from *—O—*′, *—S—*′, *—N(Q₂₀₁)-*′, a substituted or unsubstituted C₁-C₂₀ alkylene group, a substituted or unsubstituted C₂-C₂₀ alkenylene group, a substituted or unsubstituted C₃-C₁₀ cycloalkylene group, a substituted or unsubstituted C₁-C₁₀ heterocycloalkylene group, a substituted or unsubstituted C₃-C₁₀ cycloalkenylene group, a substituted or unsubstituted C₁-C₁₀ heterocycloalkenylene group, a substituted or unsubstituted C₆-C₆₀ arylene group, a substituted or unsubstituted C₁-C₆₀ heteroarylene group, a substituted or unsubstituted divalent non-aromatic condensed polycyclic group, and a substituted or unsubstituted divalent non-aromatic condensed heteropolycyclic group,

xa1 to xa4 may each independently be an integer from 0 to 3,

xa5 may be an integer from 1 to 10, and

R₂₀₁ to R₂₀₄ and Q₂₀₁ may each independently be selected from a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, and a substituted or unsubstituted monovalent non-aromatic condensed heteropolycyclic group.

For example, in Formula 202, R₂₀₁ and R₂₀₂ may be linked via a single bond, a dimethyl-methylene group, or a diphenyl-methylene group, and R₂₀₃ and R₂₀₄ may be linked via a single bond, a dimethyl-methylene group, or a diphenyl-methylene group.

In an example embodiment, in Formulae 201 and 202,

L₂₀₁ to L₂₀₅ may each independently be selected from:

a phenylene group, a pentalenylene group, an indenylene group, a naphthylene group, an azulenylene group, a heptalenylene group, an indacenylene group, an acenaphthylene group, a fluorenylene group, a spiro-bifluorenylene group, a benzofluorenylene group, a dibenzofluorenylene group, a phenalenylene group, a phenanthrenylene group, an anthracenylenle group, a fluoranthenylene group, a triphenylenylene group, a pyrenylene group, a chrysenylene group, a naphthacenylene group, a picenylene group, a perylenylene group, a pentaphenylene group, a hexacenylene group, a pentacenylene group, a rubicenylene group, a coronenylene group, an ovalenylene group, a thiophenylene group, a furanylene group, a carbazolylene group, an indolylene group, an isoindolylene group, a benzofuranylene group, a benzothiophenylene group, a dibenzofuranylene group, a dibenzothiophenylene group, a benzocarbazolylene group, a dibenzocarbazolylene group, a dibenzosilolylene group, and a pyridinylene group; and

a phenylene group, a pentalenylene group, an indenylene group, a naphthylene group, an azulenylene group, a heptalenylene group, an indacenylene group, an acenaphthylene group, a fluorenylene group, a spiro-bifluorenylene group, a benzofluorenylene group, a dibenzofluorenylene group, a phenalenylene group, a phenanthrenylene group, an anthracenylene group, a fluoranthenylene group, a triphenylenylene group, a pyrenylene group, a chrysenylene group, a naphthacenylene group, a picenylene group, a perylenylene group, a pentaphenylene group, a hexacenylene group, a pentacenylene group, a rubicenylene group, a coronenylene group, an ovalenylene group, a thiophenylene group, a furanylene group, a carbazolylene group, an indolylene group, an isoindolylene group, a benzofuranylene group, a benzothiophenylene group, a dibenzofuranylene group, a dibenzothiophenylene group, a benzocarbazolylene group, a dibenzocarbazolylene group, a dibenzosilolylene group, and a pyridinylene group, each substituted with at least one selected from deuterium, —F, —Cl, —Br, —I, a hydroxyl group, a cyano group, a nitro group, an amidino group, a hydrazino group, a hydrazono group, a C₁-C₂₀ alkyl group, a C₁-C₂₀ alkoxy group, a cyclopentyl group, a cyclohexyl group, a cycloheptyl group, a cyclopentenyl group, a cyclohexenyl group, a phenyl group, a biphenyl group, a terphenyl group, a phenyl group substituted with a C₁-C₁₀ alkyl group, a phenyl group substituted with —F, a pentalenyl group, an indenyl group, a naphthyl group, an azulenyl group, a heptalenyl group, an indacenyl group, an acenaphthyl group, a fluorenyl group, a spiro-bifluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, an ovalenyl group, a thiophenyl group, a furanyl group, a carbazolyl group, an indolyl group, an isoindolyl group, a benzofuranyl group, a benzothiophenyl group, a dibenzofuranyl group, a dibenzothiophenyl group, a benzocarbazolyl group, a dibenzocarbazolyl group, a dibenzosilolyl group, a pyridinyl group, —Si(Q₃₁)(Q₃₂)(Q₃₃), and —N(Q₃₁)(Q₃₂), and

Q₃₁ to Q₃₃ may each independently be selected from a C₁-C₁₀ alkyl group, a C₁-C₁₀ alkoxy group, a phenyl group, a biphenyl group, a terphenyl group, and a naphthyl group.

In an example embodiment, xa1 to xa4 may each independently be 0, 1, or 2.

In an example embodiment, xa5 may be 1, 2, 3, or 4.

In an example embodiment, R₂₀₁ to R₂₀₄ and Q₂₀₁ may each independently be selected from:

a phenyl group, a biphenyl group, a terphenyl group, a pentalenyl group, an indenyl group, a naphthyl group, an azulenyl group, a heptalenyl group, an indacenyl group, an acenaphthyl group, a fluorenyl group, a spiro-bifluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, an ovalenyl group, a thiophenyl group, a furanyl group, a carbazolyl group, an indolyl group, an isoindolyl group, a benzofuranyl group, a benzothiophenyl group, a dibenzofuranyl group, a dibenzothiophenyl group, a benzocarbazolyl group, a dibenzocarbazolyl group, a dibenzosilolyl group, and a pyridinyl group; and

a phenyl group, a biphenyl group, a terphenyl group, a pentalenyl group, an indenyl group, a naphthyl group, an azulenyl group, a heptalenyl group, an indacenyl group, an acenaphthyl group, a fluorenyl group, a spiro-bifluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, an ovalenyl group, a thiophenyl group, a furanyl group, a carbazolyl group, an indolyl group, an isoindolyl group, a benzofuranyl group, a benzothiophenyl group, a dibenzofuranyl group, a dibenzothiophenyl group, a benzocarbazolyl group, a dibenzocarbazolyl group, a dibenzosilolyl group, and a pyridinyl group, each substituted with at least one selected from deuterium, —F, —Cl, —Br, —I, a hydroxyl group, a cyano group, a nitro group, an amidino group, a hydrazino group, a hydrazono group, a C₁-C₂₀ alkyl group, a C₁-C₂₀ alkoxy group, a cyclopentyl group, a cyclohexyl group, a cycloheptyl group, a cyclopentenyl group, a cyclohexenyl group, a phenyl group, a biphenyl group, a terphenyl group, a phenyl group substituted with a C₁-C₁₀ alkyl group, a phenyl group substituted with —F, a pentalenyl group, an indenyl group, a naphthyl group, an azulenyl group, a heptalenyl group, an indacenyl group, an acenaphthyl group, a fluorenyl group, a spiro-bifluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, an ovalenyl group, a thiophenyl group, a furanyl group, a carbazolyl group, an indolyl group, an isoindolyl group, a benzofuranyl group, a benzothiophenyl group, a dibenzofuranyl group, a dibenzothiophenyl group, a benzocarbazolyl group, a dibenzocarbazolyl group, a dibenzosilolyl group, a pyridinyl group, —Si(Q₃₁)(Q₃₂)(Q₃₃), and —N(Q₃₁)(Q₃₂), and

Q₃₁ to Q₃₃ are the same as described above.

In an example embodiment, in Formula 201, at least one selected from R₂₀₁ to R₂₀₃ may each independently be selected from:

a fluorenyl group, a spiro-bifluorenyl group, a carbazolyl group, a dibenzofuranyl group, and a dibenzothiophenyl group; and

a fluorenyl group, a spiro-bifluorenyl group, a carbazolyl group, a dibenzofuranyl group, and a dibenzothiophenyl group, each substituted with at least one selected from deuterium, —F, —Cl, —Br, —I, a hydroxyl group, a cyano group, a nitro group, an amidino group, a hydrazino group, a hydrazono group, a C₁-C₂₀ alkyl group, a C₁-C₂₀ alkoxy group, a cyclopentyl group, a cyclohexyl group, a cycloheptyl group, a cyclopentenyl group, a cyclohexenyl group, a phenyl group, a biphenyl group, a terphenyl group, a phenyl group substituted with a C₁-C₁₀ alkyl group, a phenyl group substituted with —F, a naphthyl group, a fluorenyl group, a spiro-bifluorenyl group, a carbazolyl group, a dibenzofuranyl group, and a dibenzothiophenyl group.

In an example embodiment, in Formula 202, i) R₂₀₁ and R₂₀₂ may be linked via a single bond, and/or ii) R₂₀₃ and R₂₀₄ may be linked via a single bond.

In an example embodiment, in Formula 202, at least one selected from R₂₀₁ to R₂₀₄ may be selected from:

a carbazolyl group; and

a carbazolyl group substituted with at least one selected from deuterium, —F, —Cl, —Br, —I, a hydroxyl group, a cyano group, a nitro group, an amidino group, a hydrazino group, a hydrazono group, a C₁-C₂₀ alkyl group, a C₁-C₂₀ alkoxy group, a cyclopentyl group, a cyclohexyl group, a cycloheptyl group, a cyclopentenyl group, a cyclohexenyl group, a phenyl group, a biphenyl group, a terphenyl group, a phenyl group substituted with a C₁-C₁₀ alkyl group, a phenyl group substituted with —F, a naphthyl group, a fluorenyl group, a spiro-bifluorenyl group, a carbazolyl group, a dibenzofuranyl group, and a dibenzothiophenyl group.

The compound represented by Formula 201 may be represented by Formula 201A:

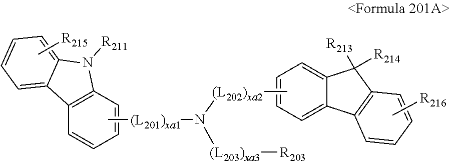

For example, the compound represented by Formula 201 may be represented by Formula 201A(1):

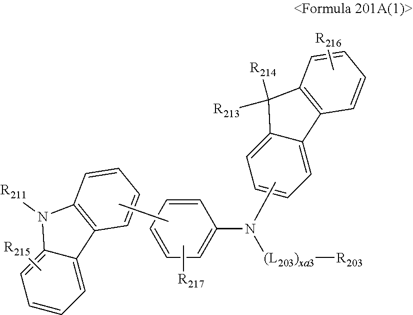

In an example embodiment, the compound represented by Formula 201 may be represented by Formula 201A-1:

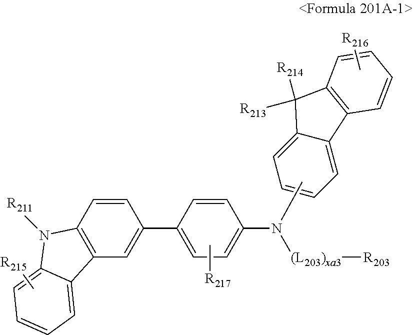

In an example embodiment, the compound represented by Formula 202 may be represented by Formula 202A:

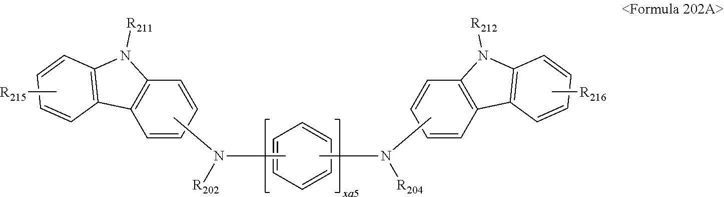

In an example embodiment, the compound represented by Formula 202 may be represented by Formula 202A-1:

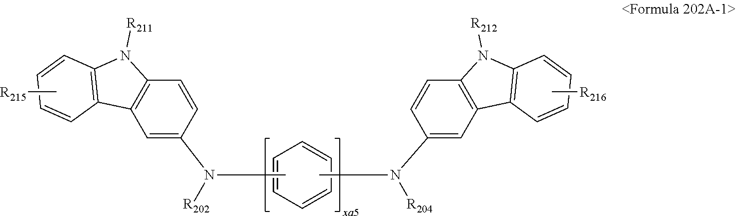

In Formulae 201A, 201A(1), 201A-1, 202A, and 202A-1,

L₂₀₁ to L₂₀₃, xa1 to xa3, xa5, and R₂₀₂ to R₂₀₄ are the same as described above,

R₂₁₁ and R₂₁₂ may each independently be defined the same as R₂₀₃.

R₂₁₃ to R₂₁₇ may each independently be selected from hydrogen, deuterium, —F, —Cl, —Br, —I, a hydroxyl group, a cyano group, a nitro group, an amidino group, a hydrazino group, a hydrazono group, a C₁-C₂₀ alkyl group, a C₁-C₂₀ alkoxy group, a cyclopentyl group, a cyclohexyl group, a cycloheptyl group, a cyclopentenyl group, a cyclohexenyl group, a phenyl group, a biphenyl group, a terphenyl group, a phenyl group substituted with a C₁-C₁₀ alkyl group, a phenyl group substituted with —F, a pentalenyl group, an indenyl group, a naphthyl group, an azulenyl group, a heptalenyl group, an indacenyl group, an acenaphthyl group, a fluorenyl group, a spiro-bifluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, an ovalenyl group, a thiophenyl group, a furanyl group, a carbazolyl group, an indolyl group, an isoindolyl group, a benzofuranyl group, a benzothiophenyl group, a dibenzofuranyl group, a dibenzothiophenyl group, a benzocarbazolyl group, a dibenzocarbazolyl group, a dibenzosilolyl group, and a pyridinyl group.

The hole transport region may include at least one compound selected from Compounds HT1 to HT39:

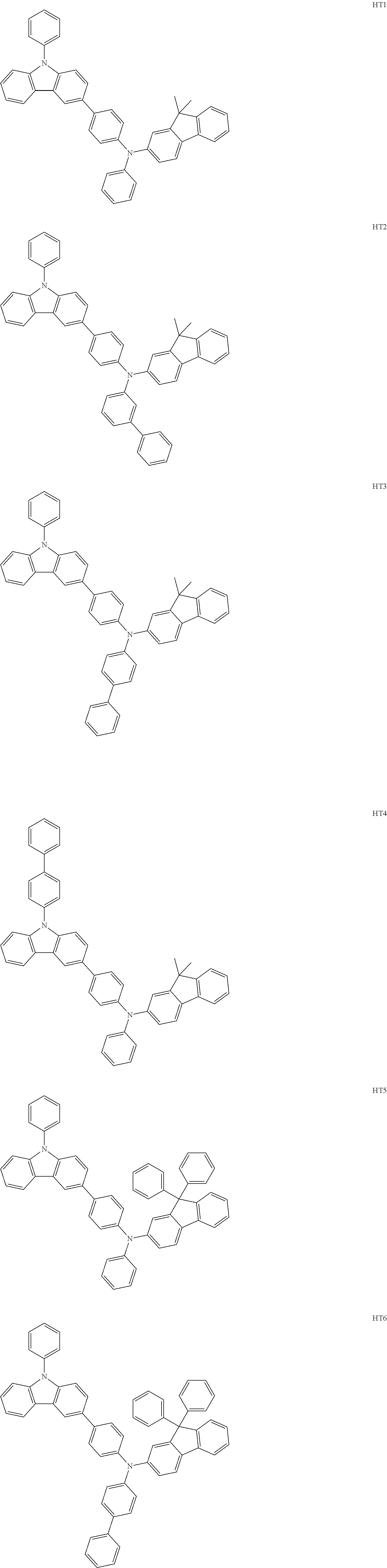

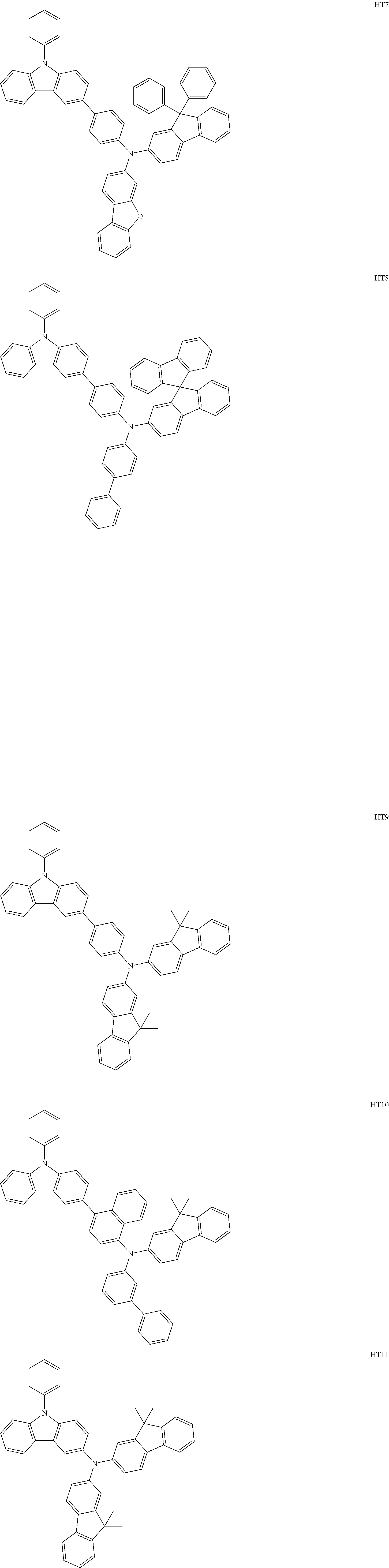

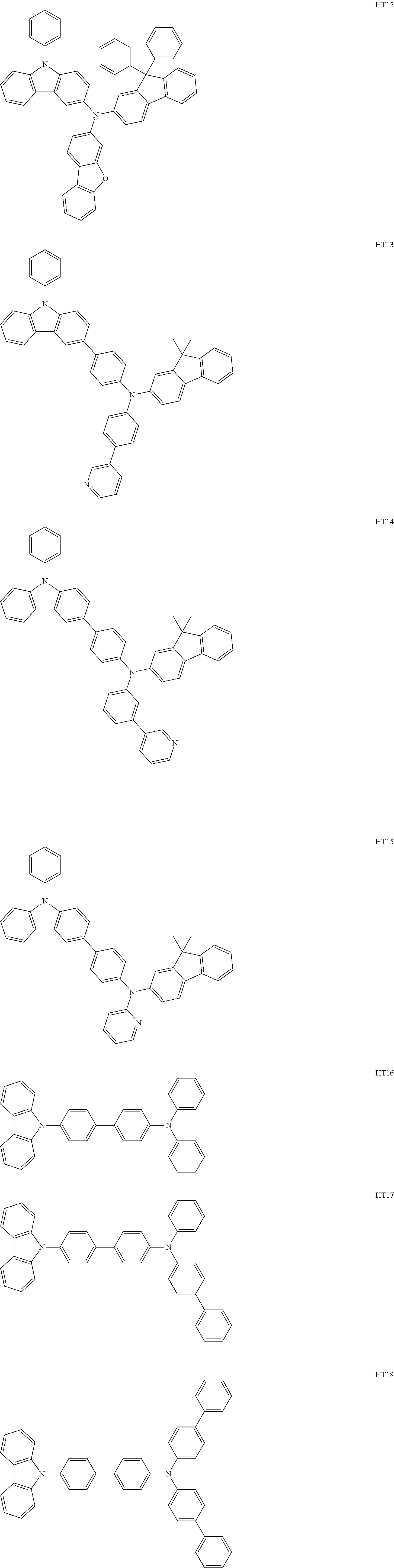

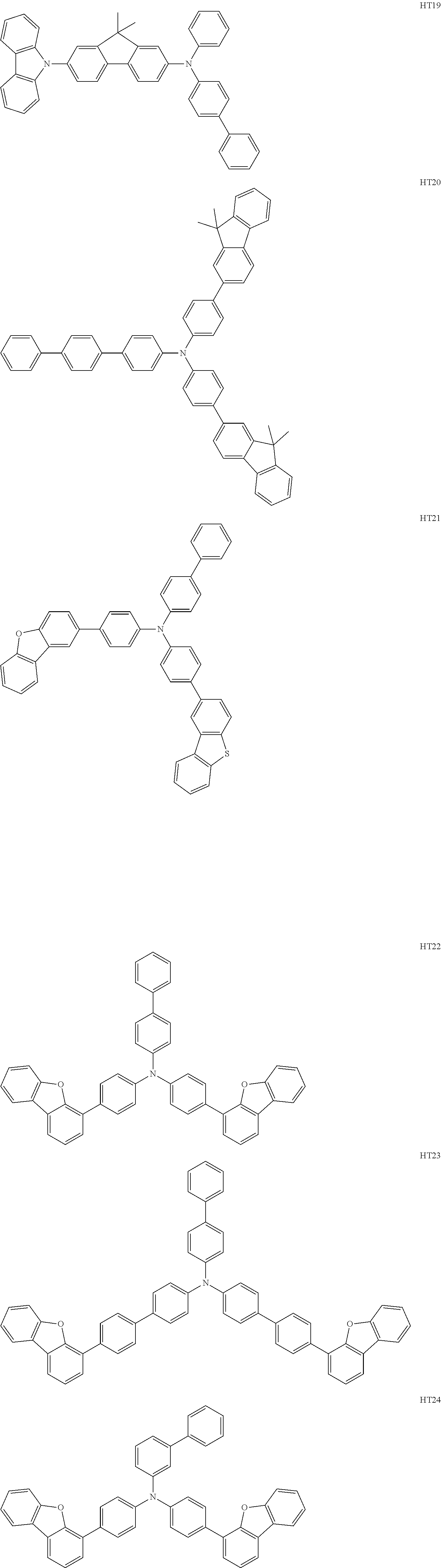

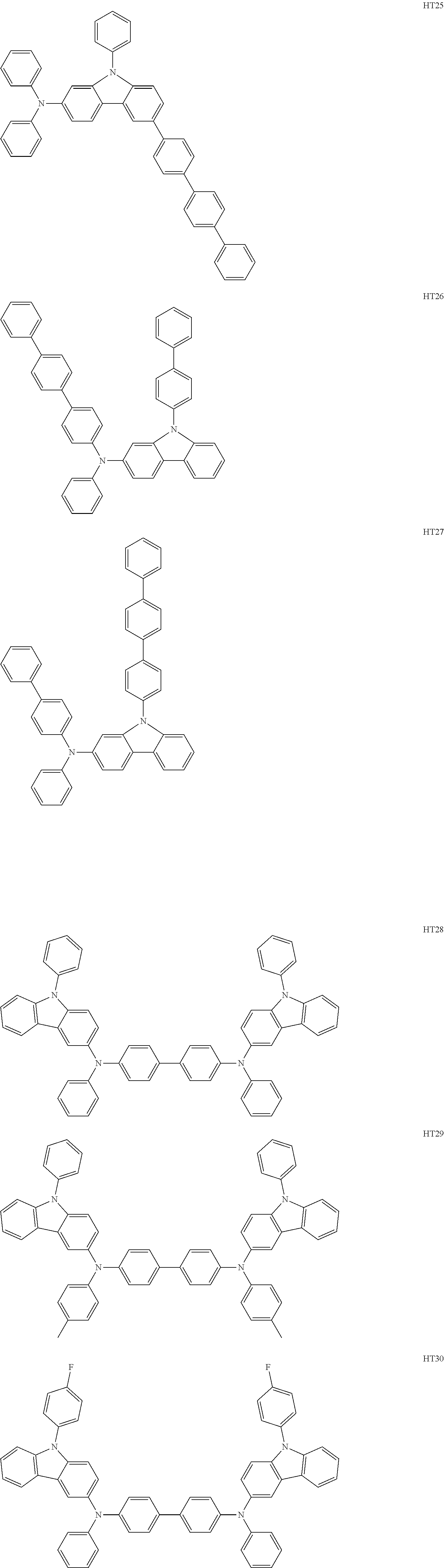

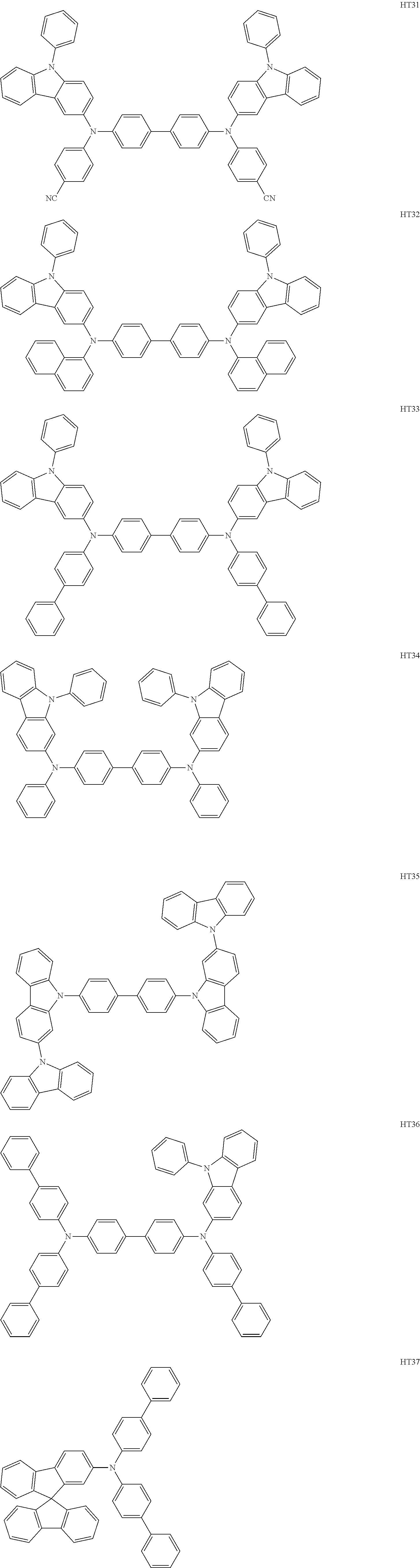

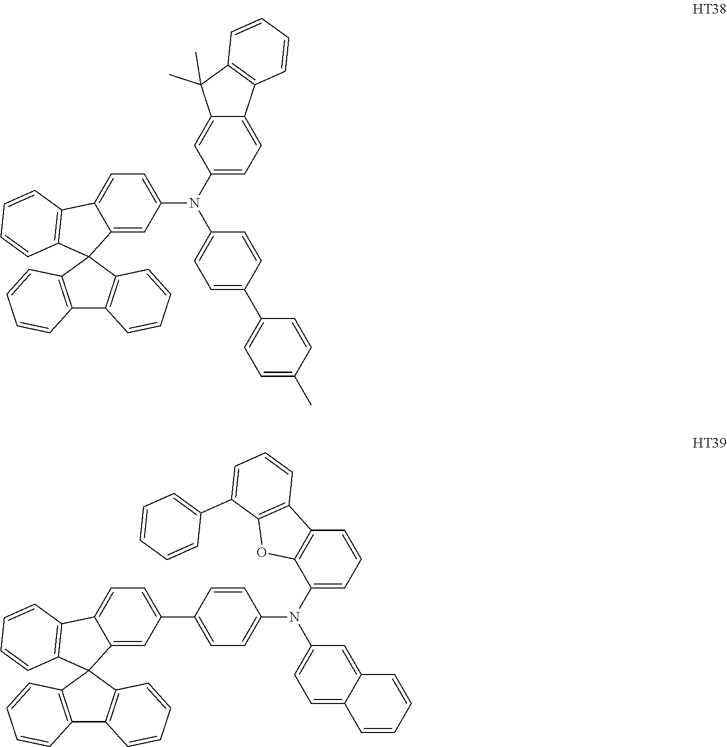

A thickness of the hole transport region may be in a range of about 100 Å to about 10,000 Å, for example, about 100 Å to about 1,000 Å. When the hole transport region includes at least one of a hole injection layer and a hole transport layer, a thickness of the hole injection layer may be in a range of about 100 Å to about 9,000 Å, for example, about 100 Å to about 1,000 Å, and a thickness of the hole transport layer may be in a range of about 50 Å to about 2,000 Å, for example about 100 Å to about 1,500 Å. When the thicknesses of the hole transport region, the hole injection layer, and the hole transport layer are within these ranges, satisfactory hole transporting characteristics may be obtained without a substantial increase in driving voltage.

The emission auxiliary layer may increase light-emission efficiency by compensating for an optical resonance distance according to the wavelength of light emitted by an emission layer, and the electron blocking layer may block the flow of electrons from an electron transport region. The emission auxiliary layer and the electron blocking layer may include the materials as described above.

[p-Dopant]

The hole transport region may further include, in addition to these materials, a charge-generation material for the improvement of conductive properties. The charge-generation material may be homogeneously or non-homogeneously dispersed in the hole transport region.

The charge-generation material may be, for example, a p-dopant.

In an example embodiment, the p-dopant may have a LUMO energy level of about −3.5 eV or less.

The p-dopant may include at least one selected from a quinone derivative, a metal oxide, and a cyano group-containing compound.

For example, the p-dopant may include at least one selected from:

a quinone derivative, such as tetracyanoquinodimethane (TCNQ) or 2,3,5,6-tetrafluoro-7,7,8,8-tetracyanoquinodimethane (F4-TCNQ);

a metal oxide, such as tungsten oxide or molybdenum oxide;

1,4,5,8,9,12-hexaazatriphenylene-hexacarbonitrile (HAT-CN); and

a compound represented by Formula 221:

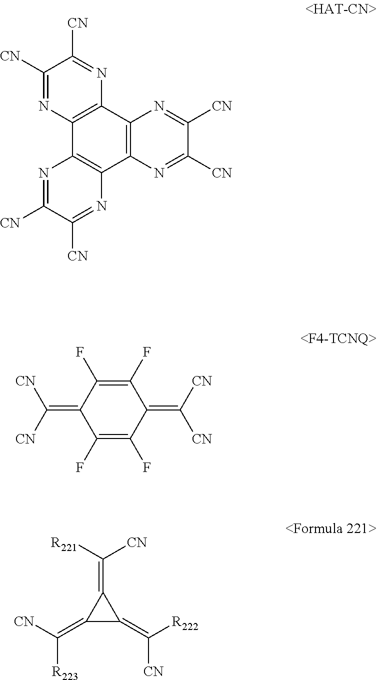

In Formula 221,

R₂₂₁ to R₂₂₃ may each independently be selected from a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₁-C₆₀ heteroaryl group, a substituted or unsubstituted monovalent non-aromatic condensed polycyclic group, and a substituted or unsubstituted monovalent non-aromatic condensed heteropolycyclic group, wherein at least one selected from R₂₂₁ to R₂₂₃ may have at least one substituent selected from a cyano group, —F, —Cl, —Br, —I, a C₁-C₂₀ alkyl group substituted with —F, a C₁-C₂₀ alkyl group substituted with —Cl, a C₁-C₂₀ alkyl group substituted with —Br, and a C₁-C₂₀ alkyl group substituted with —I.

[Emission Layer in Organic Layer 150]

When the organic light-emitting device 10 is a full-color organic light-emitting device, the emission layer may be patterned into a red emission layer, a green emission layer, or a blue emission layer, according to a sub-pixel. In an example embodiment, the emission layer may have a stacked structure of two or more layers selected from a red emission layer, a green emission layer, and a blue emission layer, in which the two or more layers contact each other or are separated from each other. In an example embodiment, the emission layer may include two or more materials selected from a red light-emitting material, a green light-emitting material, and a blue light-emitting material, in which the two or more materials are mixed with each other in a single layer to emit white light.

The emission layer may include a thermally delayed fluorescent emitter, which may include the heterocyclic compound.

The emission layer may include a host and a dopant. The dopant may include a thermally delayed fluorescent dopant, which may include the heterocyclic compound.

In the emission layer, an amount of the dopant may be in a range of about 0.01 parts to about 15 parts by weight based on 100 parts by weight of the host.

A thickness of the emission layer may be in a range of about 100 Å to about 1,000 Å, for example, about 200 Å to about 600 Å. When the thickness of the emission layer is within this range, excellent light-emission characteristics may be obtained without a substantial increase in driving voltage.

[Host in Emission Layer]

In an example embodiment, the host may include a compound represented by Formula 301:

[Ar₃₀₁]_{xb11}-[(L₃₀₁)_{xb1}-R₃₀₁]_{xb21} <Formula 301>

In Formula 301,

Ar₃₀₁ may be a substituted or unsubstituted C₅-C₆₀ carbocyclic group or a substituted or unsubstituted C₁-C₆₀ heterocyclic group,

xb11 may be 1, 2, or 3,

L₃₀₁ may each independently be selected from a substituted or unsubstituted C₃-C₁₀ cycloalkylene group, a substituted or unsubstituted C₁-C₁₀ heterocycloalkylene group, a substituted or unsubstituted C₃-C₁₀ cycloalkenylene group, a substituted or unsubstituted C₁-C₁₀ heterocycloalkenylene group, a substituted or unsubstituted C₆-C₆₀ arylene group, a substituted or unsubstituted C₁-C₆₀ heteroarylene group, a substituted or unsubstituted divalent non-aromatic condensed polycyclic group, and a substituted or unsubstituted divalent non-aromatic condensed heteropolycyclic group,

xb1 may be an integer from 0 to 5,

R₃₀₁ may be selected from deuterium, —F, —Cl, —Br, —I, a hydroxyl group, a cyano group, a nitro group, an amidino group, a hydrazino group, a hydrazono group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, a substituted or unsubstituted monovalent non-aromatic condensed heteropolycyclic group, —Si(Q₃₀₁)(Q₃₀₂)(Q₃₀₃), —N(Q₃₀₁)(Q₃₀₂), —B(Q₃₀₁)(Q₃₀₂), —C(═O)(Q₃₀₁), —S(═O)₂(Q₃₀₁), and —P(═O)(Q₃₀₁)(Q₃₀₂),

xb21 may be an integer from 1 to 5, and

Q₃₀₁ to Q₃₀₃ may each independently be selected from a C₁-C₁₀ alkyl group, a C₁-C₁₀ alkoxy group, a phenyl group, a biphenyl group, a terphenyl group, and a naphthyl group.

In an example embodiment, Ar₃₀₁ in Formula 301 may be selected from:

a naphthalene group, a fluorene group, a spiro-bifluorene group, a benzofluorene group, a dibenzofluorene group, a phenalene group, a phenanthrene group, an anthracene group, a fluoranthene group, a triphenylene group, a pyrene group, a chrysene group, a naphthacene group, a picene group, a perylene group, a pentaphene group, an indenoanthracene group, a dibenzofuran group, and a dibenzothiophene group; and

a naphthalene group, a fluorene group, a spiro-bifluorene group, a benzofluorene group, a dibenzofluorene group, a phenalene group, a phenanthrene group, an anthracene group, a fluoranthene group, a triphenylene group, a pyrene group, a chrysene group, a naphthacene group, a picene group, a perylene group, a pentaphene group, an indenoanthracene group, a dibenzofuran group, and a dibenzothiophene group, each substituted with at least one selected from deuterium, —F, —Cl, —Br, —I, a hydroxyl group, a cyano group, a nitro group, an amidino group, a hydrazino group, a hydrazono group, a C₁-C₂₀ alkyl group, a C₁-C₂₀ alkoxy group, a phenyl group, a biphenyl group, a terphenyl group, a naphthyl group, —Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), and —P(═O)(Q₃₁)(Q₃₂), and

Q₃₁ to Q₃₃ may each independently be selected from a C₁-C₁₀ alkyl group, a C₁-C₁₀ alkoxy group, a phenyl group, a biphenyl group, a terphenyl group, and a naphthyl group.

In Formula 301, when xb11 is two or more, two or more Ar₃₀₁(s) may be linked via a single bond.

In an example embodiment, the compound represented by Formula 301 may be represented by Formula 301-1 or 301-2:

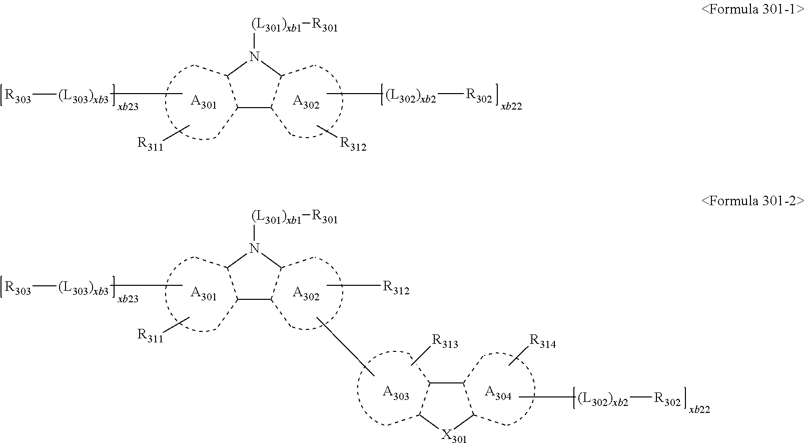

In Formulae 301-1 and 301-2,

A₃₀₁ to A₃₀₄ may each independently be selected from a benzene group, a naphthalene group, a phenanthrene group, a fluoranthene group, a triphenylene group, a pyrene group, a chrysene group, a pyridine group, a pyrimidine group, an indene group, a fluorene group, a spiro-bifluorene group, a benzofluorene group, a dibenzofluorene group, an indole group, a carbazole group, a benzocarbazole group, a dibenzocarbazole group, a furan group, a benzofuran group, a dibenzofuran group, a naphthofuran group, a benzonaphthofuran group, a dinaphthofuran group, a thiophene group, a benzothiophene group, a dibenzothiophene group, a naphthothiophene group, a benzonaphthothiophene group, and a dinaphthothiophene group,

X₃₀₁ may be O, S, or N-[(L₃₀₄)_{xb4}-R₃₀₄],

R₃₁₁ to R₃₁₄ may each independently be selected from hydrogen, deuterium, —F, —Cl, —Br, —I, a hydroxyl group, a cyano group, a nitro group, an amidino group, a hydrazino group, a hydrazono group, a C₁-C₂₀ alkyl group, a C₁-C₂₀ alkoxy group, a phenyl group, a biphenyl group, a terphenyl group, a naphthyl group —Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), and —P(═O)(Q₃₁)(Q₃₂),

xb22 and xb23 may each independently be 0, 1, or 2,

L₃₀₁, xb1, R₃₀₁, and Q₃₁ to Q₃₃ are the same as described above,

L₃₀₂ to L₃₀₄ may each independently be defined the same as L₃₀₁,

xb2 to xb4 may each independently be defined the same as xb1, and

R₃₀₂ to R₃₀₄ may each independently be defined the same as R₃₀₁.

For example, in Formulae 301, 301-1, and 301-2, L₃₀₁ to L₃₀₄ may each independently be selected from:

a phenylene group, a naphthylene group, a fluorenylene group, a spiro-bifluorenylene group, a benzofluorenylene group, a dibenzofluorenylene group, a phenanthrenylene group, an anthracenylene group, a fluoranthenylene group, a triphenylenylene group, a pyrenylene group, a chrysenylene group, a perylenylene group, a pentaphenylene group, a hexacenylene group, a pentacenylene group, a thiophenylene group, a furanylene group, a carbazolylene group, an indolylene group, an isoindolylene group, a benzofuranylene group, a benzothiophenylene group, a dibenzofuranylene group, a dibenzothiophenylene group, a benzocarbazolylene group, a dibenzocarbazolylene group, a dibenzosilolylene group, a pyridinylene group, an imidazolylene group, a pyrazolylene group, a thiazolylene group, an isothiazolylene group, an oxazolylene group, an isoxazolylene group, a thiadiazolylene group, an oxadiazolylene group, a pyrazinylene group, a pyrimidinylene group, a pyridazinylene group, a triazinylene group, a quinolinylene group, an isoquinolinylene group, a benzoquinolinylene group, a phthalazinylene group, a naphthyridinylene group, a quinoxalinylene group, a quinazolinylene group, a cinnolinylene group, a phenanthridinylene group, an acridinylene group, a phenanthrolinylene group, a phenazinylene group, a benzimidazolylene group, an isobenzothiazolylene group, a benzoxazolylene group, an isobenzoxazolylene group, a triazolylene group, a tetrazolylene group, an imidazopyridinylene group, an imidazopyrimidinylene group, and an azacarbazolylene group; and

a phenylene group, a naphthylene group, a fluorenylene group, a spiro-bifluorenylene group, a benzofluorenylene group, a dibenzofluorenylene group, a phenanthrenylene group, an anthracenylene group, a fluoranthenylene group, a triphenylenylene group, a pyrenylene group, a chrysenylene group, a perylenylene group, a pentaphenylene group, a hexacenylene group, a pentacenylene group, a thiophenylene group, a furanylene group, a carbazolylene group, an indolylene group, an isoindolylene group, a benzofuranylene group, a benzothiophenylene group, a dibenzofuranylene group, a dibenzothiophenylene group, a benzocarbazolylene group, a dibenzocarbazolylene group, a dibenzosilolylene group, a pyridinylene group, an imidazolylene group, a pyrazolylene group, a thiazolylene group, an isothiazolylene group, an oxazolylene group, an isoxazolylene group, a thiadiazolylene group, an oxadiazolylene group, a pyrazinylene group, a pyrimidinylene group, a pyridazinylene group, a triazinylene group, a quinolinylene group, an isoquinolinylene group, a benzoquinolinylene group, a phthalazinylene group, a naphthyridinylene group, a quinoxalinylene group, a quinazolinylene group, a cinnolinylene group, a phenanthridinylene group, an acridinylene group, a phenanthrolinylene group, a phenazinylene group, a benzimidazolylene group, an isobenzothiazolylene group, a benzoxazolylene group, an isobenzoxazolylene group, a triazolylene group, a tetrazolylene group, an imidazopyridinylene group, an imidazopyrimidinylene group, and an azacarbazolylene group, each substituted with at least one selected from deuterium, —F, —Cl, —Br, —I, a hydroxyl group, a cyano group, a nitro group, an amidino group, a hydrazino group, a hydrazono group, a C₁-C₂₀ alkyl group, a C₁-C₂₀ alkoxy group, a phenyl group, a biphenyl group, a terphenyl group, a naphthyl group, a fluorenyl group, a spiro-bifluorenyl group, a benzofluorenyl group, a dibenzofluorenyl group, a phenanthrenyl group, an anthracenyl group, a fluoranthenyl group, a triphenylenyl group, a pyrenyl group, a chrysenyl group, a perylenyl group, a pentaphenyl group, a hexacenyl group, a pentacenyl group, a thiophenyl group, a furanyl group, a carbazolyl group, an indolyl group, an isoindolyl group, a benzofuranyl group, a benzothiophenyl group, a dibenzofuranyl group, a dibenzothiophenyl group, a benzocarbazolyl group, a dibenzocarbazolyl group, a dibenzosilolyl group, a pyridinyl group, an imidazolyl group, a pyrazolyl group, a thiazolyl group, an isothiazolyl group, an oxazolyl group, an isoxazolyl group, a thiadiazolyl group, an oxadiazolyl group, a pyrazinyl group, a pyrimidinyl group, a pyridazinyl group, a triazinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a phenanthridinyl group, an acridinyl group, a phenanthrolinyl group, a phenazinyl group, a benzimidazolyl group, an isobenzothiazolyl group, a benzoxazolyl group, an isobenzoxazolyl group, a triazolyl group, a tetrazolyl group, an imidazopyridinyl group, an imidazopyrimidinyl group, an azacarbazolyl group, —Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), and —P(═O)(Q₃₁)(Q₃₂), and

Q₃₁ to Q₃₃ are the same as described above.

In an example embodiment, in Formulae 301, 301-1, and 301-2, R₃₀₁ to R₃₀₄ may each independently be selected from:

a phenyl group, a biphenyl group, a terphenyl group, a naphthyl group, a fluorenyl group, a spiro-bifluorenyl group, a benzofluorenyl group, a dibenzofluorenyl group, a phenanthrenyl group, an anthracenyl group, a fluoranthenyl group, a triphenylenyl group, a pyrenyl group, a chrysenyl group, a perylenyl group, a pentaphenyl group, a hexacenyl group, a pentacenyl group, a thiophenyl group, a furanyl group, a carbazolyl group, an indolyl group, an isoindolyl group, a benzofuranyl group, a benzothiophenyl group, a dibenzofuranyl group, a dibenzothiophenyl group, a benzocarbazolyl group, a dibenzocarbazolyl group, a dibenzosilolyl group, a pyridinyl group, an imidazolyl group, a pyrazolyl group, a thiazolyl group, an isothiazolyl group, an oxazolyl group, an isoxazolyl group, a thiadiazolyl group, an oxadiazolyl group, a pyrazinyl group, a pyrimidinyl group, a pyridazinyl group, a triazinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a phenanthridinyl group, an acridinyl group, a phenanthrolinyl group, a phenazinyl group, a benzimidazolyl group, an isobenzothiazolyl group, a benzoxazolyl group, an isobenzoxazolyl group, a triazolyl group, a tetrazolyl group, an imidazopyridinyl group, an imidazopyrimidinyl group, and an azacarbazolyl group; and

a phenyl group, a biphenyl group, a terphenyl group, a naphthyl group, a fluorenyl group, a spiro-bifluorenyl group, a benzofluorenyl group, a dibenzofluorenyl group, a phenanthrenyl group, an anthracenyl group, a fluoranthenyl group, a triphenylenyl group, a pyrenyl group, a chrysenyl group, a perylenyl group, a pentaphenyl group, a hexacenyl group, a pentacenyl group, a thiophenyl group, a furanyl group, a carbazolyl group, an indolyl group, an isoindolyl group, a benzofuranyl group, a benzothiophenyl group, a dibenzofuranyl group, a dibenzothiophenyl group, a benzocarbazolyl group, a dibenzocarbazolyl group, a dibenzosilolyl group, a pyridinyl group, an imidazolyl group, a pyrazolyl group, a thiazolyl group, an isothiazolyl group, an oxazolyl group, an isoxazolyl group, a thiadiazolyl group, an oxadiazolyl group, a pyrazinyl group, a pyrimidinyl group, a pyridazinyl group, a triazinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a phenanthridinyl group, an acridinyl group, a phenanthrolinyl group, a phenazinyl group, a benzimidazolyl group, an isobenzothiazolyl group, a benzoxazolyl group, an isobenzoxazolyl group, a triazolyl group, a tetrazolyl group, an imidazopyridinyl group, an imidazopyrimidinyl group, and an azacarbazolyl group, each substituted with at least one selected from deuterium, —F, —Cl, —Br, —I, a hydroxyl group, a cyano group, a nitro group, an amidino group, a hydrazino group, a hydrazono group, a C₁-C₂₀ alkyl group, a C₁-C₂₀ alkoxy group, a phenyl group, a biphenyl group, a terphenyl group, a naphthyl group, a fluorenyl group, a spiro-bifluorenyl group, a benzofluorenyl group, a dibenzofluorenyl group, a phenanthrenyl group, an anthracenyl group, a fluoranthenyl group, a triphenylenyl group, a pyrenyl group, a chrysenyl group, a perylenyl group, a pentaphenyl group, a hexacenyl group, a pentacenyl group, a thiophenyl group, a furanyl group, a carbazolyl group, an indolyl group, an isoindolyl group, a benzofuranyl group, a benzothiophenyl group, a dibenzofuranyl group, a dibenzothiophenyl group, a benzocarbazolyl group, a dibenzocarbazolyl group, a dibenzosilolyl group, a pyridinyl group, an imidazolyl group, a pyrazolyl group, a thiazolyl group, an isothiazolyl group, an oxazolyl group, an isoxazolyl group, a thiadiazolyl group, an oxadiazolyl group, a pyrazinyl group, a pyrimidinyl group, a pyridazinyl group, a triazinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a phenanthridinyl group, an acridinyl group, a phenanthrolinyl group, a phenazinyl group, a benzimidazolyl group, an isobenzothiazolyl group, a benzoxazolyl group, an isobenzoxazolyl group, a triazolyl group, a tetrazolyl group, an imidazopyridinyl group, an imidazopyrimidinyl group, an azacarbazolyl group, —Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), and —P(═O)(Q₃₁)(Q₃₂), and

Q₃₁ to Q₃₃ are the same as described above.

In an example embodiment, the host may include an alkaline earth metal complex. For example, the host may be selected from a Be complex (for example, Compound H55), a Mg complex, and a Zn complex.

The host may include at least one selected from 9,10-di-(2-naphthyl)anthracene (ADN), 2-methyl-9,10-bis(naphthalen-2-yl)anthracene (MADN), 9,10-di-(2-naphthyl)-2-t-butyl-anthracene (TBADN), 4,4′-bis(N-carbazolyl)-1,1′-biphenyl (CBP), 1,3-di-9-carbazolylbenzene (mCP), 1,3,5-tri(carbazol-9-yl)benzene (TCP), and Compounds H1 to H55:

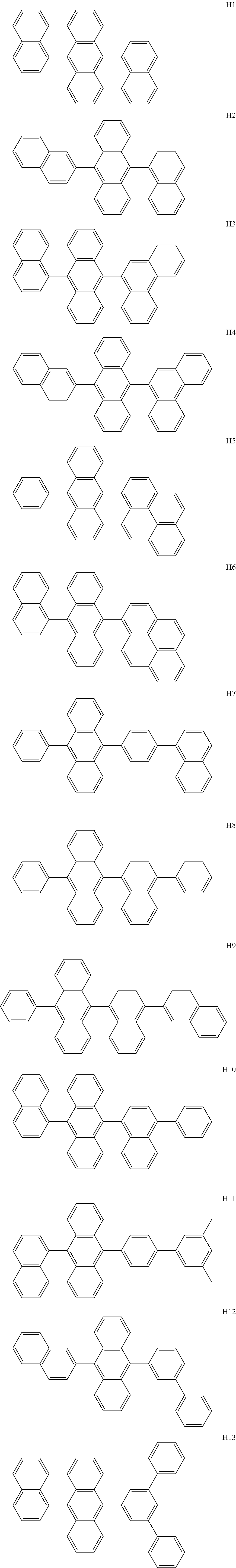

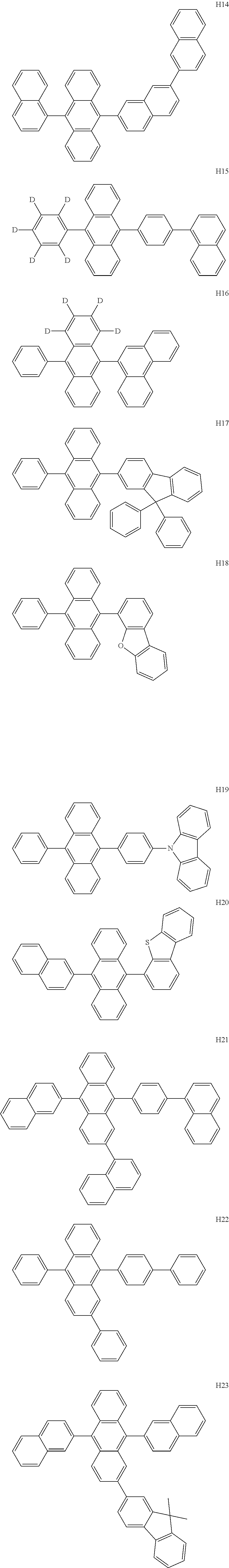

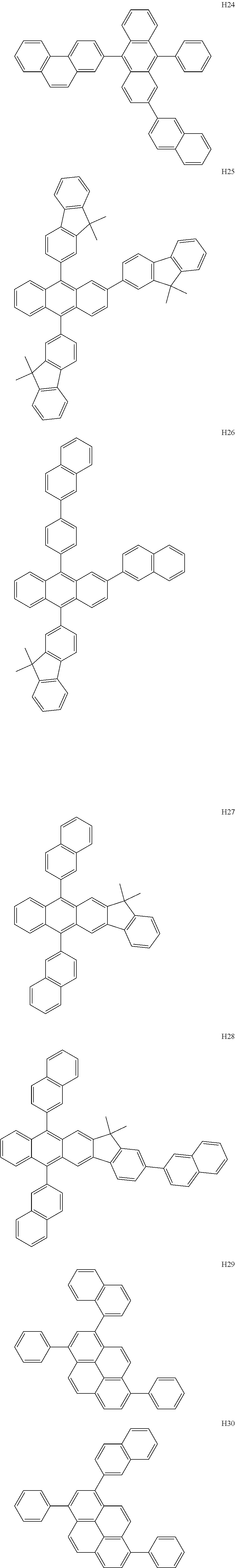

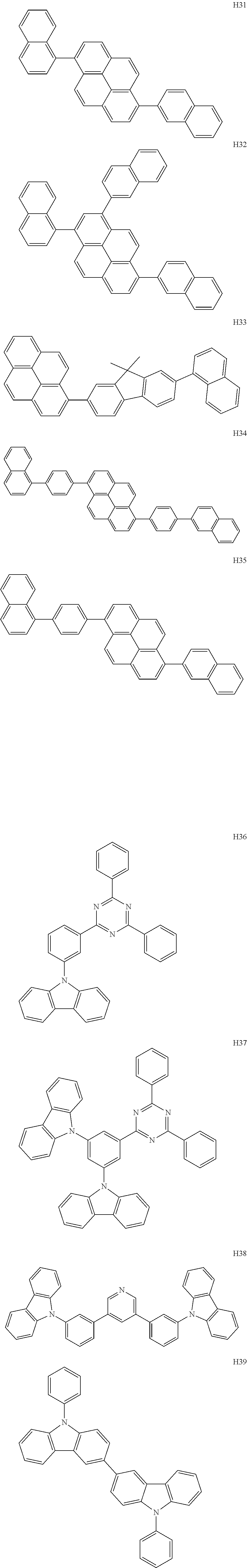

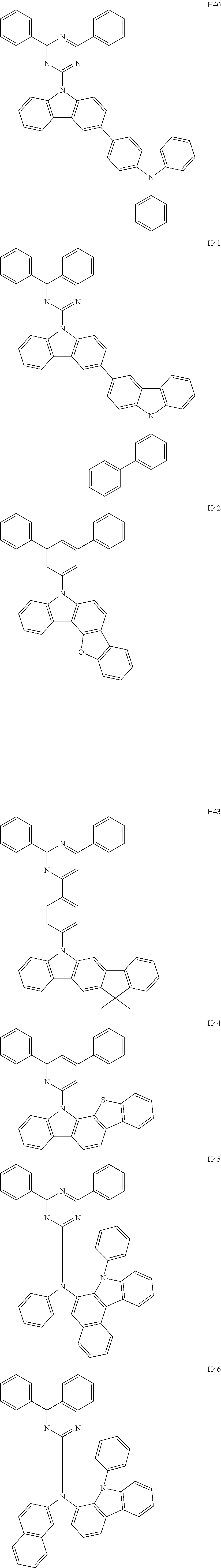

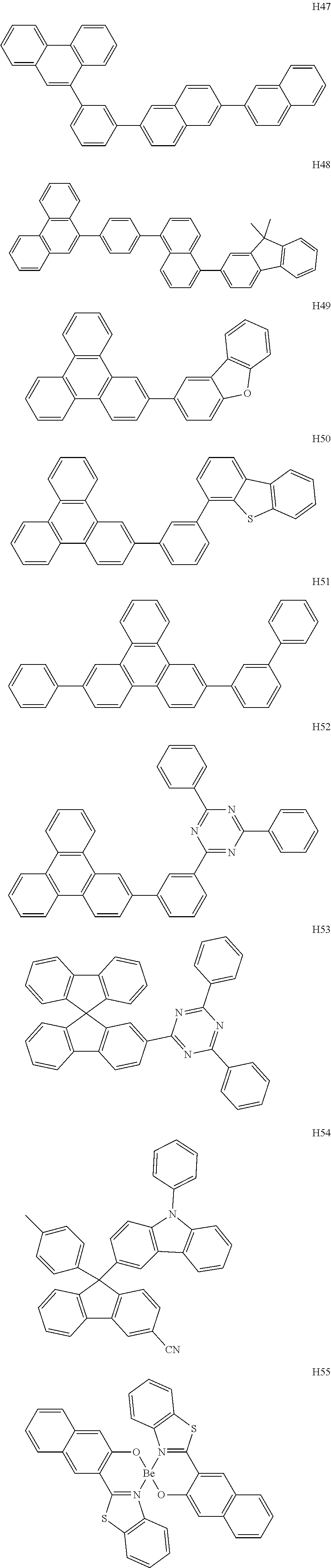

[Phosphorescent Dopant Included in Emission Layer in Organic Layer 150]

The emission layer may further include a phosphorescent dopant. The phosphorescent dopant may include an organometallic complex represented by Formula 401:

M(L₄₀₁)_{xc1}(L₄₀₂)_{xc2} <Formula 401>

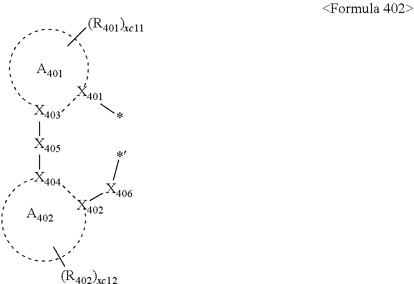

In Formulae 401 and 402,

M may be selected from iridium (Ir), platinum (Pt), palladium (Pd), osmium (Os), titanium (Ti), zirconium (Zr), hafnium (Hf), europium (Eu), terbium (Tb), rhodium (Rh), and thulium (Tm),

L₄₀₁ may be selected from ligands represented by Formula 402, and xc1 may be 1, 2, or 3, wherein, when xc1 is two or more, two or more L₄₀₁(s) may be identical to or different from each other,

L₄₀₂ may be an organic ligand, and xc2 may be an integer from 0 to 4, wherein, when xc2 is two or more, two or more L₄₀₂(s) may be identical to or different from each other,

X₄₀₁ to X₄₀₄ may each independently be nitrogen or carbon,

X₄₀₁ and X₄₀₃ may be linked via a single bond or a double bond, and X₄₀₂ and X₄₀₄ may be linked via a single bond or a double bond,

A₄₀₁ and A₄₀₂ may each independently be selected from a C₅-C₆₀ carbocyclic group or a C₁-C₆₀ heterocyclic group,

X₄₀₅ may be a single bond, *—O—*′, *—S—*′, *—C(═O)—*′, *—N(Q₄₁₁)-*′, *—C(Q₄₁₁)(Q₄₁₂)-*′, *—C(Q₄₁₁)═C(Q₄₁₂)-*′, *—C(Q₄₁₁)═*′, or *═C═*′, wherein Q₄₁₁ and Q₄₁₂ may be hydrogen, deuterium, a C₁-C₂₀ alkyl group, a C₁-C₂₀ alkoxy group, a phenyl group, a biphenyl group, a terphenyl group, or a naphthyl group,

X₄₀₆ may be a single bond, O, or S,

R₄₀₁ and R₄₀₂ may each independently be selected from hydrogen, deuterium, —F, —Cl, —Br, —I, a hydroxyl group, a cyano group, a nitro group, an amidino group, a hydrazino group, a hydrazono group, a substituted or unsubstituted C₁-C₂₀ alkyl group, a substituted or unsubstituted C₁-C₂₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, a substituted or unsubstituted monovalent non-aromatic condensed heteropolycyclic group, —Si(Q₄₀₁)(Q₄₀₂)(Q₄₀₃), —N(Q₄₀₁)(Q₄₀₂), —B(Q₄₀₁)(Q₄₀₂), —C(═O)(Q₄₀₁), —S(═O)₂(Q₄₀₁), and —P(═O)(Q₄₀₁)(Q₄₀₂), wherein Q₄₀₁ to Q₄₀₃ may each independently be selected from a C₁-C₁₀ alkyl group, a C₁-C₁₀ alkoxy group, a C₆-C₂₀ aryl group, and a C₁-C₂₀ heteroaryl group,

xc11 and xc12 may each independently be an integer from 0 to 10, and

* and *′ in Formula 402 each indicate a binding site to M in Formula 401.

In an example embodiment, A₄₀₁ and A₄₀₂ in Formula 402 may each independently be selected from a benzene group, a naphthalene group, a fluorene group, a spiro-bifluorene group, an indene group, a pyrrole group, a thiophene group, a furan group, an imidazole group, a pyrazole group, a thiazole group, an isothiazole group, an oxazole group, an isoxazole group, a pyridine group, a pyrazine group, a pyrimidine group, a pyridazine group, a quinoline group, an isoquinoline group, a benzoquinoline group, a quinoxaline group, a quinazoline group, a carbazole group, a benzimidazole group, a benzofuran group, a benzothiophene group, an isobenzothiophene group, a benzoxazole group, an isobenzoxazole group, a triazole group, a tetrazole group, an oxadiazole group, a triazine group, a dibenzofuran group, and a dibenzothiophene group.

In an example embodiment, in Formula 402, i) X₄₀₁ may be nitrogen, and X₄₀₂ may be carbon, or ii) X₄₀₁ and X₄₀₂ may each be nitrogen at the same time.

In an example embodiment, R₄₀₁ and R₄₀₂ in Formula 402 may each independently be selected from:

hydrogen, deuterium, —F, —Cl, —Br, —I, a hydroxyl group, a cyano group, a nitro group, an amidino group, a hydrazino group, a hydrazono group, a C₁-C₂₀ alkyl group, and a C₁-C₂₀ alkoxy group;

a C₁-C₂₀ alkyl group and a C₁-C₂₀ alkoxy group, each substituted with at least one selected from deuterium, —F, —Cl, —Br, —I, a hydroxyl group, a cyano group, a nitro group, an amidino group, a hydrazino group, a hydrazono group, a phenyl group, a naphthyl group, a cyclopentyl group, a cyclohexyl group, an adamantly group, a norbornanyl group, and a norbornenyl group;

a cyclopentyl group, a cyclohexyl group, an adamantly group, a norbornanyl group, a norbornenyl group, a phenyl group, a biphenyl group, a terphenyl group, a naphthyl group, a fluorenyl group, a pyridinyl group, a pyrazinyl group, a pyrimidinyl group, a pyridazinyl group, a triazinyl group, a quinolinyl group, an isoquinolinyl group, a quinoxalinyl group, a quinazolinyl group, a carbazolyl group, a dibenzofuranyl group, and a dibenzothiophenyl group;

a cyclopentyl group, a cyclohexyl group, an adamantly group, a norbornanyl group, a norbornenyl group a phenyl group, a biphenyl group, a terphenyl group, a naphthyl group, a fluorenyl group, a pyridinyl group, a pyrazinyl group, a pyrimidinyl group, a pyridazinyl group, a triazinyl group, a quinolinyl group, an isoquinolinyl group, a quinoxalinyl group, a quinazolinyl group, a carbazolyl group, a dibenzofuranyl group, and a dibenzothiophenyl group, each substituted with at least one selected from deuterium, —F, —Cl, —Br, —I, a hydroxyl group, a cyano group, a nitro group, an amidino group, a hydrazino group, a hydrazono group, a C₁-C₂₀ alkyl group, a C₁-C₂₀ alkoxy group, a cyclopentyl group, a cyclohexyl group, an adamantly group, a norbornanyl group, a norbornenyl group, a phenyl group, a biphenyl group, a terphenyl group, a naphthyl group, a fluorenyl group, a pyridinyl group, a pyrazinyl group, a pyrimidinyl group, a pyridazinyl group, a triazinyl group, a quinolinyl group, an isoquinolinyl group, a quinoxalinyl group, a quinazolinyl group, a carbazolyl group, a dibenzofuranyl group, and a dibenzothiophenyl group; and

—Si(Q₄₀₁)(Q₄₀₂)(Q₄₀₃), —N(Q₄₀₁)(Q₄₀₂), —B(Q₄₀₁)(Q₄₀₂), —C(═O)(Q₄₀₁), —S(═O)₂(Q₄₀₁), and —P(═O)(Q₄₀₁)(Q₄₀₂), and

Q₄₀₁ to Q₄₀₃ may each independently be selected from a C₁-C₁₀ alkyl group, a C₁-C₁₀ alkoxy group, a phenyl group, a biphenyl group, and a naphthyl group.

In an example embodiment, when xc1 in Formula 401 is two or more, two A₄₀₁(s) in two or more L₄₀₁(s) may be linked via X₄₀₇, which is a linking group, or two A₄₀₂(s) in two or more L₄₀₁(s) may be linked via X₄₀₈, which is a linking group (see Compounds PD1 to PD4 and PD7). X₄₀₇ and X₄₀₈ may each independently be a single bond, *—O—*′, *—S—*′, *—C(═O)—*′, *—N(Q₄₁₃)-*′, *—C(Q₄₁₃)(Q₄₁₄)-*′, or *—C(Q₄₁₃)═C(Q₄₁₄)-*′ (wherein Q₄₁₃ and Q₄₁₄ may each independently be hydrogen, deuterium, a C₁-C₂₀ alkyl group, a C₁-C₂₀ alkoxy group, a phenyl group, a biphenyl group, a terphenyl group, or a naphthyl group).

L₄₀₂ in Formula 401 may be a monovalent, divalent, or trivalent organic ligand. For example, L₄₀₂ may be selected from halogen, diketone (for example, acetylacetonate), carboxylic acid (for example, picolinate), —C(═O)—, isonitrile, —CN, and phosphorus (for example, phosphine, or phosphite).

In an example embodiment, the phosphorescent dopant may be selected from, for example, Compounds PD1 to PD25:

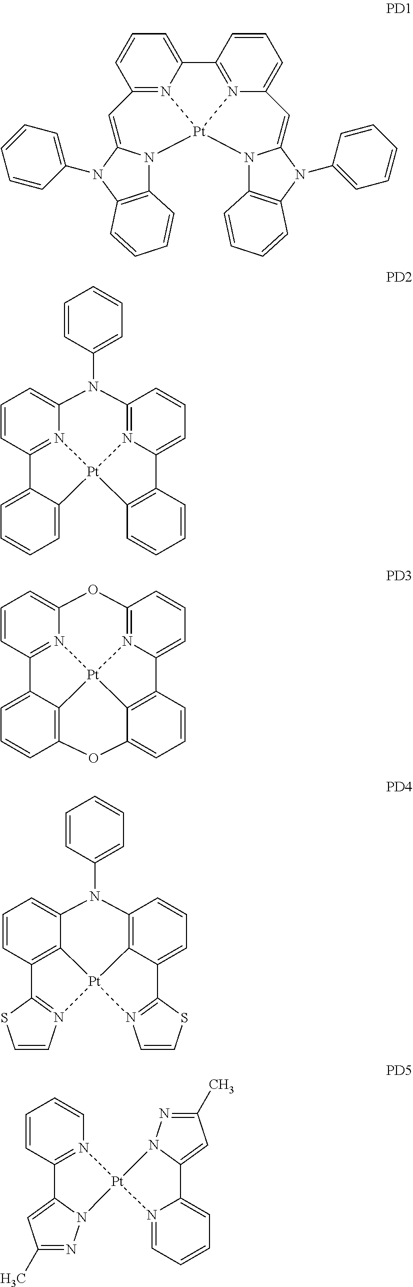

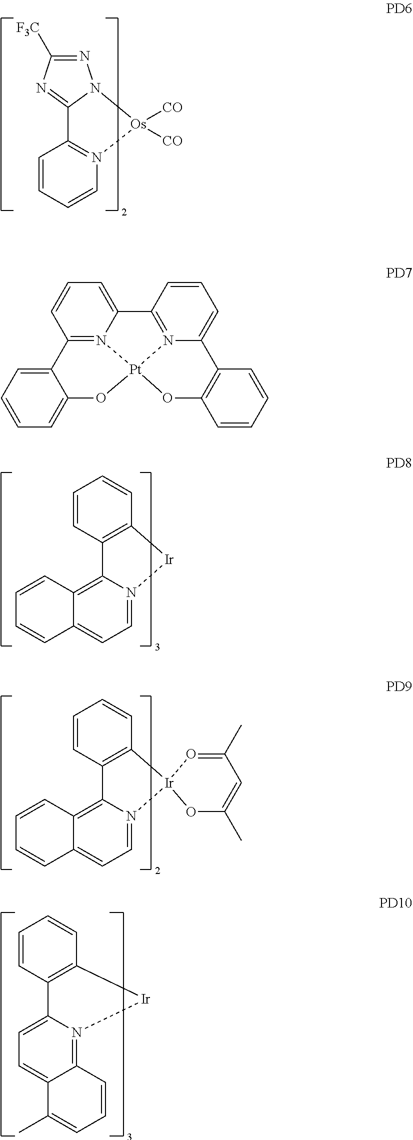

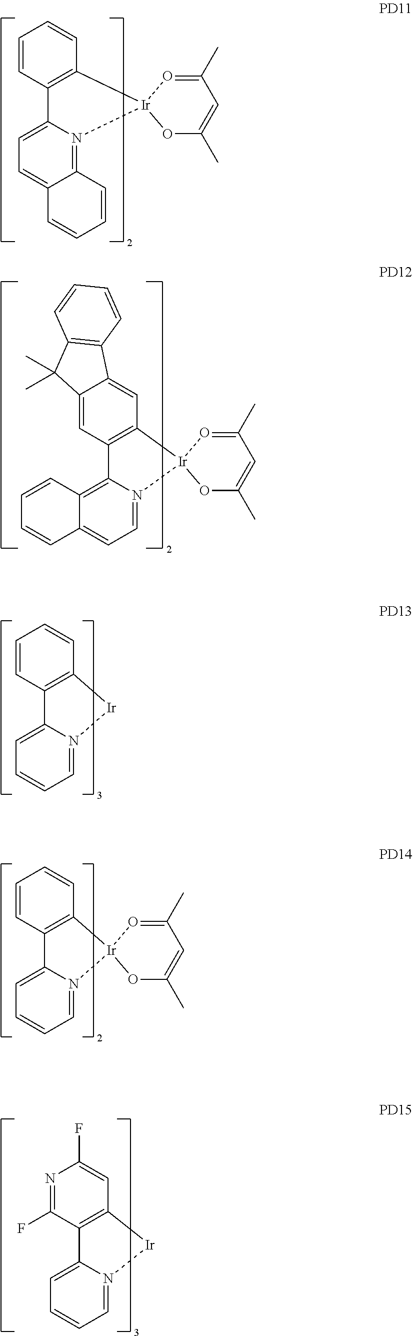

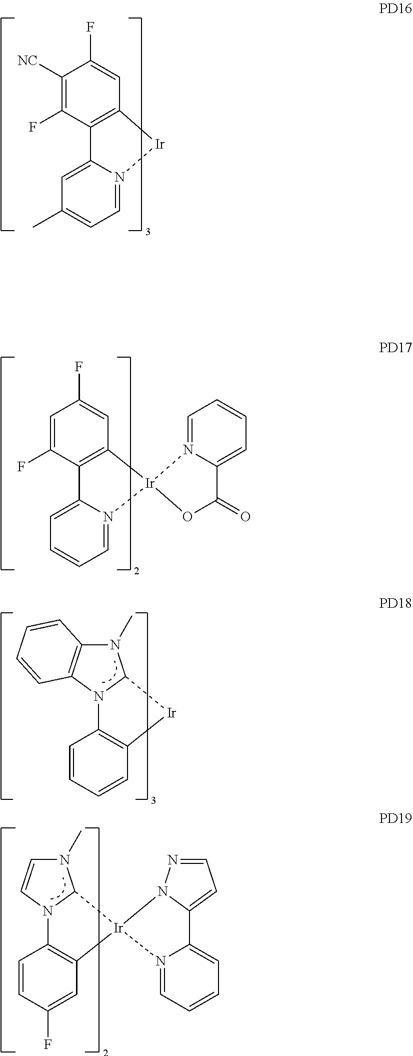

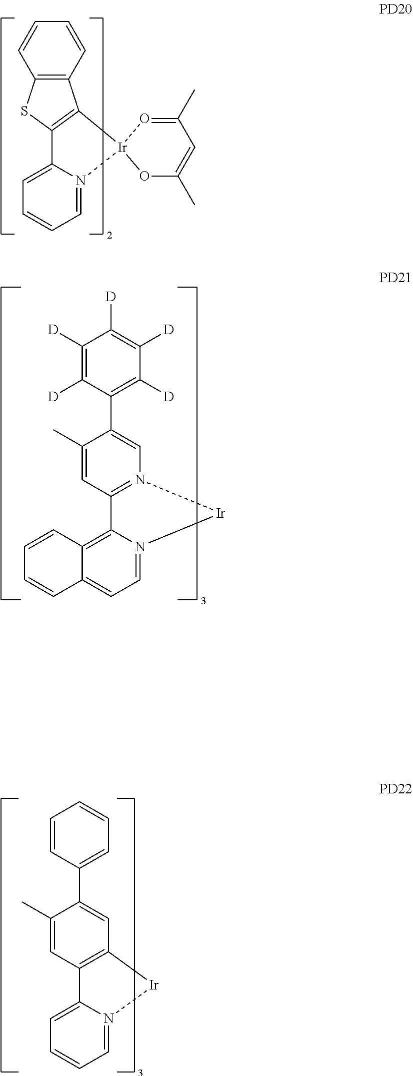

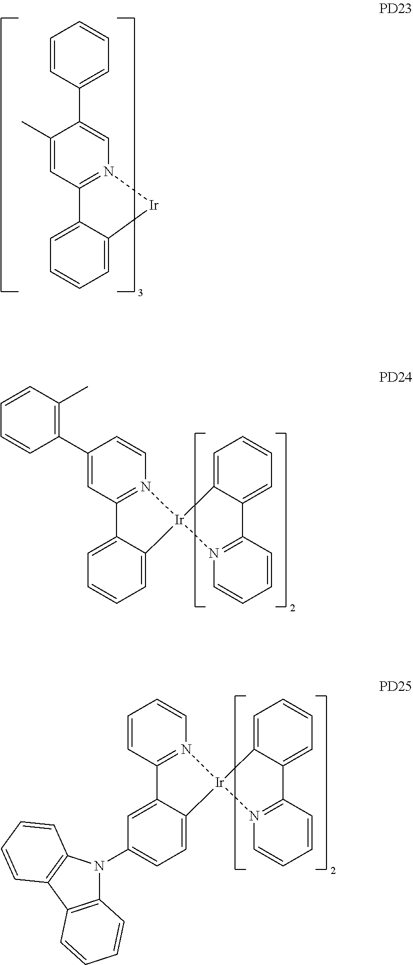

[Fluorescent Dopant in Emission Layer]

The fluorescent dopant may include a compound represented by Formula 501.

The fluorescent dopant may include an arylamine compound or a styrylamine compound.

In an example embodiment, the fluorescent dopant may include a compound represented by Formula 501 below.

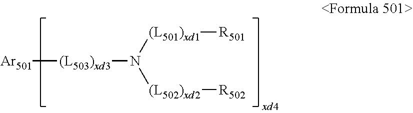

In Formula 501,

Ar₅₀₁ may be a substituted or unsubstituted C₅-C₆₀ carbocyclic group or a substituted or unsubstituted C₁-C₆₀ heterocyclic group,

L₅₀₁ to L₅₀₃ may each independently be selected from a substituted or unsubstituted C₃-C₁₀ cycloalkylene group, a substituted or unsubstituted C₁-C₁₀ heterocycloalkylene group, a substituted or unsubstituted C₃-C₁₀ cycloalkenylene group, a substituted or unsubstituted C₁-C₁₀ heterocycloalkenylene group, a substituted or unsubstituted C₆-C₆₀ arylene group, a substituted or unsubstituted C₁-C₆₀ heteroarylene group, a substituted or unsubstituted divalent non-aromatic condensed polycyclic group, and a substituted or unsubstituted divalent non-aromatic condensed heteropolycyclic group,

xd1 to xd3 may each independently be an integer from 0 to 3,

R₅₀₁ and R₅₀₂ may each independently be selected from a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, and a substituted or unsubstituted monovalent non-aromatic condensed heteropolycyclic group, and

xd4 may be an integer from 1 to 6.

In an example embodiment, Ar₅₀₁ in Formula 501 may be selected from:

a naphthalene group, a heptalene group, a fluorene group, a spiro-bifluorene group, a benzofluorene group, a dibenzofluorene group, a phenalene group, a phenanthrene group, an anthracene group, a fluoranthene group, a triphenylene group, a pyrene group, a chrysene group, a naphthacene group, a picene group, a perylene group, a pentaphene group, an indenoanthracene group, and an indenophenanthrene group; and

a naphthalene group, a heptalene group, a fluorene group, a spiro-bifluorene group, a benzofluorene group, a dibenzofluorene group, a phenalene group, a phenanthrene group, an anthracene group, a fluoranthene group, a triphenylene group, a pyrene group, a chrysene group, a naphthacene group, a picene group, a perylene group, a pentaphene group, an indenoanthracene group, and an indenophenanthrene group, each substituted with at least one selected from deuterium, —F, —Cl, —Br, —I, a hydroxyl group, a cyano group, a nitro group, an amidino group, a hydrazino group, a hydrazono group, a C₁-C₂₀ alkyl group, a C₁-C₂₀ alkoxy group, a phenyl group, a biphenyl group, a terphenyl group, and a naphthyl group.

In an example embodiment, L₅₀₁ to L₅₀₃ in Formula 501 may each independently be selected from:

a phenylene group, a naphthylene group, a fluorenylene group, a spiro-bifluorenylene group, a benzofluorenylene group, a dibenzofluorenylene group, a phenanthrenylene group, an anthracenylene group, a fluoranthenylene group, a triphenylenylene group, a pyrenylene group, a chrysenylene group, a perylenylene group, a pentaphenylene group, a hexacenylene group, a pentacenylene group, a thiophenylene group, a furanylene group, a carbazolylene group, an indolylene group, an isoindolylene group, a benzofuranylene group, a benzothiophenylene group, a dibenzofuranylene group, a dibenzothiophenylene group, a benzocarbazolylene group, a dibenzocarbazolylene group, a dibenzosilolylene group, and a pyridinylene group; and

a phenylene group, a naphthylene group, a fluorenylene group, a spiro-bifluorenylene group, a benzofluorenylene group, a dibenzofluorenylene group, a phenanthrenylene group, an anthracenylene group, a fluoranthenylene group, a triphenylenylene group, a pyrenylene group, a chrysenylene group, a perylenylene group, a pentaphenylene group, a hexacenylene group, a pentacenylene group, a thiophenylene group, a furanylene group, a carbazolylene group, an indolylene group, an isoindolylene group, a benzofuranylene group, a benzothiophenylene group, a dibenzofuranylene group, a dibenzothiophenylene group, a benzocarbazolylene group, a dibenzocarbazolylene group, a dibenzosilolylene group, and a pyridinylene group, each substituted with at least one selected from deuterium, —F, —Cl, —Br, —I, a hydroxyl group, a cyano group, a nitro group, an amidino group, a hydrazino group, a hydrazono group, a C₁-C₂₀ alkyl group, a C₁-C₂₀ alkoxy group, a phenyl group, a biphenyl group, a terphenyl group, a naphthyl group, a fluorenyl group, a spiro-bifluorenyl group, a benzofluorenyl group, a dibenzofluorenyl group, a phenanthrenyl group, an anthracenyl group, a fluoranthenyl group, a triphenylenyl group, a pyrenyl group, a chrysenyl group, a perylenyl group, a pentaphenyl group, a hexacenyl group, a pentacenyl group, a thiophenyl group, a furanyl group, a carbazolyl group, an indolyl group, an isoindolyl group, a benzofuranyl group, a benzothiophenyl group, a dibenzofuranyl group, a dibenzothiophenyl group, a benzocarbazolyl group, a dibenzocarbazolyl group, a dibenzosilolyl group, and a pyridinyl group.

In an example embodiment, R₅₀₁ and R₅₀₂ in Formula 501 may each independently be selected from:

a phenyl group, a biphenyl group, a terphenyl group, a naphthyl group, a fluorenyl group, a spiro-bifluorenyl group, a benzofluorenyl group, a dibenzofluorenyl group, a phenanthrenyl group, an anthracenyl group, a fluoranthenyl group, a triphenylenyl group, a pyrenyl group, a chrysenyl group, a perylenyl group, a pentaphenyl group, a hexacenyl group, a pentacenyl group, a thiophenyl group, a furanyl group, a carbazolyl group, an indolyl group, an isoindolyl group, a benzofuranyl group, a benzothiophenyl group, a dibenzofuranyl group, a dibenzothiophenyl group, a benzocarbazolyl group, a dibenzocarbazolyl group, a dibenzosilolyl group, and a pyridinyl group; and

a phenyl group, a biphenyl group, a terphenyl group, a naphthyl group, a fluorenyl group, a spiro-bifluorenyl group, a benzofluorenyl group, a dibenzofluorenyl group, a phenanthrenyl group, an anthracenyl group, a fluoranthenyl group, a triphenylenyl group, a pyrenyl group, a chrysenyl group, a perylenyl group, a pentaphenyl group, a hexacenyl group, a pentacenyl group, a thiophenyl group, a furanyl group, a carbazolyl group, an indolyl group, an isoindolyl group, a benzofuranyl group, a benzothiophenyl group, a dibenzofuranyl group, a dibenzothiophenyl group, a benzocarbazolyl group, a dibenzocarbazolyl group, a dibenzosilolyl group, and a pyridinyl group, each substituted with at least one selected from deuterium, —F, —Cl, —Br, —I, a hydroxyl group, a cyano group, a nitro group, an amidino group, a hydrazino group, a hydrazono group, a C₁-C₂₀ alkyl group, a C₁-C₂₀ alkoxy group, a phenyl group, a biphenyl group, a terphenyl group, a naphthyl group, a fluorenyl group, a spiro-bifluorenyl group, a benzofluorenyl group, a dibenzofluorenyl group, a phenanthrenyl group, an anthracenyl group, a fluoranthenyl group, a triphenylenyl group, a pyrenyl group, a chrysenyl group, a perylenyl group, a pentaphenyl group, a hexacenyl group, a pentacenyl group, a thiophenyl group, a furanyl group, a carbazolyl group, an indolyl group, an isoindolyl group, a benzofuranyl group, a benzothiophenyl group, a dibenzofuranyl group, a dibenzothiophenyl group, a benzocarbazolyl group, a dibenzocarbazolyl group, a dibenzosilolyl group, a pyridinyl group, and —Si(Q₃₁)(Q₃₂)(Q₃₃), and

Q₃₁ to Q₃₃ may each independently be selected from a C₁-C₁₀ alkyl group, a C₁-C₁₀ alkoxy group, a phenyl group, a biphenyl group, a terphenyl group, and a naphthyl group.

In an example embodiment, xd4 in Formula 501 may be 2.

For example, the fluorescent dopant may be selected from Compounds FD1 to FD22:

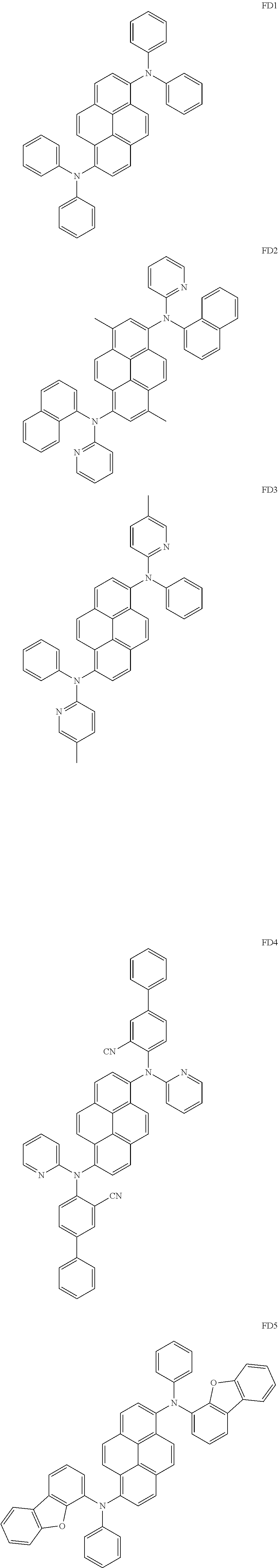

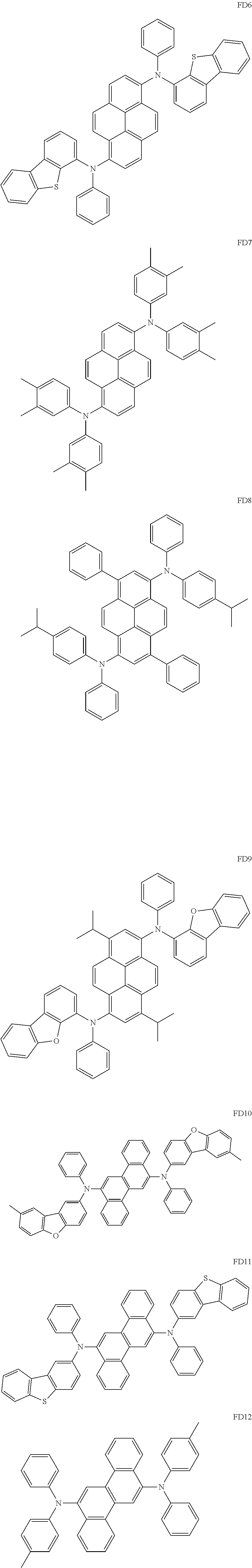

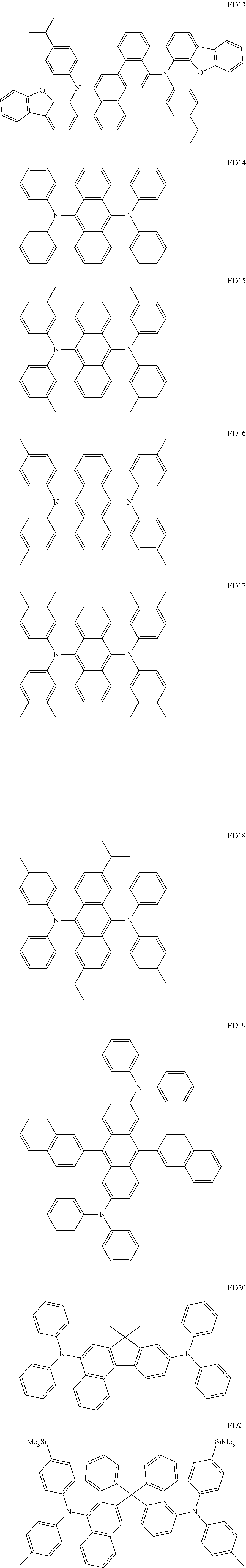

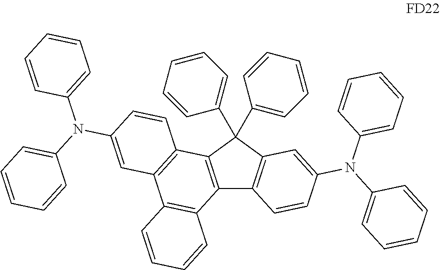 [SMILES: c1ccc(N(c2ccccc2)c2ccc3c(c2)C(c2ccccc2)(c2ccccc2)c2c-3c3ccccc3c3cc(N(c4ccccc4)c4ccccc4)ccc23)cc1]

In an example embodiment, the fluorescent dopant may be selected from the following compounds:

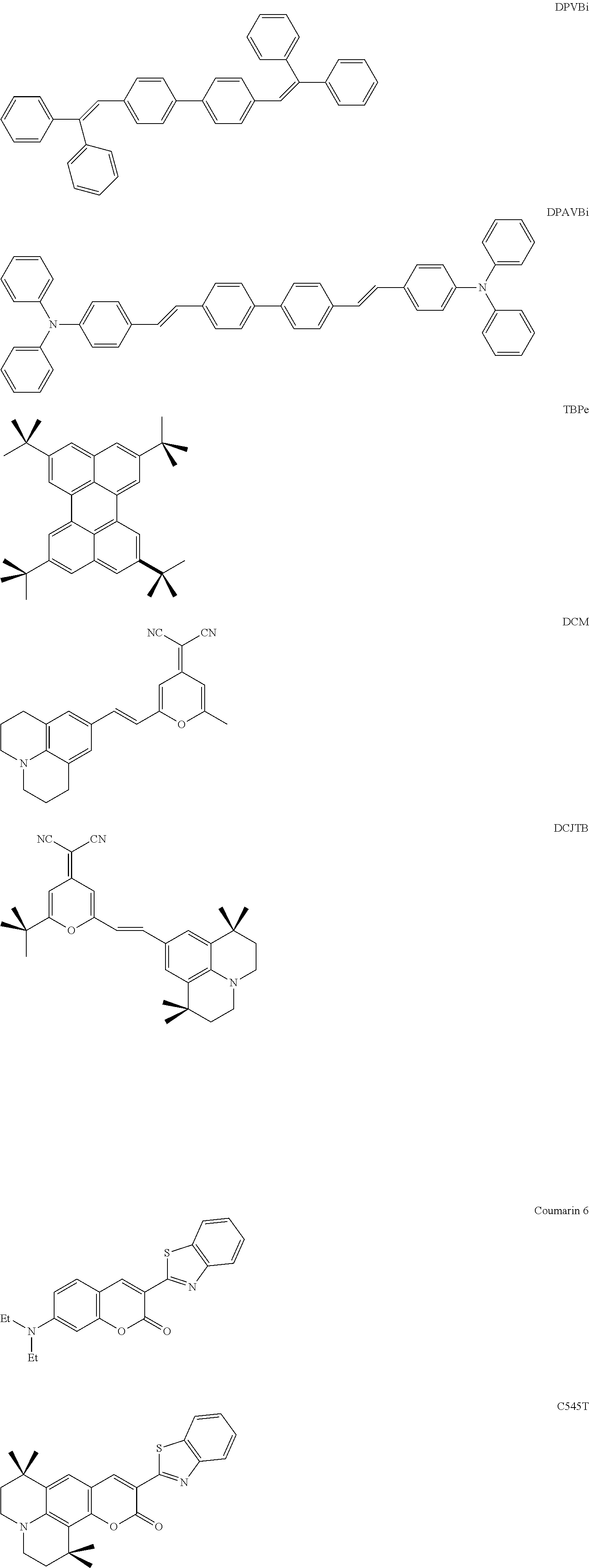

[Electron Transport Region in Organic Layer 150]

The electron transport region may have i) a single-layered structure including a single layer including a single material, ii) a single-layered structure including a single layer including a plurality of different materials, or iii) a multi-layered structure having a plurality of layers including a plurality of different materials.

The electron transport region may include at least one selected from a buffer layer, a hole blocking layer, an electron control layer, an electron transport layer, and an electron injection layer.

For example, the electron transport region may have an electron transport layer/electron injection layer structure, a hole blocking layer/electron transport layer/electron injection layer structure, an electron control layer/electron transport layer/electron injection layer structure, or a buffer layer/electron transport layer/electron injection layer structure, wherein for each structure, constituting layers are sequentially stacked from an emission layer.

The electron transport region (for example, a buffer layer, a hole blocking layer, an electron control layer, or an electron transport layer in the electron transport region) may include a metal-free compound containing at least one 7 electron-depleted nitrogen-containing ring.

The term “π electron-depleted nitrogen-containing ring” indicates a C₁-C₆₀ heterocyclic group having at least one *—N═*′ moiety as a ring-forming moiety.

For example, the “n electron-depleted nitrogen-containing ring” may be i) a 5-membered to 7-membered heteromonocyclic group having at least one *—N═*′ moiety, ii) a heteropolycyclic group in which two or more 5-membered to 7-membered heteromonocyclic groups each having at least one *—N═*′ moiety are condensed with each other, or iii) a heteropolycyclic group in which at least one of 5-membered to 7-membered heteromonocyclic groups, each having at least one *—N═*′ moiety, is condensed with at least one C₅-C₆₀ carbocyclic group.

Examples of the π electron-depleted nitrogen-containing ring include an imidazole group, a pyrazole group, a thiazole group, an isothiazole group, an oxazole group, an isoxazole group, a pyridine group, a pyrazine group, a pyrimidine group, a pyridazinyl, an indazole group, a purine group, a quinoline group, an isoquinoline group, a benzoquinoline group, a phthalazine group, a naphthyridine group, a quinoxaline group, a quinazoline group, a cinnoline group, a phenanthridine group, an acridine group, a phenanthroline group, a phenazine group, a benzimidazole group, an isobenzothiazole group, a benzoxazole group, an isobenzoxazole group, a triazole group, a tetrazole group, an oxadiazole group, a triazine group, a thiadiazol group, an imidazopyridine group, an imidazopyrimidine group, and an azacarbazole group.

For example, the electron transport region may include a compound represented by Formula 601:

[Ar₆₀₁]ₓₑ₁₁-[(L₆₀₁)ₓₑ₁-R₆₀₁]ₓₑ₂₁ <Formula 601>

In Formula 601,

Ar₆₀₁ may be a substituted or unsubstituted C₅-C₆₀ carbocyclic group or a substituted or unsubstituted C₁-C₆₀ heterocyclic group,

xe11 may be 1, 2, or 3,

L₆₀₁ may be selected from a substituted or unsubstituted C₃-C₁₀ cycloalkylene group, a substituted or unsubstituted C₁-C₁₀ heterocycloalkylene group, a substituted or unsubstituted C₃-C₁₀ cycloalkenylene group, a substituted or unsubstituted C₁-C₁₀ heterocycloalkenylene group, a substituted or unsubstituted C₆-C₆₀ arylene group, a substituted or unsubstituted C₁-C₆₀ heteroarylene group, a substituted or unsubstituted divalent non-aromatic condensed polycyclic group, and a substituted or unsubstituted divalent non-aromatic condensed heteropolycyclic group;

xe1 may be an integer from 0 to 5,

R₆₀₁ may be selected from a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, a substituted or unsubstituted monovalent non-aromatic condensed heteropolycyclic group, —Si(Q₆₀₁)(Q₆₀₂)(Q₆₀₃), —C(═O)(Q₆₀₁), —S(═O)₂(Q₆₀₁), and —P(═O)(Q₆₀₁)(Q₆₀₂),

Q₆₀₁ to Q₆₀₃ may each independently be a C₁-C₁₀ alkyl group, a C₁-C₁₀ alkoxy group, a phenyl group, a biphenyl group, a terphenyl group, or a naphthyl group, and

xe21 may be an integer from 1 to 5.

In an example embodiment, at least one of Ar₆₀₁(s) in the number of xe11 and R₆₀₁(S) in the number of xe21 may include the n electron-depleted nitrogen-containing ring.

In an example embodiment, Ar₆₀₁ in Formula 601 may be selected from:

a benzene group, a naphthalene group, a fluorene group, a spiro-bifluorene group, a benzofluorene group, a dibenzofluorene group, a phenalene group, a phenanthrene group, an anthracene group, a fluoranthene group, a triphenylene group, a pyrene group, a chrysene group, a naphthacene group, a picene group, a perylene group, a pentaphene group, an indenoanthracene group, a dibenzofuran group, a dibenzothiophene group, a carbazole group, an imidazole group, a pyrazole group, a thiazole group, an isothiazole group, an oxazole group, an isoxazole group, a pyridine group, a pyrazine group, a pyrimidine group, a pyridazine group, an indazole group, a purine group, a quinoline group, an isoquinoline group, a benzoquinoline group, a phthalazine group, a naphthyridine group, a quinoxaline group, a quinazoline group, a cinnoline group, a phenanthridine group, an acridine group, a phenanthroline group, a phenazine group, a benzimidazole group, an isobenzothiazole group, a benzoxazole group, an isobenzoxazole group, a triazole group, a tetrazole group, an oxadiazole group, a triazine group, a thiadiazole group, an imidazopyridine group, an imidazopyrimidine group, and an azacarbazole group; and

a benzene group, a naphthalene group, a fluorene group, a spiro-bifluorene group, a benzofluorene group, a dibenzofluorene group, a phenalene group, a phenanthrene group, an anthracene group, a fluoranthene group, a triphenylene group, a pyrene group, a chrysene group, a naphthacene group, a picene group, a perylene group, a pentaphene group, an indenoanthracene group, a dibenzofuran group, a dibenzothiophene group, a carbazole group, an imidazole group, a pyrazole group, a thiazole group, an isothiazole group, an oxazole group, an isoxazole group, a pyridine group, a pyrazine group, a pyrimidine group, a pyridazine group, an indazole group, a purine group, a quinoline group, an isoquinoline group, a benzoquinoline group, a phthalazine group, a naphthyridine group, a quinoxaline group, a quinazoline group, a cinnoline group, a phenanthridine group, an acridine group, a phenanthroline group, a phenazine group, a benzimidazole group, an isobenzothiazole group, a benzoxazole group, an isobenzoxazole group, a triazole group, a tetrazole group, an oxadiazole group, a triazine group, a thiadiazole group, an imidazopyridine group, an imidazopyrimidine group, and an azacarbazole group, each substituted with at least one selected from deuterium, —F, —Cl, —Br, —I, a hydroxyl group, a cyano group, a nitro group, an amidino group, a hydrazino group, a hydrazono group, a C₁-C₂₀ alkyl group, a C₁-C₂₀ alkoxy group, a phenyl group, a biphenyl group, a terphenyl group, a naphthyl group, —Si(Q₃₁)(Q₃₂)(Q₃₃), —S(═O)₂(Q₃₁), and —P(═O)(Q₃₁)(Q₃₂), and

Q₃₁ to Q₃₃ may each independently be selected from a C₁-C₁₀ alkyl group, a C₁-C₁₀ alkoxy group, a phenyl group, a biphenyl group, a terphenyl group, and a naphthyl group.

When xe11 in Formula 601 is two or more, two or more Ar₆₀₁(s) may be linked via a single bond.

In an example embodiment, Ar₆₀₁ in Formula 601 may be an anthracene group.

In an example embodiment, a compound represented by Formula 601 may be represented by Formula 601-1:

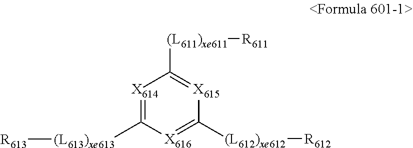

In Formula 601-1,

X₆₁₄ may be N or C(R₆₁₄), X₆₁₅ may be N or C(R₆₁₅), and X₆₁₆ may be N or C(R₆₁₆), wherein at least one selected from X₆₁₄ to X₆₁₆ may be N,

L₆₁₁ to L₆₁₃ may each independently be defined the same as with L₆₀₁,

xe611 to xe613 may each independently be defined the same as xe1,

R₆₁₁ to R₆₁₃ may each independently be defined the same as R₆₀₁, and

R₆₁₄ to R₆₁₆ may each independently be selected from hydrogen, deuterium, —F, —Cl, —Br, —I, a hydroxyl group, a cyano group, a nitro group, an amidino group, a hydrazino group, a hydrazono group, a C₁-C₂₀ alkyl group, a C₁-C₂₀ alkoxy group, a phenyl group, a biphenyl group, a terphenyl group, and a naphthyl group.

In an example embodiment, L₆₀₁ and L₆₁₁ to L₆₁₃ in Formulae 601 and 601-1 may each independently be selected from:

a phenylene group, a naphthylene group, a fluorenylene group, a spiro-bifluorenylene group, a benzofluorenylene group, a dibenzofluorenylene group, a phenanthrenylene group, an anthracenylene group, a fluoranthenylene group, a triphenylenylene group, a pyrenylene group, a chrysenylene group, a perylenylene group, a pentaphenylene group, a hexacenylene group, a pentacenylene group, a thiophenylene group, a furanylene group, a carbazolylene group, an indolylene group, an isoindolylene group, a benzofuranylene group, a benzothiophenylene group, a dibenzofuranylene group, a dibenzothiophenylene group, a benzocarbazolylene group, a dibenzocarbazolylene group, a dibenzosilolylene group, a pyridinylene group, an imidazolylene group, a pyrazolylene group, a thiazolylene group, an isothiazolylene group, an oxazolylene group, an isoxazolylene group, a thiadiazolylene group, an oxadiazolylene group, a pyrazinylene group, a pyrimidinylene group, a pyridazinylene group, a triazinylene group, a quinolinylene group, an isoquinolinylene group, a benzoquinolinylene group, a phthalazinylene group, a naphthyridinylene group, a quinoxalinylene group, a quinazolinylene group, a cinnolinylene group, a phenanthridinylene group, an acridinylene group, a phenanthrolinylene group, a phenazinylene group, a benzimidazolylene group, an isobenzothiazolylene group, a benzoxazolylene group, an isobenzoxazolylene group, a triazolylene group, a tetrazolylene group, an imidazopyridinylene group, an imidazopyrimidinylene group, and an azacarbazolylene group; and

a phenylene group, a naphthylene group, a fluorenylene group, a spiro-bifluorenylene group, a benzofluorenylene group, a dibenzofluorenylene group, a phenanthrenylene group, an anthracenylene group, a fluoranthenylene group, a triphenylenylene group, a pyrenylene group, a chrysenylene group, a perylenylene group, a pentaphenylene group, a hexacenylene group, a pentacenylene group, a thiophenylene group, a furanylene group, a carbazolylene group, an indolylene group, an isoindolylene group, a benzofuranylene group, a benzothiophenylene group, a dibenzofuranylene group, a dibenzothiophenylene group, a benzocarbazolylene group, a dibenzocarbazolylene group, a dibenzosilolylene group, a pyridinylene group, an imidazolylene group, a pyrazolylene group, a thiazolylene group, an isothiazolylene group, an oxazolylene group, an isoxazolylene group, a thiadiazolylene group, an oxadiazolylene group, a pyrazinylene group, a pyrimidinylene group, a pyridazinylene group, a triazinylene group, a quinolinylene group, an isoquinolinylene group, a benzoquinolinylene group, a phthalazinylene group, a naphthyridinylene group, a quinoxalinylene group, a quinazolinylene group, a cinnolinylene group, a phenanthridinylene group, an acridinylene group, a phenanthrolinylene group, a phenazinylene group, a benzimidazolylene group, an isobenzothiazolylene group, a benzoxazolylene group, an isobenzoxazolylene group, a triazolylene group, a tetrazolylene group, an imidazopyridinylene group, an imidazopyrimidinylene group, and an azacarbazolylene group, each substituted with at least one selected from deuterium, —F, —Cl, —Br, —I, a hydroxyl group, a cyano group, a nitro group, an amidino group, a hydrazino group, a hydrazono group, a C₁-C₂₀ alkyl group, a C₁-C₂₀ alkoxy group, a phenyl group, a biphenyl group, a terphenyl group, a naphthyl group, a fluorenyl group, a spiro-bifluorenyl group, a benzofluorenyl group, a dibenzofluorenyl group, a phenanthrenyl group, an anthracenyl group, a fluoranthenyl group, a triphenylenyl group, a pyrenyl group, a chrysenyl group, a perylenyl group, a pentaphenyl group, a hexacenyl group, a pentacenyl group, a thiophenyl group, a furanyl group, a carbazolyl group, an indolyl group, an isoindolyl group, a benzofuranyl group, a benzothiophenyl group, a dibenzofuranyl group, a dibenzothiophenyl group, a benzocarbazolyl group, a dibenzocarbazolyl group, a dibenzosilolyl group, a pyridinyl group, an imidazolyl group, a pyrazolyl group, a thiazolyl group, an isothiazolyl group, an oxazolyl group, an isoxazolyl group, a thiadiazolyl group, an oxadiazolyl group, a pyrazinyl group, a pyrimidinyl group, a pyridazinyl group, a triazinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a phenanthridinyl group, an acridinyl group, a phenanthrolinyl group, a phenazinyl group, a benzimidazolyl group, an isobenzothiazolyl group, a benzoxazolyl group, an isobenzoxazolyl group, a triazolyl group, a tetrazolyl group, an imidazopyridinyl group, an imidazopyrimidinyl group, and an azacarbazolyl group.

In an example embodiment, xe1 and xe611 to xe613 in Formulae 601 and 601-1 may each independently be 0, 1, or 2.

In an example embodiment, R₆₀₁ and R₆₁₁ to R₆₁₃ in Formulae 601 and 601-1 may each independently be selected from:

a phenyl group, a biphenyl group, a terphenyl group, a naphthyl group, a fluorenyl group, a spiro-bifluorenyl group, a benzofluorenyl group, a dibenzofluorenyl group, a phenanthrenyl group, an anthracenyl group, a fluoranthenyl group, a triphenylenyl group, a pyrenyl group, a chrysenyl group, a perylenyl group, a pentaphenyl group, a hexacenyl group, a pentacenyl group, a thiophenyl group, a furanyl group, a carbazolyl group, an indolyl group, an isoindolyl group, a benzofuranyl group, a benzothiophenyl group, a dibenzofuranyl group, a dibenzothiophenyl group, a benzocarbazolyl group, a dibenzocarbazolyl group, a dibenzosilolyl group, a pyridinyl group, an imidazolyl group, a pyrazolyl group, a thiazolyl group, an isothiazolyl group, an oxazolyl group, an isoxazolyl group, a thiadiazolyl group, an oxadiazolyl group, a pyrazinyl group, a pyrimidinyl group, a pyridazinyl group, a triazinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a phenanthridinyl group, an acridinyl group, a phenanthrolinyl group, a phenazinyl group, a benzimidazolyl group, an isobenzothiazolyl group, a benzoxazolyl group, an isobenzoxazolyl group, a triazolyl group, a tetrazolyl group, an imidazopyridinyl group, an imidazopyrimidinyl group, and an azacarbazolyl group;

a phenyl group, a biphenyl group, a terphenyl group, a naphthyl group, a fluorenyl group, a spiro-bifluorenyl group, a benzofluorenyl group, a dibenzofluorenyl group, a phenanthrenyl group, an anthracenyl group, a fluoranthenyl group, a triphenylenyl group, a pyrenyl group, a chrysenyl group, a perylenyl group, a pentaphenyl group, a hexacenyl group, a pentacenyl group, a thiophenyl group, a furanyl group, a carbazolyl group, an indolyl group, an isoindolyl group, a benzofuranyl group, a benzothiophenyl group, a dibenzofuranyl group, a dibenzothiophenyl group, a benzocarbazolyl group, a dibenzocarbazolyl group, a dibenzosilolyl group, a pyridinyl group, an imidazolyl group, a pyrazolyl group, a thiazolyl group, an isothiazolyl group, an oxazolyl group, an isoxazolyl group, a thiadiazolyl group, an oxadiazolyl group, a pyrazinyl group, a pyrimidinyl group, a pyridazinyl group, a triazinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a phenanthridinyl group, an acridinyl group, a phenanthrolinyl group, a phenazinyl group, a benzimidazolyl group, an isobenzothiazolyl group, a benzoxazolyl group, an isobenzoxazolyl group, a triazolyl group, a tetrazolyl group, an imidazopyridinyl group, an imidazopyrimidinyl group, and an azacarbazolyl group, each substituted with at least one selected from deuterium, —F, —Cl, —Br, —I, a hydroxyl group, a cyano group, a nitro group, an amidino group, a hydrazino group, a hydrazono group, a C₁-C₂₀ alkyl group, a C₁-C₂₀ alkoxy group, a phenyl group, a biphenyl group, a terphenyl group, a naphthyl group, a fluorenyl group, a spiro-bifluorenyl group, a benzofluorenyl group, a dibenzofluorenyl group, a phenanthrenyl group, an anthracenyl group, a fluoranthenyl group, a triphenylenyl group, a pyrenyl group, a chrysenyl group, a perylenyl group, a pentaphenyl group, a hexacenyl group, a pentacenyl group, a thiophenyl group, a furanyl group, a carbazolyl group, an indolyl group, an isoindolyl group, a benzofuranyl group, a benzothiophenyl group, a dibenzofuranyl group, a dibenzothiophenyl group, a benzocarbazolyl group, a dibenzocarbazolyl group, a dibenzosilolyl group, a pyridinyl group, an imidazolyl group, a pyrazolyl group, a thiazolyl group, an isothiazolyl group, an oxazolyl group, an isoxazolyl group, a thiadiazolyl group, an oxadiazolyl group, a pyrazinyl group, a pyrimidinyl group, a pyridazinyl group, a triazinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a phenanthridinyl group, an acridinyl group, a phenanthrolinyl group, a phenazinyl group, a benzimidazolyl group, an isobenzothiazolyl group, a benzoxazolyl group, an isobenzoxazolyl group, a triazolyl group, a tetrazolyl group, an imidazopyridinyl group, an imidazopyrimidinyl group, and an azacarbazolyl group; and

—S(═O)₂(Q₆₀₁) and —P(═O)(Q₆₀₁)(Q₆₀₂), and

Q₆₀₁ and Q₆₀₂ are the same as described above.

The electron transport region may include at least one compound selected from Compounds ET1 to ET36:

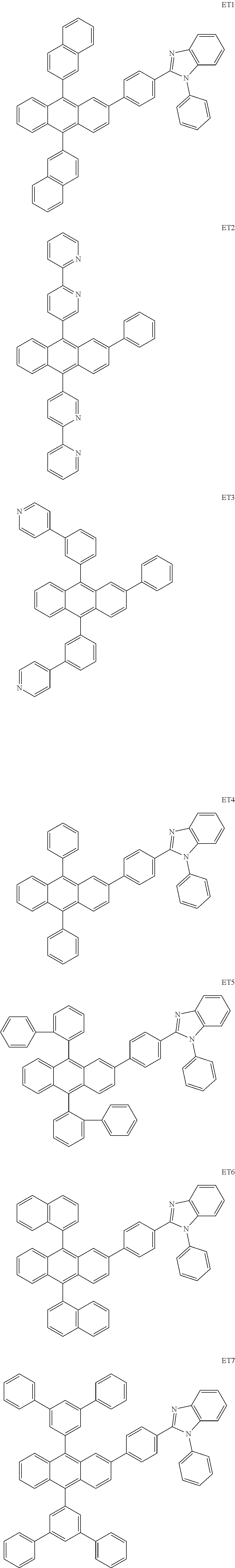

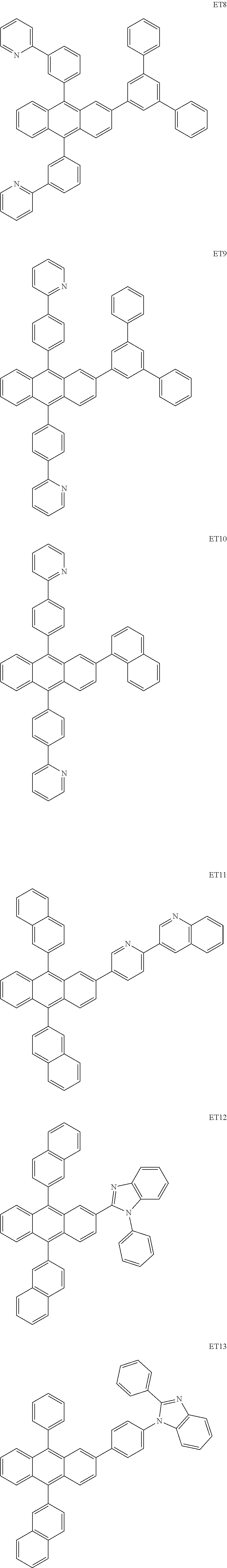

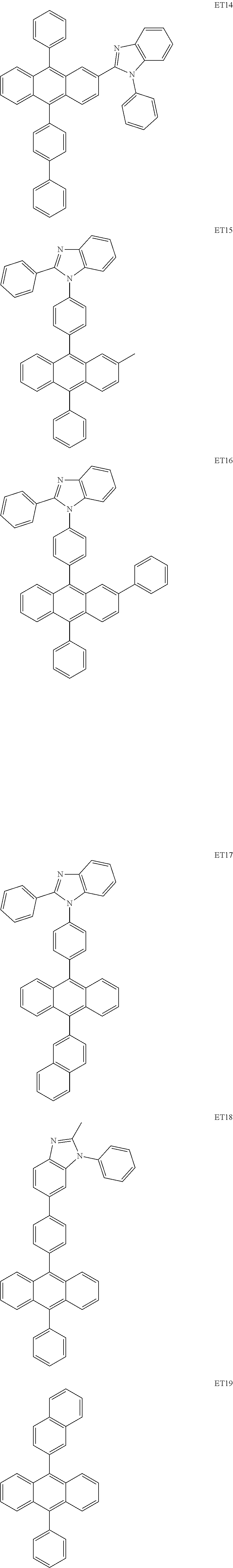

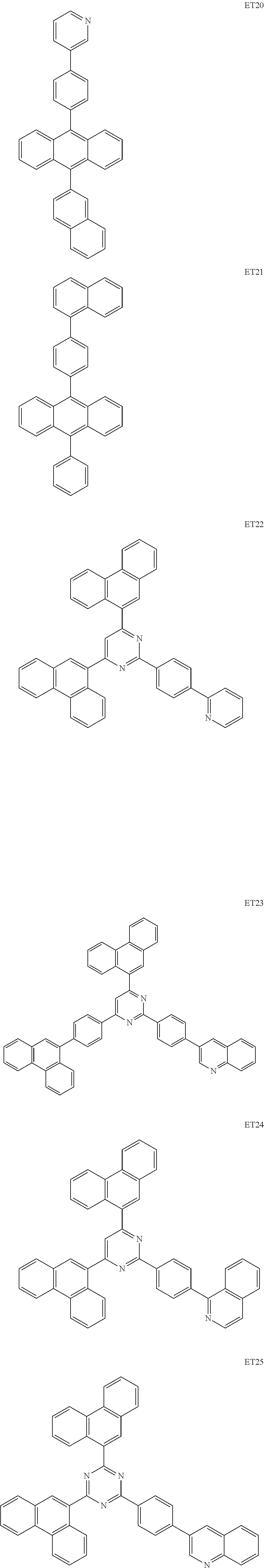

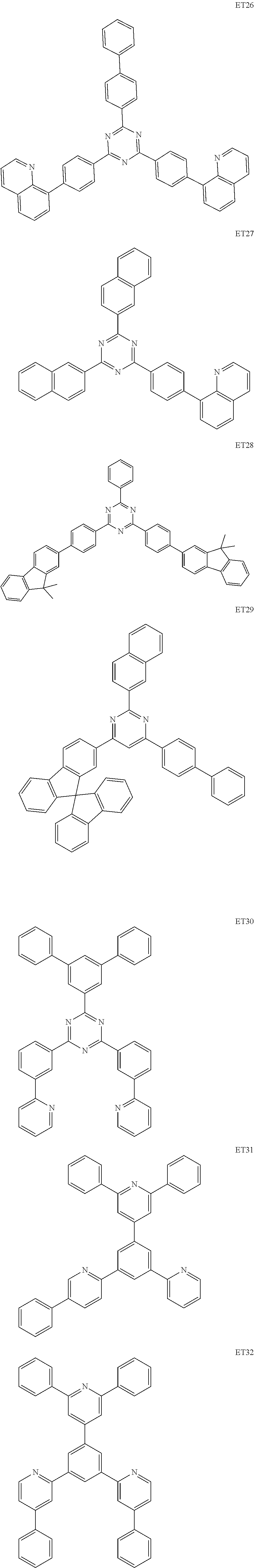

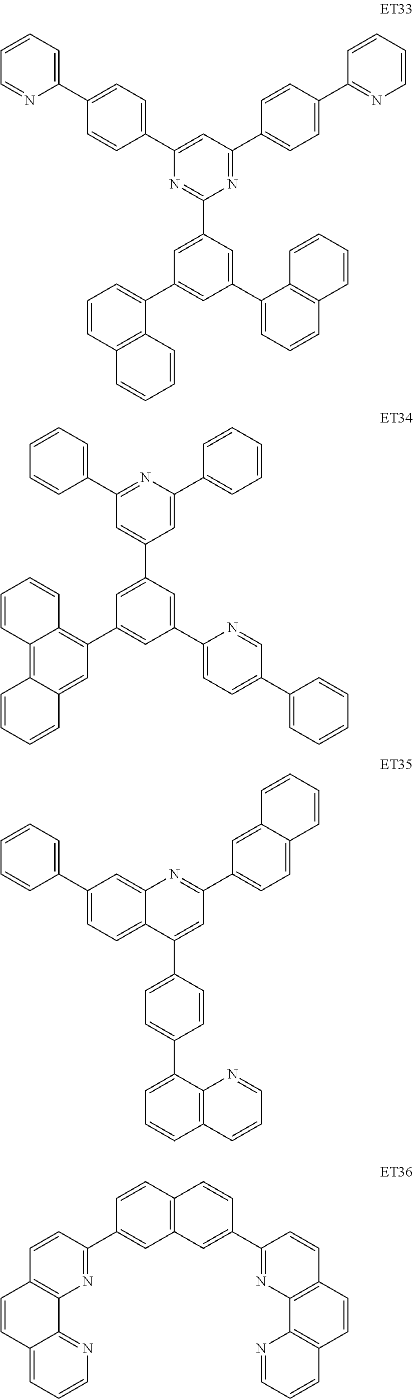

In an example embodiment, the electron transport region may include at least one compound selected from 2,9-dimethyl-4,7-diphenyl-1,10-phenanthroline (BCP), 4,7-diphenyl-1,10-phenanthroline (Bphen), Alq₃, Balq, 3-(biphenyl-4-yl)-5-(4-tert-butylphenyl)-4-phenyl-4H-1,2,4-triazole (TAZ), and NTAZ.

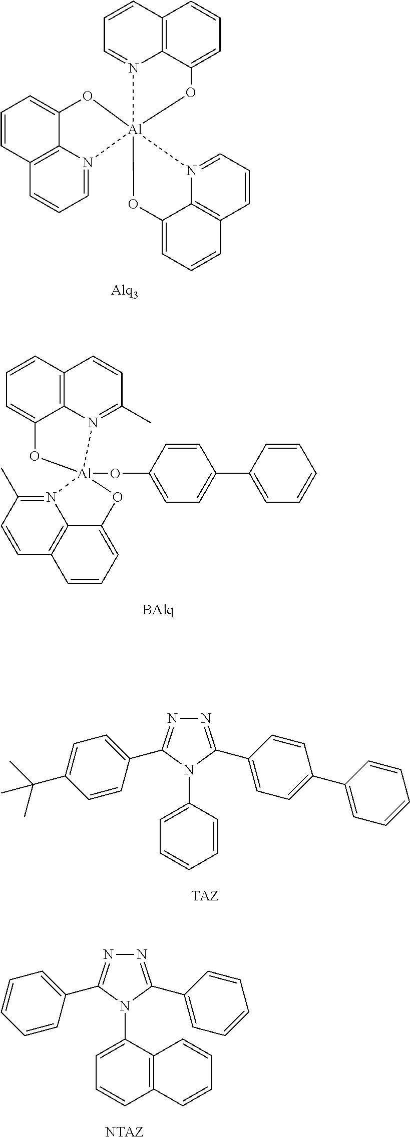

A thickness of the buffer layer, the hole blocking layer, or the electron control layer may be in a range of about 20 Å to about 1,000 Å, for example, about 30 Å to about 300 Å. When the thicknesses of the buffer layer, the hole blocking layer, and the electron control layer are within these ranges, the electron transport region may have excellent hole blocking characteristics or electron control characteristics without a substantial increase in driving voltage.

A thickness of the electron transport layer may be in a range of about 100 Å to about 1,000 Å, for example, about 150 Å to about 500 Å. When the thickness of the electron transport layer is within the range described above, the electron transport layer may have satisfactory electron transport characteristics without a substantial increase in driving voltage.

The electron transport region (for example, the electron transport layer in the electron transport region) may further include, in addition to the materials described above, a metal-containing material.

The metal-containing material may include at least one selected from alkali metal complex and alkaline earth-metal complex. The alkali metal complex may include a metal ion selected from a Li ion, a Na ion, a K ion, a Rb ion, and a Cs ion, and the alkaline earth-metal complex may include a metal ion selected from a Be ion, a Mg ion, a Ca ion, a Sr ion, and a Ba ion. A ligand coordinated with the metal ion of the alkali metal complex or the alkaline earth-metal complex may be selected from a hydroxy quinoline, a hydroxy isoquinoline, a hydroxy benzoquinoline, a hydroxy acridine, a hydroxy phenanthridine, a hydroxy phenyloxazole, a hydroxy phenylthiazole, a hydroxy diphenyloxadiazole, a hydroxy diphenylthiadiazol, a hydroxy phenylpyridine, a hydroxy phenylbenzimidazole, a hydroxy phenylbenzothiazole, a bipyridine, a phenanthroline, and a cyclopentadiene.

For example, the metal-containing material may include a Li complex. The Li complex may include, for example, Compound ET-D1 (lithium quinolate, LiQ) or ET-D2.

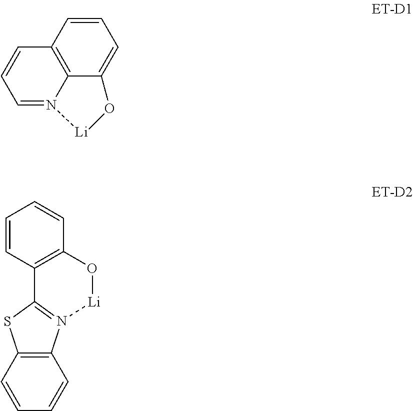

The electron transport region may include an electron injection layer that facilitates injection of electrons from the second electrode 190. The electron injection layer may directly contact the second electrode 190.

The electron injection layer may have i) a single-layered structure including a single layer including a single material, ii) a single-layered structure including a single layer including a plurality of different materials, or iii) a multi-layered structure having a plurality of layers including a plurality of different materials.

The electron injection layer may include an alkali metal, an alkaline earth metal, a rare earth metal, an alkali metal compound, an alkaline earth-metal compound, a rare earth metal compound, an alkali metal complex, an alkaline earth-metal complex, a rare earth metal complex, or a combinations thereof.

The alkali metal may be selected from Li, Na, K, Rb, and Cs. In an example embodiment, the alkali metal may be Li, Na, or Cs. In an example embodiment, the alkali metal may be Li or Cs.

The alkaline earth metal may be selected from Mg, Ca, Sr, and Ba.

The rare earth metal may be selected from Sc, Y, Ce, Tb, Yb, and Gd.

The alkali metal compound, the alkaline earth-metal compound, and the rare earth metal compound may be selected from oxides and halides (for example, fluorides, chlorides, bromides, or iodides) of the alkali metal, the alkaline earth-metal, and the rare earth metal.

The alkali metal compound may be selected from alkali metal oxides, such as Li₂O, Cs₂O, or K₂O, and alkali metal halides, such as LiF, NaF, CsF, KF, LiI, Nal, CsI, or KI. In an example embodiment, the alkali metal compound may be selected from LiF, Li₂O, NaF, LiI, Nal, CsI, and KI.

The alkaline earth-metal compound may be selected from alkaline earth-metal oxides, such as BaO, SrO, CaO, BaₓSr₁₋ₓO (0<x<1), or BaₓCa₁₋ₓO (0<x<1). In an example embodiment, the alkaline earth-metal compound may be selected from BaO, SrO, and CaO.

The rare earth metal compound may be selected from YbF₃, ScF₃, ScO₃, Y₂O₃, Ce₂O₃, GdF₃, and TbF₃. In an example embodiment, the rare earth metal compound may be selected from YbF₃, ScF₃, TbF₃, YbI₃, ScI₃, and TbI₃.

The alkali metal complex, the alkaline earth-metal complex, and the rare earth metal complex may include an ion of alkali metal, alkaline earth-metal, and rare earth metal as described above, and a ligand coordinated with a metal ion of the alkali metal complex, the alkaline earth-metal complex, or the rare earth metal complex may be selected from hydroxy quinoline, hydroxy isoquinoline, hydroxy benzoquinoline, hydroxy acridine, hydroxy phenanthridine, hydroxy phenyloxazole, hydroxy phenylthiazole, hydroxy diphenyloxadiazole, hydroxy diphenylthiadiazol, hydroxy phenylpyridine, hydroxy phenylbenzimidazole, hydroxy phenylbenzothiazole, bipyridine, phenanthroline, and cyclopentadiene.

The electron injection layer may include or consist of an alkali metal, an alkaline earth metal, a rare earth metal, an alkali metal compound, an alkaline earth-metal compound, a rare earth metal compound, an alkali metal complex, an alkaline earth-metal complex, a rare earth metal complex, or a combinations thereof, as described above. In an example embodiment, the electron injection layer may further include an organic material. When the electron injection layer further includes an organic material, an alkali metal, an alkaline earth metal, a rare earth metal, an alkali metal compound, an alkaline earth-metal compound, a rare earth metal compound, an alkali metal complex, an alkaline earth-metal complex, a rare earth metal complex, or a combinations thereof may be homogeneously or non-homogeneously dispersed in a matrix including the organic material.

A thickness of the electron injection layer may be in a range of about 1 Å to about 100 Å, for example, about 3 Å to about 90 Å. When the thickness of the electron injection layer is within the range described above, the electron injection layer may have satisfactory electron injection characteristics without a substantial increase in driving voltage.

[Second electrode 190]

The second electrode 190 may be disposed on the organic layer 150 having such a structure. The second electrode 190 may be a cathode which is an electron injection electrode, and in this regard, a material for forming the second electrode 190 may be selected from metal, an alloy, an electrically conductive compound, and a combination thereof, which have a relatively low work function.

The second electrode 190 may include at least one selected from lithium (Li), silver (Ag), magnesium (Mg), aluminum (Al), aluminum-lithium (Al—Li), calcium (Ca), magnesium-indium (Mg—In), magnesium-silver (Mg—Ag), ITO, and IZO. The second electrode 190 may be a transmissive electrode, a semi-transmissive electrode, or a reflective electrode.

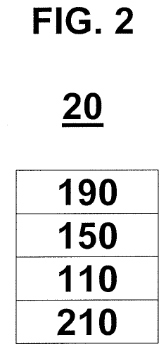

The second electrode 190 may have a single-layered structure, or a multi-layered structure including two or more layers.

[Description of FIGS. 2 to 4]

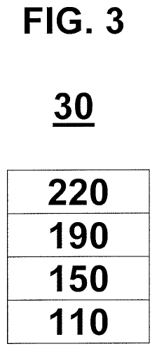

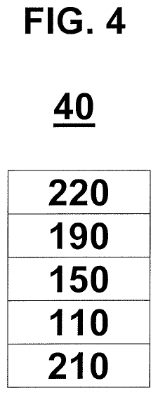

An organic light-emitting device 20 of FIG. 2 includes a first capping layer 210, a first electrode 110, an organic layer 150, and a second electrode 190 which are sequentially stacked in this stated order, an organic light-emitting device 30 of FIG. 3 includes a first electrode 110, an organic layer 150, a second electrode 190, and a second capping layer 220 which are sequentially stacked in this stated order, and an organic light-emitting device 40 of FIG. 4 includes a first capping layer 210, a first electrode 110, an organic layer 150, a second electrode 190, and a second capping layer 220.

Regarding FIGS. 2 to 4, the first electrode 110, the organic layer 150, and the second electrode 190 may be understood by referring to the description presented in connection with FIG. 1.

In the organic layer 150 of each of the organic light-emitting devices 20 and 40, light generated in an emission layer may pass through the first electrode 110, which is a semi-transmissive electrode or a transmissive electrode, and the first capping layer 210 toward the outside, and in the organic layer 150 of each of the organic light-emitting devices 30 and 40, light generated in an emission layer may pass through the second electrode 190, which is a semi-transmissive electrode or a transmissive electrode, and the second capping layer 220 toward the outside.

The first capping layer 210 and the second capping layer 220 may increase external luminescent efficiency according to the principle of constructive interference.

The first capping layer 210 and the second capping layer 220 may each independently be an organic capping layer including an organic material, an inorganic capping layer including an inorganic material, or a composite capping layer including an organic material and an inorganic material.

At least one selected from the first capping layer 210 and the second capping layer 220 may each independently include at least one material selected from carbocyclic compounds, heterocyclic compounds, amine-based compounds, porphyrine derivatives, phthalocyanine derivatives, a naphthalocyanine derivatives, alkali metal complexes, and alkaline earth metal complexes. The carbocyclic compound, the heterocyclic compound, and the amine-based compound may be optionally substituted with a substituent containing at least one element selected from O, N, S, Se, Si, F, Cl, Br, and I. In an example embodiment, at least one selected from the first capping layer 210 and the second capping layer 220 may each independently include an amine-based compound.

In an example embodiment, at least one selected from the first capping layer 210 and the second capping layer 220 may each independently include the compound represented by Formula 201 or the compound represented by Formula 202.

In an example embodiment, at least one selected from the first capping layer 210 and the second capping layer 220 may each independently include a compound selected from Compounds HT28 to HT33 and Compounds CP1 to CP5:

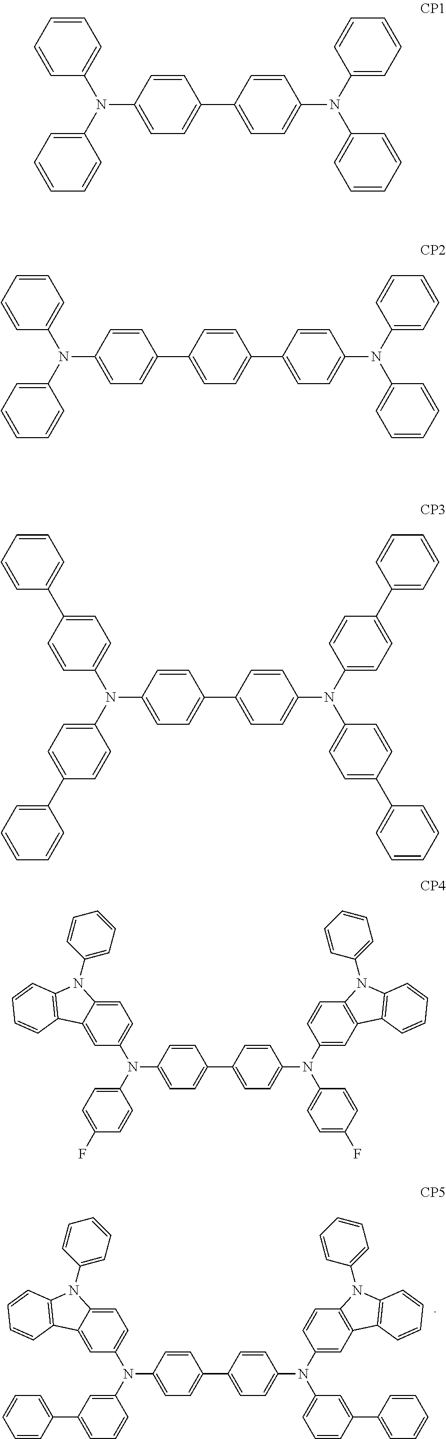 [SMILES: c1ccc(N(c2ccccc2)c2ccc(-c3ccc(N(c4ccccc4)c4ccccc4)cc3)cc2)cc1 ; c1ccc(N(c2ccccc2)c2ccc(-c3ccc(-c4ccc(N(c5ccccc5)c5ccccc5)cc4)cc3)cc2)cc1 ; c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; Fc1ccc(N(c2ccc(-c3ccc(N(c4ccc(F)cc4)c4ccc5c(c4)c4ccccc4n5-c4ccccc4)cc3)cc2)c2ccc3c(c2)c2ccccc2n3-c2ccccc2)cc1 ; c1ccc(-c2cccc(N(c3ccc(-c4ccc(N(c5cccc(-c6ccccc6)c5)c5ccc6c(c5)c5ccccc5n6-c5ccccc5)cc4)cc3)c3ccc4c(c3)c3ccccc3n4-c3ccccc3)c2)cc1]

Layers constituting the hole transport region, an emission layer, and layers constituting the electron transport region may be formed in a certain region by using one or more suitable methods selected from vacuum deposition, spin coating, casting, Langmuir-Blodgett (LB) deposition, ink-jet printing, laser-printing, and laser-induced thermal imaging.

When layers constituting the hole transport region, an emission layer, and layers constituting the electron transport region are formed by vacuum deposition, the deposition may be performed at a deposition temperature of about 100° C. to about 500° C., a vacuum degree of about 10⁻⁸ torr to about 10⁻³ torr, and a deposition speed of about 0.01 Å/sec to about 100 Å/sec by taking into account a material to be included in a layer to be formed, and the structure of a layer to be formed.

When layers constituting the hole transport region, an emission layer, and layers constituting the electron transport region are formed by spin coating, the spin coating may be performed at a coating speed of about 2,000 rpm to about 5,000 rpm and at a heat treatment temperature of about 80° C. to about 200° C. by taking into account a material to be included in a layer to be formed, and the structure of a layer to be formed.

### General Definition of Substituents

The term “C₁-C₆₀ alkyl group” as used herein refers to a linear or branched aliphatic saturated hydrocarbon monovalent group having 1 to 60 carbon atoms, and examples thereof include a methyl group, an ethyl group, a propyl group, an isobutyl group, a sec-butyl group, a tert-butyl group, a pentyl group, an isoamyl group, and a hexyl group. The term “C₁-C₆₀ alkylene group” as used herein refers to a divalent group having the same structure as the C₁-C₆₀ alkyl group.

The term “C₂-C₆₀ alkenyl group” as used herein refers to a hydrocarbon group having at least one double bond in the middle or at the terminus of the C₂-C₆₀ alkyl group, and examples thereof include an ethenyl group, a propenyl group, and a butenyl group. The term “C₂-C₆₀ alkenylene group” as used herein refers to a divalent group having the same structure as the C₂-C₆₀ alkenyl group.

The term “C₂-C₆₀ alkynyl group” as used herein refers to a hydrocarbon group having at least one triple bond in the middle or at the terminus of the C₂-C₆₀ alkyl group, and examples thereof include an ethynyl group, and a propynyl group. The term “C₂-C₆₀ alkynylene group” as used herein refers to a divalent group having the same structure as the C₂-C₆₀ alkynyl group.

The term “C₁-C₆₀ alkoxy group” as used herein refers to a monovalent group represented by —OA₁₀₁ (wherein A₁₀₁ is the C₁-C₆₀ alkyl group), and examples thereof include a methoxy group, an ethoxy group, and an isopropyloxy group.

The term “C₃-C₁₀ cycloalkyl group” as used herein refers to a monovalent saturated hydrocarbon monocyclic group having 3 to 10 carbon atoms, and examples thereof include a cyclopropyl group, a cyclobutyl group, a cyclopentyl group, a cyclohexyl group, and a cycloheptyl group. The term “C₃-C₁₀ cycloalkylene group” as used herein refers to a divalent group having the same structure as the C₃-C₁₀ cycloalkyl group.

The term “C₁-C₁₀ heterocycloalkyl group” as used herein refers to a monovalent monocyclic group having at least one heteroatom selected from N, O, Si, P, and S as a ring-forming atom and 1 to 10 carbon atoms, and examples thereof include a 1,2,3,4-oxatriazolidinyl group, a tetrahydrofuranyl group, and a tetrahydrothiophenyl group. The term “C₁-C₁₀ heterocycloalkylene group” as used herein refers to a divalent group having the same structure as the C₁-C₁₀ heterocycloalkyl group.

The term C₃-C₁₀ cycloalkenyl group used herein refers to a monovalent monocyclic group that has 3 to 10 carbon atoms and at least one double bond in the ring thereof and no aromaticity, and examples thereof include a cyclopentenyl group, a cyclohexenyl group, and a cycloheptenyl group. The term “C₃-C₁₀ cycloalkenylene group” as used herein refers to a divalent group having the same structure as the C₃-C₁₀ cycloalkenyl group.

The term “C₁-C₁₀ heterocycloalkenyl group” as used herein refers to a monovalent monocyclic group that has at least one heteroatom selected from N, O, Si, P, and S as a ring-forming atom, 1 to 10 carbon atoms, and at least one double bond in its ring. Non-limiting examples of the C₁-C₁₀ heterocycloalkenyl group include a 4,5-dihydro-1,2,3,4-oxatriazolyl group, a 2,3-dihydrofuranyl group, and a 2,3-dihydrothiophenyl group. The term “C₁-C₁₀ heterocycloalkenylene group” as used herein refers to a divalent group having the same structure as the C₁-C₁₀ heterocycloalkenyl group.

The term “C₆-C₆₀ aryl group” as used herein refers to a monovalent group having a carbocyclic aromatic system having 6 to 60 carbon atoms, and a C₆-C₆₀ arylene group used herein refers to a divalent group having a carbocyclic aromatic system having 6 to 60 carbon atoms. Non-limiting examples of the C₆-C₆₀ aryl group include a phenyl group, a naphthyl group, an anthracenyl group, a phenanthrenyl group, a pyrenyl group, and a chrysenyl group. When the C₆-C₆₀ aryl group and the C₆-C₆₀ arylene group each include two or more rings, the rings may be fused to each other.

The term “C₁-C₆₀ heteroaryl group” as used herein refers to a monovalent group having a heterocyclic aromatic system that has at least one heteroatom selected from N, O, Si, P, and S as a ring-forming atom, in addition to 1 to 60 carbon atoms. The term “C₁-C₆₀ heteroarylene group” as used herein refers to a divalent group having a heterocyclic aromatic system that has at least one heteroatom selected from N, O, Si, P, and S as a ring-forming atom, in addition to 1 to 60 carbon atoms. Non-limiting examples of the C₁-C₆₀ heteroaryl group include a pyridinyl group, a pyrimidinyl group, a pyrazinyl group, a pyridazinyl group, a triazinyl group, a quinolinyl group, and an isoquinolinyl group. When the C₁-C₆₀ heteroaryl group and the C₁-C₆₀ heteroarylene group each include two or more rings, the rings may be condensed with each other.

The term “C₆-C₆₀ aryloxy group” as used herein indicates —OA₁₀₂ (wherein A₁₀₂ is the C₆-C₆₀ aryl group), and the term “C₆-C₆₀ arylthio group” used herein indicates —SA₁₀₃ (wherein A₁₀₃ is the C₆-C₆₀ aryl group).

The term “C₁-C₆₀ heteroaryloxy group” as used herein refers to —OA₁₀₄ (wherein A₁₀₄ is the C₁-C₆₀ heteroaryl group), and the term “C₁-C₆₀ heteroarylthio group” used herein indicates —SA₁₀₅ (wherein A₁₀₅ is the C₁-C₆₀ heteroaryl group).

The term “monovalent non-aromatic condensed polycyclic group” as used herein refers to a monovalent group (for example, having 8 to 60 carbon atoms) having two or more rings condensed with each other, only carbon atoms as ring-forming atoms, and containing at least one non-aromatic ring. A detailed example of the monovalent non-aromatic condensed polycyclic group is a fluorenyl group. The term “divalent non-aromatic condensed polycyclic group” as used herein refers to a divalent group having the same structure as the monovalent non-aromatic condensed polycyclic group.

The term “monovalent non-aromatic condensed heteropolycyclic group” as used herein refers to a monovalent group (for example, having 1 to 60 carbon atoms) having two or more rings condensed to each other, at least one heteroatom selected from N, O, Si, P, and S, other than carbon atoms, as a ring-forming atom, and containing at least one non-aromatic ring. An example of the monovalent non-aromatic condensed heteropolycyclic group is a carbazolyl group. The term “divalent non-aromatic condensed heteropolycyclic group” as used herein refers to a divalent group having the same structure as the monovalent non-aromatic condensed heteropolycyclic group.

The term “C₅-C₆₀ carbocyclic group” as used herein refers to a monocyclic or polycyclic group having 5 to 60 carbon atoms in which a ring-forming atom is a carbon atom only. The C₅-C₆₀ carbocyclic group may be an aromatic carbocyclic group or a non-aromatic carbocyclic group. The C₅-C₆₀ carbocyclic group may be a ring, such as benzene, a monovalent group, such as a phenyl group, or a divalent group, such as a phenylene group. In an example embodiment, depending on the number of substituents connected to the C₅-C₆₀ carbocyclic group, the C₅-C₆₀ carbocyclic group may be a trivalent group or a quadrivalent group.

The term “C₁-C₆₀ heterocyclic group” as used herein refers to a group having the same structure as the C₅-C₆₀ carbocyclic group, except that as a ring-forming atom, at least one heteroatom selected from N, O, Si, P, and S is used in addition to carbon (the number of carbon atoms may be in a range of 1 to 60).

In the present specification, at least one substituent of the substituted C₅-C₆₀ carbocyclic group, the substituted C₇-C₆₀ carbocyclic group, the substituted C₁-C₆₀ heterocyclic group, the substituted C₁-C₂₀ alkylene group, the substituted C₂-C₂₀ alkenylene group, the substituted C₃-C₁₀ cycloalkylene group, the substituted C₁-C₁₀ heterocycloalkylene group, the substituted C₃-C₁₀ cycloalkenylene group, the substituted C₁-C₁₀ heterocycloalkenylene group, the substituted C₆-C₆₀ arylene group, the substituted C₁-C₆₀ heteroarylene group, the substituted divalent non-aromatic condensed polycyclic group, the substituted divalent non-aromatic condensed heteropolycyclic group, the substituted C₁-C₆₀ alkyl group, the substituted C₂-C₆₀ alkenyl group, the substituted C₂-C₆₀ alkynyl group, the substituted C₁-C₆₀ alkoxy group, the substituted C₃-C₁₀ cycloalkyl group, the substituted C₁-C₁₀ heterocycloalkyl group, the substituted C₃-C₁₀ cycloalkenyl group, the substituted C₁-C₁₀ heterocycloalkenyl group, the substituted C₆-C₆₀ aryl group, the substituted C₆-C₆₀ aryloxy group, the substituted C₆-C₆₀ arylthio group, the substituted C₁-C₆₀ heteroaryl group, the substituted C₁-C₆₀ heteroaryloxy group, the substituted C₁-C₆₀ heteroarylthio group, the substituted monovalent non-aromatic condensed polycyclic group, and the substituted monovalent non-aromatic condensed heteropolycyclic group may be selected from:

deuterium, —F, —Cl, —Br, —I, a hydroxyl group, a cyano group, a nitro group, an amidino group, a hydrazino group, a hydrazono group, a C₁-C₆₀ alkyl group, a C₂-C₆₀ alkenyl group, a C₂-C₆₀ alkynyl group, and a C₁-C₆₀ alkoxy group;

a C₁-C₆₀ alkyl group, a C₂-C₆₀ alkenyl group, a C₂-C₆₀ alkynyl group, and a C₁-C₆₀ alkoxy group, each substituted with at least one selected from deuterium, —F, —Cl, —Br, —I, a hydroxyl group, a cyano group, a nitro group, an amidino group, a hydrazino group, a hydrazono group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), and —P(═O)(Q₁₁)(Q₁₂);

a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group;

a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group, each substituted with at least one selected from deuterium, —F, —Cl, —Br, —I, a hydroxyl group, a cyano group, a nitro group, an amidino group, a hydrazino group, a hydrazono group, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), and —P(═O)(Q₂₁)(Q₂₂); and

—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), and —P(═O)(Q₃₁)(Q₃₂), and

Q₁₁ to Q₁₃, Q₂₁ to Q₂₃, and Q₃₁ to Q₃₃ may each independently be selected from hydrogen, deuterium, —F, —Cl, —Br, —I, a hydroxyl group, a cyano group, a nitro group, an amidino group, a hydrazino group, a hydrazono group, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₁-C₆₀ heteroaryl group, a monovalent non-aromatic condensed polycyclic group, a monovalent non-aromatic condensed heteropolycyclic group, a biphenyl group, and a terphenyl group.

The term “Ph” as used herein represents a phenyl group, the term “Me” as used herein represents a methyl group, the term “Et” as used herein represents an ethyl group, the term “ter-Bu” or “Bu^{t},” as used herein, represents a tert-butyl group, and the term “OMe” as used herein represents a methoxy group.

The term “biphenyl group” as used herein refers to a “phenyl group substituted with a phenyl group. The “biphenyl group” is a “substituted phenyl group” having a “C₆-C₆₀ aryl group” as a substituent.

The term “terphenyl group” as used herein refers to a “phenyl group substituted with a biphenyl group. The “terphenyl group” is a “phenyl group” having, as a substituent, a “C₆-C₆₀ aryl group substituted with a C₆-C₆₀ aryl group.”

* and *′ as used herein, unless defined otherwise, each refer to a binding site to a neighboring atom in a corresponding formula.

The following Examples and Comparative Examples are provided in order to highlight characteristics of one or more embodiments, but it will be understood that the Examples and Comparative Examples are not to be construed as limiting the scope of the embodiments, nor are the Comparative Examples to be construed as being outside the scope of the embodiments. Further, it will be understood that the embodiments are not limited to the particular details described in the Examples and Comparative Examples.

The expression “B was used instead of A” used in describing Synthesis Examples means that an identical number of molar equivalents of B was used in place of molar equivalents of A.

### EXAMPLES

### Synthesis Examples

### Synthesis Example 1: Synthesis of Compound 5

(1) Synthesis of Intermediate 5(1)

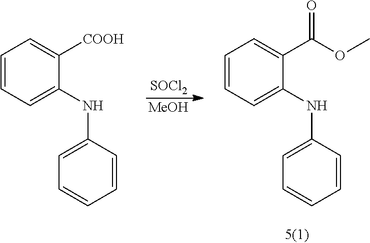

10 g (1.0 eq, 47 mmol) of 2-(phenylamino)benzoic acid was added to a reaction container and vacuum-dried, and the reaction container was filled with nitrogen gas. 60 mL of methanol was added thereto and stirred at a temperature of 60° C. under reflux. 6.1 mL (2.0 eq, 94 mmol) of SOCl₂ was slowly added dropwise thereto while maintaining the temperature of 60° C. The reaction mixture was stirred at a temperature of 60° C. for 12 hours under reflux. The reaction container was cooled to room temperature, and saturated NaHCO₃ solution was added thereto to terminate the reaction. After the reaction was terminated, an organic layer was extracted therefrom by using ethyl acetate (EA) and distilled water. The extracted organic layer was dried by using anhydrous sodium sulfate and filtered through Celite, and a solvent was evaporated therefrom. Then, column chromatography was performed thereon to obtain 8.5 g (yield of 80%) of Intermediate 5(1), that is, methyl 2-(phenylamino)benzoate.

¹H NMR (500 MHz, CDCl₃) δ 9.52 (s, 1H), 7.91 (d, J=8.0 Hz, 1H), 7.26 (t, J=7.8 Hz, 2H), 7.23-7.19 (m, 3H), 7.18 (s, 1H), 7.01 (t, J=7.3 Hz, 1H), 6.70-6.58 (m, 1H), 3.78 (s, 3H).

(2) Synthesis of Intermediate 5(2)

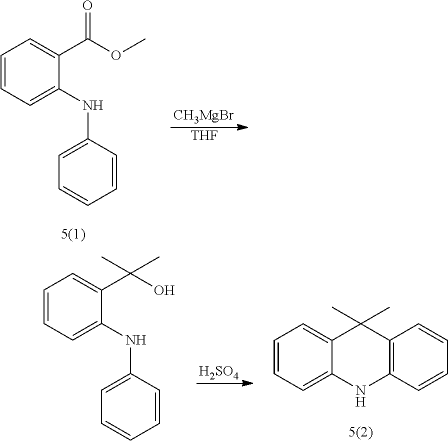

8.5 g (1.0 eq, 37 mmol) of methyl 2-(phenylamino)benzoate was added to a reaction container (flask) and vacuum-dried, and the reaction container was filled with nitrogen gas. 400 mL of tetrahydrofuran (THF) was added thereto, and 50 mL (4.0 eq, 150 mmol) of 3.0 M methylmagnesium bromide was slowly added dropwise thereto and stirred for 3 hours. The reaction was terminated by using an aqueous ammonium chloride solution, and an organic layer was extracted therefrom by using distilled water and ethyl acetate and then dried. The dried reaction mixture was added to the reaction container, and sulfuric acid was added thereto and stirred at room temperature for 30 minutes. After the reaction was completed, the reaction product was neutralized by using aqueous sodium hydroxide solution, and an organic layer was extracted therefrom by using distilled water and ethyl acetate. The extracted organic layer was dried by using anhydrous sodium sulfate and filtered through Celite, and a solvent was evaporated therefrom. Then, column chromatography was performed thereon to obtain 4.9 g (yield of 63%) of Intermediate 5(2), that is, 9,10-dihydro-9,9-dimethylacridine.

¹H NMR (500 MHz, CDCl₃) δ 7.38 (d, J=7.7 Hz, 2H), 7.10 (t, J=7.2 Hz, 2H), 6.92 (d, J=6.8 Hz, 2H), 6.69 (d, J=7.5 Hz, 2H), 6.13 (s, 1H), 1.58 (s, 6H).

(3) Synthesis of Intermediate 5(3)

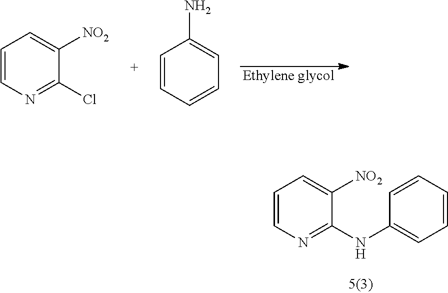

10 g (1.0 eq, 63 mmol) of 2-chloro-3-nitropyridine was added to a reaction container and vacuum-dried, and the reaction container was filled with nitrogen gas. Then, 5.76 mL (1.0 eq, 63 mmol) of aniline and 100 mL of ethylene glycol were added thereto and stirred at a temperature of 140° C. for 8 hours. After the reaction was completed, the reaction container was cooled to room temperature, and an organic layer was extracted therefrom by using distilled water and ethyl acetate. Then extracted organic layer was dried by using anhydrous magnesium sulfate and filtered through Celite, and a solvent was evaporated therefrom. Then, column chromatography was performed thereon to obtain 12 g (yield of 87%) of Intermediate 5(3), that is, 3-nitro-N-phenylpyridin-2-amine.

¹H NMR (500 MHz, CDCl₃) δ 10.12 (s, 1H), 8.53 (dd, J=8.3, 1.8 Hz, 1H), 8.49 (dd, J=4.5, 1.8 Hz, 1H), 7.65 (dt, J=8.7, 1.6 Hz, 2H), 7.42-7.38 (m, 2H), 7.21-7.17 (m, 1H), 6.83 (dd, J=8.3, 4.5 Hz, 1H).

(4) Synthesis of Intermediate 5(4)

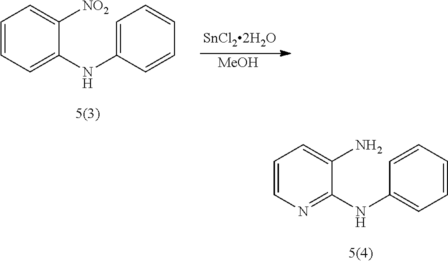 [SMILES: "MeOH" ; O=[N+]([O-])c1ccccc1Nc1ccccc1 ; Nc1cccnc1Nc1ccccc1]

12 g (1.0 eq, 55 mmol) of 3-nitro-N-phenylpyridin-2-amine and 37 g (3.0 eq, 160 mmol) of SnCl₂.2H₂O were added to a reaction container and vacuum-dried, and the reaction container was filled with nitrogen gas. 300 mL of methanol was added thereto and stirred at a temperature of 60° C. for 2 hours under reflux. The reaction mixture was neutralized by adding NaHCO₃ aqueous solution thereto, and the reaction was terminated. An organic layer was extracted therefrom by using distilled water and ethyl acetate. The extracted organic layer was dried by using anhydrous sodium sulfate and filtered through Celite, and a solvent was evaporated therefrom. Then, recrystallization using dichloromethane (MC) and hexane was performed thereon to obtain 8.2 g (yield of 81%) of Intermediate 5(4), that is, N²-phenylpyridine-2,3-diamine.

¹H NMR (500 MHz, CDCl₃) δ 7.84 (dd, J=4.9, 1.5 Hz, 1H), 7.31-7.24 (m, 4H), 7.02 (dd, J=7.6, 1.6 Hz, 1H), 6.96 (tt, J=7.1, 1.6 Hz, 1H), 6.78 (dd, J=7.6, 4.9 Hz, 1H), 6.19 (s, 1H), 3.41 (s, 2H).

(5) Synthesis of Intermediate 5(5)

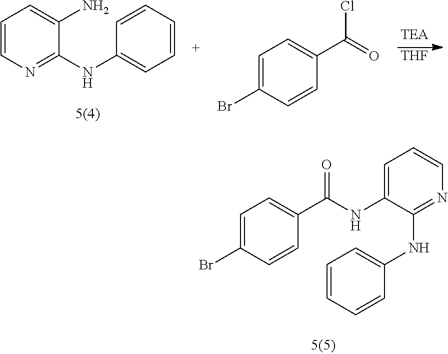 [SMILES: Nc1cccnc1Nc1ccccc1 ; O=C(Cl)c1ccc(Br)cc1 ; O=C(Nc1cccnc1Nc1ccccc1)c1ccc(Br)cc1 ; "THF"]

0.5 g (1.0 eq, 2.7 mmol) of N²-phenylpyridine-2,3-diamine and 0.59 g (1.0 eq, 2.7 mmol) of 4-bromobenzoyl chloride were added to a reaction container and vacuum-dried, and the reaction container was filled with nitrogen gas. 10 mL of THF was added thereto and stirred. Then, the reaction container was cooled to a temperature of 0° C. 0.4 mL of triethylamine (TEA) was slowly added dropwise thereto while maintaining the temperature of 0° C., and stirred at room temperature for 4 hours. After the reaction was completed, an organic layer was extracted therefrom by using distilled water and ethyl acetate. The extracted organic layer was dried by using anhydrous magnesium sulfate and filtered through Celite, and a solvent was evaporated therefrom. Then, recrystallization using dichloromethane and hexane was performed thereon to obtain 0.6 g (yield of 61%) of Intermediate 5(5), that is, 4-bromo-N-(2-(phenylamino)pyridin-3-yl)benzamide.

¹H NMR (500 MHz, CDCl₃) δ 8.24 (d, J=7.4 Hz, 1H), 8.19 (dd, J=4.8, 1.6 Hz, 1H), 7.69 (s, 1H), 7.58-7.49 (m, 2H), 7.44-7.37 (m, 2H), 7.33-7.27 (m, 2H), 7.11 (d, J=7.8 Hz, 2H), 7.07-6.98 (m, 2H), 6.53 (s, 1H).

(6) Synthesis of Intermediate 5(6)

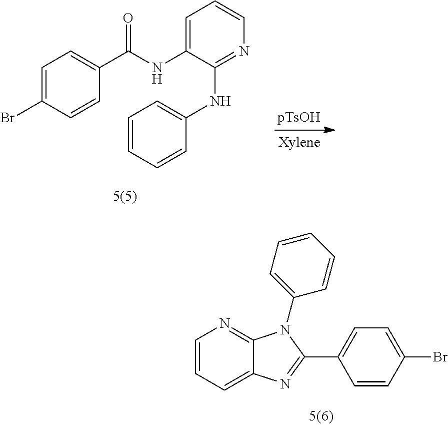 [SMILES: O=C(Nc1cccnc1Nc1ccccc1)c1ccc(Br)cc1 ; Brc1ccc(-c2nc3cccnc3n2-c2ccccc2)cc1]

0.60 g (1.0 eq, 1.6 mmol) of 4-bromo-N-(2-(phenylamino)pyridin-3-yl)benzamide and 0.10 g (0.33 eq, 0.54 mmol) of p-toluenesulfonic acid were added to a reaction container and vacuum-dried, and the reaction container was filled with nitrogen gas. 8 mL ofxylene was added thereto and stirred at a temperature of 140° C. for 5 hours under reflux. After the reaction was completed, the reaction container was cooled to room temperature, and hexane was added thereto to obtain a solid crystal. The obtained solid was dissolved in chloroform, and recrystallization using ethanol was performed thereon to obtain 0.5 g (yield of 87.7%) of Intermediate 5(6), that is, 2-(4-bromophenyl)-3-phenyl-3H-imidazo[4,5-b]pyridine.

¹H NMR (500 MHz, CDCl₃) δ 8.49 (dd, J=4.7, 1.3 Hz, 1H), 7.83 (d, J=8.1 Hz, 1H), 7.62-7.54 (m, 3H), 7.51 (s, 3H), 7.43 (dd, J=8.1, 4.8 Hz, 1H), 7.41-7.37 (m, 2H), 7.19 (d, J=8.0 Hz, 1H).

(7) Synthesis of Compound 5

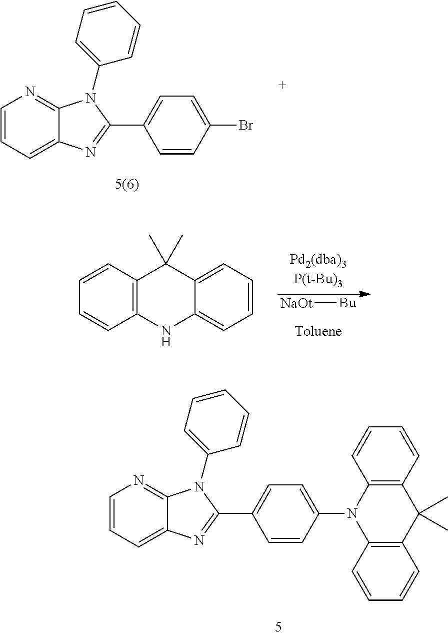

0.50 g (1.0 eq, 1.4 mmol) of 2-(4-bromophenyl)-3-phenyl-3H-imidazo[4,5-b]pyridine, 0.33 g (1.1 eq, 1.6 mmol) of 9,10-dihydro-9,9-dimethylacridine, 0.13 g (0.10 eq, 0.14 mmol) of tris(dibenzylideneacetone)dipalladium(0) (Pd₂(dba)₃), 0.060 g (0.20 eq, 0.29 mmol) of tri-tert-butylphosphine, and 0.52 g (4.0 eq, 5.7 mmol) of sodium tert-butoxide were added to a reaction container and vacuum-dried, and the reaction container was filled with nitrogen gas. 8.5 mL of toluene was added to the reaction container, and compounds were dissolved therein and stirred at a temperature of 110° C. for 3 hours under reflux. After the reaction was completed, an organic layer was extracted therefrom by using distilled water and ethyl acetate. The extracted organic layer was dried by using anhydrous magnesium sulfate and filtered through Celite, and a solvent was evaporated therefrom. After column chromatography, recrystallization using dichloromethane and hexane was performed thereon to obtain 0.54 g (yield of 79%) of a final Compound 5 (9,10-dihydro-9,9-dimethyl-10-(4-(3-phenyl-3H-imidazo[4,5-b]pyridin-2-yl)phenyl)acridine).

¹H NMR (500 MHz, CDCl₃) δ 8.44 (dd, J=4.8, 1.4 Hz, 1H), 8.19 (dd, J=8.0, 1.4 Hz, 1H), 7.91-7.80 (m, 2H), 7.63-7.55 (m, 2H), 7.54-7.43 (m, 5H), 7.37-7.31 (m, 3H), 6.95 (dtd, J=19.7, 7.3, 1.5 Hz, 4H), 6.26 (dd, J=8.0, 1.3 Hz, 2H), 1.66 (s, 6H). APCI-MS (m/z): 479 [M+].

### Synthesis Example 2: Synthesis of Compound 7

(1) Synthesis of Intermediate 7(1)

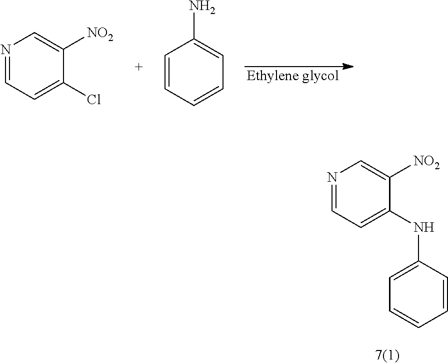

0.50 g (1.0 eq, 3.2 mmol) of 4-chloro-3-nitropyridine was added to a reaction container and vacuum-dried, and the reaction container was filled with nitrogen gas. 10 mL of ethylene glycol was added to the reaction container and stirred to dissolve the compounds. 0.58 ml (2.0 eq, 6.3 mmol) of aniline was added thereto and stirred at a temperature of 140° C. for 8 hours under reflux. After the reaction was completed, the reaction product was washed, and an organic layer was extracted therefrom by using ethyl acetate. The extracted organic layer was dried by using magnesium sulfate and filtered through Celite, and a solvent was evaporated therefrom. Then, column chromatography was performed thereon to obtain 0.57 g (yield of 83%) of Intermediate 7(1), that is, 3-nitro-N-phenylpyridin-4-amine.

¹H NMR: 9.78-9.49 (s, 1H), 9.29 (s, 1H), 8.25 (dd, J=6.1, 0.5 Hz, 1H), 7.58-7.42 (m, 2H), 7.36 (dd, J=10.8, 4.2 Hz, 1H), 7.32-7.27 (m, 2H), 6.95 (d, J=6.2 Hz, 1H).

(2) Synthesis of Intermediate 7(2)

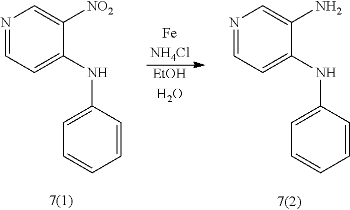 [SMILES: "EtOHH2O" ; O=[N+]([O-])c1cnccc1Nc1ccccc1 ; "FeNH4Cl" ; Nc1cnccc1Nc1ccccc1]

0.57 g (1.0 eq, 2.6 mmol) of 3-nitro-N-phenylpyridin-4-amine was added to a reaction container and vacuum-dried, and the reaction container was filled with nitrogen gas. 38 mL of ethanol was added to the reaction container and stirred to dissolve the compounds, and the compounds are stirred under reflux. 1.5 g (10 eq, 26 mmol) of iron was added thereto and stirred to well spread in the solution. Then, 1.4 g (10 eq, 26 mmol) of ammonium chloride was dissolved in 11 mL of distilled water, added thereto, and stirred for 2 hours under reflux. After the reaction was completed, the reaction product was filtered through Celite and washed by using distilled water, and an organic layer was extracted therefrom by using ethyl acetate. The extracted organic layer was dried by using magnesium sulfate and filtered through Celite, and a solvent was evaporated therefrom. Then, recrystallization using dichloromethane and hexane was performed thereon to obtain 0.39 g (yield of 80%) of Intermediate 7(2), that is, N⁴-phenylpyridine-3,4-diamine.

¹H NMR: 8.01 (d, J=58.6 Hz, 2H), 7.34 (t, J=7.8 Hz, 2H), 7.17-7.05 (m, 3H), 7.01 (s, 1H), 5.97 (s, 1H), 3.39 (s, 2H).

(3) Synthesis of Intermediate 7(3)

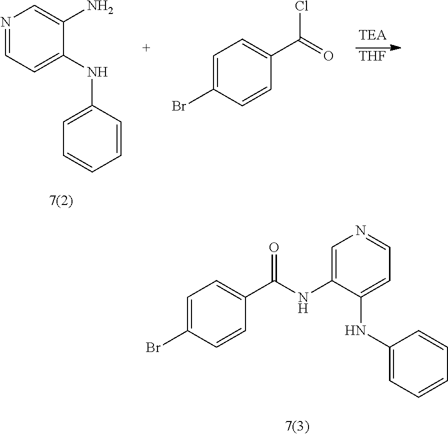 [SMILES: Nc1cnccc1Nc1ccccc1 ; O=C(Cl)c1ccc(Br)cc1 ; "THF" ; O=C(Nc1cnccc1Nc1ccccc1)c1ccc(Br)cc1]

0.39 g (1.0 eq, 1.8 mmol) of N⁴-phenylpyridine-3,4-diamine and 0.40 g (1.0 eq, 1.8 mmol) of 4-bromobenzoyl chloride were added to a reaction container and vacuum-dried, and the reaction container was filled with nitrogen gas. 8 mL of THF was added to the reaction container, and the compounds were dissolved therein. Then, 0.3 mL of triethylamine was slowly added dropwise thereto and stirred. After the reaction was completed, a precipitate obtained by washing with distilled water was filtered to obtain 0.54 g (yield of 82%) of Intermediate 7(3), that is, 4-bromo-N-(4-(phenylamino)pyridin-3-yl)benzamide.

¹H NMR: 9.34 (s, 1H), 8.75 (d, J=6.4 Hz, 1H), 7.94 (d, J=8.1 Hz, 1H), 7.88 (d, J=7.7 Hz, 1H), 7.69-7.62 (m, 2H), 7.58-7.49 (m, 2H), 7.51-7.43 (m, 2H), 7.39-7.32 (m, 2H), 7.20 (d, J=7.8 Hz, 2H).

(4) Synthesis of Intermediate 7(4)

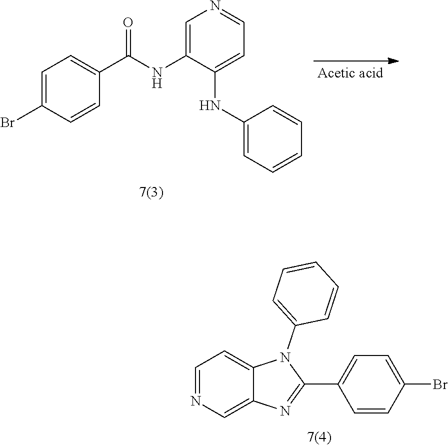 [SMILES: O=C(Nc1cnccc1Nc1ccccc1)c1ccc(Br)cc1 ; Brc1ccc(-c2nc3cnccc3n2-c2ccccc2)cc1]

0.50 g (1.0 eq, 1.4 mmol) of 4-bromo-N-(4-(phenylamino)pyridin-3-yl)benzamide was added to a reaction container and vacuum-dried, and the reaction container was filled with nitrogen gas. 10 mL of acetic acid was added to the reaction container to dissolve the compounds, and the compounds were stirred at a temperature of 120° C. under reflux. After the reaction was completed, the reaction product was neutralized by sodium carbonate aqueous solution and washed with distilled water, and an organic layer was extracted therefrom by using ethyl acetate. The extracted organic layer was dried by using magnesium sulfate and filtered through Celite, and a solvent was evaporated therefrom. Then, recrystallization using dichloromethane and hexane was performed thereon to obtain 0.28 g (yield of 67%) of Intermediate 7(4), that is, 2-(4-bromophenyl)-1-phenyl-1H-imidazo[4,5-c]pyridine.

¹H NMR: 9.21 (s, 1H), 8.45 (d, J=4.9 Hz, 1H), 7.63-7.51 (m, 3H), 7.51-7.40 (m, 4H), 7.35-7.27 (m, 2H), 7.20 (d, J=5.5 Hz, 1H).

(5) Synthesis of Compound 7

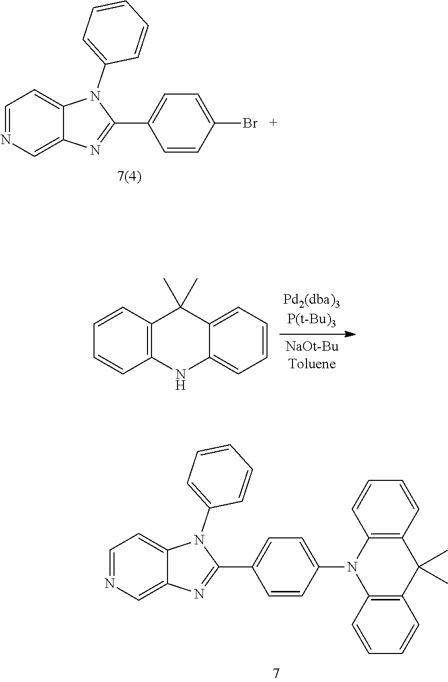

0.50 g (1.0 eq, 1.4 mmol) of 2-(4-bromophenyl)-1-phenyl-1H-imidazo[4,5-c]pyridine, 0.33 g (1.1 eq, 1.6 mmol) of 9,9-dimethyl-9,10-dihydroacridine, 0.13 g (0.10 eq, 0.14 mmol) of tris(dibenzylideneacetone)dipalladium(0), and 0.52 g (4.0 eq, 5.7 mmol) of sodium tert-butoxide were added to a reaction container and vacuum-dried, and the reaction container was filled with nitrogen gas. 9 mL of toluene was added to the reaction container to dissolve the compounds. 0.060 g (0.20 eq, 0.28 mmol) of tri-tert-butylphosphine was added thereto and stirred at a temperature of 110° C. for 2 hours under reflux. After the reaction was completed, the reaction product was washed with distilled water, and an organic layer was extracted therefrom by using ethyl acetate. The extracted organic layer was dried by using magnesium sulfate and filtered through Celite, and a solvent was evaporated therefrom. Then, after column chromatography, recrystallization using ethyl acetate and hexane was performed thereon to obtain 0.35 g (yield of 52%) of a final Compound 7 (9,9-dimethyl-10-(4-(1-phenyl-1H-imidazo[4,5-c]pyridin-2-yl)phenyl)-9,10-dihydroacridine).

¹H NMR: 9.24 (s, 1H), 8.47 (d, J=5.1 Hz, 1H), 7.97-7.71 (m, 2H), 7.67-7.49 (m, 3H), 7.48-7.42 (m, 2H), 7.43-7.37 (m, 2H), 7.35-7.29 (m, 2H), 7.23 (t, J=6.7 Hz, 1H), 7.03-6.86 (m, 4H), 6.25 (dd, J=8.0, 1.4 Hz, 2H), 1.66 (s, 6H). APCI-MS (m/z): 479[M+].

### Synthesis Example 3: Synthesis of Compound 9

(1) Synthesis of Intermediate 9(1)

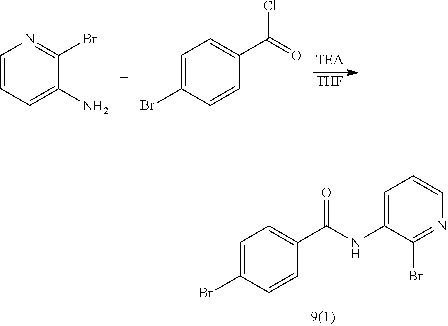

4.5 g (1.0 eq, 26 mmol) of 2-bromopyridine-3-amine and 8.6 g (1.5 eq, 39 mmol) of 4-bromobenzoyl chloride were added to a reaction container and vacuum-dried, and the reaction container was filled with nitrogen gas. 72 mL of THF was added thereto, stirred, and cooled to a temperature of 0° C. 6.0 mL of triethylamine was slowly added dropwise thereto while maintaining the temperature of 0° C., and stirred at room temperature for 4 hours. After the reaction was completed, an organic layer was extracted therefrom by using distilled water and ethyl acetate. The extracted organic layer was dried by using anhydrous magnesium sulfate and filtered through Celite, and a solvent was evaporated therefrom. Then, column chromatography and recrystallization using dichloromethane and hexane were performed thereon to obtain 9.2 g (yield of 97%) of Intermediate 9(1), that is, 4-bromo-N-(2-bromopyridin-3-yl)benzamide.

¹H NMR (500 MHz, CDCl₃) δ 8.83 (dd, J=8.1, 1.8 Hz, 1H), 8.43 (s, 1H), 8.15 (dd, J=4.6, 1.8 Hz, 1H), 7.82-7.77 (m, 2H), 7.72-7.67 (m, 2H), 7.34 (dd, J=8.1, 4.6 Hz, 1H).

(2) Synthesis of Intermediate 9(2)

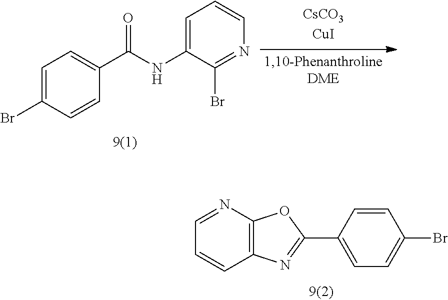 [SMILES: O=C(Nc1cccnc1Br)c1ccc(Br)cc1 ; Brc1ccc(-c2nc3cccnc3o2)cc1]

9.4 g (1 eq, 26 mmol) of 4-bromo-N-(2-bromopyridin-3-yl)benzamide, 13 g (1.5 eq, 40 mmol) of cesium carbonate, 0.50 g (0.10 eq, 2.6 mmol) of copper iodine(I), and 0.96 g (0.20 eq, 5.3 mmol) of 1,10-phenanthroline were added to a reaction container and vacuum-dried, and the reaction container was filled with nitrogen gas. 210 mL of dimethoxyethane (DME) was added thereto and stirred at a temperature of 80° C. for 24 hours under reflux. After the reaction was completed, an organic layer was extracted therefrom by using distilled water and ethyl acetate. The extracted organic layer was dried by using anhydrous magnesium sulfate and filtered through Celite, and a solvent was evaporated therefrom. Then, column chromatography was performed thereon to obtain 5.2 g (yield of 72%) of Intermediate 9(2), that is, 2-(4-bromophenyl)oxazolo[5,4-b]pyridine.

¹H NMR (500 MHz, CDCl₃) δ 8.37 (dd, J=4.9, 1.5 Hz, 1H), 8.20-8.14 (m, 2H), 8.08 (dd, J=7.8, 1.6 Hz, 1H), 7.73-7.68 (m, 2H), 7.37 (dd, J=7.8, 4.9 Hz, 1H).

(3) Synthesis of Compound 9

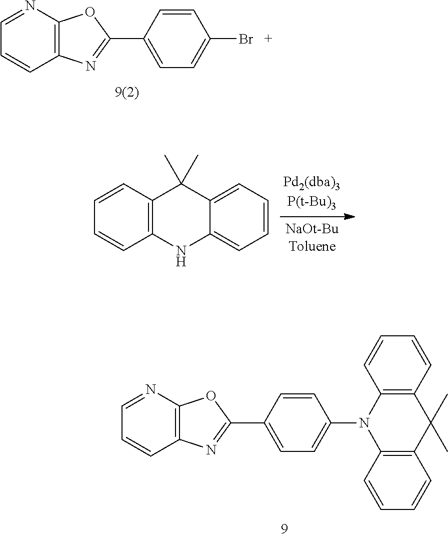

3.0 g (1.0 eq, 11 mmol) of 2-(4-bromophenyl)oxazolo[5,4-b]pyridine, 2.5 g (1.1 eq, 12 mmol) of 9,10-dihydro-9,9-dimethylacridine, 1.0 g (0.050 eq, 1.1 mnol) of tris(dibenzylideneacetone) dipalladium(0), 0.22 g (0.10 eq, 1.1 mmol) of tri-tert-butylphosphine, and 4.0 g (4.0 eq, 44 mmol) of sodium tert-butoxide were added to a reaction container and vacuum-dried, and the reaction container was filled with nitrogen gas. 50 mL of toluene was added to the reaction container to dissolve the compounds, and the compounds were stirred at a temperature of 30° C. for 1 hour under reflux. After the reaction was completed, an organic layer was extracted therefrom by using distilled water and ethyl acetate. The extracted organic layer was dried by using anhydrous magnesium sulfate and filtered through Celite, and a solvent was evaporated therefrom. After column chromatography, recrystallization using dichloromethane and hexane was performed thereon to obtain 2.7 g (yield of 61%) of a final Compound 9 (9,10-dihydro-9,9-dimethyl-10-(4-(oxazolo[5,4-b]pyridin-2-yl)phenyl)acridine).

¹H NMR (500 MHz, CDCl₃) δ 8.55 (d, J=8.5 Hz, 2H), 8.40 (dd, J=5.0, 1.5 Hz, 1H), 8.12 (dd, J=7.8, 1.5 Hz, 1H), 7.56 (d, J=8.5 Hz, 2H), 7.48 (dd, J=7.5, 1.6 Hz, 2H), 7.40 (dd, J=7.8, 5.0 Hz, 1H), 6.98 (dtd, J=18.2, 7.3, 1.4 Hz, 4H), 6.34 (dt, J=12.9, 6.5 Hz, 2H), 1.71 (s, 6H), APCI-MS (m/z): 403 [M+].

### Synthesis Example 4: Synthesis of Compound 11

(1) Synthesis of Intermediate 11(1)

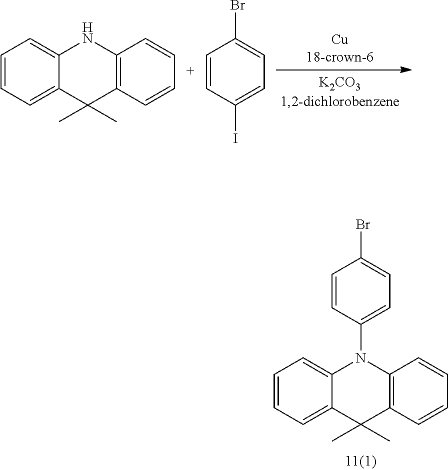

3.0 g (1.0 eq, 14 mmol) of 9,10-dihydro-9,9-dimethylacridine, 5.3 g (1.3 eq, 19 mmol) of 4-bromoiodobenzene, 0.30 g (0.080 eq, 1.2 mmol) of 18-crown-6, 0.47 g (0.52 Zeq, 7.5 mmol) of copper (powder), and 7.9 g (4.0 eq, 57 mmol) of potassium carbonate were added to a reaction container and vacuum-dried, and the reaction container was filled with nitrogen gas. 20 mL of 1,2-dichlorobenzene was added to the reaction container and stirred at a temperature of 190° C. for 2 hours under reflux. After the reaction was completed, an organic layer was extracted therefrom by using distilled water and ethyl acetate. The extracted organic layer was dried by using anhydrous magnesium sulfate and filtered through Celite, and a solvent was evaporated therefrom. Then, column chromatography was performed thereon to obtain 3.8 g (yield of 72%) of Intermediate 11(1), that is, 10-(4-bromophenyl)-9,10-dihydro-9,9-dimethylacridine.

¹H NMR (500 MHz, CDCl₃) δ 7.78-7.73 (m, 2H), 7.45 (dd, J=7.6, 1.6 Hz, 2H), 7.24-7.20 (m, 2H), 7.00-6.91 (m, 4H), 6.25 (dd, J=8.1, 1.4 Hz, 2H), 1.70-1.66 (s, 6H).

(2) Synthesis of Intermediate 11(2)

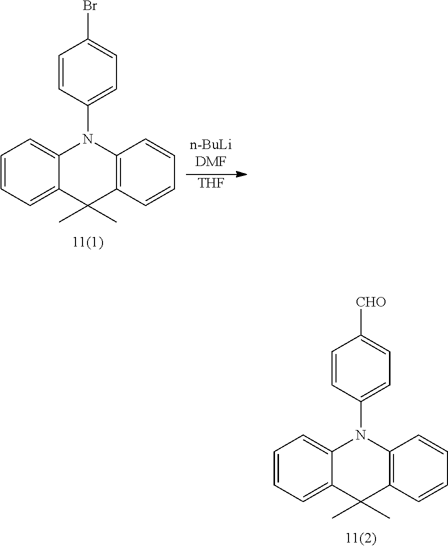 [SMILES: CC1(C)c2ccccc2N(c2ccc(Br)cc2)c2ccccc21 ; CC1(C)c2ccccc2N(c2ccc(C=O)cc2)c2ccccc21 ; "THF"]

3.8 g (1.0 eq, 5.2 mmol) of 110-(4-bromophenyl)-9,10-dihydro-9,9-dimethylacridine was added to a reaction container and vacuum-dried, and the reaction container was filled with nitrogen gas. 8 mL of THF was added thereto to dissolve the compounds, and the reaction container was cooled to a temperature of −78° C. by using dry ice. 6.2 mL (1.5 eq, 7.7 mmol) of 1.6 M n-butyllithium was slowly added dropwise to the reaction container while maintaining the temperature of −78° C., and stirred for 90 minutes. 1.2 mL (1.5 eq, 7.7 mmol) of dimethylformamide (DMF) was added to the reaction container and stirred at room temperature for 3 hours. After the reaction was completed, an organic layer was extracted therefrom by using distilled water and ethyl acetate. The extracted organic layer was dried by using anhydrous magnesium sulfate and filtered through Celite, and a solvent was evaporated therefrom. Then, column chromatography was performed thereon to obtain 2.4 g (yield of 74%) of Intermediate 11(2), that is, 4-(9,9-dimethylacridin-10(9H)-yl)benzaldehyde.

¹H NMR (500 MHz, CDCl₃) δ 10.12 (s, 1H), 8.17-8.10 (m, 2H), 7.56-7.52 (m, 2H), 7.51-7.47 (m, 2H), 7.02-6.96 (m, 4H), 6.36-6.30 (m, 2H), 1.69 (s, 6H).

(3) Synthesis of Compound 11

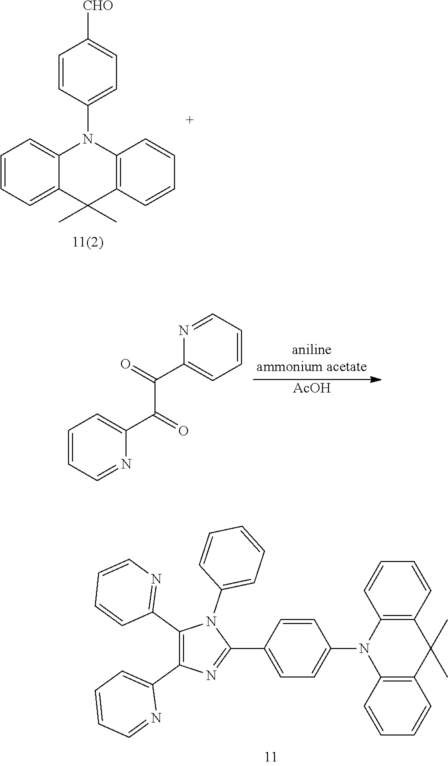

0.74 g (1 eq, 2.4 mmol) of 4-(9,9-dimethylacridin-10(9H)-yl)benzaldehyde, 0.50 g (1.0 eq, 2.4 mmol) of 2,2′-pyridil, 1.1 mL (5.0 eq, 12 mmol) of aniline, and 2.3 g (12 eq, 29 mmol) of ammonium acetate were added to a reaction container and vacuum-dried, and the reaction container was filled with nitrogen gas. 15 mL of acetic acid was added thereto and stirred at a temperature of 120° C. for 4 hours under reflux. After the reaction was completed, the reaction product was neutralized by using aqueous sodium hydroxide solution and washed with distilled water, and an organic layer was extracted therefrom by using chloroform. The extracted organic layer was dried by using anhydrous magnesium sulfate and filtered through Celite, and a solvent was evaporated therefrom. Then, column chromatography and recrystallization using dichloromethane and hexane were performed thereon to obtain 0.61 g (yield of 45%) of a final Compound 11 (9,10-dihydro-9,9-dimethyl-10-(4-(1-phenyl-4,5-di(pyridin-2-yl)-1H-imidazol-2-yl)phenyl)acridine).

¹H NMR (500 MHz, CDCl₃) δ 8.49 (d, J=25.3 Hz, 2H), 7.87 (d, J=8.0 Hz, 1H), 7.74-7.69 (m, 2H), 7.65 (t, J=6.9 Hz, 1H), 7.62-7.55 (m, 1H), 7.45-7.37 (m, 3H), 7.33-7.28 (m, 3H), 7.25-7.19 (m, 4H), 7.16-7.09 (m, 2H), 6.94 (dtd, J=23.4, 7.3, 1.5 Hz, 4H), 6.24 (dd, J=8.1, 1.3 Hz, 2H), 1.66 (s, 6H), APCI-MS (m/z): 582 [M+].

### Synthesis Example 5: Synthesis of Compound 13

(1) Synthesis of Compound 13(1)

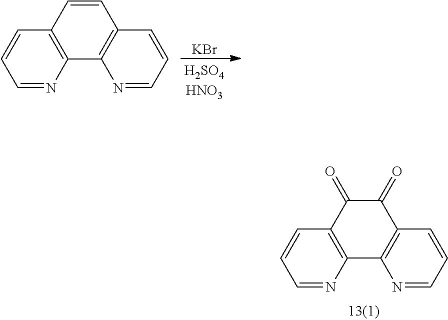

2.0 g (1.0 eq, 11 mmol) of 1,10-phenanthroline and 2.1 g (1.6 eq, 17 mmol) of potassium bromide were added to a reaction container and vacuum-dried, and the reaction container was filled with nitrogen gas. The reaction container was cooled to a temperature of 0° C., 40 mL of a mixed solution containing 20 mL of sulfuric acid and 20 mL of nitric acid was slowly added dropwise to the reaction container and stirred to dissolve the compounds, and the compounds were stirred at a temperature of 100° C. for 3 hours under reflux. After the reaction was completed, the reaction product was neutralized by using sodium carbonate and washed with distilled water, and an organic layer was extracted therefrom by dichloromethane. The extracted organic layer was dried by using magnesium sulfate and filtered through Celite, and a solvent was evaporated therefrom. Then, recrystallization using dichloromethane and ethanol was performed thereon to obtain 1.3 g (yield of 54%) of Intermediate 13(1), that is, 1,10-phenanthroline-5,6-dione.

¹H NMR: 9.13 (dd, J=4.7, 1.8 Hz, 2H), 8.52 (dd, J=7.9, 1.8 Hz, 2H), 7.60 (dd, J=7.9, 4.7 Hz, 2H).

(2) Synthesis of Compound 13

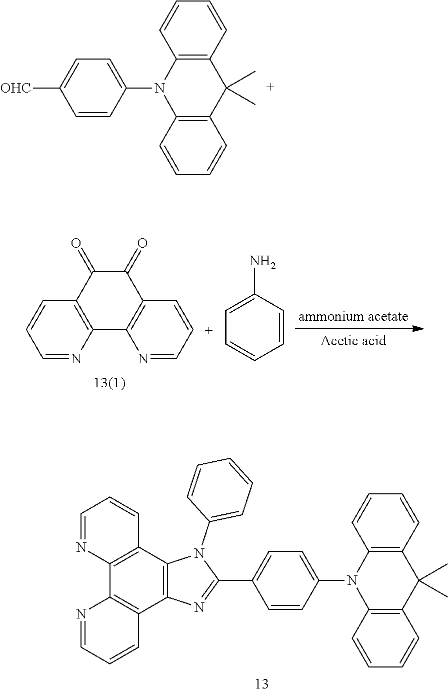 [SMILES: CC1(C)c2ccccc2N(c2ccc(C=O)cc2)c2ccccc21 ; O=C1C(=O)c2cccnc2C2=C1C=[C+]C=N2 ; Nc1ccccc1 ; CC1(C)c2ccccc2N(c2ccc(-c3nc4c5cccnc5c5ncccc5c4n3-c3ccccc3)cc2)c2ccccc21]

0.50 g (1.0 eq, 2.4 mmol) of 1,10-phenanthroline-5,6-dione, 0.74 g (1.0 eq, 2.4 mmol) of 4-(9,9-dimethylacridin-10(9H)-yl)benzaldehyde, 1.1 mL (5.0 eq, 12 mmol) of aniline, and 2.1 g (12 eq, 29 mmol) of ammonium acetate were added to a reaction container and vacuum-dried, and the reaction container was filled with nitrogen gas. 15 mL of acetic acid was added to the reaction container to dissolve the compounds, and the compounds were stirred at a temperature of 130° C. for 16 hours under reflux. After the reaction was completed, the reaction product was neutralized by using sodium carbonate and washed with distilled water, and an organic layer was extracted therefrom by using ethyl acetate. The extracted organic layer was dried by using magnesium sulfate and filtered through Celite, and a solvent was evaporated therefrom. After column chromatography, recrystallization using ethanol was performed thereon to obtain 0.50 g (yield of 37%) of a final Compound 13 (2-(4-(9,9-dimethylacridin-10(9H)-yl)phenyl)-1-phenyl-1H-imidazo[4,5-f][1,10]phenanthroline).

¹H NMR: 9.21 (ddd, J=9.8, 6.2, 1.8 Hz, 2H), 9.07 (dt, J=23.2, 11.6 Hz, 1H), 7.91-7.83 (m, 2H), 7.79 (dd, J=8.1, 4.4 Hz, 1H), 7.74-7.68 (m, 3H), 7.67-7.60 (m, 2H), 7.51 (dd, J=8.4, 1.6 Hz, 1H), 7.48-7.43 (m, 2H), 7.32 (ddd, J=8.5, 7.5, 3.1 Hz, 3H), 7.01-6.87 (m, 4H), 6.29-6.17 (m, 2H), 1.36 (dd, J=17.1, 5.7 Hz, 6H). APCI-MS (m/z): 580 [M+].

### Synthesis Example 6: Synthesis of Compound 15

(1) Synthesis of Intermediate 15(1)

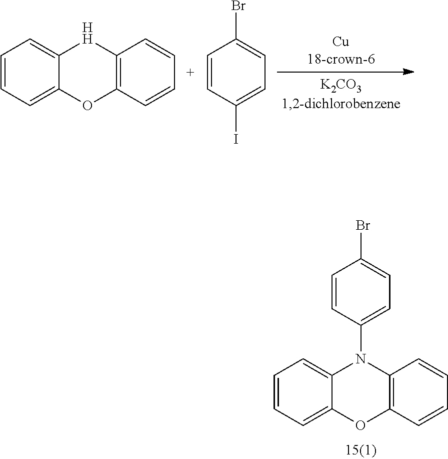

0.80 g (1.0 eq, 4.4 mmol) of 10H-phenoxazine, 1.6 g (1.3 eq, 5.7 mmol) of 4-bromoiodobenzene, 0.090 g (0.080 eq, 0.35 mmol) of 18-crown-6, 0.14 g (0.52 eq, 2.3 mmol) of copper (powder), and 2.4 g (4.0 eq, 17 mmol) of potassium carbonate were added to a reaction container and vacuum-dried, and the reaction container was filled with nitrogen gas. 11 mL of 1,2-dichlorobenzene were added to the reaction container and stirred at a temperature of 190° C. for 2 hours under reflux. After the reaction was completed, an organic layer was extracted therefrom by using distilled water and ethyl acetate. The extracted organic layer was dried by using anhydrous magnesium sulfate and filtered through Celite, and a solvent was evaporated therefrom. Then, column chromatography was performed thereon to obtain 1.1 g (yield of 72%) of Intermediate 14(1), that is, 10-(4-bromophenyl)-10H-phenoxazine.

¹H NMR (500 MHz, CDCl₃) δ 7.75-7.70 (m, 2H), 7.25-7.22 (m, 2H), 6.67 (dtd, J=9.2, 7.8, 1.6 Hz, 4H), 6.60 (td, J=7.6, 1.7 Hz, 2H), 5.94-5.88 (m, 2H).

(2) Synthesis of Intermediate 15(2)

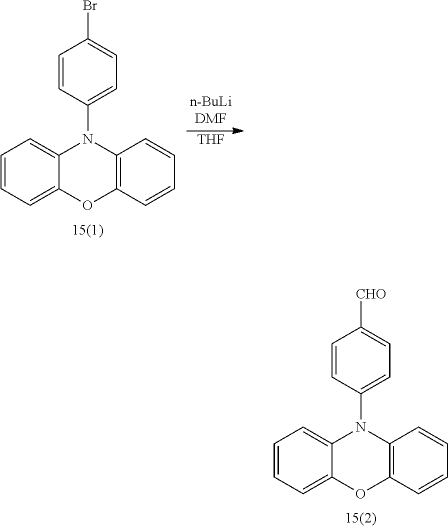 [SMILES: Brc1ccc(N2c3ccccc3Oc3ccccc32)cc1 ; O=Cc1ccc(N2c3ccccc3Oc3ccccc32)cc1 ; "THF"]

0.80 g (1.0 eq, 2.4 mmol) of 10-(4-bromophenyl)-10H-phenoxazine was added to a reaction container and vacuum-dried, and the reaction container was filled with nitrogen gas. 8 mL of THF was added thereto to dissolve the compounds, and the reaction container was cooled to a temperature of −78° C. by using dry ice. 2.2 mL (1.5 eq, 3.5 mmol) of 1.6 M n-butyllithium was slowly added dropwise to the reaction container while maintaining the temperature of −78° C., and stirred for 90 minutes. 0.27 m1 (1.5 eq, 3.5 mmol) of dimethylformamide was added to the reaction container and stirred at room temperature for 3 hours. After the reaction was completed, an organic layer was extracted therefrom by using distilled water and ethyl acetate. The extracted organic layer was dried by using anhydrous magnesium sulfate and filtered through Celite, and a solvent was evaporated therefrom. Then, column chromatography was performed thereon to obtain 0.40 g (yield of 59%) of Intermediate 14(2), that is, 4-(10H-phenoxazin-10-yl)benzaldehyde.

¹H NMR (500 MHz, CDCl₃) δ 10.11 (s, 1H), 8.16-8.08 (m, 2H), 7.62-7.51 (m, 2H), 6.71 (dtd, J=9.3, 7.9, 1.6 Hz, 4H), 6.61 (ddd, J=17.8, 10.1, 6.1 Hz, 2H), 5.96 (dd, J=8.0, 1.4 Hz, 2H).

(3) Synthesis of Compound 15

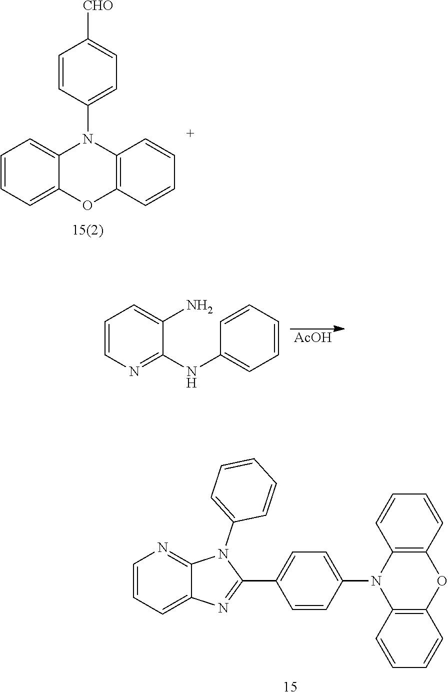 [SMILES: O=Cc1ccc(N2c3ccccc3Oc3ccccc32)cc1 ; "AcOH" ; Nc1cccnc1Nc1ccccc1 ; c1ccc(-n2c(-c3ccc(N4c5ccccc5Oc5ccccc54)cc3)nc3cccnc32)cc1]

0.28 g (1.2 eq, 0.97 mmol) of 4-(10H-phenoxazin-10-yl)benzaldehyde and 0.15 g (1.0 eq, 0.81 mmol) of N2-phenylpyridine-2,3-diamine were added to a reaction container and vacuum-dried, and the reaction container was filled with nitrogen gas. 5 mL of acetic acid was added thereto and stirred at a temperature of 120° C. for 3 hours under reflux. After the reaction was completed, the reaction product was neutralized by using sodium carbonate, and an organic layer was extracted therefrom by using distilled water and ethyl acetate. The extracted organic layer was dried by using anhydrous magnesium sulfate and filtered through Celite, and a solvent was evaporated therefrom. After column chromatography, recrystallization using dichloromethane and hexane was performed thereon to obtain 0.25 g (yield of 69%) of a final Compound 14 (10-(4-(3-phenyl-3H-imidazo[4,5-b]pyridin-2-yl)phenyl)-10H-phenoxazine).

¹H NMR (500 MHz, CDCl₃) δ 8.42 (dd, J=4.8, 1.5 Hz, 1H), 8.17 (dd, J=8.0, 1.5 Hz, 1H), 7.85-7.81 (m, 2H), 7.59-7.52 (m, 2H), 7.52-7.48 (m, 1H), 7.44 (dt, J=8.7, 2.7 Hz, 2H), 7.35-7.30 (m, 2H), 7.27-7.23 (m, 1H), 6.69-6.61 (m, 4H), 6.58 (td, J=7.6, 1.7 Hz, 2H), 5.92 (dd, J=7.9, 1.4 Hz, 2H). APCI-MS (m/z): 453 [M+].

### Synthesis Example 7: Synthesis of Compound 25

(1) Synthesis of Intermediate 25(1)

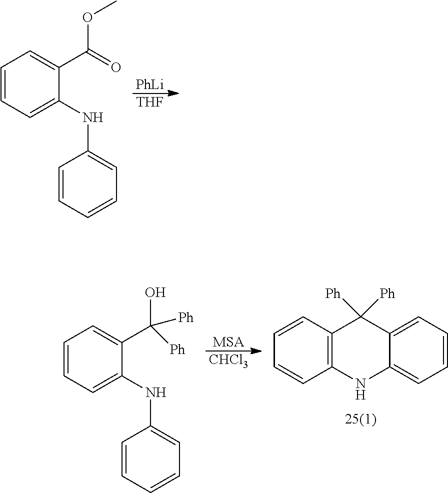

1.0 g (1.0 eq, 4.4 mmol) of methyl 2-(phenylamino)benzoate was added to a reaction container and vacuum-dried, and the reaction container was filled with nitrogen gas. 17 mL of THF was added to the reaction container and stirred to dissolve the compounds, and the reaction container was cooled to a temperature of −78° C. 1.9 M phenyllithium (3.0 eq, 7.0 ml) was slowly added dropwise thereto and stirred at a temperature of −78° C. for 1 hour. The reaction solution was stirred at a temperature of 0° C. for 2 hours and then additionally stirred at room temperature for 4 hours. After the reaction was completed, the reaction product was washed with distilled water, and an organic layer was extracted therefrom by using chloroform. The extracted organic layer was dried by using magnesium sulfate and filtered through Celite, and a solvent was evaporated therefrom. The dried reaction product was added to a reaction container, and 20 mL of chloroform was added thereto to dissolve the compound. 1 mL of methanesulfonic acid (MSA) was added thereto and stirred for 1 hour under reflux. After the reaction was completed, the reaction product was neutralized by using sodium bicarbonate aqueous solution, and an organic layer was extracted therefrom by using distilled water and chloroform. The extracted organic layer was dried by using anhydrous magnesium sulfate and filtered through Celite, and a solvent was evaporated therefrom. After column chromatography, recrystallization using chloroform and hexane was performed thereon to obtain 1.1 g (yield of 73%) of Intermediate 25(1), that is, 9,9-diphenyl-9,10-dihydroacridine.

¹H NMR: 7.26-7.15 (m, 8H), 6.96-6.94 (m, 4H), 6.89 (td, J=7.5 Hz, 1.2 Hz, 2H), 6.83 (dd, J=7.8 Hz, 1.5 Hz, 2H), 6.80 (dd, J=7.9 Hz, 1.1 Hz, 2H), 6.28 (s, 1H).

(2) Synthesis of Compound 25

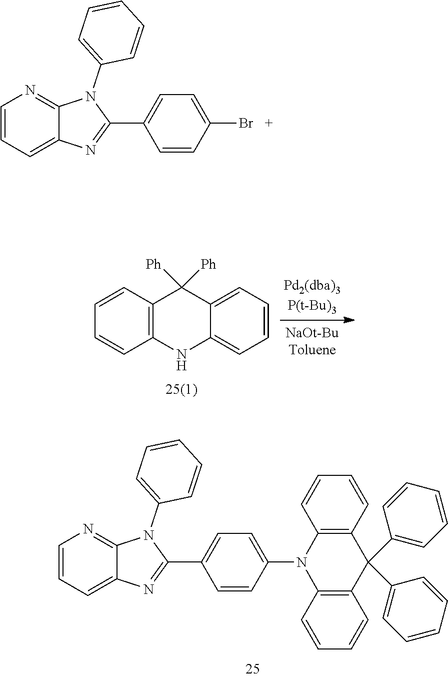

0.50 g (1.0 eq, 1.4 mmol) of 2-(4-bromophenyl)-3-phenyl-3H-imidazo[4,5-b]pyridine, 0.52 g (1.1 eq, 1.6 mmol) of 9,9-diphenyl-9,10-dihydroacridine, 0.13 g (0.10 eq, 0.14 mmol) of tris(dibenzylideneacetone)dipalladium(0), and 0.52 g (4.0 eq, 5.7 mmol) of sodium tert-butoxide were added to a reaction container and vacuum-dried, and the reaction container was filled with nitrogen gas. 9 mL of toluene was added to the reaction container to dissolve the compounds, and 0.060 g (0.20 eq, 0.28 mmol) of tri-tert-butylphosphine was added thereto and stirred at a temperature of 110° C. for 2 hours under reflux. After the reaction was completed, the reaction product was washed with distilled water, and an organic layer was extracted therefrom by using ethyl acetate. The extracted organic layer was dried by using magnesium sulfate and filtered through Celite, and a solvent was evaporated therefrom. After column chromatography, recrystallization using dichloromethane and hexane was performed thereon to obtain 0.57 g (yield of 66%) of a final Compound 25 (9,9-diphenyl-10-(4-(3-phenyl-3H-imidazo[4,5-b]pyridin-2-yl)phenyl)-9,10-dihydroacridine).

¹H NMR: 8.46-8.38 (m, 1H), 8.17 (dt, J=10.5, 5.3 Hz, 1H), 7.81-7.72 (m, 2H), 7.60-7.53 (m, 2H), 7.53-7.47 (m, 1H), 7.47-7.41 (m, 2H), 7.34 (dd, J=8.0, 4.8 Hz, 1H), 7.25-7.20 (m, 6H), 7.11-7.06 (m, 2H), 7.06-7.01 (m, 2H), 7.00-6.94 (m, 4H), 6.92-6.86 (m, 4H), 6.39 (d, J=8.2 Hz, 2H). APCI-MS (m/z): 603 [M+]

### Synthesis Example 8: Synthesis of Compound 74

(1) Synthesis of Intermediate 74(1)

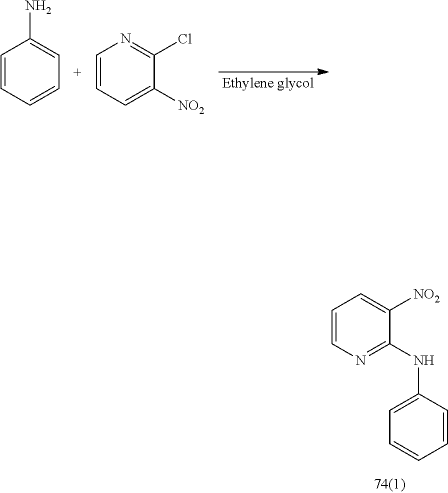

10 g (1.0 eq, 63 mmol) of 2-chloro-3-nitropyridine was added to a reaction container and vacuum-dried, and the reaction container was filled with nitrogen gas. Then, 5.8 mL (1.0 eq, 63 mmol) of aniline and 100 mL of ethylene glycol were added thereto and stirred at a temperature of 140° C. for 8 hours under reflux. After the reaction was completed, the reaction container was cooled to room temperature, and an organic layer was extracted therefrom by using distilled water and ethyl acetate. The extracted organic layer was dried by using anhydrous magnesium sulfate and filtered through Celite, and a solvent was evaporated therefrom. Then, column chromatography was performed thereon to obtain 12 g (yield of 87%) of Intermediate 74(1), that is, 3-nitro-N-phenylpyridin-2-amine.

¹H NMR (500 MHz, CDCl₃) δ 10.12 (s, 1H), 8.53 (dd, J=8.3, 1.8 Hz, 1H), 8.49 (dd, J=4.5, 1.8 Hz, 1H), 7.65 (dt, J=8.7, 1.6 Hz, 2H), 7.42-7.38 (m, 2H), 7.21-7.17 (m, 1H), 6.83 (dd, J=8.3, 4.5 Hz, 1H).

(2) Synthesis of Intermediate 74(2)

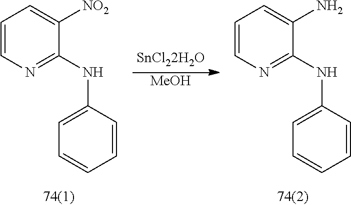 [SMILES: "MeOH" ; O=[N+]([O-])c1cccnc1Nc1ccccc1 ; Nc1cccnc1Nc1ccccc1]

12 g (1.0 eq, 55 mmol) of 3-nitro-N-phenylpyridin-2-amine and 37 g (3.0 eq, 160 mmol) of SnCl₂.2H₂Owere added to a reaction container and vacuum-dried, and the reaction container was filled with nitrogen gas. 300 mL of methanol was added thereto and stirred at a temperature of 60° C. for 2 hours under reflux. NaHCO₃ aqueous solution was added thereto to complete the reaction, and an organic layer was extracted therefrom by using distilled water and ethyl acetate. The extracted organic layer was dried by using anhydrous magnesium sulfate and filtered through Celite, and a solvent was evaporated therefrom. Then, recrystallization using dichloromethane and hexane was performed thereon to obtain 8.2 g (yield of 81%) of Intermediate 74(2), that is, N²-phenylpyridine-2,3-diamine.

¹H NMR (500 MHz, CDCl₃) δ 7.84 (dd, J=4.9, 1.5 Hz, 1H), 7.31-7.24 (m, 4H), 7.02 (dd, J=7.6, 1.6 Hz, 1H), 6.96 (tt, J=7.1, 1.6 Hz, 1H), 6.78 (dd, J=7.6, 4.9 Hz, 1H), 6.19 (s, 1H), 3.41 (s, 2H).

(3) Synthesis of Intermediate 74(3)

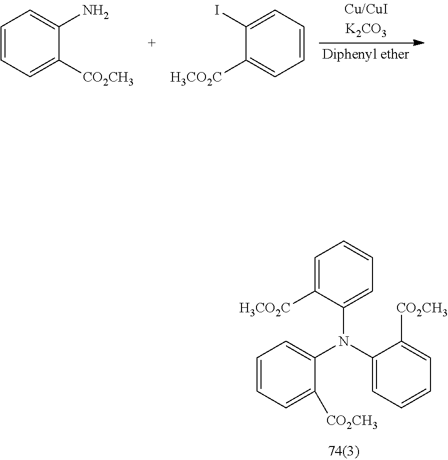 [SMILES: COC(=O)c1ccccc1N ; COC(=O)c1ccccc1I ; COC(=O)c1ccccc1N(c1ccccc1C(=O)OC)c1ccccc1C(=O)OC]

methyl anthranilate (9.0 mL, 0.070 mol), methyl 2-iodobenzoate (30 mL, 0.20 mol), K₂CO₃ (22 g, 0.16 mol), Cu (0.90 g, 14 mmol), and CuI (1.3 g, 6.8 mmol) were added to a reaction container and vacuum-dried, and the reaction container was filled with nitrogen gas. Then, diphenylether (80 mL) was added thereto and stirred at a temperature of 190° C. for 6 days under reflux. A solvent was removed therefrom under reduced pressure, and a solid obtained therefrom was dissolved by using EA and methanol. Then, column chromatography was performed thereon to obtain Intermediate 74(3), that is, a yellow solid triester tertiary amine (22 g, yield of 75%).

¹H NMR (500 MHz, CDCl₃) δ 7.58 (d, J 7.7 Hz, 3H), 7.35 (t, J 7.6 Hz, 3H), 7.0-7.14 (m, 6H), 3.37 (s, 9H).

(4) Synthesis of Intermediate 74(4)

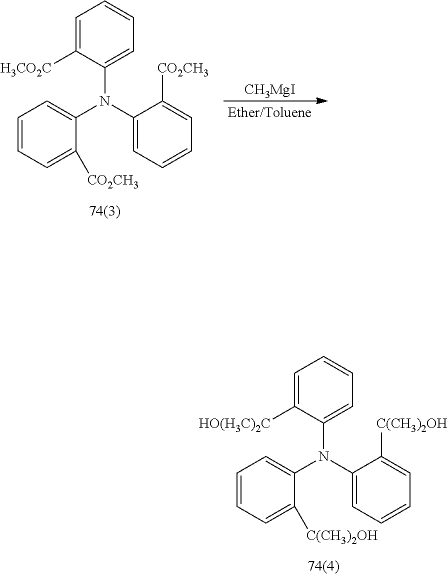

45 mL (15 eq, 89 mmol) of methyl magnesium iodide (2 M) was vacuum-dried, added to a reaction container filled with nitrogen gas, and then stirred. 2.5 g (1.0 eq, 2.0 mmol) of Intermediate 74(3), that is, triester tertiary amine, were dissolved in 70 mL of toluene and slowly added to the reaction container. The reaction solution was stirred at a temperature of 110° C. for 15 hours under reflux. After the reaction was completed, the reaction product was cooled to room temperature, and the reaction was slowly terminated through sulfuric acid. The reaction product was washed with distilled water, and an organic layer was extracted therefrom by using MC. The extracted organic layer was dried by using magnesium sulfate and filtered through Celite. Then, column chromatography (EA:hexane=1:5) was performed thereon to obtain 0.90 g (yield of 36%) of Intermediate 74(4), that is, a white solid triol amine.

¹H-NMR (500 MHz, CDCl₃) δ 7.32 (d, J 9.0 Hz, 3H), 7.0-7.15 (m, 6H), 6.68 (d, J 7.3 Hz, 3H), 5.37 (s, 3H), 1.67 (s, 9H), 0.84 (s, 9H).

(5) Synthesis of Intermediate 74(5)

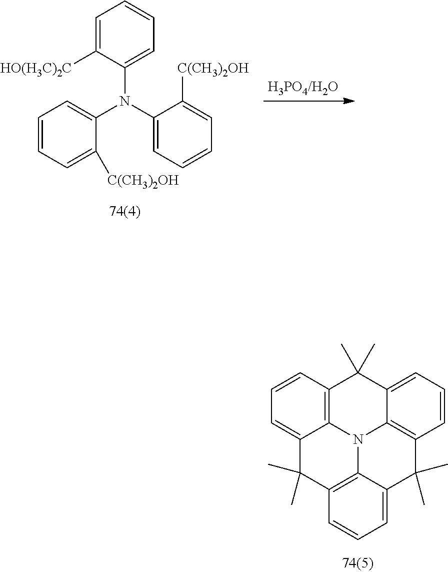

Intermediate 74(4), that is, triol amine (0.90 g, 2.2 mmol) was added to a reaction container, and 85% aqueous phosphoric acid solution (10 mL) was added thereto and stirred at room temperature for 2 hours. After the reaction was completed, the reaction was terminated through NaOH, the reaction product was washed with distilled water, and an organic layer was extracted therefrom by using MC. Then, column chromatography using hexane was performed thereon to obtain 0.46 g (yield of 59%) of Intermediate 74(5), that is, a white solid triaryl amine.

¹H NMR (500 MHz, CDCl₃) δ 7.37 (d, J 7.7 Hz, 6H), 7.12 (t, J 7.8 Hz, 3H), 1.63 (s, 18H).

(6) Synthesis of Intermediate 74(6)

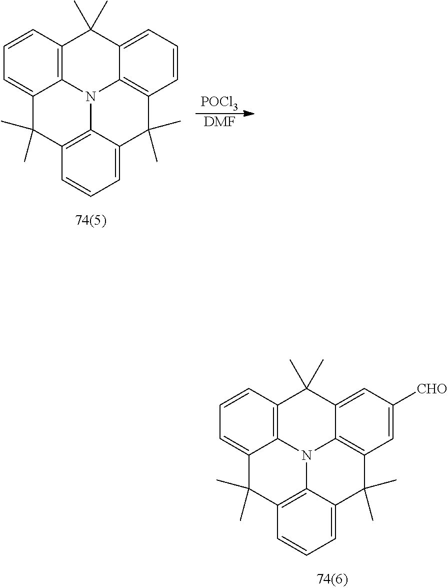

0.40 g (1.0 eq, 1.1 mmol) of Intermediate 74(5), that is, triaryl amine was added to a reaction container and vacuum-dried, and the reaction container was filled with nitrogen gas. 40 mL of DMF was added thereto, and the reaction container was cooled to a temperature of 0° C. by using ice. 1.7 mL of POCl₃ and 5 mL of DMF were mixed in another reaction container, and the reaction container was cooled to a temperature of 0° C. The mixture was slowly added dropwise and stirred at room temperature until the color thereof was changed to brown. Then, the reaction container was heated to a temperature of 80° C. and heated and stirred for 15 hours. After the reaction was completed, the reaction was terminated through sodium acetate, and the reaction solution was extracted therefrom by using MC. The reaction product was dried by using magnesium sulfate and filtered through Celite, and column chromatography was performed thereon to obtain 0.29 g (yield of 67%) of Intermediate 74(6), that is, a yellow solid 4,4,8,8,12,12-hexamethyl-8,12-dihydro-4H-benzo[9,1]quinolizinoacridine-2-carbaldehyde.

¹H NMR (300 MHz, CDCl₃) δ 9.93 (s, 1H), 7.89 (s, 2H), 7.42 (dd, J 7.6, 2.3 Hz, 4H), 7.20 (t, J 7.6 Hz, 2H), 1.67 (s, 6H), 1.65 (s, 12H).

(7) Synthesis of Compound 74

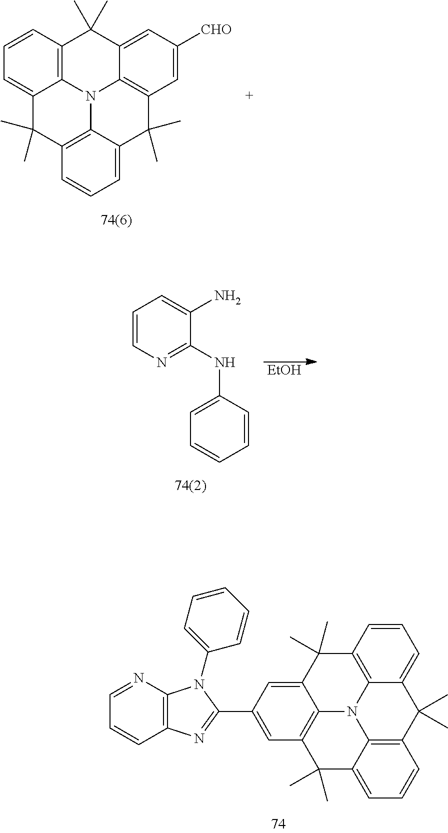 [SMILES: "EtOH" ; CC1(C)c2cccc3c2N2c4c1cccc4C(C)(C)c1cc(C=O)cc(c12)C3(C)C ; Nc1cccnc1Nc1ccccc1 ; CC1(C)c2cccc3c2N2c4c1cccc4C(C)(C)c1cc(-c4nc5cccnc5n4-c4ccccc4)cc(c12)C3(C)C]

0.14 g (1.0 eq, 7.6 mmol) of N²-phenylpyridine-2,3-diamine and 0.33 g (1.1 eq, 0.83 mmol) of 4,4,8,8,12,12-hexamethyl-8,12-dihydro-4H-benzo[9,1]quinolizinoacridine-2-carbaldehyde were added to a reaction container and vacuum-dried, and the reaction container was filled with nitrogen gas. Then, 10 mL of ethanol was added thereto and stirred at a temperature of 80° C. for 3 days under reflux. After the reaction was completed, the reaction product was washed with distilled water, and an organic layer was extracted therefrom by using EA. After column chromatography (EA:hexane 1:4), recrystallization using MC and hexane was performed thereon to obtain 0.11 g (yield of 26%) of a final Compound 74 that was a yellow solid.

¹H NMR (500 MHz, CDCl₃) δ 8.36 (dd, J=4.8, 1.3 Hz, 1H), 8.17 (dd, J=8.0, 1.3 Hz, 1H), 7.65 (s, 2H), 7.55 (t, J=7.6 Hz, 2H), 7.48 (d, J=7.4 Hz, 1H), 7.44 (dd, J=6.8, 5.5 Hz, 2H), 7.38 (dd, J=7.7, 1.4 Hz, 2H), 7.33 (dd, J=7.7, 1.4 Hz, 2H), 7.29 (dd, J=8.0, 4.8 Hz, 1H), 7.13 (t, J=7.7 Hz, 2H), 1.63 (d, J=7.4 Hz, 6H), 1.45 (s, 12H))

### Synthesis Example 9: Synthesis of Compound 83

(1) Synthesis of Intermediate 83(1)

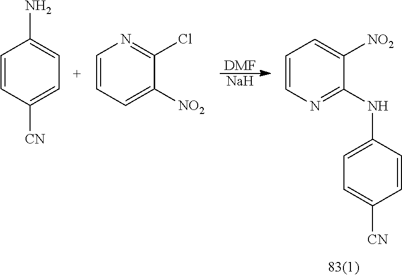

2.0 g (1.0 eq, 17 mmol) of 4-aminobenzonitrile and 0.81 g (2.0 eq, 34 mmol) of sodium hydride were added to a reaction container and vacuum-dried, and the reaction container was filled with nitrogen gas. DMF was added thereto and stirred for about 30 minutes, and 2-chloro-3-nitropyridine was added thereto and stirred at a temperature of 100° C. for 9 hours under reflux. After the reaction was completed, the reaction was terminated by using distilled water. The reaction product was washed, and an organic layer was extracted therefrom by using ethyl acetate. The extracted organic layer was dried by using magnesium sulfate and filtered through Celite, and a solvent was evaporated therefrom. Then, column chromatography was performed thereon to obtain 0.95 g (yield of 31%) of Intermediate 83(1), that is, 4-(3-nitropyridin-2-ylamino)benzonitrile.

¹H NMR (500 MHz, CDCl₃) δ 10.36 (s, 1H), 8.85-8.29 (m, 2H), 8.08-7.76 (m, 2H), 7.81-7.50 (m, 2H), 7.02 (ddd, J=19.9, 8.4, 4.6 Hz, 1H).

(2) Synthesis of Intermediate 83(2)

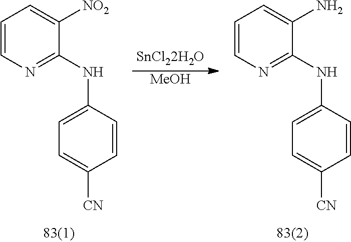 [SMILES: "MeOH" ; N#Cc1ccc(Nc2ncccc2[N+](=O)[O-])cc1 ; N#Cc1ccc(Nc2ncccc2N)cc1]

0.50 g (1.0 eq, 2.1 mmol) of 4-(3-nitropyridin-2-ylamino)benzonitrile and 15 mL of MeOH were added to a reaction container, and 1.9 g (4.0 eq, 8.3 mmol) of tin(II) chloride dihydrate was added thereto little by little while slowly increasing the temperature. Then, the reaction mixture was stirred at a temperature of 80° C. for 1 hour under reflux. After the reaction was completed, the reaction was terminated by using sodium bicarbonate, the reaction product was washed, and an organic layer was extracted therefrom by using ethyl acetate. The extracted organic layer was dried by using magnesium sulfate and filtered through Celite, and a solvent was evaporated therefrom. Then, column chromatography was performed thereon to obtain 0.42 g (yield of 96%) of Intermediate 83(2) that is, 4-(3-aminopyridin-2-ylamino)benzonitrile.

¹H NMR (500 MHz, CDCl₃) δ 7.94-7.81 (m, 1H), 7.61-7.52 (m, 2H), 7.41-7.34 (m, 2H), 7.13-7.04 (m, 1H), 6.92-6.82 (m, 1H), 6.52 (s, 1H), 3.45 (s, 2H).

(3) Synthesis of Intermediate 83(3)

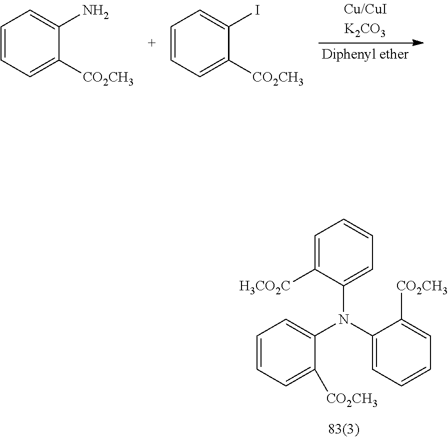 [SMILES: COC(=O)c1ccccc1N ; COC(=O)c1ccccc1I ; COC(=O)c1ccccc1N(c1ccccc1C(=O)OC)c1ccccc1C(=O)OC]

Methyl anthranilate (9.0 mL, 0.070 mol), methyl 2-iodobenzoate (30 mL, 0.20 mol), K₂CO₃ (22 g, 0.16 mol), Cu (0.90 g, 14 mmol), and CuI (1.3 g, 6.8 mmol) were added to a reaction container and vacuum-dried, and the reaction container was filled with nitrogen gas. Then, diphenylether (80 mL) was added thereto and stirred at a temperature of 190° C. for 6 days. A solvent was removed therefrom under reduced pressure, and a solid obtained therefrom was dissolved by using EA and methanol. Then, column chromatography was performed thereon to obtain 22 g (yield of 75%) of Intermediate 83(3), that is, a yellow solid triester tertiary amine.

¹H NMR (500 MHz, CDCl₃) δ 7.58 (d, J 7.7 Hz, 3H), 7.35 (t, J 7.6 Hz, 3H), 7.0-7.14 (m, 6H), 3.37 (s, 9H).

(4) Synthesis of Intermediate 83(4)

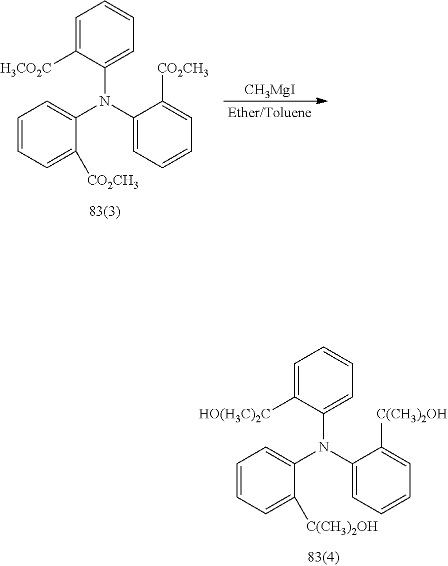

45 mL (15 eq, 89 mmol) of methyl magnesium iodide (2M) was vacuum-dried, added to a reaction container filled with nitrogen gas, and then stirred. 2.5 g (1.0 eq, 2.0 mmol) of Intermediate 83(3), that is, triester tertiary amine, was dissolved in 70 ml of toluene and slowly added to the reaction container. The reaction mixture was stirred at a temperature of 110° C. for 15 hours under reflux. After the reaction was completed, the reaction product was cooled to room temperature, and the reaction was slowly terminated through sulfuric acid. Then, the reaction product was washed with distilled water, and an organic layer was extracted therefrom by using MC. The extracted organic layer was dried by using magnesium sulfate and filtered through Celite. Then, column chromatography (EA:hexane=1:5) was performed thereon to obtain 0.90 g (yield of 36%) of Intermediate 83(4), that is, a white solid triol amine.

¹H NMR (500 MHz, CDCl₃) δ 7.32 (d, J 9.0 Hz, 3H), 7.0-7.15 (m, 6H), 6.68 (d, J 7.3 Hz, 3H), 5.37 (s, 3H), 1.67 (s, 9H), 0.84 (s, 9H).

(5) Synthesis of Intermediate 83(5)

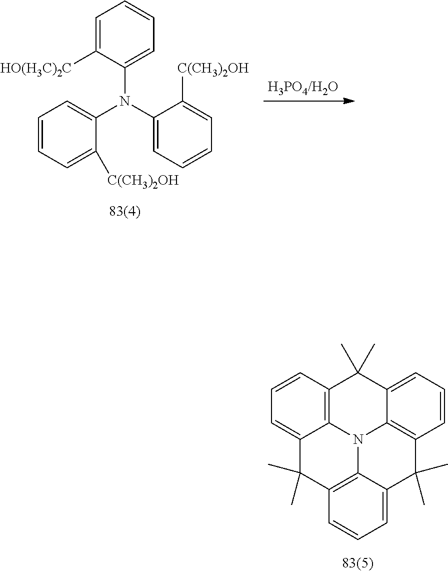

Intermediate 83(4), that is, triol amine (0.90 g, 2.2 mmol), was added to a reaction container, 85% aqueous phosphoric acid solution (10 mL) was added thereto, and the reaction mixture was stirred at room temperature for 2 hours. After the reaction was completed, the reaction was terminated through NaOH. Then, the reaction product was washed with distilled water, and an organic layer was extracted by using MC. Then, chromatography using hexane was performed thereon to obtain 0.46 g (yield of 59%) of Intermediate 83(5), that is, a white solid triaryl amine.

¹H NMR (500 MHz, CDCl₃) δ 7.37 (d, J 7.7 Hz, 6H), 7.12 (t, J 7.8 Hz, 3H), 1.63 (s, 18H).

(6) Synthesis of Intermediate 83(6)

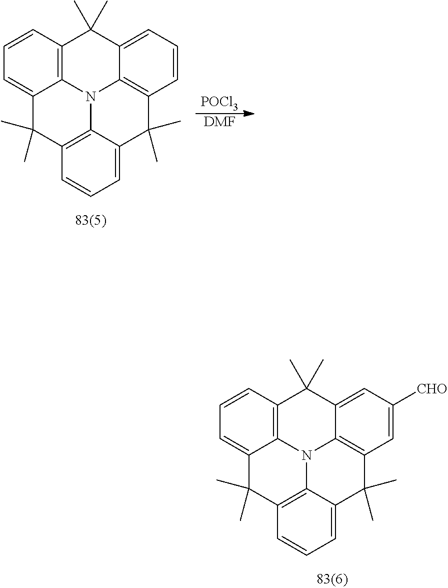

0.40 g (1.0 eq, 1.1 mmol) of Intermediate 83(5), that is, triaryl amine, was added to a reaction container and vacuum-dried, and the reaction container was filled with nitrogen gas. 40 mL of DMF was added thereto, and the reaction container was cooled to a temperature of 0° C. by using ice. 1.7 mL of POCl₃ and 5 mL of DMF were mixed in another reaction container, and the reaction container was cooled to a temperature of 0° C. The reaction mixture was slowly added dropwise thereto and stirred at room temperature until the reaction mixture became brown. Then, the reaction container was heated to a temperature of 80° C., and the reaction mixture was stirred for 15 hours. After the reaction was completed, the reaction was terminated through sodium acetate, and a reaction solution was extracted therefrom by using MC. The extracted reaction solution was dried by using magnesium sulfate and filtered through Celite. Then, column chromatography was performed thereon to obtain 0.29 g (yield of 67%) of Intermediate 83(6), that is, a yellow solid 4,4,8,8,12,12-hexamethyl-8,12-dihydro-4H-benzo[9,1]quinolizinoacridine-2-carbaldehyde.

¹H NMR (300 MHz, CDCl₃) δ 9.93 (s, 1H), 7.89 (s, 2H), 7.42 (dd, J 7.6, 2.3 Hz, 4H), 7.20 (t, J 7.6 Hz, 2H), 1.67 (s, 6H), 1.65 (s, 12H).

(7) Synthesis of Compound 83

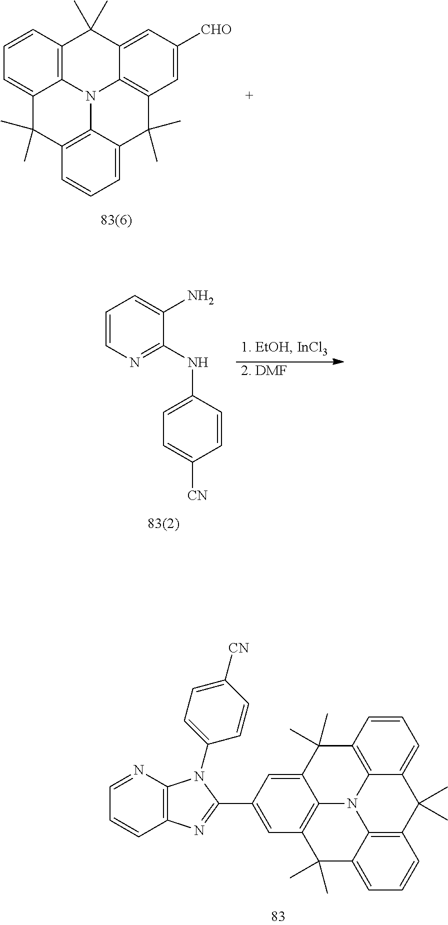 [SMILES: CC1(C)c2cccc3c2N2c4c1cccc4C(C)(C)c1cc(C=O)cc(c12)C3(C)C ; N#Cc1ccc(Nc2ncccc2N)cc1 ; "2. DMF" ; CC1(C)c2cccc3c2N2c4c1cccc4C(C)(C)c1cc(-c4nc5cccnc5n4-c4ccc(C#N)cc4)cc(c12)C3(C)C]

0.16 g (1.1 eq, 7.6 mmol) of 4-(3-aminopyridin-2-ylamino)benzonitrile, 0.28 g (1.0 eq, 0.71 mmol) of 4,4,8,8,12,12-hexamethyl-8,12-dihydro-4H-benzo[9,1]quinolizinoacridine-2-carbaldehyde, and 0.020 g (0.10 eq, 0.070 mmol) of Indium chloride were added to a reaction container and vacuum-dried, and the reaction container was filled with nitrogen gas. Then, 10 mL of ethanol was added thereto and stirred at a temperature of 80° C. for hours under reflux. After the reaction was completed, a solvent was removed therefrom under reduced pressure. Then, a solid obtained therefrom was added to a reaction container and vacuum-dried, and the reaction container was filled with nitrogen gas. 10 mL of DMF was added to the reaction container and stirred at a temperature of 100° C. for 9 hours under reflux. After the reaction was completed, an organic layer was extracted therefrom by using ethyl acetate. The extracted organic layer was dried by using magnesium sulfate and filtered through Celite. Then, column chromatography (EA:hexane 1:4) was performed thereon to obtain 0.20 g (yield of 49%) of a final Compound 83.

¹H NMR (500 MHz, CDCl₃) δ 8.36 (dd, J=4.8, 1.4 Hz, 1H), 8.19 (dd, J=8.0, 1.4 Hz, 1H), 7.85-7.78 (m, 2H), 7.62-7.49 (m, 4H), 7.44-7.32 (m, 5H), 7.16 (t, J=7.7 Hz, 2H), 1.66 (s, 6H), 1.46 (s, 12H).

Synthesis methods of compounds other than the above-described Compounds may also be easily recognized by those of skill in the art by referring to the synthesis mechanisms and methods described above.

### Example 1

Referring to the compounds illustrated below, an ITO glass substrate was cut to a size of 50 mm×50 mm×0.5 mm, sonicated with isopropyl alcohol and pure water each for 10 minutes, and then cleansed by exposure to ultraviolet rays and ozone for 10 minutes. Then, the ITO glass substrate was provided to a vacuum deposition apparatus, a material NPB was vacuum-deposited on the ITO glass substrate to form a hole injection layer having a thickness of 40 Å, and a hole transport material mCP was vacuum-deposited on the hole injection layer to form a hole transport layer having a thickness of 10 Å. Compound 9 and mCBP were co-deposited on the hole transport layer at a weight ratio of 15:85 to form an emission layer having a thickness of 200 Å. Then, ETL1 was deposited on the emission layer to form an electron transport layer having a thickness of 300 Å, and Al was vacuum-deposited on the electron transport layer to form an Al electrode having a thickness of 1,200 Å, thereby completing the manufacture of an organic light-emitting device.

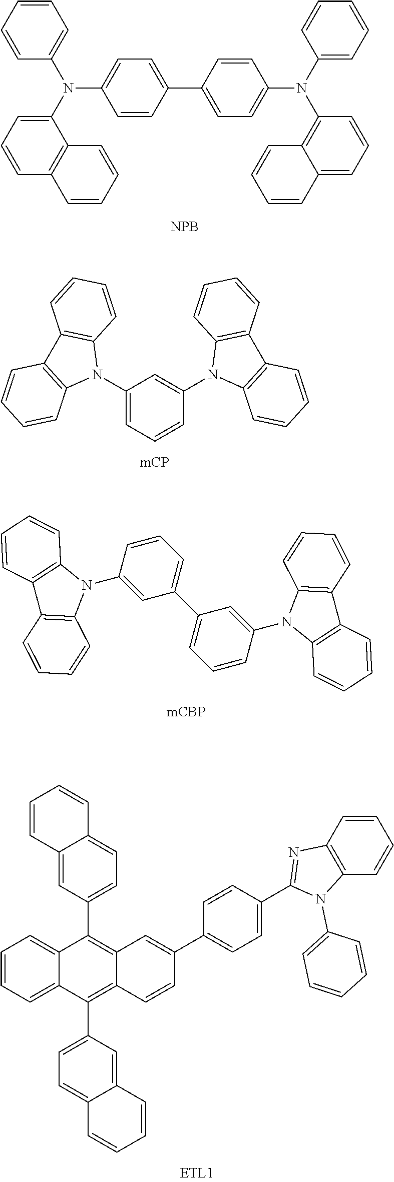 [SMILES: c1ccc(N(c2ccc(-c3ccc(N(c4ccccc4)c4cccc5ccccc45)cc3)cc2)c2cccc3ccccc23)cc1 ; "NPB" ; c1cc(-n2c3ccccc3c3ccccc32)cc(-n2c3ccccc3c3ccccc32)c1 ; c1cc(-c2cccc(-n3c4ccccc4c4ccccc43)c2)cc(-n2c3ccccc3c3ccccc32)c1 ; c1ccc(-n2c(-c3ccc(-c4ccc5c(-c6ccc7ccccc7c6)c6ccccc6c(-c6ccc7ccccc7c6)c5c4)cc3)nc3ccccc32)cc1]

### Examples 2 to 7 and Comparative Examples 1 to 4

Organic light-emitting devices were manufactured in the same manner as in Example 1, except that the compounds shown in Table 1 were each used instead of Compound 9 in forming an emission layer.

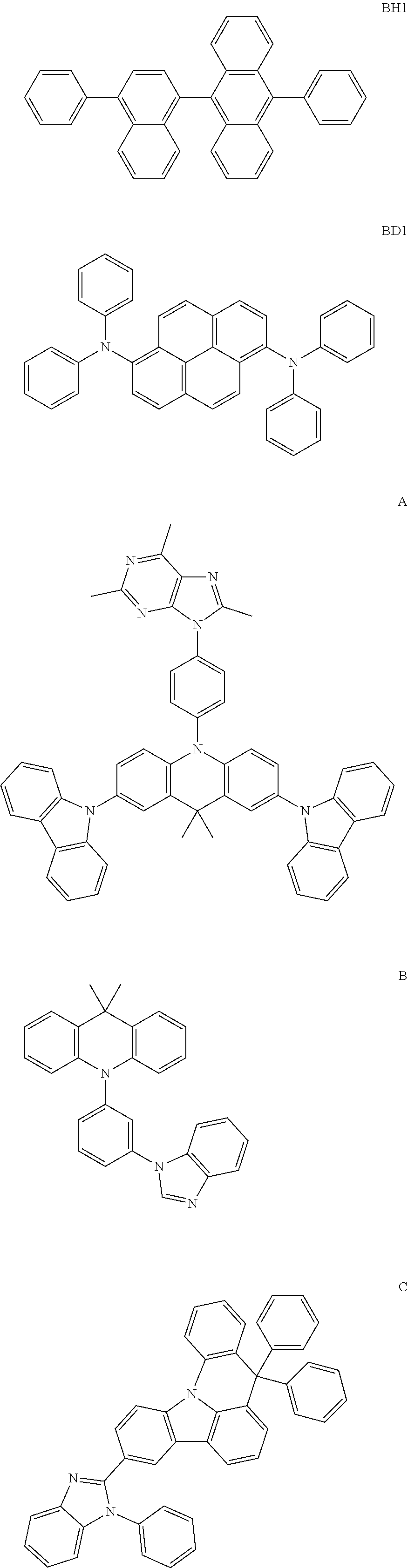

### Evaluation Example

The driving voltage, current efficiency, and external quantum efficiency of the organic light-emitting devices manufactured according to Examples 1 to 7 and Comparative Examples 1 to 4 were measured at a current density of 10 mA/cm² by using a current-voltage meter (Keithley SMU 236) and a luminance meter PR650, and results thereof are shown in Table 1.

TABLE 1

| | | | | External |
|---|---|---|---|---|
| | Emission | Driving | Current | quantum |
| | layer | voltage | efficiency | efficiency |
| | compound | (V) | (cd/Ay) | (%) |
| Example 1 | Compound 9/mCBP | 4.3 | 13.5 | 5.4 |
| Example 2 | Compound 11/mCBP | 4.2 | 13.9 | 5.4 |
| Example 3 | Compound 13/mCBP | 5.6 | 13.8 | 3.9 |
| Example 4 | Compound 15/mCBP | 5.4 | 13.9 | 4.3 |
| Example 5 | Compound 25/mCBP | 7.5 | 17.3 | 6.6 |
| Example 6 | Compound 74/mCBP | 5.5 | 15.9 | 5.9 |
| Example 7 | Compound 83/mCBP | 5.2 | 18.0 | 5.5 |
| Comparative | Compound BD1/ | 7.5 | 6.5 | 3.5 |
| Example 1 | Compound BH1 | | | |
| Comparative | Compound A/mCBP | 5.1 | 13.5 | 5.3 |
| Example 2 | | | | |
| Comparative | Compound B/mCBP | 5.9 | 10.8 | 3.6 |
| Example 3 | | | | |
| Comparative | Compound C/mCBP | 4.8 | 12.6 | 4.0 |
| Example 4 | | | | |

Referring to Table 1, it can be seen that the organic light-emitting devices using compounds according to example embodiments in the emission layer had a driving voltage at an equal or lower level and external quantum efficiency at an equal or higher level and has improved current efficiency, as compared with those of the organic light-emitting devices of Comparative Examples 1 to 4.

As described above, an organic light-emitting device including a heterocyclic compound according to an embodiment may have a low driving voltage, high efficiency, and a long lifespan.

Example embodiments have been disclosed herein, and although specific terms are employed, they are used and are to be interpreted in a generic and descriptive sense only and not for purpose of limitation. In some instances, as would be apparent to one of ordinary skill in the art as of the filing of the present application, features, characteristics, and/or elements described in connection with a particular embodiment may be used singly or in combination with features, characteristics, and/or elements described in connection with other embodiments unless otherwise specifically indicated. Accordingly, it will be understood by those of skill in the art that various changes in form and details may be made without departing from the spirit and scope of the present invention as set forth in the claims.

What is claimed is:

1. An organic light-emitting device, comprising:
a first electrode;
a second electrode facing the first electrode; and
an organic layer between the first electrode and the second electrode and including an emission layer, the organic layer including a heterocyclic compound represented by Formula 1: 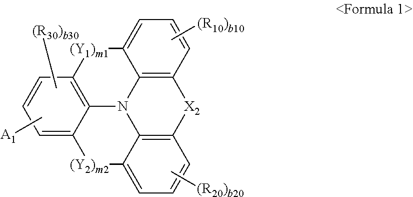
wherein, in Formula 1,
A₁ is a group represented by Formula 2, 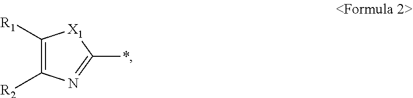
in Formulae 1 and 2,
X₁ is O, S, or N(R₃),
X₂ is O, S, N(R₄), C(R₄)(R₅), or Si(R₄)(R₅),
Y₁ and Y₂ are each independently C(R₆)(R₇) or Si(R₆)(R₇),
m1 is 0 or 1,
m2 is 0 or 1,
R₃ is hydrogen, deuterium, —F, —Cl, —Br, —I, a hydroxyl group, a cyano group, a nitro group, an amidino group, a hydrazino group, a hydrazono group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₃-C₁₀ cycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted monovalent non-aromatic condensed polycyclic group, —Si(Q₁)(Q₂)(Q₃), —B(Q₁)(Q₂), —C(═O)(Q₁), —N(Q₁)(Q₂), —P(═O)(Q₁)(Q₂), or —S(═O)₂(Q₁),
R₁, R₂, R₄, R₅, R₆, R₇, R₁₀, R₂₀, and R₃₀ are each independently hydrogen, deuterium, —F, —Cl, —Br, —I, a hydroxyl group, a cyano group, a nitro group, an amidino group, a hydrazino group, a hydrazono group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, a substituted or unsubstituted monovalent non-aromatic condensed heteropolycyclic group, —Si(Q₁)(Q₂)(Q₃), —B(Q₁)(Q₂), —C(═O)(Q₁), —N(Q₁)(Q₂), —P(═O)(Q₁)(Q₂), or —S(═O)₂(Q₁),
two or more neighboring substituents of R₁ to R₇, R₁₀, R₂₀, and R₃₀ are optionally linked to form a substituted or unsubstituted C₅-C₆₀ carbocyclic group or a substituted or unsubstituted C₁-C₆₀ heterocyclic group,
when m1 and m2 are each 1, R₁ and R₂ are optionally linked to form a substituted or unsubstituted C₇-C₆₀ carbocyclic group or a substituted or unsubstituted C₁-C₆₀ heterocyclic group,
b10, b20, and b30 are each independently an integer from 1 to 4,
at least one substituent of the substituted C₅-C₆₀ carbocyclic group, the substituted C₇-C₆₀ carbocyclic group, the substituted C₁-C₆₀ heterocyclic group, the substituted C₁-C₆₀ alkyl group, the substituted C₂-C₆₀ alkenyl group, the substituted C₂-C₆₀ alkynyl group, the substituted C₁-C₆₀ alkoxy group, the substituted C₃-C₁₀ cycloalkyl group, the substituted C₁-C₁₀ heterocycloalkyl group, the substituted C₃-C₁₀ cycloalkenyl group, the substituted C₁-C₁₀ heterocycloalkenyl group, the substituted C₆-C₆₀ aryl group, the substituted C₆-C₆₀ aryloxy group, the substituted C₆-C₆₀ arylthio group, the substituted C₁-C₆₀ heteroaryl group, the substituted monovalent non-aromatic condensed polycyclic group, and the substituted monovalent non-aromatic condensed heteropolycyclic group is:
deuterium, —F, —Cl, —Br, —I, a hydroxyl group, a cyano group, a nitro group, an amidino group, a hydrazino group, a hydrazono group, a C₁-C₆₀ alkyl group, a C₂-C₆₀ alkenyl group, a C₂-C₆₀ alkynyl group, or a C₁-C₆₀ alkoxy group;
a C₁-C₆₀ alkyl group, a C₂-C₆₀ alkenyl group, a C₂-C₆₀ alkynyl group, or a C₁-C₆₀ alkoxy group, each substituted with deuterium, —F, —Cl, —Br, —I, a hydroxyl group, a cyano group, a nitro group, an amidino group, a hydrazino group, a hydrazono group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —Si(Q₁₁)(Q₁₂)(Q 13), —N(Q₁₁)(Q₁₂), —B(Q₁₁)(Q₁₂), —C(═O)(Q₁₁), —S(═O)₂(Q₁₁), or —P(═O)(Q₁₁)(Q₁₂);
a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, or a monovalent non-aromatic condensed heteropolycyclic group;
a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, or a monovalent non-aromatic condensed heteropolycyclic group, each substituted with deuterium, —F, —Cl, —Br, —I, a hydroxyl group, a cyano group, a nitro group, an amidino group, a hydrazino group, a hydrazono group, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), or —P(═O)(Q₂₁)(Q₂₂); or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁ to Q₃, Q₁₁ to Q₁₃, Q₂₁ to Q₂₃, and Q₃₁ to Q₃₃ are each independently hydrogen, deuterium, —F, —Cl, —Br, —I, a hydroxyl group, a cyano group, a nitro group, an amidino group, a hydrazino group, a hydrazono group, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₁-C₆₀ heteroaryl group, a monovalent non-aromatic condensed polycyclic group, a monovalent non-aromatic condensed heteropolycyclic group, a biphenyl group, or a terphenyl group.

2. The organic light-emitting device as claimed in claim 1, wherein:
the first electrode is an anode,
the second electrode is a cathode,
the organic layer further includes a hole transport region between the first electrode and the emission layer and an electron transport region between the emission layer and the second electrode,
the hole transport region includes a hole injection layer, a hole transport layer, an emission auxiliary layer, an electron blocking layer, or a combination thereof, and
the electron transport region includes a hole blocking layer, an electron transport layer, an electron injection layer, or a combination thereof.

3. The organic light-emitting device as claimed in claim 1, wherein the emission layer includes the heterocyclic compound.

4. The organic light-emitting device as claimed in claim 3, wherein:
the emission layer consists of the heterocyclic compound; or
the emission layer further includes a host, and an amount of the heterocyclic compound is in a range of about 0.1 parts by weight to about 50 parts by weight based on 100 parts by weight of the emission layer.

5. The organic light-emitting device as claimed in claim 3, wherein the heterocyclic compound included in the emission layer satisfies Equation 1:

|E_{D, S1}−E_{D, T1}|≤0.3 eV, <Equation 1>
wherein, in Equation 1,
E_{D, S1} is singlet energy level (eV) of the heterocyclic compound, and
E_{D, T1} is triplet energy level (eV) of the heterocyclic compound.

6. The organic light-emitting device as claimed in claim 3, wherein:
the heterocyclic compound included in the emission layer is a thermally delayed fluorescent emitter, and
the emission layer emits delayed fluorescence.

7. The organic light-emitting device as claimed in claim 3, wherein the emission layer does not include an organometallic compound.

8. A heterocyclic compound represented by Formula 1: 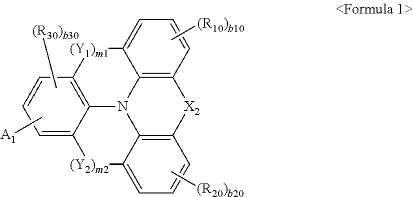
wherein, in Formula 1,
A₁ is a group represented by Formula 2, 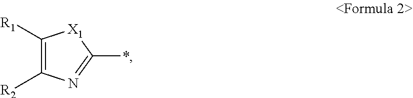
in Formulae 1 and 2,
X₁ is O, S, or N(R₃),
X₂ is O, S, N(R₄), C(R₄)(R₅), or Si(R₄)(R₅),
Y₁ and Y₂ are each independently C(R₆)(R₇) or Si(R₆)(R₇),
m1 is 0 or 1,
m2 is 0 or 1,
R₃ is hydrogen, deuterium, —F, —Cl, —Br, —I, a hydroxyl group, a cyano group, a nitro group, an amidino group, a hydrazino group, a hydrazono group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₃-C₁₀ cycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted monovalent non-aromatic condensed polycyclic group, —Si(Q₁)(Q₂)(Q₃), —B(Q₁)(Q₂), —C(═O)(Q₁), —N(Q₁)(Q₂), —P(═O)(Q₁)(Q₂), or —S(═O)₂(Q₁),
R₁, R₂, R₄, R₅, R₆, R₇, R₁₀, R₂₀, and R₃₀ are each independently hydrogen, deuterium, —F, —Cl, —Br, —I, a hydroxyl group, a cyano group, a nitro group, an amidino group, a hydrazino group, a hydrazono group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, a substituted or unsubstituted monovalent non-aromatic condensed heteropolycyclic group, —Si(Q₁)(Q₂)(Q₃), —B(Q₁)(Q₂), —C(═O)(Q₁), —N(Q₁)(Q₂), —P(═O)(Q₁)(Q₂), or —S(═O)₂(Q₁),
two or more neighboring substituents among R₁ to R₇, R₁₀, R₂₀, and R₃₀ are optionally linked to form a substituted or unsubstituted C₅-C₆₀ carbocyclic group or a substituted or unsubstituted C₁-C₆₀ heterocyclic group,
when m1 and m2 are each 1, R₁ and R₂ are optionally linked to form a substituted or unsubstituted C₇-C₆₀ carbocyclic group or a substituted or unsubstituted C₁-C₆₀ heterocyclic group,
b10, b20, and b30 are each independently an integer from 1 to 4,
at least one substituent of the substituted C₅-C₆₀ carbocyclic group, the substituted C₇-C₆₀ carbocyclic group, the substituted C₁-C₆₀ heterocyclic group, the substituted C₁-C₆₀ alkyl group, the substituted C₂-C₆₀ alkenyl group, the substituted C₂-C₆₀ alkynyl group, the substituted C₁-C₆₀ alkoxy group, the substituted C₃-C₁₀ cycloalkyl group, the substituted C₁-C₁₀ heterocycloalkyl group, the substituted C₃-C₁₀ cycloalkenyl group, the substituted C₁-C₁₀ heterocycloalkenyl group, the substituted C₆-C₆₀ aryl group, the substituted C₆-C₆₀ aryloxy group, the substituted C₆-C₆₀ arylthio group, the substituted C₁-C₆₀ heteroaryl group, the substituted monovalent non-aromatic condensed polycyclic group, and the substituted monovalent non-aromatic condensed heteropolycyclic group is:
deuterium, —F, —Cl, —Br, —I, a hydroxyl group, a cyano group, a nitro group, an amidino group, a hydrazino group, a hydrazono group, a C₁-C₆₀ alkyl group, a C₂-C₆₀ alkenyl group, a C₂-C₆₀ alkynyl group, or a C₁-C₆₀ alkoxy group;
a C₁-C₆₀ alkyl group, a C₂-C₆₀ alkenyl group, a C₂-C₆₀ alkynyl group, or a C₁-C₆₀ alkoxy group, each substituted with deuterium, —F, —Cl, —Br, —I, a hydroxyl group, a cyano group, a nitro group, an amidino group, a hydrazino group, a hydrazono group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), or —P(═O)(Q₁₁)(Q₁₂);
a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, or a monovalent non-aromatic condensed heteropolycyclic group;
a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, or a monovalent non-aromatic condensed heteropolycyclic group, each substituted with deuterium, —F, —Cl, —Br, —I, a hydroxyl group, a cyano group, a nitro group, an amidino group, a hydrazino group, a hydrazono group, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), or —P(═O)(Q₂₁)(Q₂₂); or
—Si(Q₃₁)(Q₃₂)(Q₃₃), —N(Q₃₁)(Q₃₂), —B(Q₃)(Q₃₂), —C(═O)(Q₃₁), —S(═O)₂(Q₃₁), or —P(═O)(Q₃₁)(Q₃₂), and
Q₁ to Q₃, Q₁₁ to Q₁₃, Q₂₁ to Q₂₃, and Q₃₁ to Q₃₃ are each independently hydrogen, deuterium, —F, —Cl, —Br, —I, a hydroxyl group, a cyano group, a nitro group, an amidino group, a hydrazino group, a hydrazono group, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₁-C₆₀ heteroaryl group, a monovalent non-aromatic condensed polycyclic group, a monovalent non-aromatic condensed heteropolycyclic group, a biphenyl group, or a terphenyl group.

9. The heterocyclic compound as claimed in claim 8, wherein A₁ in Formula 1 is represented by one of Formulae 2-1 to 2-3: 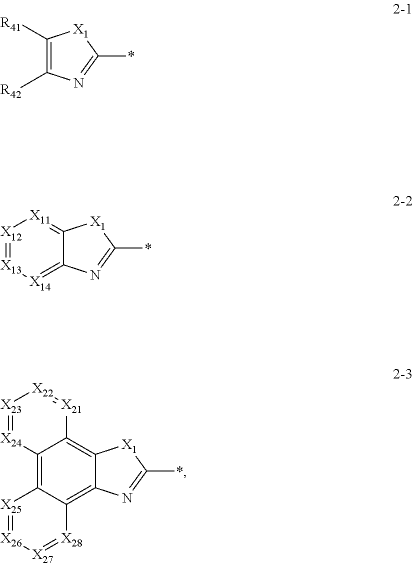
wherein, in Formulae 2-1 to 2-3,
X₁ is the same as described in claim 8,
X₁₁ is N or C(R₅₁), X₁₂ is N or C(R₅₂), X₁₃ is N or C(R₅₃), and X₁₄ is N or C(R₅₄), X₂₁ is N or C(R₆₁), X₂₂ is N or C(R₆₂), X₂₃ is N or C(R₆₃), X₂₄ is N or C(R₆₄), X₂₅ is N or C(R₆₅), X₂₆ is N or C(R₆₆), X₂₇ is N or C(R₆₇), and X₂₈ is N or C(R₆₈),
when m1 and m2 in Formula 1 are each 1, at least one of X₁₁ to X₁₄ is N,
indicates a binding site to a neighboring atom,
R₄₁, R₄₂, R₅₁ to R₅₄, and R₆₁ to R₆₈ are each independently:
hydrogen, deuterium, —F, —Cl, —Br, —I, a methyl group, an ethyl group, an n-propyl group, an isopropyl group, an n-butyl group, a sec-butyl group, an isobutyl group, a tert-butyl group, an ethenyl group, a propenyl group, a butenyl group, a methoxy group, an ethoxy group, an n-propoxy group, an isopropoxy group, an n-butoxy group, a sec-butoxy group, an isobutoxy group, or a tert-butoxy group;
a methyl group, an ethyl group, an n-propyl group, an isopropyl group, an n-butyl group, a sec-butyl group, an isobutyl group, a tert-butyl group, a methoxy group, an ethoxy group, an n-propoxy group, an isopropoxy group, an n-butoxy group, a sec-butoxy group, an isobutoxy group, or a tert-butoxy group, each substituted with deuterium, —F, —Cl, —Br, —I, a cyano group, a phenyl group, or a biphenyl group; or
a cyclopentyl group, a cyclohexyl group, a cycloheptyl group, a cyclopentenyl group, a cyclohexenyl group, a phenyl group, a biphenyl group, a terphenyl group, a pentalenyl group, an indenyl group, a naphthyl group, an azulenyl group, an indacenyl group, an acenaphthyl group, a fluorenyl group, a spiro-bifluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a perylenyl group, a pentacenyl group, a pyrrolyl group, a thiophenyl group, a furanyl group, a silolyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an indolyl group, an isoindolyl group, an indazolyl group, a purinyl group, a quinolinyl group, an isoquinolinyl group, a benzoquinolinyl group, an isoquinolinyl group, a phthalazinyl group, a naphthyridinyl group, a quinoxalinyl group, a benzoquinoxalinyl group, a quinazolinyl group, a benzoquinazolinyl group, a cinnolinyl group, a phenanthridinyl group, an acridinyl group, a phenanthrolinyl group, a phenazinyl group, a benzimidazolyl group, a benzofuranyl group, a benzothiophenyl group, a benzosilolyl group, a benzothiazolyl group, a benzoisothiazolyl group, a benzoxazolyl group, a benzoisoxazolyl group, a triazolyl group, a tetrazolyl group, a thiadiazolyl group, an oxadiazolyl group, a triazinyl group, a carbazolyl group, a dibenzofuranyl group, a dibenzothiophenyl group, a dibenzosilolyl group, a benzocarbazolyl group, a naphthobenzofuranyl group, a naphthobenzothiophenyl group, a naphthobenzosilolyl group, a dibenzocarbazolyl group, a dinaphthofuranyl group, a dinaphthothiophenyl group, a dinaphtho silolyl group, an imidazopyridinyl group, an imidazopyrimidinyl group, an oxazolopyridinyl group, a thiazolopyridinyl group, a benzonaphthyridinyl group, an azafluorenyl group, an azaspiro-bifluorenyl group, an azacarbazolyl group, an azadibenzofuranyl group, an azadibenzothiophenyl group, an azadibenzosilolyl group, an indenopyrrolyl group, an indolopyrrolyl group, an indenocarbazolyl group, or an indolocarbazolyl group.

10. The heterocyclic compound as claimed in claim 8, wherein:
m1 and m2 are each 0, or
m1 and m2 are each 1.

11. The heterocyclic compound as claimed in claim 9, wherein R₃ is:
hydrogen, deuterium, —F, —Cl, —Br, —I, a methyl group, an ethyl group, an n-propyl group, an isopropyl group, an n-butyl group, a sec-butyl group, an isobutyl group, a tert-butyl group, an ethenyl group, a propenyl group, a butenyl group, a methoxy group, an ethoxy group, an n-propoxy group, an isopropoxy group, an n-butoxy group, a sec-butoxy group, an isobutoxy group, or a tert-butoxy group;
a methyl group, an ethyl group, an n-propyl group, an isopropyl group, an n-butyl group, a sec-butyl group, an isobutyl group, a tert-butyl group, a methoxy group, an ethoxy group, an n-propoxy group, an isopropoxy group, an n-butoxy group, a sec-butoxy group, an isobutoxy group, or a tert-butoxy group, each substituted with deuterium, —F, —Cl, —Br, —I, a cyano group, a phenyl group, or a biphenyl group;
a cyclopentyl group, a cyclohexyl group, a cycloheptyl group, a cyclopentenyl group, a cyclohexenyl group, a phenyl group, a biphenyl group, a terphenyl group, a pentalenyl group, an indenyl group, a naphthyl group, an azulenyl group, an indacenyl group, an acenaphthyl group, a fluorenyl group, a spiro-bifluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a perylenyl group, or a pentacenyl group; or
a cyclopentyl group, a cyclohexyl group, a cycloheptyl group, a cyclopentenyl group, a cyclohexenyl group, a phenyl group, a biphenyl group, a terphenyl group, a pentalenyl group, an indenyl group, a naphthyl group, an azulenyl group, an indacenyl group, an acenaphthyl group, a fluorenyl group, a spiro-bifluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a perylenyl group, or a pentacenyl group, each substituted with deuterium, —F, —Cl, —Br, —I, a cyano group, a methyl group, an ethyl group, an n-propyl group, an isopropyl group, an n-butyl group, a sec-butyl group, an isobutyl group, a tert-butyl group, a methoxy group, an ethoxy group, an n-propoxy group, an isopropoxy group, an n-butoxy group, a sec-butoxy group, an isobutoxy group, a tert-butoxy group, a cyclopentyl group, a cyclohexyl group, a cycloheptyl group, a cyclopentenyl group, a cyclohexenyl group, a phenyl group, a biphenyl group, a terphenyl group, a pentalenyl group, an indenyl group, a naphthyl group, an azulenyl group, an indacenyl group, an acenaphthyl group, a fluorenyl group, a spiro-bifluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a perylenyl group, a pentacenyl group, a pyrrolyl group, a thiophenyl group, a furanyl group, a silolyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an indolyl group, an isoindolyl group, an indazolyl group, a purinyl group, a quinolinyl group, an isoquinolinyl group, a benzoquinolinyl group, a phthalazinyl group, a naphthyridinyl group, a quinoxalinyl group, a benzoquinoxalinyl group, a quinazolinyl group, a benzoquinazolinyl group, a cinnolinyl group, a phenanthridinyl group, an acridinyl group, a phenanthrolinyl group, a phenazinyl group, a benzimidazolyl group, a benzofuranyl group, a benzothiophenyl group, a benzosilolyl group, a benzothiazolyl group, a benzoisothiazolyl group, a benzoxazolyl group, a benzoisoxazolyl group, a triazolyl group, a tetrazolyl group, a thiadiazolyl group, an oxadiazolyl group, a triazinyl group, a carbazolyl group, a dibenzofuranyl group, a dibenzothiophenyl group, a dibenzosilolyl group, a benzocarbazolyl group, a naphthobenzofuranyl group, a naphthobenzothiophenyl group, a naphthobenzosilolyl group, a dibenzocarbazolyl group, a dinaphthofuranyl group, a dinaphthothiophenyl group, a dinaphtho silolyl group, an imidazopyridinyl group, an imidazopyrimidinyl group, an oxazolopyridinyl group, a thiazolopyridinyl group, a benzonaphthyridinyl group, an azafluorenyl group, an azaspiro-bifluorenyl group, an azacarbazolyl group, an azadibenzofuranyl group, an azadibenzothiophenyl group, an azadibenzosilolyl group, an indenopyrrolyl group, an indolopyrrolyl group, an indenocarbazolyl group, an indolocarbazolyl group, —Si(Q₃₁)(Q₃₂)(Q₃₃), —N(Q₃₁)(Q₃₂), —B(Q₃₁)(Q₃₂), —C(═O)(Q₃₁), —S(═O)(Q₃₁), —S(═O)₂(Q₃₁), —P(═O)(Q₃₁)(Q₃₂), or —P(═S)(Q₃₁)(Q₃₂).

12. The heterocyclic compound as claimed in claim 8, wherein R₃ is:
hydrogen, deuterium, —F, —Cl, —Br, —I, a cyano group, a C₁-C₂₀ alkyl group, or a C₁-C₂₀ alkoxy group;
a C₁-C₂₀ alkyl group or a C₁-C₂₀ alkoxy group, each substituted with deuterium, —F, —Cl, —Br, —I, a cyano group, a phenyl group, or a biphenyl group; or
a group represented by one of Formulae 7-1 to 7-12: 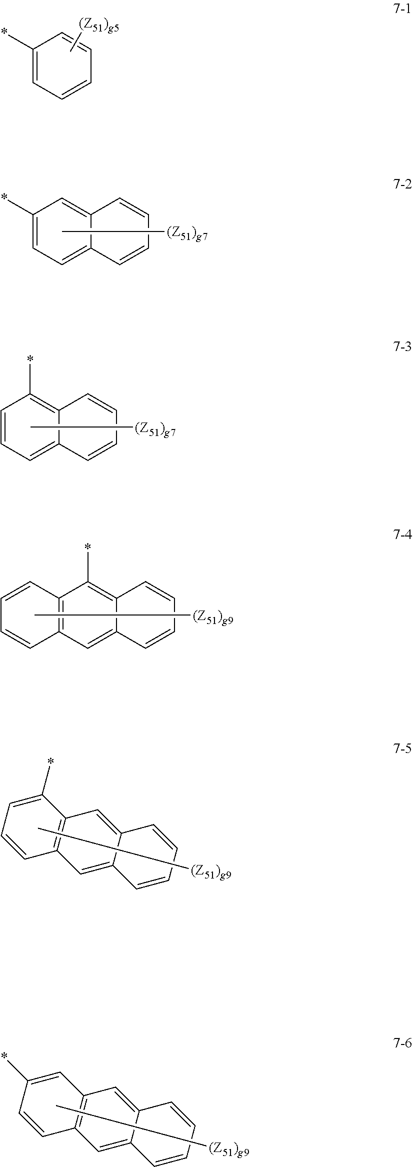 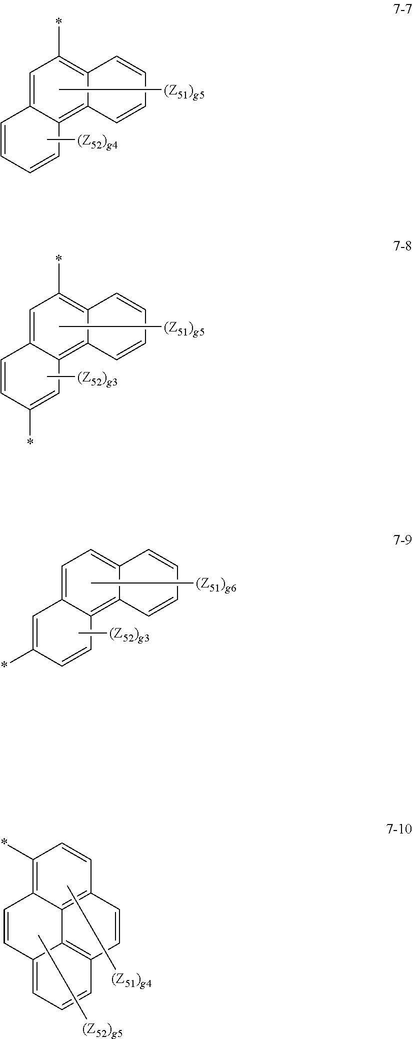 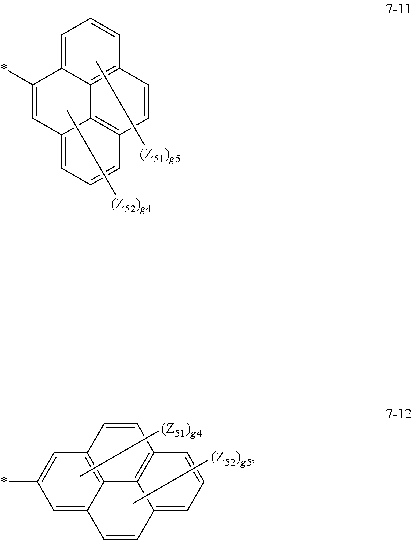
wherein, in Formulae 7-1 to 7-12,
Y₅₁ is O, S, C(Z₅₃)(Z₅₄), N(Z₅₃), or Si(Z₅₃)(Z₅₄),
Z₅₁ to Z₅₄ are each independently hydrogen, deuterium, —F, —Cl, —Br, —I, a hydroxyl group, a cyano group, a nitro group, an amidino group, a hydrazino group, a hydrazono group, a C₁-C₂₀ alkyl group, a C₁-C₂₀ alkenyl group, a C₁-C₂₀ alkynyl group, a C₁-C₂₀ alkoxy group, a phenyl group, a biphenyl group, a terphenyl group, a naphthyl group, a fluorenyl group, a spiro-bifluorenyl group, a phenanthrenyl group, an anthracenyl group, a triphenylenyl group, a pyridinyl group, a pyrimidinyl group, a carbazolyl group, or a triazinyl group,
g3 is an integer from 1 to 3,
g4 is an integer from 1 to 4,
g5 is an integer from 1 to 5,
g6 is an integer from 1 to 6,
g7 is an integer from 1 to 7,
g9 is an integer from 1 to 9, and
indicates a binding site to a neighboring atom.

13. The heterocyclic compound as claimed in claim 8, wherein R₁, R₂, R₄, R₅, R₆, R₇, R₁₀, R₂₀, and R₃₀ are each independently:
hydrogen, deuterium, —F, —Cl, —Br, —I, a methyl group, an ethyl group, an n-propyl group, an isopropyl group, an n-butyl group, a sec-butyl group, an isobutyl group, a tert-butyl group, an ethenyl group, a propenyl group, a butenyl group, a methoxy group, an ethoxy group, an n-propoxy group, an isopropoxy group, an n-butoxy group, a sec-butoxy group, an isobutoxy group, or a tert-butoxy group;
a methyl group, an ethyl group, an n-propyl group, an isopropyl group, an n-butyl group, a sec-butyl group, an isobutyl group, a tert-butyl group, a methoxy group, an ethoxy group, an n-propoxy group, an isopropoxy group, an n-butoxy group, a sec-butoxy group, an isobutoxy group, or a tert-butoxy group, each substituted with deuterium, —F, —Cl, —Br, —I, a cyano group, a phenyl group, or a biphenyl group;
a cyclopentyl group, a cyclohexyl group, a cycloheptyl group, a cyclopentenyl group, a cyclohexenyl group, a phenyl group, a biphenyl group, a terphenyl group, a pentalenyl group, an indenyl group, a naphthyl group, an azulenyl group, an indacenyl group, an acenaphthyl group, a fluorenyl group, a spiro-bifluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a perylenyl group, a pentacenyl group, a pyrrolyl group, a thiophenyl group, a furanyl group, a silolyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an indolyl group, an isoindolyl group, an indazolyl group, a purinyl group, a quinolinyl group, an isoquinolinyl group, a benzoquinolinyl group, a phthalazinyl group, a naphthyridinyl group, a quinoxalinyl group, a benzoquinoxalinyl group, a quinazolinyl group, a benzoquinazolinyl group, a cinnolinyl group, a phenanthridinyl group, an acridinyl group, a phenanthrolinyl group, a phenazinyl group, a benzimidazolyl group, a benzofuranyl group, a benzothiophenyl group, a benzosilolyl group, a benzothiazolyl group, a benzoisothiazolyl group, a benzoxazolyl group, a benzoisoxazolyl group, a triazolyl group, a tetrazolyl group, a thiadiazolyl group, an oxadiazolyl group, a triazinyl group, a carbazolyl group, a dibenzofuranyl group, a dibenzothiophenyl group, a dibenzosilolyl group, a benzocarbazolyl group, a naphthobenzofuranyl group, a naphthobenzothiophenyl group, a naphthobenzosilolyl group, a dibenzocarbazolyl group, a dinaphthofuranyl group, a dinaphthothiophenyl group, a dinaphtho silolyl group, an imidazopyridinyl group, an imidazopyrimidinyl group, an oxazolopyridinyl group, a thiazolopyridinyl group, a benzonaphthyridinyl group, an azafluorenyl group, an azaspiro-bifluorenyl group, an azacarbazolyl group, an azadibenzofuranyl group, an azadibenzothiophenyl group, an azadibenzosilolyl group, an indenopyrrolyl group, an indolopyrrolyl group, an indenocarbazolyl group, or an indolocarbazolyl group; or
a cyclopentyl group, a cyclohexyl group, a cycloheptyl group, a cyclopentenyl group, a cyclohexenyl group, a phenyl group, a biphenyl group, a terphenyl group, a pentalenyl group, an indenyl group, a naphthyl group, an azulenyl group, an indacenyl group, an acenaphthyl group, a fluorenyl group, a spiro-bifluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a perylenyl group, a pentacenyl group, a pyrrolyl group, a thiophenyl group, a furanyl group, a silolyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an indolyl group, an isoindolyl group, an indazolyl group, a purinyl group, a quinolinyl group, an isoquinolinyl group, a benzoquinolinyl group, a phthalazinyl group, a naphthyridinyl group, a quinoxalinyl group, a benzoquinoxalinyl group, a quinazolinyl group, a benzoquinazolinyl group, a cinnolinyl group, a phenanthridinyl group, an acridinyl group, a phenanthrolinyl group, a phenazinyl group, a benzimidazolyl group, a benzofuranyl group, a benzothiophenyl group, a benzosilolyl group, a benzothiazolyl group, a benzoisothiazolyl group, a benzoxazolyl group, a benzoisoxazolyl group, a triazolyl group, a tetrazolyl group, a thiadiazolyl group, an oxadiazolyl group, a triazinyl group, a carbazolyl group, a dibenzofuranyl group, a dibenzothiophenyl group, a dibenzosilolyl group, a benzocarbazolyl group, a naphthobenzofuranyl group, a naphthobenzothiophenyl group, a naphthobenzosilolyl group, a dibenzocarbazolyl group, a dinaphthofuranyl group, a dinaphthothiophenyl group, a dinaphtho silolyl group, an imidazopyridinyl group, an imidazopyrimidinyl group, an oxazolopyridinyl group, a thiazolopyridinyl group, a benzonaphthyridinyl group, an azafluorenyl group, an azaspiro-bifluorenyl group, an azacarbazolyl group, an azadibenzofuranyl group, an azadibenzothiophenyl group, an azadibenzosilolyl group, an indenopyrrolyl group, an indolopyrrolyl group, an indenocarbazolyl group, or an indolocarbazolyl group, each substituted with deuterium, —F, —Cl, —Br, —I, a cyano group, a methyl group, an ethyl group, an n-propyl group, an isopropyl group, an n-butyl group, a sec-butyl group, an isobutyl group, a tert-butyl group, a methoxy group, an ethoxy group, an n-propoxy group, an isopropoxy group, an n-butoxy group, a sec-butoxy group, an isobutoxy group, a tert-butoxy group, a cyclopentyl group, a cyclohexyl group, a cycloheptyl group, a cyclopentenyl group, a cyclohexenyl group, a phenyl group, a biphenyl group, a terphenyl group, a pentalenyl group, an indenyl group, a naphthyl group, an azulenyl group, an indacenyl group, an acenaphthyl group, a fluorenyl group, a spiro-bifluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a perylenyl group, a pentacenyl group, a pyrrolyl group, a thiophenyl group, a furanyl group, a silolyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an indolyl group, an isoindolyl group, an indazolyl group, a purinyl group, a quinolinyl group, an isoquinolinyl group, a benzoquinolinyl group, a phthalazinyl group, a naphthyridinyl group, a quinoxalinyl group, a benzoquinoxalinyl group, a quinazolinyl group, a benzoquinazolinyl group, a cinnolinyl group, a phenanthridinyl group, an acridinyl group, a phenanthrolinyl group, a phenazinyl group, a benzimidazolyl group, a benzofuranyl group, a benzothiophenyl group, a benzosilolyl group, a benzothiazolyl group, a benzoisothiazolyl group, a benzoxazolyl group, a benzoisoxazolyl group, a triazolyl group, a tetrazolyl group, a thiadiazolyl group, an oxadiazolyl group, a triazinyl group, a carbazolyl group, a dibenzofuranyl group, a dibenzothiophenyl group, a dibenzosilolyl group, a benzocarbazolyl group, a naphthobenzofuranyl group, a naphthobenzothiophenyl group, a naphthobenzosilolyl group, a dibenzocarbazolyl group, a dinaphthofuranyl group, a dinaphthothiophenyl group, a dinaphtho silolyl group, an imidazopyridinyl group, an imidazopyrimidinyl group, an oxazolopyridinyl group, a thiazolopyridinyl group, a benzonaphthyridinyl group, an azafluorenyl group, an azaspiro-bifluorenyl group, an azacarbazolyl group, an azadibenzofuranyl group, an azadibenzothiophenyl group, an azadibenzosilolyl group, an indenopyrrolyl group, an indolopyrrolyl group, an indenocarbazolyl group, an indolocarbazolyl group, —Si(Q₃₁)(Q₃₂)(Q₃₃), —N(Q₃₁)(Q₃₂), —B(Q₃₁)(Q₃₂), —C(═O)(Q₃₁), —S(═O)(Q₃), —S(═O)₂(Q₃₁), —P(═O)(Q₃₁)(Q₃₂), or —P(═S)(Q₃₁)(Q₃₂), or
two or more neighboring substituents among R₁, R₂, R₄, R₅, R₆, R₇, R₁₀, R₂₀, and R₃₀ are optionally linked to form a substituted or unsubstituted C₅-C₆₀ carbocyclic group or a substituted or unsubstituted C₁-C₆₀ heterocyclic group.

14. The heterocyclic compound as claimed in claim 8, wherein:
R₁, R₂, R₄, R₅, R₆, R₇, R₁₀, R₂₀, and R₃₀ are each independently:
hydrogen, deuterium, —F, —Cl, —Br, —I, a methyl group, an ethyl group, an n-propyl group, an isopropyl group, an n-butyl group, a sec-butyl group, an isobutyl group, a tert-butyl group, an ethenyl group, a propenyl group, a butenyl group, a methoxy group, an ethoxy group, an n-propoxy group, an isopropoxy group, an n-butoxy group, a sec-butoxy group, an isobutoxy group, or a tert-butoxy group; or
a group represented by one of Formulae 5-1 to 5-26 and Formulae 6-1 to 6-55, or two or more neighboring substituents among R₁, R₂, R₄, R₅, R₆, R₇, R₁₀, R₂₀, and R₃₀ are optionally linked to form a substituted or unsubstituted C₅-C₆₀ carbocyclic group or a substituted or unsubstituted C₁-C₆₀ heterocyclic group, 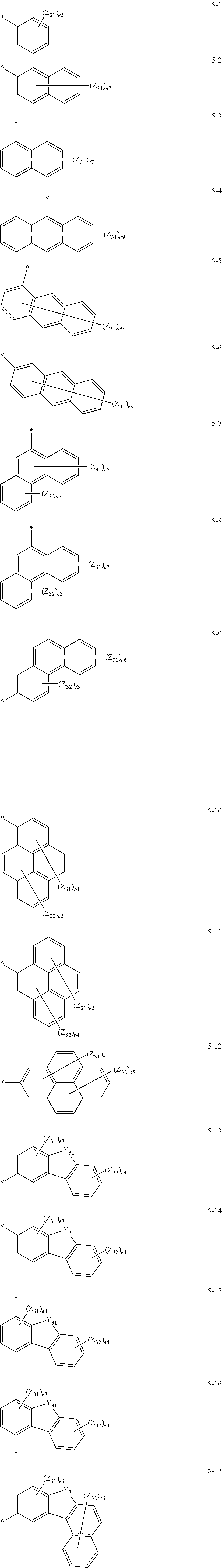 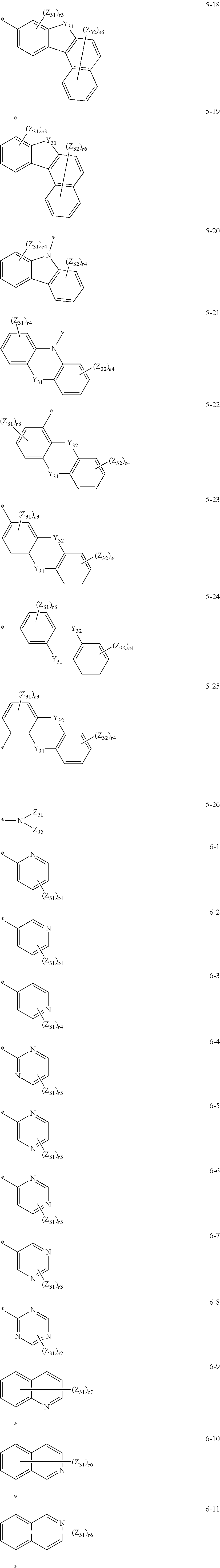 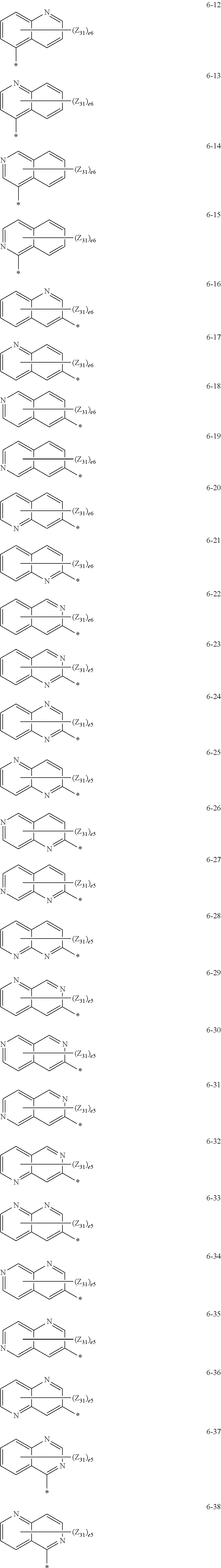 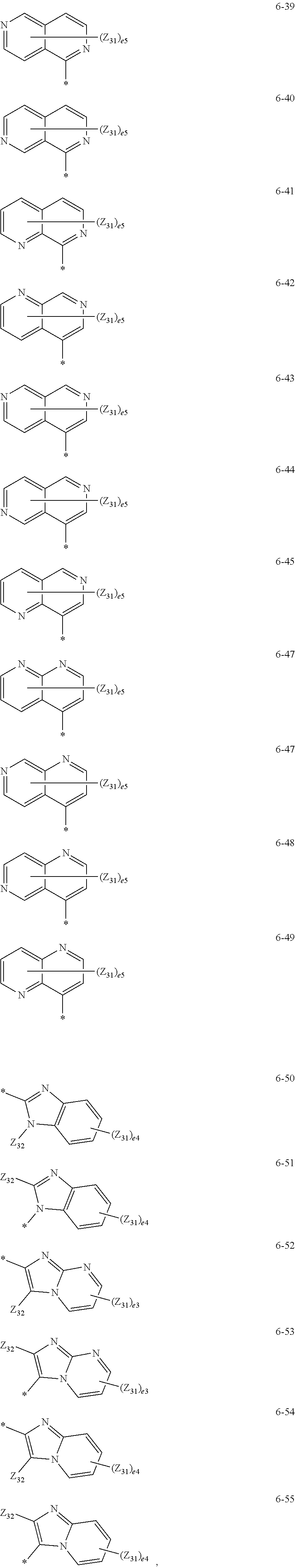
wherein, in Formulae 5-1 to 5-26 and Formulae 6-1 to 6-55,
Y₃₁ is O, S, C(Z₃₄)(Z₃₅), N(Z₃₄), or Si(Z₃₄)(Z₃₅),
Z₃₁ to Z₃₅ are each independently hydrogen, deuterium, —F, —Cl, —Br, —I, a hydroxyl group, a cyano group, a nitro group, an amidino group, a hydrazino group, a hydrazono group, a C₁-C₂₀ alkyl group, a C₁-C₂₀ alkenyl group, a C₁-C₂₀ alkynyl group, a C₁-C₂₀ alkoxy group, a phenyl group, a biphenyl group, a terphenyl group, a naphthyl group, a fluorenyl group, a spiro-bifluorenyl group, a phenanthrenyl group, an anthracenyl group, a triphenylenyl group, a pyridinyl group, a pyrimidinyl group, a carbazolyl group, or a triazinyl group,
e2 is 1 or 2,
e3 is an integer from 1 to 3,
e4 is an integer from 1 to 4,
e5 is an integer from 1 to 5,
e6 is an integer from 1 to 6,
e7 is an integer from 1 to 7,
e9 is an integer from 1 to 9, and
indicates a binding site to a neighboring atom.

15. The heterocyclic compound as claimed in claim 14, wherein:
R₁ and R₂ are each independently a group represented by one of Formulae 5-1 to 5-26 and Formulae 6-1 to 6-55, or
R₁ and R2 are optionally linked to form a ring group, the ring group being:
a benzene group, a naphthalene group, a phenanthrene group, an anthracene group, a triphenylene group, a pyrene group, a chrysene group, a pyridine group, a pyrazine group, a pyrimidine group, a triazine group, a quinoline group, an isoquinoline group, a benzoquinoline group, a benzoisoquinoline group, a 2,6-naphthyridine group, a 1,8-naphthyridine group, a 1,5-naphthyridine group, a 1,6-naphthyridine group, a 1,7-naphthyridine group, a 2,7-naphthyridine group, a quinoxaline group, a quinazoline group, a phenanthridine group, or a phenanthroline group; or
a benzene group, a naphthalene group, a phenanthrene group, an anthracene group, a triphenylene group, a pyrene group, a chrysene group, a pyridine group, a pyrazine group, a pyrimidine group, a triazine group, a quinoline group, an isoquinoline group, a benzoquinoline group, a benzoisoquinoline group, a 2,6-naphthyridine group, a 1,8-naphthyridine group, a 1,5-naphthyridine group, a 1,6-naphthyridine group, a 1,7-naphthyridine group, a 2,7-naphthyridine group, a quinoxaline group, a quinazoline group, a phenanthridine group, or a phenanthroline group, each substituted with deuterium, —F, —Cl, —Br, —I, a cyano group, a methyl group, an ethyl group, an n-propyl group, an isopropyl group, an n-butyl group, a sec-butyl group, an isobutyl group, a tert-butyl group, a methoxy group, an ethoxy group, an n-propoxy group, an isopropoxy group, an n-butoxy group, a sec-butoxy group, an isobutoxy group, or a tert-butoxy group.

16. The heterocyclic compound as claimed in claim 8, wherein:
R₄ and R₅ are each independently:
hydrogen, deuterium, —F, —Cl, —Br, —I, a methyl group, an ethyl group, an n-propyl group, an isopropyl group, an n-butyl group, a sec-butyl group, an isobutyl group, a tert-butyl group, an ethenyl group, a propenyl group, a butenyl group, a methoxy group, an ethoxy group, an n-propoxy group, an isopropoxy group, an n-butoxy group, a sec-butoxy group, an isobutoxy group, or a tert-butoxy group;
a methyl group, an ethyl group, an n-propyl group, an isopropyl group, an n-butyl group, a sec-butyl group, an isobutyl group, a tert-butyl group, a methoxy group, an ethoxy group, an n-propoxy group, an isopropoxy group, an n-butoxy group, a sec-butoxy group, an isobutoxy group, or a tert-butoxy group, each substituted with deuterium, —F, —Cl, —Br, —I, a cyano group, a phenyl group, or a biphenyl group;
a phenyl group, a biphenyl group, a terphenyl group, a pentalenyl group, an indenyl group, a naphthyl group, an azulenyl group, an indacenyl group, an acenaphthyl group, a fluorenyl group, a spiro-bifluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a perylenyl group, a pentacenyl group, a carbazolyl group, a dibenzofuranyl group, a dibenzothiophenyl group, or a dibenzosilolyl group; or
a cyclopentyl group, a cyclohexyl group, a cycloheptyl group, a cyclopentenyl group, a cyclohexenyl group, a phenyl group, a biphenyl group, a terphenyl group, a pentalenyl group, an indenyl group, a naphthyl group, an azulenyl group, an indacenyl group, an acenaphthyl group, a fluorenyl group, a spiro-bifluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a perylenyl group, a pentacenyl group, a carbazolyl group, a dibenzofuranyl group, a dibenzothiophenyl group, or a dibenzosilolyl group, each substituted with deuterium, —F, —Cl, —Br, —I, a cyano group, a methyl group, an ethyl group, an n-propyl group, an isopropyl group, an n-butyl group, a sec-butyl group, an isobutyl group, a tert-butyl group, a methoxy group, an ethoxy group, an n-propoxy group, an isopropoxy group, an n-butoxy group, a sec-butoxy group, an isobutoxy group, or a tert-butoxy group, or
R₄ and R₅ are optionally linked to form a group represented by Formula 9: 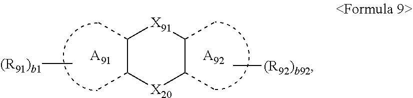
wherein, in Formula 9,
X₂₀ is C in X₂ of Formula 1 or Si in X₂ of Formula 1,
X₉₁ is a single bond, O, S, Se, N(R₉₃), B(R₉₃) C(R₉₃)(R₉₄), or Si(R₉₃)(R₉₄),
A₉₁ and A₉₂ are each independently a benzene group, a naphthalene group, a phenanthrene group, an anthracene group, a pyridine group, a pyrazine group, a pyrimidine group, a quinoline group, an isoquinoline group, a benzoquinoline group, a benzoisoquinoline group, a dibenzofuran group, a dibenzothiophene group, a fluorene group, or a carbazole group,
R₉₁ to R₉₄ are each independently hydrogen, deuterium, —F, —Cl, —Br, —I, a hydroxyl group, a cyano group, a nitro group, an amidino group, a hydrazino group, a hydrazono group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted C₁-C₆₀ heteroaryloxy group, a substituted or unsubstituted C₁-C₆₀ heteroarylthio group, a substituted or unsubstituted monovalent non-aromatic condensed polycyclic group, a substituted or unsubstituted monovalent non-aromatic condensed heteropolycyclic group, —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
Q₁ to Q₃ are each independently hydrogen, deuterium, —F, —Cl, —Br, —I, a hydroxyl group, a cyano group, a nitro group, an amidino group, a hydrazino group, a hydrazono group, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₁-C₆₀ heteroaryl group, a monovalent non-aromatic condensed polycyclic group, a monovalent non-aromatic condensed heteropolycyclic group, a biphenyl group, or a terphenyl group, and
b91 and b92 are each independently an integer from 1 to 10.

17. The heterocyclic compound as claimed in claim 8, wherein R₆ and R₇ are each independently:
hydrogen, deuterium, —F, —Cl, —Br, —I, a cyano group, a methyl group, an ethyl group, an n-propyl group, an isopropyl group, an n-butyl group, a sec-butyl group, an isobutyl group, a tert-butyl group, an ethenyl group, a propenyl group, a butenyl group, a methoxy group, an ethoxy group, an n-propoxy group, an isopropoxy group, an n-butoxy group, a sec-butoxy group, an isobutoxy group, or a tert-butoxy group; or
a methyl group, an ethyl group, an n-propyl group, an isopropyl group, an n-butyl group, a sec-butyl group, an isobutyl group, a tert-butyl group, a methoxy group, an ethoxy group, an n-propoxy group, an isopropoxy group, an n-butoxy group, a sec-butoxy group, an isobutoxy group, or a tert-butoxy group, each substituted with deuterium, —F, —Cl, —Br, —I, a cyano group, a phenyl group, or a biphenyl group.

18. The heterocyclic compound as claimed in claim 8, wherein R₁₀, R₂₀, and R₃₀ are each independently:
hydrogen, deuterium, —F, —Cl, —Br, —I, a methyl group, an ethyl group, an n-propyl group, an isopropyl group, an n-butyl group, a sec-butyl group, an isobutyl group, or a tert-butyl group;
a methyl group, an ethyl group, an n-propyl group, an isopropyl group, an n-butyl group, a sec-butyl group, an isobutyl group, or a tert-butyl group, each substituted with deuterium, —F, —Cl, —Br, —I, a cyano group, a phenyl group, or a biphenyl group;
a phenyl group, a biphenyl group, a terphenyl group, a naphthyl group, a fluorenyl group, a spiro-bifluorenyl group, a benzofluorenyl group, a dibenzofluorenyl group, a carbazolyl group, a dibenzofuranyl group, a dibenzothiophenyl group, or a dibenzosilolyl group;
a phenyl group, a biphenyl group, a terphenyl group, a naphthyl group, a fluorenyl group, a spiro-bifluorenyl group, a benzofluorenyl group, a dibenzofluorenyl group, a carbazolyl group, a dibenzofuranyl group, a dibenzothiophenyl group, or a dibenzosilolyl group, each substituted with deuterium, —F, —Cl, —Br, —I, a cyano group, a methyl group, an ethyl group, an n-propyl group, an isopropyl group, an n-butyl group, a sec-butyl group, an isobutyl group, a tert-butyl group, a phenyl group, a biphenyl group, a terphenyl group, a naphthyl group, a fluorenyl group, a spiro-bifluorenyl group, a benzofluorenyl group, a dibenzofluorenyl group, a carbazolyl group, a dibenzofuranyl group, a dibenzothiophenyl group, or a dibenzosilolyl group; or
—N(Q₁₁)(Q₁₂).

19. The heterocyclic compound as claimed in claim 8, wherein the heterocyclic compound represented by Formula 1 is represented by Formula 10A or 10B: 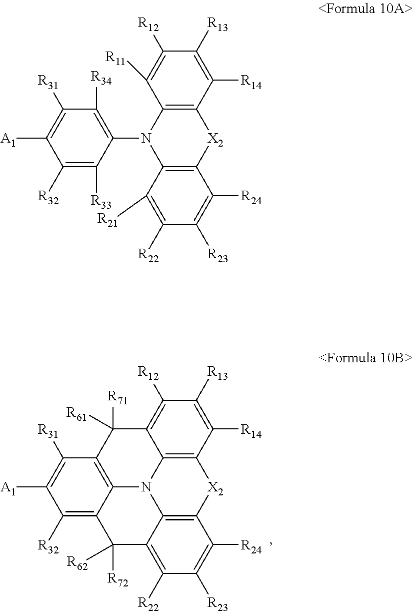
wherein, in Formulae 10A and 10B,
A₁ and X₂ are the same as described in claim 8,
R₁₁ to R₁₄ are each independently defined the same as R₁₀ in claim 8,
R₂₁ to R₂₄ are each independently defined the same as R₂₀ in claim 8,
R₃₁ to R₃₄ are each independently defined the same as R₃₀ in claim 8,
R₆₁ and R₆₂ are each independently defined the same as R₆ in claim 8, and
R₇₁ and R₇₂ are each independently defined the same as R₇ in claim 8.

20. The heterocyclic compound as claimed in claim 8, wherein the heterocyclic compound is one of Compounds 1 to 94: 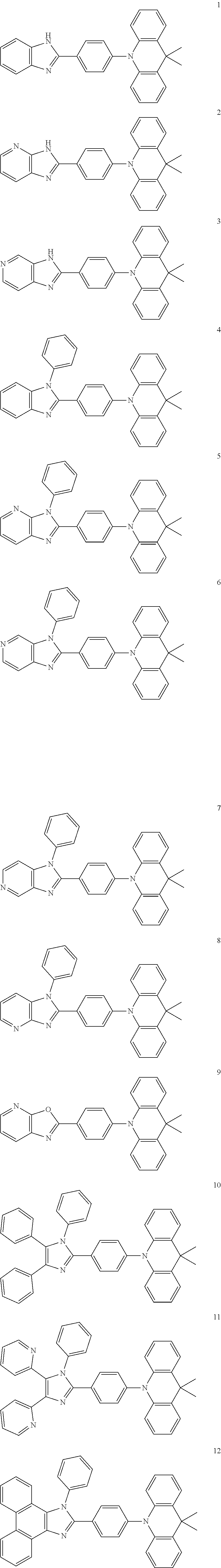 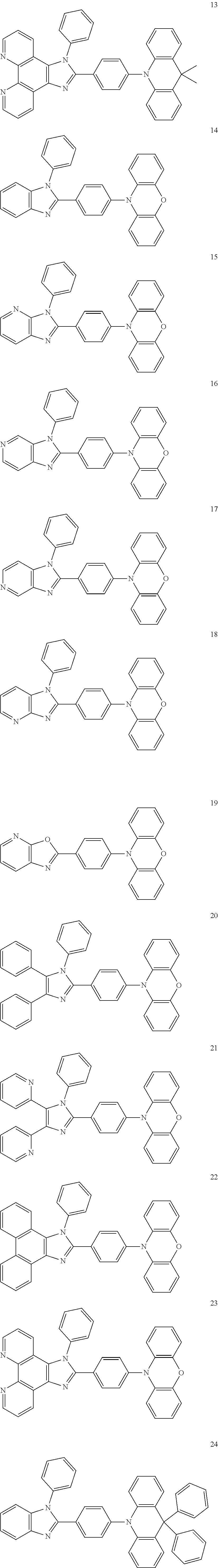 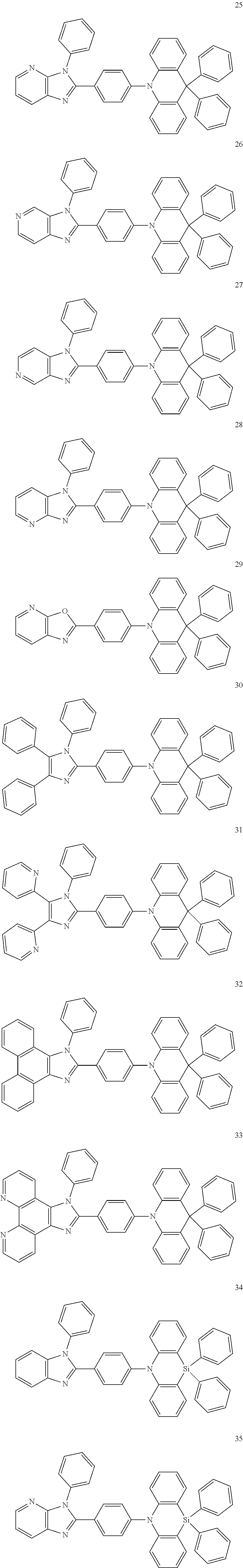 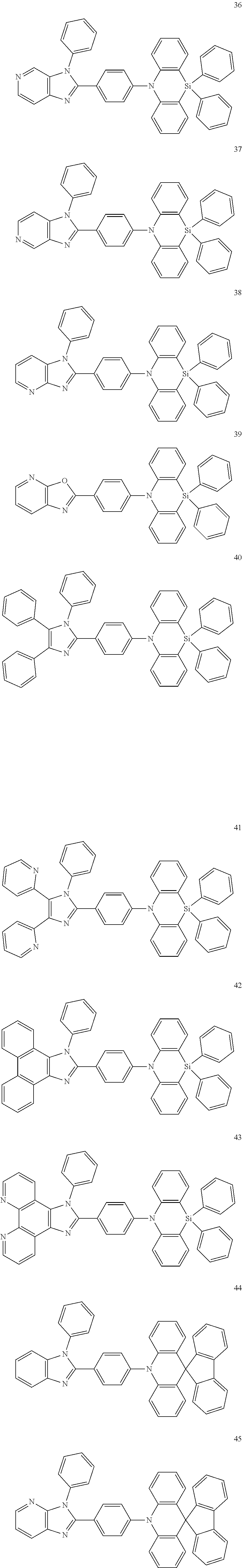 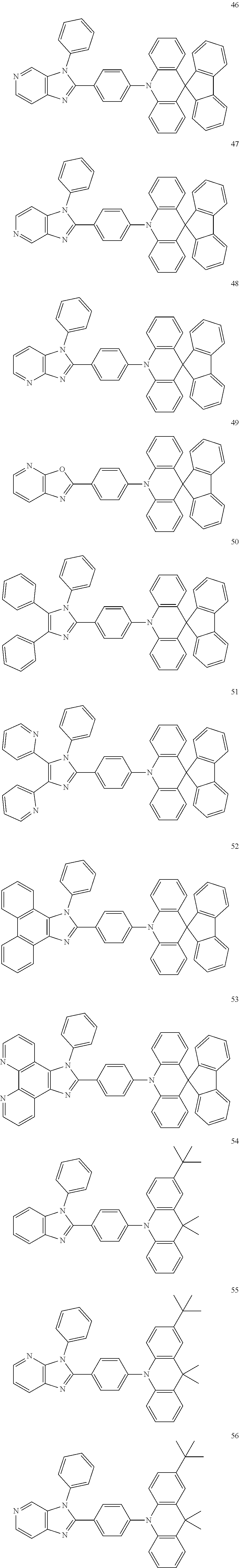 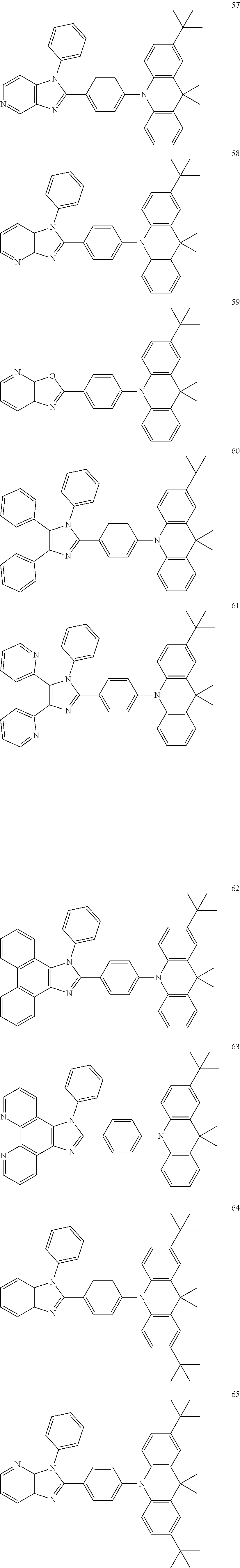 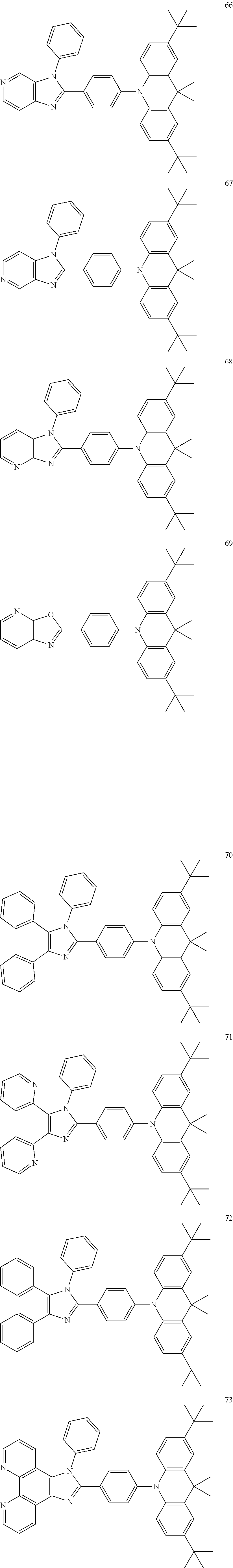 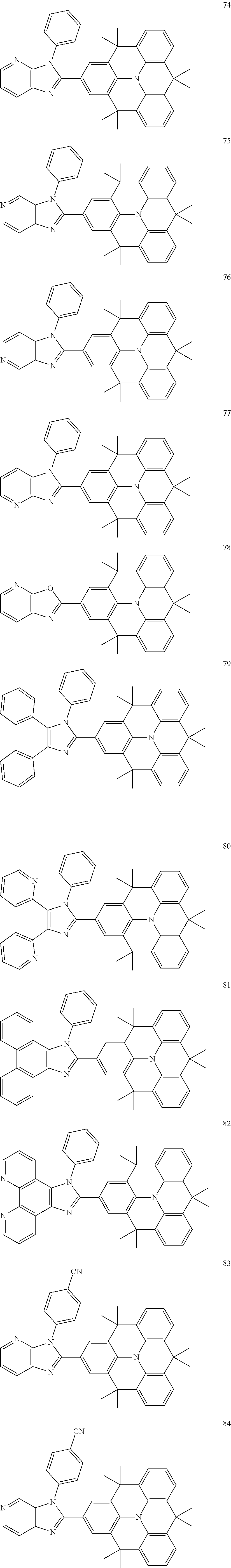 [chemical structure]
